(19) United States
(12) Patent Application Publication
(10) Pub. No.: US 2024/0277803 A1
(43) Pub. Date: Aug. 22, 2024

(54) NME INHIBITORS AND METHODS OF USING NME INHIBITORS

(71) Applicant: Minerva Biotechnologies Corporation, Waltham, MA (US)

(72) Inventors: Cynthia Bamdad, Boston, MA (US); Benoit Smagghe, Honolulu, HI (US)

(21) Appl. No.: 18/439,331

(22) Filed: Feb. 12, 2024

Related U.S. Application Data
(63) Continuation of application No. 17/548,312, filed on Dec. 10, 2021, now abandoned.
Application No. 17/548,312 is a continuation of application No. 17/306,477, filed on May 3, 2021, now abandoned.
Application No. 17/306,477 is a continuation of application No. 15/910,894, filed on Mar. 2, 2018, now abandoned.
Application No. 15/910,894 is a division of application No. 14/831,825, filed on Aug. 20, 2015, now abandoned.
Application No. 14/831,825 is a continuation of application No. PCT/US2014/017515, filed on Feb. 20, 2014.
Application No. PCT/US2014/017515 is a continuation-in-part of application No. PCT/US2013/050563, filed on Jul. 15, 2013.
Application No. PCT/US2013/050563 is a continuation-in-part of application No. PCT/US2013/051899, filed on Jul. 24, 2013.
Application No. PCT/US2013/051899 is a continuation-in-part of application No. PCT/US2013/055015, filed on Aug. 14, 2013.
(60) Provisional application No. 61/767,206, filed on Feb. 20, 2013.
(60) Provisional application No. 61/768,992, filed on Feb. 25, 2013.
(60) Provisional application No. 61/774,558, filed on Mar. 7, 2013.
(60) Provisional application No. 61/837,560, filed on Jun. 20, 2013.
(60) Provisional application No. 61/865,092, filed on Aug. 12, 2013.
(60) Provisional application No. 61/894,365, filed on Oct. 22, 2013.
(60) Provisional application No. 61/901,343, filed on Nov. 7, 2013.
(60) Provisional application No. 61/925,190, filed on Jan. 8, 2014.
(60) Provisional application No. 61/925,601, filed on Jan. 9, 2014.
(60) Provisional application No. 61/938,051, filed on Feb. 10, 2014.

(51) Int. Cl. A61K 38/17; A01K 67/0271; A61K 38/45; A61K 39/395; A61K 49/00; A61P 35/00; C07K 14/47; C07K 16/18; C07K 16/30; C07K 16/40; C12N 9/12; G01N 33/50; G01N 33/574

(52) U.S. Cl. CPC A61K 38/1735; A61P 35/00; A01K 67/0271; A01K 2267/0331; A61K 38/45; A61K 39/3955; A61K 49/0008; C07K 14/4727; C07K 16/18; C07K 16/3092; C07K 16/40; C12N 9/1229; C12Y 207/04006; G01N 33/5073; G01N 33/574; G01N 2333/71; G01N 2500/10; G01N 2800/56

(57) ABSTRACT

The present application discloses inhibitors of NME family of proteins.

### BACKGROUND OF THE INVENTION

### Sequence Listing

The instant application contains a Sequence Listing which has been submitted electronically in XML format and is hereby incorporated by reference in its entirety. Said XML copy, created on Feb. 12, 2024, is named 56699-728_304_SL.xml and is 281,920 bytes in size.

### FIELD OF THE INVENTION

The present application relates to inhibitors of NME family of proteins. The present application also relates to a method of using the inhibitors.

### GENERAL BACKGROUND AND STATE OF THE ART

In recent years, anti-cancer drugs that are cytotoxic agents have been replaced or have been augmented by ‘smart’ drugs that target a particular molecule that directly or indirectly promotes cancer cell growth. Ideally, the targeted molecule is expressed more in cancer cells than in healthy cells. Even more preferred would be drugs that target a molecule that is almost exclusively expressed in cancer cells or cancerous tissues and is not expressed in healthy human adult tissues. In that case, the target molecule could be effectively disabled without significantly harming the patient's healthy tissues.

The inventors previously reported their discovery that NME proteins are ligands of the MUC1* growth factor receptor and that these ligand-receptor pairs mediate the growth of both stem cells and cancer cells (Mahanta et al, 2008, Hikita et al, 2008, Smagghe et al, 2013). Before that time, NM23-H1 and NM23-H2 (NME and NME2) had been implicated as having a role in differentiation, however the literature was full of contradictory reports (Lombardi et al, 1995). Primarily, NM23 had been identified as the inhibiting factor that prevented leukemia cells from reaching terminal differentiation, which is a hallmark of the disease (Okabe-Kado, J., et al. 1985, Okabe-Kado, J., et al. 1992, Okabe-Kado, J., et al. 1995). However, prior to the inventor's disclosure that NM23-H1 and H-2 were ligands of the MUC1* growth factor receptor which promoted stem and cancer cell growth via ligand induced dimerization of MUC1*'s extracellular domain, it was not known how NM23 was involved in differentiation or more importantly that it had to be a dimer, or dimerize its target receptor, to be active. The inventors showed that dimeric NM23 binds to and dimerizes MUC1* on cancer cells and stem cells and promotes cancer growth and survival or growth and pluripotency, respectively. NM23 tetramers or hexamers do not bind to the PSMGFR region of the MUC1* receptor and have the opposite function as the dimers. Hexameric NM23 induces differentiation of stem cells.

Similarly, many researchers attempted to develop drugs that targeted MUC1. However, until the inventors discovered that it was the cleaved form called MUC1*, with an extracellular domain consisting primarily of the PSMGFR sequence, that functions as a growth factor receptor and activated by ligand-induced dimerization, it was unknown how MUC1 was related to cancer if at all. In fact, essentially all other attempts at developing anti-cancer therapeutics aimed at MUC1 targeted the tandem repeats of the extracellular domain, which the inventors showed is shed and released from the cell surface. Up until that time, the conventional wisdom was that MUC1 was cleaved, but the cleaved portion that contained the tandem repeats came down and bound to the transmembrane fragment that remained attached to the cells surface, forming a heterodimer (Ligtenberg et al, 1990, Baruch A et al. (1999). The inventors showed that to be untrue as double staining experiments of cancerous tissues, using antibodies that only recognize the cleaved form, MUC1*, or antibodies that only bind to the shed region (tandem repeats or ‘core’) revealed that antibodies that stained the cleaved form did not co-localize with antibodies that bound to the tandem repeats. In fact most membrane staining of cancerous tissues was negative or minimally positive for MUC1 with intact tandem repeat domain, but highly positive for the clipped MUC1* form. These experiments showed that when MUC1 is cleaved, the bulky extracellular domain is released from the cell surface (Mahanta, et al, 2008).

In addition to anti-cancer drugs, there have been many failed attempts at developing anti-cancer vaccines. The problem is that the body's immune system would create antibodies against ‘self’ which would destroy the target on the healthy tissue as well as on any future cancerous tissues. Several attempts have been made to develop anti-cancer vaccines that target MUC1. However, in each failed attempt, the portion of the MUC1 molecule that was targeted was the ‘core’ also known as the tandem repeat domain which the inventors previously showed is shed from the surface of cancer cells (Kroemer G et al, 2013).

### SUMMARY OF THE INVENTION

In one aspect, the invention is directed to antibodies that preferentially recognize cancer cells but not healthy cells where MUC1 is clipped to a growth factor receptor form.

In another aspect, the invention is directed to antibodies that target NME proteins.

In another aspect of the invention, the antibodies target NME proteins that are preferentially expressed in early life and to a much lesser degree in adult life. Preferably, these NME proteins are present at high levels in stem cells but not in adult cells.

In another aspect of the invention, the antibodies target NME proteins that are preferentially expressed in the very early stages of embryogenesis or in naïve state stem cells but not expressed or expressed at low levels in adult tissues.

In another aspect of the invention, antibodies are generated that target NME1

In another aspect of the invention, antibodies are generated that target NME6

In another aspect of the invention, antibodies are generated that target NME1 or NME6, wherein they inhibit dimerization.

In another aspect of the invention, antibodies are generated that target NME7.

In one aspect of the invention, the antibodies that are generated which recognize an NME protein, also inhibit its dimerization. In another aspect of the invention, the antibodies that are generated which recognize an NME protein, inhibit its interaction with MUC1. In yet another aspect of the invention, the antibodies that are generated which recognize an NME protein, inhibit its interaction with MUC1* or its interaction with the PSMGFR peptide.

In yet another aspect of the invention, antibodies are generated that bind to MUC1* and inhibit its interaction with NME proteins. In one aspect, they inhibit the interaction between MUC1* and NME7 but not between MUC1* and NME1.

In one aspect of the invention, antibodies are generated outside of the patient, for example, in an animal, in a cell, or artificially generated including using phage display and binding assays. In another aspect of the invention, the antibodies are generated in the patient, wherein portions of the targeted proteins are given alone or in combinations, wherein an adjuvant may be added for use as a vaccine.

In one aspect, the present invention is direct to an agent that inhibits function of an NME family member protein. The agent may be an antibody, such as Fab, monovalent, bivalent or IgM, bi-specific, human or humanized. Or, the agent may be a small molecule. In one aspect, the function of the NME family member protein that may be sought to be inhibited may be the ability of the NME family member protein to: promote stem cell proliferation and/or inhibit differentiation; promote cancer cell proliferation and/or inhibit differentiation; bind to MUC1*; bind to DNA; act as a transcription factor; be secreted by a cell; or form a dimer. In particular, the NME family member may be preferably NME7 or NME7-AB.

The agent may be an antibody that inhibits tumorigenic activity of NME7 or NME7AB. Preferably, the NME family member may be a variant of NME7 having a molecular weight between 25 and 33 kDa. Alternatively, the NME family member may be NME6 or NME1.

In another aspect, the invention is directed to a method for treating a patient with cancer or at risk of developing cancer comprising administering to the patient an effective amount of an agent that inhibits tumorigenic activity of an NME family member protein. The NME family member protein may be preferably, NME7, NME6, or NME1. In one embodiment, the agent may inhibit NME7 activity but not NME1 activity. In another embodiment, the agent may inhibit binding between NME7 and MUC1*. Or, the agent may inhibit binding between NME7 and its cognate nucleic acid binding site. In still another embodiment, the agent may be an antibody.

In another aspect, the invention is directed to a method for treating a patient with cancer or at risk of developing cancer comprising administering to the patient an effective amount of NME1 as a hexamer. The NME1 polypeptide may be a mutant or variant that prefers hexamer state.

In yet another aspect, the invention is directed to a method for treating a patient with cancer or at risk of developing cancer comprising administering to the patient an effective amount of NME6 as a monomer. In one embodiment, NME6 may be a mutant or a variant that prefers monomer state.

In still yet another aspect, the invention is directed to a method for treating a patient with cancer or at risk of developing cancer comprising administering to the patient an effective amount of NME1 as a monomer. NME1 may be a mutant or variant that prefers monomer state.

In another aspect, the invention is directed to a method for treating a patient with cancer or at risk of developing cancer comprising administering to the patient an effective amount of a peptide or peptide mimic that inhibits the interaction of the NME family member with its cognate receptor. In one embodiment, the cognate receptor may be MUC1. In another embodiment, the peptide may be derived from the MUC1* portion of MUC1, PSMGFR, N-10 PSMGFR, N-15 PSMGFR, or N-20 PSMGFR.

In another aspect, the invention is directed to a method for classifying cancers or stratifying patients, having or suspected of having cancer, comprising the steps of: (i) analyzing a patient sample for the presence of stem or progenitor cell genes or gene products; and (ii) grouping patients who share similar expression or expression levels of stem or progenitor cell genes or gene products.

The method may further include the step of (iii) treating the patient with agents that inhibit those stem or progenitor cell genes or gene products. Alternatively, the method may include the steps of (iii) analyzing the stem or progenitor genes or gene products to assess severity of the cancer, wherein expression of, or higher expression of, genes or gene products that are characteristic of earlier stem or progenitor states indicate more aggressive cancers and expression of, or higher expression of, genes or gene products that are characteristic of later progenitor states indicate less aggressive cancers; (iv) designing therapy commensurate with treating patient with cancer more or less aggressive cancer as determined in step (iii); and (v) treat patient with therapy in accordance with the design in step (iv). In such methods, the patient sample may be blood, bodily fluid, or biopsy. And the genes or gene products may be NME family proteins. In one embodiment, the genes or gene product indicative of an earlier stem cell state may be NME7 or NME6.

In another aspect, the invention is directed to an agent that inhibits the interaction of an NME family member protein and a MUC1 transmembrane protein whose extracellular domain is devoid of the tandem repeat domain, wherein the agent binds to MUC1* on cancer cells with a higher affinity than its binding to the MUC1 transmembrane protein whose extracellular domain is devoid of the tandem repeat domain present on healthy cells in an adult. In one embodiment, the agent may include without limitation, an antibody, natural product, synthetic chemical or nucleic acid. In one embodiment, the NME family member protein may be NME7, NME6 or bacterial NME.

In another aspect, the invention is directed to a method of inhibiting interaction of an NME family member protein and a MUC1 transmembrane protein whose extracellular domain is devoid of the tandem repeat domain in a cell, comprising contacting the cell with an agent that binds to MUC1* on cancer cells with a higher affinity than its binding to the MUC1 transmembrane protein whose extracellular domain is devoid of the tandem repeat domain on healthy cells in an adult. In one embodiment, the agent may include without limitation, an antibody, natural product, synthetic chemical or nucleic acid. In one embodiment, the NME family member protein may be NME7, NME6 or bacterial NME.

In another aspect, the invention is directed to a method of identifying an agent that inhibits the interaction of an NME family member protein and a MUC1 transmembrane protein whose extracellular domain is devoid of the tandem repeat domain, which steps may include determining affinity of the agent for MUC1* present on cancer cells, determining affinity of the agent for MUC1* present on stem or progenitor cells, and selecting an agent that binds to MUC1* present on cancer cells better than its ability to bind to MUC1* present on stem or progenitor cells, thus identifying the agent. In one embodiment, the agent may include without limitation, an antibody, natural product, synthetic chemical or nucleic acid. In another embodiment, the stem or progenitor cells may be embryonic stem cells, iPS cells, cord blood cells, bone marrow cells or hematopoietic progenitor cells. In one embodiment, the NME family member protein may be NME7, NME6 or bacterial NME.

In another aspect, the invention is directed to a transgenic mammal that expresses human NME protein in the germ cells and somatic cells, wherein the germ cells and somatic cells contain a nucleic acid encoding human NME introduced into said mammal. Thus, the human NME may be recombinantly expressed in the transgenic mammal. Of course, the transgenic mammal may not be human. In the transgenic mammal, the NME protein may be preferably inducibly expressed. The NME protein may be preferably NME7 or NME7-AB.

In yet another aspect, the invention is directed to a method of generating a mammal that responds to cancer in a way that more closely resembles the response of a human wherein the mammal is a mammal in which human NME protein is expressed. The cancer may be spontaneously generated or implanted from cultured cells or from a human being. In one embodiment, the NME protein may be NME1 dimer or NME7 monomer. In another aspect, the mammal may be transgenic, wherein the mammal may express human MUC1 or MUC1* or NME protein in the germ cells and somatic cells, wherein the germ cells and somatic cells contain a recombinant human MUC1 or MUC1* or NME protein gene sequence introduced into said mammal. Preferably, the NME protein is inducibly expressed. Still preferably, the NME protein may be NME7 or NME7-AB.

In another aspect, the invention is directed to a method for increasing engraftment of human tumors in mammals, comprising mixing the human tumor cells with NME1 dimers or NME7 monomers prior to injecting the cells into the test mammals.

In yet another aspect, the invention is directed to a method for generating an antibody comprising injecting an NME family protein or peptide fragment or fragments thereof into a mammal and harvesting the antibody or antibody producing cell. Preferably the NME family protein may be NME7 or NME7-AB or NME1. Preferably, the peptide fragment may be selected from SEQ ID NOS:88-140, more preferably 88-133, more preferably 88-121.

In another aspect, the invention is directed to a method of generating or selecting an antibody or antibody-like molecule that specifically binds to NME family protein or peptide fragment thereof, comprising: (i) screening an antibody library or library of antibody fragments or epitopes with the NME family protein or peptide fragment; (ii) assaying for binding to the NME family protein or a peptide fragment thereof; and (iii) identifying the specifically bound antibody or antibody-like molecule. The method may further comprise engineering the identified antibody or antibody-like molecule for administration to a patient for the treatment or prevention of cancer using methods well known in the art. The NME family protein may be NME7 or NME7-AB or NME1. Preferably, the peptide fragment may be selected from SEQ ID NOS:88-140, more preferably 88-133, more preferably 88-121.

In yet another aspect, the present invention is directed to a method of preventing cancer by vaccinating a person with an NME family protein or peptide fragment or fragments thereof. In one embodiment, the peptide fragment or fragments may include one or more peptides whose sequence is present in an NME family protein, which is optionally mixed with a carrier, adjuvant or attached to an immunogenic agent. The NME family protein may be NME1, NME6, NME7 or NME7-AB. Preferably, the peptide fragment may be selected from SEQ ID NOS:88-140, more preferably 88-133, more preferably 88-121. In a preferred embodiment, the peptide sequence is not a fragment of human NME-H1 protein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The present invention will become more fully understood from the detailed description given herein below, and the accompanying drawings which are given by way of illustration only, and thus are not limitative of the present invention, and wherein;

FIG. 1 is a graph of cancer cell growth measured as a function of bivalent or monovalent antibody concentration, showing that it is dimerization of the MUC1* receptor that stimulates growth. The growth of MUC1-positive breast cancer cells, ZR-75-30, was stimulated by the addition of bivalent (Ab) Anti-MUC1* and inhibited by the addition of the monovalent Fab. The addition of bivalent antibody produces the characteristic bell-shaped growth curve indicative of growth factor receptor dimerization. The growth of MUC1-negative HEK 293 cells was not impacted by either the bivalent or monovalent Fab Anti-MUC1*. When the bivalent antibody was added in excess, there is one bivalent antibody bound to each receptor rather than one bivalent antibody dimerizing every two receptors and thus inhibits growth.

FIG. 2 is a graph of tumor volume measurement of T47D breast cancer cells, implanted into nu/nu female mice, after treatment with either vehicle or the Fab of MN-E6 anti-MUC1* antibody. The efficacy of anti-MUC1* E6 antibody was found to be statistically significant in reducing tumor volume with p values of 0.0001.

FIG. 3A shows a Western blot gel showing the expression of NME1 in the cell lysate of: 1) BGO1V human embryonic stem cells cultured in NM23-H1 dimers over a surface coated with a MUC1* antibody surface (MN-C3 mab); 2) BGO1V human embryonic stem cells cultured according to standard protocol in bFGF over a layer of mouse feeder cells (MEFs); 3) T47D breast cancer cells cultured by standard method in RPMI media; and 4) recombinant human NM23-H1 wild type, “wt.”

FIG. 3B shows a Western blot gel showing the expression of NME7 in the cell lysate of: 1) BGO1V human embryonic stem cells cultured in NM23-H1 dimers over a surface coated with a MUC1* antibody surface (MN-C3 mab); 2) BGO1V human embryonic stem cells cultured according to standard protocol in bFGF over a layer of mouse feeder cells (MEFs); 3) T47D breast cancer cells cultured by standard method in RPMI media; and 4) recombinant human NM23-H1 wild type, “wt.”

FIG. 3C shows the results of a “pull-down” or an immuno-precipitation assay in which the cell lysates were separately incubated with beads to which was added an antibody to the MUC1 cytoplasmic tail, “Ab-5”. Species captured by binding to the MUC1* peptide were separated by SDS-PAGE and blotted with antibodies against NME1.

FIG. 3D shows the results of a “pull-down” or an immuno-precipitation assay in which the cell lysates were separately incubated with beads to which was added an antibody to the MUC1 cytoplasmic tail, “Ab-5”. Species captured by binding to the MUC1* peptide were separated by SDS-PAGE and blotted with antibodies against NME7. Same experiments were conducted with NME6 but data is not shown.

FIGS. 4A-4C show photos of Western blots in which cell lysates from T47D breast cancer cells, BGO1V and HES-3 human ES cells and human SC101-A1 iPS cells were probed for the presence of NME1 (FIG. 4A), NME6 (FIG. 4B) or NME7 (FIG. 4C). NME1 in all cell lines ran with an apparent molecular weight of ˜17 kDa (FIG. 4A). Species that reacted with an NME6-specific antibody were not visualized in the absence of Super Signal enhancement (FIG. 4B). In all cell lines, NME7˜33 kDa species and the 42 kDa species (FIG. 4C) could be detected in all but the HES-3 cell line (cultured in FGF).

FIG. 4D and FIG. 4E show photos of Western blots enhanced using Super Signal in which cell lysates from T47D breast cancer cells, BGO1V and HES-3 human ES cells and human SC101-A1 iPS cells were probed for the presence of NME6 (FIG. 4D) or NME7 (FIG. 4E). Species that reacted with an NME6-specific antibody were detected in all cell lines except the HES-3 cell line, when visualization was enhanced using Super Signal (FIG. 4D). In all cell lines, NME7˜33 kDa species and the 42 kDa species (FIG. 4E) could be detected in all but the HES-3 cell line (cultured in FGF).

FIGS. 5A-5C show panels of photos of Western blots of human embryonic stem (ES) cells (FIG. 5A) and induced pluripotent stem (iPS) cells in a conditioned media (FIG. 5B) and lysate (FIG. 5C) probed for the presence of NME7. Westerns show the presence of three forms of NME7 in the cell lysates. One with an apparent molecular weight of ˜42 kDa (full length), ˜33 kDa (NME7-AB domains devoid of the N-terminal DH domain) and a small ˜25 kDa species. However, only the lower molecular weight species are in the conditioned media (FIG. 5B).

FIG. 6A is an elution profile of size exclusion chromatography purification of NME7-AB.

FIG. 6B is non-reducing SDS-PAGE gel from NME7-AB peak fractions.

FIG. 6C is the elution profile of size exclusion chromatography of the purified NME7-AB.

FIG. 7A shows a photograph of nanoparticle binding assays wherein a MUC1* extra cellular domain peptide is immobilized onto SAM-coated nanoparticles, and NME proteins are added free in solution. A color change from pink to blue indicates that the protein free in solution can simultaneously bind to two peptides on two different nanoparticles. FIG. 7A shows that NME7 in solution has two binding sites for the MUC1* peptide. The Fab of the anti-MUC1* antibody fully inhibits the binding, showing that particle aggregation is due to the specific interaction of MUC1* peptide and NME7.

FIG. 7B shows a photograph of nanoparticle binding assays wherein a MUC1* extra cellular domain peptide is immobilized onto SAM-coated nanoparticles, and NME proteins are added free in solution. A color change from pink to blue indicates that the protein free in solution can simultaneously bind to two peptides on two different nanoparticles. FIG. 7B shows a photograph of nanoparticle binding assays with NME7-AB added free in solution over a wider range of concentrations.

FIG. 7C shows a photograph of nanoparticle binding assays wherein a MUC1* extra cellular domain peptide is immobilized onto SAM-coated nanoparticles, and NME proteins are added free in solution. A color change from pink to blue indicates that the protein free in solution can simultaneously bind to two peptides on two different nanoparticles. FIG. 7C shows a photograph of nanoparticle binding assays when all proteins are added in solution.

FIG. 8A shows a graph of HRP signal from ELISA sandwich assay showing NME7-AB dimerizes MUC1* extra cellular domain peptide.

FIG. 8B shows a second MUC1* peptide with a C-terminal His-tag or Biotin tag was added and visualized by HRP labeled antibody to either His-tag or HRP labeled streptavidin.

FIG. 9A is a 4× magnified photograph of human iPS stem cells cultured in recombinant NME7-AB on Day 1 post-plating.

FIG. 9B is a 4× magnified photograph of human iPS stem cells cultured in recombinant NM23 (NME1) purified dimers on Day 1 post-plating.

FIG. 9C is a 10× magnified photograph of human iPS stem cells cultured in recombinant NME7-AB on Day 1 post-plating.

FIG. 9D is a 10× magnified photograph of human iPS stem cells cultured in recombinant NM23 (NME1) purified dimers on Day 1 post-plating.

FIG. 10A is a 4× magnified photograph of human iPS stem cells cultured in recombinant NME7-AB on Day 3 post-plating.

FIG. 10B is a 4× magnified photograph of human iPS stem cells cultured in recombinant NM23 (NME1) purified dimers on Day 3 post-plating.

FIG. 10C is a 10× magnified photograph of human iPS stem cells cultured in recombinant NME7-AB on Day 3 post-plating.

FIG. 10D is a 10× magnified photograph of human iPS stem cells cultured in recombinant NM23 (NME1) purified dimers on Day 3 post-plating.

FIG. 11A shows photos of an immunocytochemistry experiment showing that human HES-3 stem cells cultured for 10 or more passages in NME7-AB are positive for the pluripotency marker NANOG.

FIG. 11B shows photos of an immunocytochemistry experiment showing that human HES-3 stem cells cultured for 10 or more passages in NME7-AB are positive for the pluripotency marker OCT3/4.

FIG. 11C shows photos of an immunocytochemistry experiment showing that human HES-3 stem cells cultured for 10 or more passages in NME7-AB are positive for the pluripotency marker Tra 1-81.

FIG. 11D shows photos of an immunocytochemistry experiment showing that human HES-3 stem cells cultured for 10 or more passages in NME7-AB are positive for the pluripotency marker SSEA4 (FIG. 11D).

FIGS. 12A-12C show photos of HES-3 embryonic stem cells stained with an antibody that recognizes tri-methylated Lysine 27 on Histone 3 which forms a condensed dot if the cell has progressed from the naïve state to the primed state wherein a X chromosome is inactivated (XaXi) as opposed to both X's active (XaXa). FIG. 12A shows that the cells were first cultured in FGF media on MEF feeder cells (XaXi). FIG. 12B shows that the cells were then grown in NME7 for 10 passages (XaXa). FIG. 12C shows that the cells were then moved (back into FGF-MEFs for 4 passages (XaXi).

FIG. 12D shows a close-up of FIG. 12B. The stained stem cells were grown in NME7 for 10 passages (XaXa).

FIG. 13A shows photos of MUC1*-positive cancer cells treated with nothing.

FIG. 13B shows photos of MUC1*-positive cancer cells treated with taxol.

FIG. 13C shows photos of MUC1*-positive cancer cells treated with 350 ng/ml an anti-NME7 antibody.

FIG. 13D shows photos of MUC1*-positive cancer cells treated with 175 ng/ml of an anti-NME7 antibody.

FIG. 13E shows photos of MUC1*-positive cancer cells treated with 87.5 ng/ml of an anti-NME7 antibody.

FIG. 13F shows a graph showing cell count of MUC1*-positive cancer cells in response to treatment with either nothing, taxol, or an anti-NME7 antibody at 48 hours.

FIG. 13G shows and a dot-blot used to estimate antibody concentration used in the cancer cell inhibition experiment.

FIG. 14A shows the 48 hour results of cancer cells cultured in media alone.

FIG. 14B shows the 48 hour results of an experiment using taxol to inhibit cancer cell growth.

FIG. 14C shows the 48 hour results of an experiment using 350 ng/ml of an anti-NME7 antibody to inhibit cancer cell growth.

FIG. 14D shows the 48 hour results of an experiment using 175 ng/ml of an anti-NME7 antibody to inhibit cancer cell growth.

FIG. 14E shows the 48 hour results of an experiment using 87.5 ng/ml of an anti-NME7 antibody to inhibit cancer cell growth.

FIG. 14F shows the 48 hour results of an experiment using 43.8 ng/ml of an anti-NME7 antibody to inhibit cancer cell growth.

FIG. 14G shows the 48 hour results of an experiment using 21.9 ng/ml of an anti-NME7 antibody to inhibit cancer cell growth.

FIG. 14H shows the 48 hour results of an experiment using 10.9 ng/ml of an anti-NME7 antibody to inhibit cancer cell growth.

FIG. 14I shows the 48 hour results of an experiment using 5.5 ng/ml of an anti-NME7 antibody to inhibit cancer cell growth.

FIG. 14J shows the 48 hour results of an experiment using 2.7 ng/ml of an anti-NME7 antibody to inhibit cancer cell growth.

FIG. 14K shows a graph of cell number obtained using a calcein am assay.

FIG. 15A shows the 96 hour results of cancer cells cultured in media alone.

FIG. 15B shows the 96 hour results of an experiment using taxol to inhibit cancer cell growth.

FIG. 15C shows the 96 hour results of an experiment using 350 ng/ml of an anti-NME7 antibody to inhibit cancer cell growth.

FIG. 15D shows the 96 hour results of an experiment using 175 ng/ml of an anti-NME7 antibody to inhibit cancer cell growth.

FIG. 15E shows the 96 hour results of an experiment using 87.5 ng/ml of an anti-NME7 antibody to inhibit cancer cell growth.

FIG. 15F shows the 96 hour results of an experiment using 43.8 ng/ml of an anti-NME7 antibody to inhibit cancer cell growth.

FIG. 15G shows the 96 hour results of an experiment using 21.9 ng/ml of an anti-NME7 antibody to inhibit cancer cell growth.

FIG. 15H shows the 96 hour results of an experiment using 10.9 ng/ml of an anti-NME7 antibody to inhibit cancer cell growth.

FIG. 15I shows the 96 hour results of an experiment using 5.5 ng/ml of an anti-NME7 antibody to inhibit cancer cell growth.

FIG. 15J shows the 96 hour results of an experiment using 2.7 ng/ml of an anti-NME7 antibody to inhibit cancer cell growth.

FIG. 15K shows a graph of cell number obtained using a calcein am assay. The graph and the photos show anti-NME7 antibodies inhibit cancer cell growth at concentrations as low as in the nanomolar range.

FIG. 16 shows a native, non-denaturing gel that shows the multimerization state of NM23-WT versus three different preparations of recombinant NM23-S120G.

FIG. 17A shows photographs of non-reducing gels of NM23-WT, NM23-S120G-mixed, NM23-S120G-hexamer and NM23-S120G-dimer, showing the multimerization state of the wild type protein and the three different preparations of the S120G mutant.

FIG. 17B shows Surface Plasmon Resonance (SPR) measurements of different NM23 multimers binding to MUC1* extra cellular domain peptide (PSMGFR) attached to the SPR chip surface.

FIG. 17C shows a photograph of a nanoparticle experiment showing that only NM23 dimers bind to the cognate receptor MUC1*. MUC1* extra cellular domain peptide was immobilized onto gold nanoparticles.

FIG. 17D shows the ability of the NM23_{S120G}-dimer to support pluripotent stem cell growth.

FIG. 17E shows the ability of the NM23_{S120G}-hexamer to support pluripotent stem cell growth.

FIG. 17F shows the ability of the NM23-wt to support pluripotent stem cell growth.

FIG. 17G shows the ability of the NM23_{S120G}-dimer+MUC1* peptide to support pluripotent stem cell growth.

FIG. 18 shows a cartoon of the timing of the expression of NME7, NME1 dimer and NME1 hexamer and the expression levels of their associated cancer/stem factors resulting from analysis of the experiments described herein.

FIG. 19A shows a graph of the results of ELISA assays in which recombinant NME6-wt is separated by FPLC into monomers or multimers and assayed by ELISA for ability to bind to a surface of PSMGFR peptide.

FIG. 19B shows a graph of the results when the NME6 multimers were dissociated by dilution in SDS according to a fraction of the CMC (critical micelle concentration), then assayed by ELISA for ability to bind to a surface of PSMGFR peptide.

FIG. 19C shows NME6 mutants that are designed to prefer dimerization and were generated by mimicking the NME1 S120G mutation that prefers dimer formation and is S139G in NME6 by alignment. A second mutant was made by mutating residues such that human NME6 is converted in that critical area to look like sea sponge NME6 which has been reported to exist as a dimer. These recombinant mutants were expressed and purified then assayed for the ability to bind to a surface of PSMGFR peptide.

FIG. 19D is a photo of a polyacrylamide gel evidencing expression of NME6 wt proteins.

FIG. 19E is a photo of a polyacrylamide gel evidencing expression of NME6 bearing S139G mutation, corresponding to the S120G mutation in NME1.

FIG. 19F is a photo of a polyacrylamide gel evidencing expression of human NME6 bearing mutations S139A, V142D, and V143A to mimic sea sponge NME6 that was reported to be a dimer.

FIG. 19G shows photos of a polyacrylamide gel evidencing expression of a single chain human NME6 having 2 domains joined by a (GSSS)₃ linker (SEQ ID NO: 166), with 12% reducing SDS-PAGE.

FIG. 19H shows photos of a polyacrylamide gel evidencing expression of a single chain human NME6 having 2 domains joined by a (GSSS)₃ (SEQ ID NO: 166) linker, with 12% non-reducing SDS-PAGE.

FIG. 19I shows a pull-down assay that was performed using an antibody against the C-terminus of MUC1. Proteins that were bound to MUC1 were separated on a gel, then probed with an antibody against NME6. The gel shows that in T47D breast cancer cells, BGolv and HES-3 human embryonic stem cells, human iPS cells all expressed NME6 that bound to MUC1.

FIG. 20A shows a photo of a Western blot in which cell lysates from T47D breast cancer cells, BGO1V and HES-3 human ES cells and human SC101-A1 iPS cells were probed for the presence of NME7.

FIG. 20B shows a photo of a Western blot in which nuclear fractions from T47D breast cancer cells, BGO1V and HES-3 human ES cells and human SC101-A1 iPS cells were probed for the presence of NME7.

FIG. 20C shows a photo of a Western blot in which cell lysates from T47D breast cancer cells, BGO1V and HES-3 human ES cells and human SC101-A1 iPS cells were probed for the presence of NME1.

FIG. 20D shows a photo of a Western blot in which nuclear fractions from T47D breast cancer cells, BGO1V and HES-3 human ES cells and human SC101-A1 iPS cells were probed for the presence of NME1.

FIG. 21 is a graph of real time PCR measurements of NME1, NME6, NME7 and MUC1 in MUC1-positive T47D breast cancer cells, MUC1-positive DU145 prostate cancer cells and MUC1-negative PC3 prostate cancer cells. Measurements are relative to 18S ribosomal RNA and normalized to the measurements of the T47D cells. Both MUC1-positive cancer cell lines are high in NME7. The MUC1-negative cell line has no detectable NME1, NME7 or MUC1 but has very high expression of NME6.

FIG. 22 is a photo of a Western blot wherein stem cell lysates (odd numbered lanes) or cell conditioned media (even numbered lanes) were probed for the presence of NME7. iPS (induced pluripotent stem) cells were cultured in FGF over MEFs (lanes 1,2), NM23-H1 dimers over an anti-MUC1* antibody (C3) surface (lanes 3,4) or NME7 over an anti-MUC1* antibody (C3) surface (lanes 5-8). HES-3 (human embryonic stem) cells were cultured in FGF over MEFs (lanes 9,10), NM23-H1 dimers over an anti-MUC1* antibody (C3) surface (lanes 11,12) or NME7 over an anti-MUC1* antibody (C3) surface (lanes 13,14). Mouse embryonic fibroblast (MEFs) cells were also probed (lanes 15,16). The Western blot shows that the cell lysates contain an NME7 species with molecular weight of ˜42 kDa, which corresponds to the full-length protein. However, the secreted species runs with an apparent MW of ˜33 kDa, which corresponds to an NME7 species that is devoid of the N-terminal leader sequence.

FIGS. 23A and 23B are photos of the same Western blot shown in FIG. 22 that was then stripped and probed for the presence of histidine-tagged species which would identify recombinant NM23-H1, ˜17 kDa and NME7-AB 33 kDa, in which stem cells in lanes 3-8 and 11-14 were cultured. Minimal staining resulted, indicating that the major NME7 species detected in FIG. 22 was the native NME7 produced and processed by the stem cells.

FIG. 24A is a photo of Western blots of various cell lysates and corresponding conditioned media probed for the presence of NME7 using a mouse monoclonal antibody

FIG. 24B is a photo of Western blots of various cell lysates and corresponding conditioned media probed for the presence of another monoclonal antibody that only recognizes the N-terminal DM10 sequence. The lack of binding of the DM10 specific antibody to the ˜33 kDa NME7 species in the samples from the conditioned media of the cells indicates that the secreted form of NME7 is devoid of most if not all of the N-terminal DM10 leader sequence.

FIG. 25A shows a polyacrylamide gel of NME from the bacterium Halomonas Sp. 593, which was expressed in E. coli and expressed as a soluble protein and natural dimer.

FIG. 25B shows that in an ELISA assay NME from Halomonas Sp. 593 bound to the PSMGFR peptide of the MUC1* extra cellular domain.

FIG. 26 shows a polyacrylamide gel of NME from the bacterium Porphyromonas gingivalis W83.

FIG. 27A shows sequence alignment of Halomonas Sp 593 bacterial NME to human NME-H1. FIG. 27A discloses SEQ ID NOS 159-160, respectively, in order of appearance.

FIG. 27B shows sequence alignment of Halomonas Sp 593 bacterial NME to human NME7-A domain. FIG. 27B discloses SEQ ID NOS 161-162, respectively, in order of appearance.

FIG. 27C shows sequence alignment of Halomonas Sp 953 bacterial NME to human NME7-B domain. FIG. 27C discloses SEQ ID NOS 163-164, respectively, in order of appearance.

FIG. 28A is a photograph of human embryonic stem cells cultured in bacterial NME from Halomonas Sp 593 at 10× magnification.

FIG. 28B is a photograph of human embryonic stem cells cultured in bacterial NME from Halomonas Sp 593 at 20× magnification.

FIG. 28C is a photograph of human embryonic stem cells cultured in bacterial NME from Halomonas Sp 593 at 10× magnification.

FIG. 28D is a photograph of human embryonic stem cells cultured in bacterial NME from Halomonas Sp 593 at 20× magnification.

FIG. 29 is a graph of RT-PCR data measuring expression of the stem/cancer cell marker OCT4 after human fibroblast cells were cultured in a serum free media containing either human NME7-AB, human NME1 dimer or bacterial NME from Halomonas Sp 593.

FIG. 30 is a graph of RT-PCR measurement of the expression levels of the stem/cancer genes OCT4 and NANOG in fibroblasts that have been cultured in the presence of human NME7-AB, human NME1 or bacterial NME from Halomonas Sp 593, ‘HSP 593’. In some cases, a rho kinase inhibitor ‘ROCi’ was added to make non-adherent cells (those becoming stem/cancer-like) adhere to the surface.

FIG. 31 shows photographs of human fibroblast cells after 18 days in culture in a serum-free media containing human NME1 in dimer form at 4× magnification.

FIG. 32 shows photographs of human fibroblast cells after 18 days in culture in a serum-free media containing human NME1 in dimer form at 20× magnification.

FIG. 33 shows photographs of human fibroblast cells after 18 days in culture in a serum-free media containing bacterial NME from Halomonas Sp 593 at 4× magnification.

FIG. 34 shows photographs of human fibroblast cells after 18 days in culture in a serum-free media containing bacterial NME from Halomonas Sp 593 at 20× magnification.

FIG. 35 shows photographs of human fibroblast cells after 18 days in culture in a serum-free media containing human NME7-AB at 4× magnification.

FIG. 36 shows photographs of human fibroblast cells after 18 days in culture in a serum-free media containing human NME7-AB at 20× magnification.

FIG. 37 shows photographs of human fibroblast cells after 18 days in standard media without NME protein at 4× magnification.

FIG. 38 shows photographs of human fibroblast cells after 18 days in standard media without NME protein at 20× magnification.

FIG. 39 is a graph of RT-PCR measurement of the expression levels of transcription factors BRD4 and co-factor JMJD6 in the earliest stage naïve human stem cells compared to the later stage primed stem cells.

FIG. 40 is a graph of RT-PCR measurement of the expression levels of the chromatin rearrangement factors that are suppressed when fibroblasts revert to an induced pluripotent state while others are suppressed in naive stem cells and in some cancer cells. Expression levels of the chromatin rearrangement genes Brd4, JMJD6, Mbd3 and CHD4 were measured in fibroblasts that have been cultured in the presence of human NME7-AB, human NME1 or bacterial NME from Halomonas Sp 593, ‘HSP 593’. In some cases, a rho kinase inhibitor ‘ROCi’ was added to make non-adherent cells (those becoming stem/cancer-like) adhere to the surface.

FIG. 41 is a composite graph of RT-PCR measurements of the expression levels of the stem/cancer genes in fibroblasts that have been cultured in the presence of human NME7-AB, human NME1 or bacterial NME from Halomonas Sp 593, ‘HSP 593’. In some cases, a rho kinase inhibitor ‘ROCi’ was added to make non-adherent cells (those becoming stem/cancer-like) adhere to the surface.

FIG. 42 is a composite graph of RT-PCR measurements of the expression levels of the stem/cancer genes in fibroblasts that have been cultured in the presence of human NME7-AB, human NME1 or bacterial NME from Halomonas Sp 593, ‘HSP 593’, with the Y-axis compressed to better show differences in genes having smaller changes. In some cases, a rho kinase inhibitor ‘ROCi’ was added to make non-adherent cells (those becoming stem/cancer-like) adhere to the surface.

FIG. 43 is a graph of RT-PCR measurements of gene expression for stem cell markers and cancer stem cell markers for T47D cancer cells after being cultured in traditional media or a media containing NME7, wherein cells that became non-adherent (floaters) were analyzed separate from those that remained adherent.

FIG. 44 is a graph of RT-PCR measurements of gene expression for stem cell marker SOX2 and cancer stem cell marker CXCR4 for T47D cancer cells. Cells were cultured either in traditional media or a media containing NME1 dimers or NME7 (NME7-AB). Cell types that were separately analyzed were floating cells, cells plus Rho kinase inhibitor (+Ri), which made all cells adhere, or cells that remained adherent after floaters were removed which was in the absence of rho kinase inhibitor (−Ri).

FIG. 45 is a graph of RT-PCR measurements of gene expression for a variety of stem and putative cancer stem cell markers for T47D breast cancer cells. Cells were cultured either in traditional media or a media containing NME1 dimers (“NM23”) or NME7 (NME7-AB). Cell types that were separately analyzed were floating cells, cells plus Rho kinase inhibitor (+Ri), which made all cells adhere, or cells that remained adherent after floaters were removed which was in the absence of rho kinase inhibitor (−Ri).

FIG. 46 is a graph of RT-PCR measurements of gene expression for a variety of stem and putative cancer stem cell markers for DU145 prostate cancer cells. Cells were cultured either in traditional media or a media containing NME1 dimers (“NM23”) or NME7 (NME7-AB). Rho kinase inhibitor was not used because by passage 2, cells remained adherent.

FIG. 47 is a graph of RT-PCR measurements of gene expression for a variety of stem and putative cancer stem cell markers for MUC1 negative PC3 prostate cancer cells. Cells were cultured either in traditional media or a media containing NME1 dimers (“NM23”) or NME7 (NME7-AB). Rho kinase inhibitor was not used because by passage 2, cells remained adherent.

FIG. 48 is a graph of RT-PCR measurement of the expression levels of reported ‘cancer stem cell’ or ‘tumor initiating cell’ markers CDH1 (E-cadherin), CXCR4, NANOG, OCT4 and SOX2, along with MUC1 in T47D breast cancer cells following culture in a minimal serum-free base media wherein the only factors that were added were either the ‘2i’ inhibitors (GSK3-beta and MEK inhibitors) or human recombinant NME7-AB. The cells that were analyzed were those that started growing anchorage-independently, ‘floaters’.

FIG. 49 is a graph of RT-PCR measurement of the expression levels of transcription factors BRD4 and co-factor JMJD6, reported to suppress NME7 and induce NME1, respectively, and chromatin re-arrangement factors MBD3 and CHD4, reported to block induction of stem cell pluripotency, in T47D breast cancer cells following culture in a minimal serum-free base media wherein the only factors that were added were either the ‘2i’ inhibitors (GSK3-beta and MEK inhibitors) or human recombinant NME7-AB.

FIG. 50 is a cartoon of the interaction map of NME7 and associated factors resulting from analysis of the experiments described herein.

FIG. 51 is a graph of tumor volumes measured over time. T47D breast cancer cells were implanted using the standard method (dashed line) or wherein the cells were mixed 50/50 vol/vol with NME7-AB and after 14 days, those mice were injected daily with NME7-AB.

FIG. 52 shows a graph of a quantitative PCR assay that measured expression of RNA for MMP14, MMP16 and ADAM17, which can cleave MUC1-full-length to MUC1*, in either cultured cancer cells (T47D), human embryonic stem cells (HES) cultured in either FGF, NM23-H1 dimers or NME7.

FIG. 53A is a graph of RT-PCR measurement, showing a maximum of 6.5 RQ, of the expression levels of cleavage enzymes MMP14, MMP16 and ADAM17 in HES-3 human embryonic stem cells grown in FGF, HES-3 cells grown in human NME7-AB, HES-3 cells grown in NME1 dimers, T47D breast cancer cells in vitro, T47D breast cancer cells implanted into an animal, DU145 prostate cancer cells in vitro, DU145 cells implanted into an animal, and 1500 breast cancer cells implanted into an animal, all normalized to HES-3 cells grown in FGF on MEFs.

FIG. 53B is a graph of RT-PCR measurement, showing a maximum of 2.0 RQ, of the expression levels of cleavage enzymes MMP14, MMP16 and ADAM17 in HES-3 human embryonic stem cells grown in FGF, HES-3 cells grown in human NME7-AB, HES-3 cells grown in NME1 dimers, T47D breast cancer cells in vitro, T47D breast cancer cells implanted into an animal, DU145 prostate cancer cells in vitro, DU145 cells implanted into an animal, and 1500 breast cancer cells implanted into an animal, all normalized to HES-3 cells grown in FGF on MEFs.

FIG. 54 is a graph of RT-PCR measurement of the expression levels of cleavage enzymes MMP14, MMP16 and ADAM17 in T47D breast cancer cells in vitro, HES-3 human embryonic stem cells grown in FGF, HES-3 cells grown in human NME7-AB, HES-3 cells grown in NME1 dimers, all normalized to T47D breast cancer cells in vitro.

FIG. 55 is a graph of tumor volume measurement of DU145 hormone refractory prostate cancer cells, implanted into NOD/SCID male mice, after 60 days of treatment with either vehicle or the Fab of MN-E6 anti-MUC1* antibody. From Day 60 to Day 70 the treatment groups were switched. The efficacy of anti-MUC1* E6 antibody was found to be statistically significant in reducing tumor volume with p values of 0.0001.

FIG. 56 is a graph of RT-PCR measurement of the expression levels of cleavage enzymes MMP14 and MMP16 in tumors excised from DU145 hormone refractory prostate cancer cells, implanted into NOD/SCID male mice, after 60 days of treatment with either vehicle or the Fab of MN-E6 anti-MUC1* antibody. Although treatment blocking the MUC1* growth factor receptor decreased expression of both cleavage enzymes only MMP14 was statistically significant.

FIG. 57A is a photograph of a Western blot probing for MUC1* in tumors excised from DU145 hormone refractory prostate cancer cells, implanted into NOD/SCID male mice, after 60 days of treatment with either vehicle or the Fab of MN-E6 anti-MUC1* antibody. The photo shows that in anti-MUC1* Fab treated mice, there is less MUC1*, i.e. less MUC1 cleavage in the treated group.

FIG. 57B is a graph of RT-PCR measurement of the expression levels of microRNA-145 in tumors excised from DU145 hormone refractory prostate cancer cells, implanted into NOD/SCID male mice, after 60 days of treatment with either vehicle or the Fab of MN-E6 anti-MUC1* antibody. The graph shows that on average, miR-145, which signals a stem cell to differentiate, is increased in the treated group compared to the control group.

FIG. 58A show the results of a FACS experiments wherein live T47D cancer cells were probed with 40 ug/ml of MN-C2 monoclonal antibody that was selected based on binding preference to the N-10 peptide. MN-C2 monoclonal antibody shows strong binding to live T47D breast cancer cells and is cancer cell specific.

FIG. 58B show the results of a FACS experiments wherein live T47D cancer cells were probed with 20 ug/ml of MN-C2 monoclonal antibody that was selected based on binding preference to the N-10 peptide. MN-C2 monoclonal antibody shows strong binding to live T47D breast cancer cells and is cancer cell specific.

FIG. 58C show the results of a FACS experiments wherein live T47D cancer cells were probed with 10 ug/ml of MN-C2 monoclonal antibody that was selected based on binding preference to the N-10 peptide. MN-C2 monoclonal antibody shows strong binding to live T47D breast cancer cells and is cancer cell specific.

FIG. 58D show the results of a FACS experiments wherein live T47D cancer cells were probed with 5 ug/ml of MN-C2 monoclonal antibody that was selected based on binding preference to the N-10 peptide. MN-C2 monoclonal antibody shows strong binding to live T47D breast cancer cells and is cancer cell specific.

FIG. 58E show the results of a FACS experiments wherein live T47D cancer cells were probed with 40 ug/ml of MN-C3 monoclonal antibody that was selected based on binding preference to the C-10 peptide. MN-C3 monoclonal antibody shows no binding to live T47D breast cancer cells and is stem cell specific.

FIG. 58F show the results of a FACS experiments wherein live T47D cancer cells were probed with 40 ug/ml of MN-C3 monoclonal antibody that was selected based on binding preference to the C-10 peptide. MN-C3 monoclonal antibody shows no binding to live T47D breast cancer cells and is stem cell specific.

FIG. 58G show the results of a FACS experiments wherein live T47D cancer cells were probed with 40 ug/ml of MN-C3 monoclonal antibody that was selected based on binding preference to the C-10 peptide. MN-C3 monoclonal antibody shows no binding to live T47D breast cancer cells and is stem cell specific.

FIG. 58H show the results of a FACS experiments wherein live T47D cancer cells were probed with 40 ug/ml of MN-C3 monoclonal antibody that was selected based on binding preference to the C-10 peptide. MN-C3 monoclonal antibody shows no binding to live T47D breast cancer cells and is stem cell specific.

FIG. 58I shows the results of FACS experiments wherein live DU145 prostate cancer cells were probed with cancer cell specific monoclonal antibodies MN-C2.

FIG. 58J shows the results of FACS experiments wherein live DU145 prostate cancer cells were probed with stem cell specific monoclonal antibodies MN-C3 Stem cell specific MN-C3 does not bind to DU145 prostate cancer cells.

FIG. 58K is the graph of another FACS experiment that shows that cancer cell specific monoclonal antibodies MN-C2 and MN-E6 binds to DU145 prostate cancer cells, while the stem cell specific MN-C3 does not.

FIGS. 59A-59C show the results of FACS experiments wherein either stem cells or cancer cells were probed with either stem cell specific anti-MUC1* monoclonal antibodies or cancer cell specific monoclonal antibodies. Stem cell specific MN-C3 monoclonal shows strong binding to BGO1v human embryonic stem cells (FIG. 59A) but shows no binding to T47D breast cancer cells (FIG. 59B), or to DU145 prostate cancer cells (FIG. 59C).

FIG. 59D is a graph of another FACS experiment that shows that stem cell specific MN-C3 monoclonal shows strong binding to stem cells but does not bind to T47D breast cancer cells, 1500 breast cancer cell line, DU145 prostate cancer cells, or MUC1-negative PC3 prostate cancer.

FIG. 60A shows a photo of DU145 prostate cancer cells cultured in ordinary media to which nothing was added.

FIG. 60B shows a photo of DU145 prostate cancer cells cultured in ordinary media to which the Fab of stem cell specific MN-C3 was added. If the antibody recognized MUC1* as it appears on the cancer cells, the Fab of the antibody would block the dimerization of MUC1* and induce cell death. As can be seen in the photo, the Fabs of the stem cell specific antibodies had no effect on the cancer cell growth.

FIG. 60C shows a photo of DU145 prostate cancer cells cultured in ordinary media to which the Fab of stem cell specific MN-C8 was added. If the antibody recognized MUC1* as it appears on the cancer cells, the Fab of the antibody would block the dimerization of MUC1* and induce cell death. As can be seen in the photo, the Fabs of the stem cell specific antibodies had no effect on the cancer cell growth.

FIG. 60D shows a photo of DU145 prostate cancer cells cultured in ordinary media to which the Fab of cancer cell specific MN-C2 was added. If the antibody recognized MUC1* as it appears on the cancer cells, the Fab of the antibody would block the dimerization of MUC1* and induce cell death. As can be seen in the photos, the Fabs of the cancer cells specific antibodies effectively killed the cancer cells.

FIG. 60E shows a photo of DU145 prostate cancer cells cultured in ordinary media to which the Fab of cancer cell specific MN-E6 was added. If the antibody recognized MUC1* as it appears on the cancer cells, the Fab of the antibody would block the dimerization of MUC1* and induce cell death. As can be seen in the photos, the Fabs of the cancer cells specific antibodies effectively killed the cancer cells.

FIG. 61 is a sequence alignment between human NME1 and human NME7-A or -B domain. FIG. 61 discloses SEQ ID NOS 159, 161, 159, and 165, respectively, in order of appearance.

FIG. 62 lists immunogenic peptides from human NME7 with low sequence identity to NME1. The listed peptide sequences are identified as being immunogenic peptides giving rise to antibodies that target human NME7 but not human NME1. The sequences were chosen for their lack of sequence homology to human NME1 and are useful as NME7 specific peptides for generating antibodies to inhibit NME7 for the treatment or prevention of cancers.

FIG. 63 lists immunogenic peptides from human NME7 that may be important for structural integrity or for binding to MUC1*. Bivalent and bi-specific antibodies wherein each variable region binds to a different peptide portion of NME7 are preferred. Such peptides may be generated by using more than one peptide to generate the antibody specific to both. The peptides are useful as NME7 specific peptides for generating antibodies to inhibit NME7 for the treatment or prevention of cancers.

FIG. 64 lists immunogenic peptides from human NME1 that may be important for structural integrity or for binding to MUC1*. The listed peptide sequences are from human NME1 and were selected for their high homology to human NME7 as well as for their homology to other bacterial NME proteins that are able to mimic its function. In particular, peptides 50 to 53 have high homology to human NME7-A or —B and also to HSP 593.

### DETAILED DESCRIPTION OF THE PREFERRED EMBODIMENTS

### Definitions

As used herein, the “MUC1*” extra cellular domain is defined primarily by the PSMGFR sequence (GTINVHDVETQFNQYKTEAASRYNLTISDVSVSDVPFPFSAQSGA (SEQ ID NO:6)). Because the exact site of MUC1 cleavage depends on the enzyme that clips it, and that the cleavage enzyme varies depending on cell type, tissue type or the time in the evolution of the cell, the exact sequence of the MUC1* extra cellular domain may vary at the N-terminus.

As used herein, the term “PSMGFR” is an acronym for Primary Sequence of MUC1 Growth Factor Receptor as set forth as GTINVHDVETQFNQYKTEAASRYNLTISDVSVSDVPFPFSAQSGA (SEQ ID NO:6). In this regard, the “N-number” as in “N-10 PSMGFR”, “N-15 PSMGFR”, or “N-20 PSMGFR” refers to the number of amino acid residues that have been deleted at the N-terminal end of PSMGFR. Likewise “C-number” as in “C-10 PSMGFR”, “C-15 PSMGFR”, or “C-20 PSMGFR” refers to the number of amino acid residues that have been deleted at the C-terminal end of PSMGFR.

As used herein, the “extracellular domain of MUC1*” refers to the extracellular portion of a MUC1 protein that is devoid of the tandem repeat domain. In most cases, MUC1* is a cleavage product wherein the MUC1* portion consists of a short extracellular domain devoid of tandem repeats, a transmembrane domain and a cytoplasmic tail. The precise location of cleavage of MUC1 is not known perhaps because it appears that it can be cleaved by more than one enzyme. The extracellular domain of MUC1* will include most of the PSMGFR sequence but may have an additional 10-20 N-terminal amino acids.

As used herein, “NME family proteins” or “NME family member proteins”, numbered 1-10, are proteins grouped together because they all have at least one NDPK (nucleotide diphosphate kinase) domain. In some cases, the NDPK domain is not functional in terms of being able to catalyze the conversion of ATP to ADP. NME proteins were formally known as NM23 proteins, numbered H1, H2 and so on. Herein, the terms NM23 and NME are interchangeable. Herein, terms NME1, NME2, NME6 and NME7 are used to refer to the native protein as well as NME variants. In some cases these variants are more soluble, express better in E. coli or are more soluble than the native sequence protein. For example, NME7 as used in the specification can mean the native protein or a variant, such as NME7-AB that has superior commercial applicability because variations allow high yield expression of the soluble, properly folded protein in E. coli. “NME1” as referred to herein is interchangeable with “NM23-H1”. It is also intended that the invention not be limited by the exact sequence of the NME proteins. The mutant NME1-S120G, also called NM23-S120G, are used interchangeably throughout the application. The S120G mutants and the P96S mutant are preferred because of their preference for dimer formation, but may be referred to herein as NM23 dimers or NME1 dimers.

NME7 as referred to herein is intended to mean native NME7 having a molecular weight of about 42 kDa, a cleaved form having a molecular weight between 25 and 33 kDa, a variant devoid of the DM10 leader sequence, NME7-AB or a recombinant NME7 protein, or variants thereof whose sequence may be altered to allow for efficient expression or that increase yield, solubility or other characteristics that make the NME7 more effective or commercially more viable.

The present invention discloses antibodies and antibody variants that modulate a pathway involving MUC1* wherein one set of antibodies preferentially binds to MUC1* as it exists on stem cells but does not recognize MUC1* on cancer cells as well and another set of antibodies that preferentially binds to MUC1* as it exists on cancer cells but does not recognize MUC1* on stem cells as well. The present invention further discloses methods for identifying other antibodies that fall into these categories. The invention further discloses methods for using the first set of antibodies, hereafter referred to as “stem cell antibodies”, for stimulating stem cell growth in vitro and in vivo. The invention also discloses methods for using the second set of antibodies, hereafter referred to as “cancer cell antibodies”, for inhibiting cancer cell growth in vitro and in vivo.

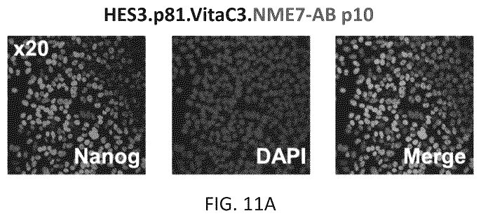

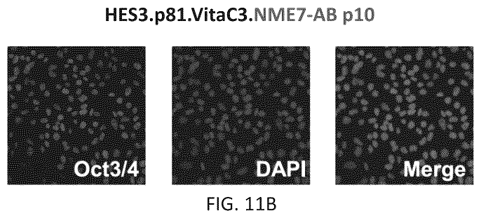

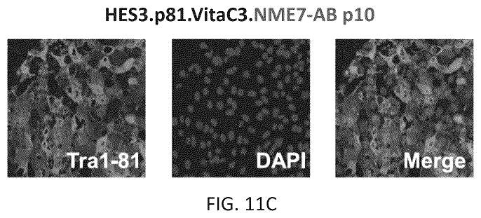

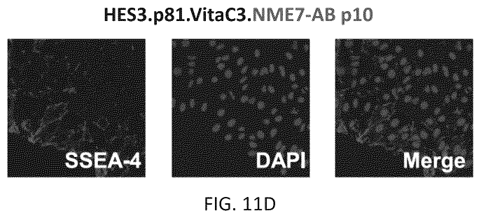

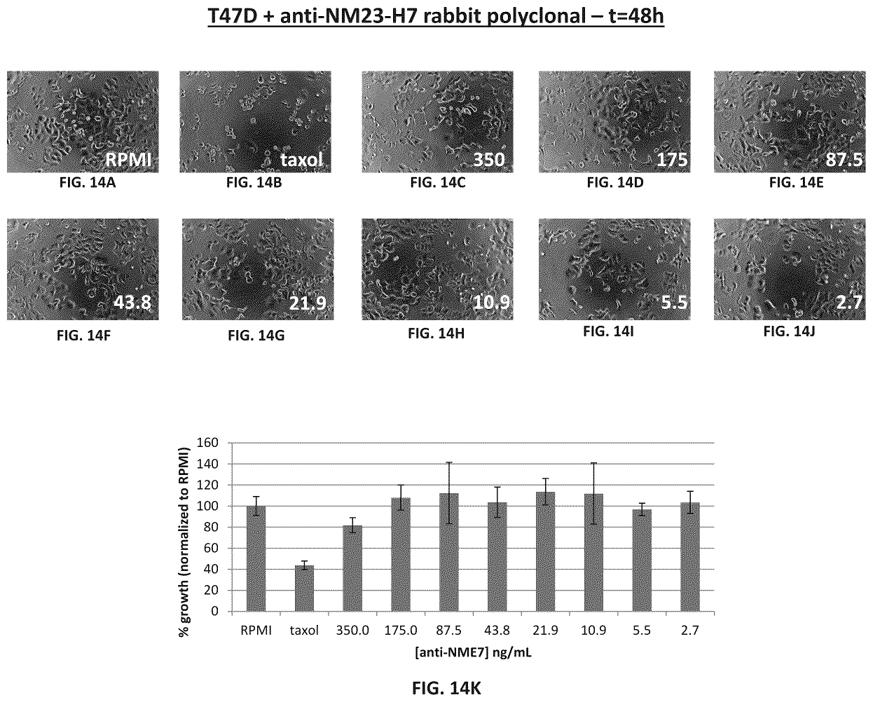

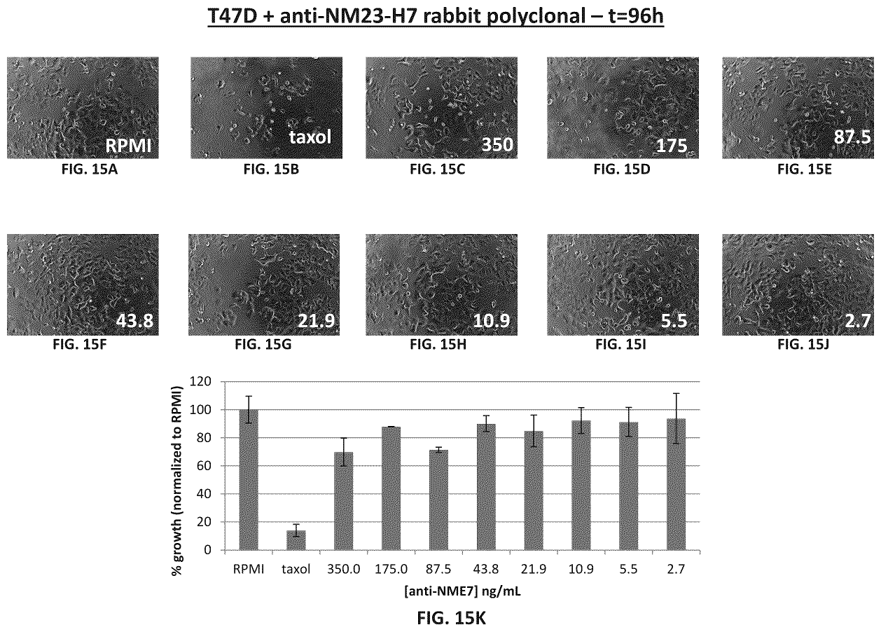

In the present application, the cancer specific antibodies MIN-C2 (also referred to herein as well as in the applications from which the present application claims priority as “C2”) or MIN-E6 (also referred to herein as well as in the applications from which the present application claims priority as “E6”) are the same antibodies structurally and sequence-wise as referred to in the present application as in other applications by the Applicant. A description of these antibodies and their CDR sequences can be found in WO2010/042562 (PCT/US2009/059754), filed Oct. 6, 2009. In particular, see FIGS. 11 to 16 therein.

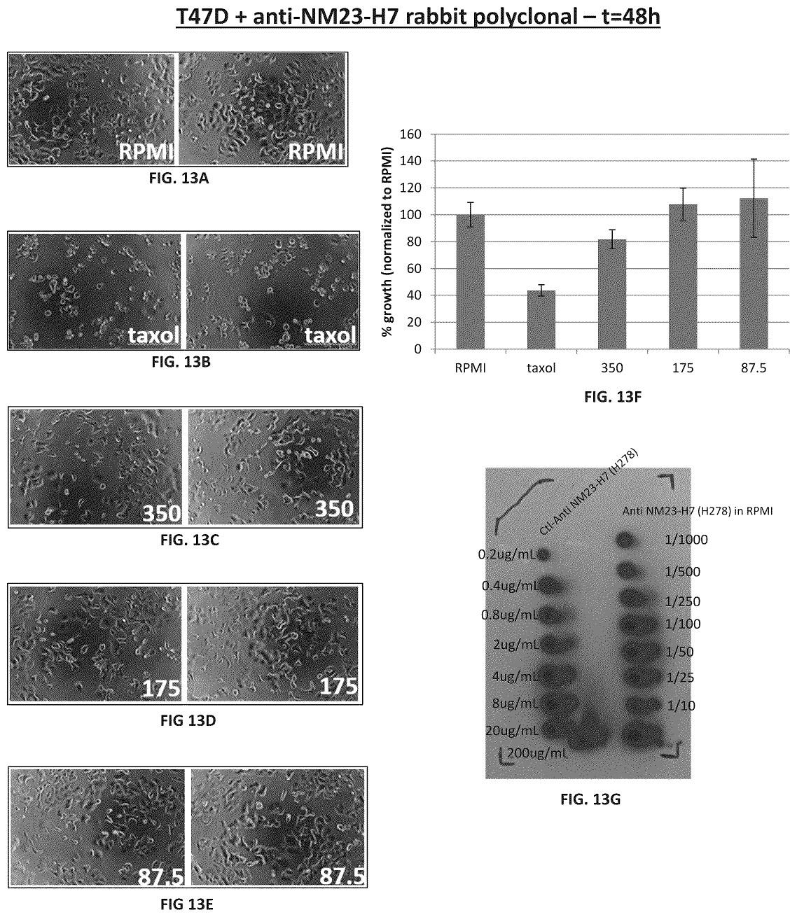

Likewise, the stem cell specific antibodies 2D6C3 (also referred to herein as well as in the applications from which the present application claims priority as “C3”) or MN-C3 or 2D6C8 (also referred to herein as well as in the applications from which the present application claims priority as “C8”) or MN-C8 are the same antibodies structurally and sequence-wise as referred to in the present application as in other applications by the Applicant. A description of these antibodies and their CDR sequences can be found in WO2012/126013 (PCT/US2012/059754), filed Mar. 19, 2012. In particular, see FIGS. 13 to 18 therein.

As used herein, an “effective amount of an agent to inhibit an NME family member protein” refers to the effective amount the agent in hindering the activating interaction between the NME family member protein and its cognate receptor such as MUC1 or MUC1*.

As used herein, “high homology” is considered to be at least 30%, 35%, 40%, 45%, 50%, 55%, 60%, 65%, 70%, 75%, 80%, 85%, 90%, 95%, or 97% identity in a designated overlapping region between any two polypeptides.

As used herein, “low homology” is considered lower than 25%, 20%, 15%, 10%, or 5% identity in a designated overlapping region between any two polypeptides.

As used herein, in reference to an agent being referred to as a “small molecule”, it may be a synthetic chemical or chemically based molecule having a molecular weight between 50 Da and 2000 Da, more preferably between 150 Da and 1000 Da, still more preferably between 200 Da and 750 Da.

As used herein, in reference to an agent being referred to as a “natural product”, it may be chemical molecule or a biological molecule, so long as the molecule exists in nature.

As used herein, “2i inhibitor” refers to small molecule inhibitors of GSK3-beta and MEK of the MAP kinase signaling pathway. The name 2i was coined in a research article (Silva J et al 2008), however herein “2i” refers to any inhibitor of either GSK3-beta or MEK, as there are many small molecules or biological agents that if they inhibit these targets, have the same effect on pluripotency or tumorigenesis.

As used herein, FGF, FGF-2 or bFGF refer to fibroblast growth factor.

As used herein, “Rho associated kinase inhibitors” may be small molecules, peptides or proteins (Rath N, et al, 2012). Rho kinase inhibitors are abbreviated here and elsewhere as ROCi or ROCKi, or Ri. The use of specific rho kinase inhibitors are meant to be exemplary and can be substituted for any other rho kinase inhibitor.

As used herein, the term “cancer stem cells” or “tumor initiating cells” refers to cancer cells that express levels of genes that have been linked to a more metastatic state or more aggressive cancers. The terms “cancer stem cells” or “tumor initiating cells” can also refer to cancer cells for which far fewer cells are required to give rise to a tumor when transplanted into an animal. Cancer stem cells and tumor initiating cells are often resistant to chemotherapy drugs.

As used herein, the terms “stem/cancer”, “cancer-like”, “stem-like” refers to a state in which cells acquire characteristics of stem cells or cancer cells, share important elements of the gene expression profile of stem cells, cancer cells or cancer stem cells. Stem-like cells may be somatic cells undergoing induction to a less mature state, such as increasing expression of pluripotency genes. Stem-like cells also refers to cells that have undergone some de-differentiation or are in a meta-stable state from which they can alter their terminal differentiation. Cancer like cells may be cancer cells that have not yet been fully characterized but display morphology and characteristics of cancer cells, such as being able to grow anchorage-independently or being able to give rise to a tumor in an animal.

As used herein, the term “antibody-like” means a molecule that may be engineered such that it contains portions of antibodies but is not an antibody that would naturally occur in nature. Examples include but are not limited to CAR (chimeric antigen receptor) T cell technology and the Ylanthia® technology. The CAR technology uses an antibody epitope fused to a portion of a T cell so that the body's immune system is directed to attack a specific target protein or cell. The Ylanthia® technology consists of an “antibody-like” library that is a collection of synthetic human fabs that are then screened for binding to peptide epitopes from target proteins. The selected Fab regions can then be engineered into a scaffold or framework so that they resemble antibodies.

### Sequence Listing Free Text

As regards the use of nucleotide symbols other than a, g, c, t, they follow the convention set forth in WIPO Standard ST.25, Appendix 2, Table 1, wherein k represents t or g; n represents a, c, t or g; m represents a or c; r represents a or g; s represents c or g; w represents a or t and y represents c or t.

(SEQ ID NO: 1)
MTPGTQSPFF LLLLLTVLTV VTGSGHASST PGGEKETSAT
QRSSVPSSTE KNAVSMTSSV LSSHSPGSGS STTQGQDVTL
APATEPASGS AATWGQDVTS VPVTRPALGS TTPPAHDVTS
APDNKPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDNRPALGS TAPPVHNVTS
ASGSASGSAS TLVHNGTSAR ATTTPASKST PFSIPSHHSD
TPTTLASHST KTDASSTHHS SVPPLTSSNH STSPQLSTGV
SFFFLSFHIS NLQFNSSLED PSTDYYQELQ RDISEMFLQI
YKQGGFLGLS NIKFRPGSVV VQLTLAFREG TINVHDVETQ
FNQYKTEAAS RYNLTISDVS VSDVPFPFSA QSGAGVPGWG
IALLVLVCVL VALAIVYLIA LAVCQCRRKN YGQLDIFPAR
DTYHPMSEYP TYHTHGRYVP PSSTDRSPYE KVSAGNGGSS
LSYTNPAVAA ASANL

describes full-length MUC1 Receptor (Mucin 1 precursor, Genbank Accession number: P15941).

(SEQ ID NO: 2)
MTPGTQSPFFLLLLLTVLT
(SEQ ID NO: 3)
MTPGTQSPFFLLLLLTVLT VVTA
(SEQ ID NO: 4)
MTPGTQSPFFLLLLLTVLT VVTG

SEQ ID NOS:2, 3 and 4 describe N-terminal MUC-1 signaling sequence for directing MUC1 receptor and truncated isoforms to cell membrane surface. Up to 3 amino acid residues may be absent at C-terminal end as indicated by variants in SEQ ID NOS:2, 3 and 4.

(SEQ ID NO: 5)
GTINVHDVETQFNQYKTEAASRYNLTISDVSVSDVPFPFSAQSGAGVPGW
GIALLVLVCVLVALAIVYLIALAVCQCRRKNYGQLDIFPARDTYHPMSEY
PTYHTHGRYVPPSSTDRSPYEKVSAGNGGSSLSYTNPAVAAASANL

describes a truncated MUC1 receptor isoform having nat-PSMGFR at its N-terminus and including the transmembrane and cytoplasmic sequences of a full-length MUC1 receptor.

(SEQ ID NO: 6)
GTINVHDVETQFNQYKTEAASRYNLTISDVSVSDVPFPFSAQSGA

describes Native Primary Sequence of the MUC1 Growth Factor Receptor (nat-PSMGFR—an example of “PSMGFR”):

(SEQ ID NO: 7)
TINVHDVETQFNQYKTEAASRYNLTISDVSVSDVPFPFSAQSGA

describes Native Primary Sequence of the MUC1 Growth Factor Receptor (nat-PSMGFR—An example of “PSMGFR”), having a single amino acid deletion at the N-terminus of SEQ ID NO:6).

(SEQ ID NO: 8)
GTINVHDVETQFNQYKTEAASPYNLTISDVSVSDVPFPFSAQSGA

describes “SPY” functional variant of the native Primary Sequence of the MUC1 Growth Factor Receptor having enhanced stability (var-PSMGFR—An example of “PSMGFR”).

(SEQ ID NO: 9)
TINVHDVETQFNQYKTEAASPYNLTISDVSVSDVPFPFSAQSGA

describes “SPY” functional variant of the native Primary Sequence of the MUC1 Growth Factor Receptor having enhanced stability (var-PSMGFR—An example of “PSMGFR”), having a single amino acid deletion at the C-terminus of SEQ ID NO:8).

(SEQ ID NO: 10)
tgtcagtgccgccgaaagaactacgggcagctggacatctttccagcccg
ggatacctaccatcctatgagcgagtaccccacctaccacacccatgggc
gctatgtgccccctagcagtaccgatcgtagcccctatgagaaggtttct
gcaggtaacggtggcagcagcctctcttacacaaacccagcagtggcagc
cgcttctgccaacttg

describes MUC1 cytoplasmic domain nucleotide sequence.

(SEQ ID NO: 11)
CQCRRKNYGQLDIFPARDTYHPMSEYPTYHTHGRYVPPSSTDRSPYEKVS
AGNGGSSLSYTNPAVAAASANL

describes MUC1 cytoplasmic domain amino acid sequence.

(SEQ ID NO: 12)
gagatcctgagacaatgaatcatagtgaaagattcgttttcattgcagag
tggtatgatccaaatgcttcacttcttcgacgttatgagcttttatttta
cccaggggatggatctgttgaaatgcatgatgtaaagaatcatcgcacct
ttttaaagcggaccaaatatgataacctgcacttggaagatttatttata
ggcaacaaagtgaatgtcttttctcgacaactggtattaattgactatgg
ggatcaatatacagctcgccagctgggcagtaggaaagaaaaaacgctag
ccctaattaaaccagatgcaatatcaaaggctggagaaataattgaaata
ataaacaaagctggatttactataaccaaactcaaaatgatgatgctttc
aaggaaagaagcattggattttcatgtagatcaccagtcaagaccctttt
tcaatgagctgatccagtttattacaactggtcctattattgccatggag
attttaagagatgatgctatatgtgaatggaaaagactgctgggacctgc
aaactctggagtggcacgcacagatgcttctgaaagcattagagccctct
ttggaacagatggcataagaaatgcagcgcatggccctgattcttttgct
tctgcggccagagaaatggagttgttttttccttcaagtggaggttgtgg
gccggcaaacactgctaaatttactaattgtacctgttgcattgttaaac
cccatgctgtcagtgaaggtatgttgaatacactatattcagtacatttt
gttaataggagagcaatgtttattttcttgatgtactttatgtatagaaa
ataa

describes NME7 nucleotide sequence (NME7: GENBANK ACCESSION AB209049).

(SEQ ID NO: 13)
DPETMNHSERFVFIAEWYDPNASLLRRYELLFYPGDGSVEMHDVKNHRTF
LKRTKYDNLHLEDLFIGNKVNVFSRQLVLIDYGDQYTARQLGSRKEKTLA
LIKPDAISKAGEIIEIINKAGFTITKLKMMMLSRKEALDFHVDHQSRPFF
NELIQFITTGPIIAMEILRDDAICEWKRLLGPANSGVARTDASESIRALF
GTDGIRNAAHGPDSFASAAREMELFFPSSGGCGPANTAKFTNCTCCIVKP
HAVSEGMLNTLYSVHFVNRRAMFIFLMYFMYRK

describes NME7 amino acid sequence (NME7: GENBANK ACCESSION AB209049).

(SEQ ID NO: 14)
atggtgctactgtctactttagggatcgtctttcaaggcgaggggcctcc
tatctcaagctgtgatacaggaaccatggccaactgtgagcgtaccttca
ttgcgatcaaaccagatggggtccagcggggtcttgtgggagagattatc
aagcgttttgagcagaaaggattccgccttgttggtctgaaattcatgca
agcttccgaagatcttctcaaggaacactacgttgacctgaaggaccgtc
cattctttgccggcctggtgaaatacatgcactcagggccggtagttgcc
atggtctgggaggggctgaatgtggtgaagacgggccgagtcatgctcgg
ggagaccaaccctgcagactccaagcctgggaccatccgtggagacttct
gcatacaagttggcaggaacattatacatggcagtgattctgtggagagt
gcagagaaggagatcggcttgtggtttcaccctgaggaactggtagatta
cacgagctgtgctcagaactggatctatgaatga

describes NM23-H1 nucleotide sequence (NM23-H1: GENBANK ACCESSION AF487339).

(SEQ ID NO: 15)
MVLLSTLGIVFQGEGPPISSCDTGTMANCERTFIAIKPDGVQRGLVGEII
KRFEQKGFRLVGLKFMQASEDLLKEHYVDLKDRPFFAGLVKYMHSGPVVA
MVWEGLNVVKTGRVMLGETNPADSKPGTIRGDFCIQVGRNIIHGSDSVES
AEKEIGLWFHPEELVDYTSCAQNWIYE

NM23-H1 describes amino acid sequence (NM23-H1: GENBANK ACCESSION AF487339).

(SEQ ID NO: 16)
atggtgctactgtctactttagggatcgtctttcaaggcgaggggcctcc
tatctcaagctgtgatacaggaaccatggccaactgtgagcgtaccttca
ttgcgatcaaaccagatggggtccagcggggtcttgtgggagagattatc
aagcgttttgagcagaaaggattccgccttgttggtctgaaattcatgca
agcttccgaagatcttctcaaggaacactacgttgacctgaaggaccgtc
cattctttgccggcctggtgaaatacatgcactcagggccggtagttgcc
atggtctgggaggggctgaatgtggtgaagacgggccgagtcatgctcgg
ggagaccaaccctgcagactccaagcctgggaccatccgtggagacttct
gcatacaagttggcaggaacattatacatggcggtgattctgtggagagt
gcagagaaggagatcggcttgtggtttcaccctgaggaactggtagatta
cacgagctgtgctcagaactggatctatgaatga

describes NM23-H1 S120G mutant nucleotide sequence (NM23-H1: GENBANK ACCESSION AF487339).

(SEQ ID NO: 17)
MVLLSTLGIVFQGEGPPISSCDTGTMANCERTFIAIKPDGVQRGLVGEII
KRFEQKGFRLVGLKFMQASEDLLKEHYVDLKDRPFFAGLVKYMHSGPVVA
MVWEGLNVVKTGRVMLGETNPADSKPGTIRGDFCIQVGRNIIHGGDSVES
AEKEIGLWFHPEELVDYTSCAQNWIYE

describes NM23-H1 S120G mutant amino acid sequence (NM23-H1: GENBANK ACCESSION AF487339).

(SEQ ID NO: 18)
atggccaacctggagcgcaccttcatcgccatcaagccggacggcgtgca
gcgcggcctggtgggcgagatcatcaagcgcttcgagcagaagggattcc
gcctcgtggccatgaagttcctccgggcctctgaagaacacctgaagcag
cactacattgacctgaaagaccgaccattcttccctgggctggtgaagta
catgaactcagggccggttgtggccatggtctgggaggggctgaacgtgg
tgaagacaggccgagtgatgcttggggagaccaatccagcagattcaaag
ccaggcaccattcgtggggacttctgcattcaggttggcaggaacatcat
tcatggcagtgattcagtaaaaagtgctgaaaaagaaatcagcctatggt
ttaagcctgaagaactggttgactacaagtcttgtgctcatgactgggtc
tatgaataa

describes NM23-H2 nucleotide sequence (NM23-H2: GENBANK ACCESSION AK313448).

(SEQ ID NO: 19)
MANLERTFIAIKPDGVQRGLVGEIIKRFEQKGFRLVAMKFLRASEEHLKQ
HYIDLKDRPFFPGLVKYMNSGPVVAMVWEGLNVVKTGRVMLGETNPADSK
PGTIRGDFCIQVGRNIIHGSDSVKSAEKEISLWFKPEELVDYKSCAHDWV
YE

describes NM23-H2 amino acid sequence (NM23-H2: GENBANK ACCESSION AK313448).

Human NM23-H7-2 sequence optimized for E. coli expression:

(DNA)
(SEQ ID NO: 20)
atgcatgacgttaaaaatcaccgtacctttctgaaacgcacgaaatatga
taatctgcatctggaagacctgtttattggcaacaaagtcaatgtgttct
ctcgtcagctggtgctgatcgattatggcgaccagtacaccgcgcgtcaa
ctgggtagtcgcaaagaaaaaacgctggccctgattaaaccggatgcaat
ctccaaagctggcgaaattatcgaaattatcaacaaagcgggtttcacca
tcacgaaactgaaaatgatgatgctgagccgtaaagaagccctggatttt
catgtcgaccaccagtctcgcccgtttttcaatgaactgattcaattcat
caccacgggtccgattatcgcaatggaaattctgcgtgatgacgctatct
gcgaatggaaacgcctgctgggcccggcaaactcaggtgttgcgcgtacc
gatgccagtgaatccattcgcgctctgtttggcaccgatggtatccgtaa
tgcagcacatggtccggactcattcgcatcggcagctcgtgaaatggaac
tgtttttcccgagctctggcggttgcggtccggcaaacaccgccaaattt
accaattgtacgtgctgtattgtcaaaccgcacgcagtgtcagaaggcct
gctgggtaaaattctgatggcaatccgtgatgctggctttgaaatctcgg
ccatgcagatgttcaacatggaccgcgttaacgtcgaagaattctacgaa
gtttacaaaggcgtggttaccgaatatcacgatatggttacggaaatgta
ctccggtccgtgcgtcgcgatggaaattcagcaaaacaatgccaccaaaa
cgtttcgtgaattctgtggtccggcagatccggaaatcgcacgtcatctg
cgtccgggtaccctgcgcgcaatttttggtaaaacgaaaatccagaacgc
tgtgcactgtaccgatctgccggaagacggtctgctggaagttcaatact
ttttcaaaattctggataattga
(amino acids)
(SEQ ID NO: 21)
MHDVKNHRTFLKRTKYDNLHLEDLFIGNKVNVFSRQLVLIDYGDQYTARQ
LGSRKEKTLALIKPDAISKAGEIIEIINKAGFTITKLKMMMLSRKEALDF
HVDHQSRPFFNELIQFITTGPIIAMEILRDDAICEWKRLLGPANSGVART
DASESIRALFGTDGIRNAAHGPDSFASAAREMELFFPSSGGCGPANTAKF
TNCTCCIVKPHAVSEGLLGKILMAIRDAGFEISAMQMFNMDRVNVEEFYE
VYKGVVTEYHDMVTEMYSGPCVAMEIQQNNATKTFREFCGPADPEIARHL
RPGTLRAIFGKTKIQNAVHCTDLPEDGLLEVQYFFKILDN-

Human NME7-A:

(DNA)
(SEQ ID NO: 22)
atggaaaaaacgctagccctaattaaaccagatgcaatatcaaaggctgg
agaaataattgaaataataaacaaagctggatttactataaccaaactca
aaatgatgatgctttcaaggaaagaagcattggattttcatgtagatcac
cagtcaagaccctttttcaatgagctgatccagtttattacaactggtcc
tattattgccatggagattttaagagatgatgctatatgtgaatggaaaa
gactgctgggacctgcaaactctggagtggcacgcacagatgcttctgaa
agcattagagccctctttggaacagatggcataagaaatgcagcgcatgg
ccctgattcttttgcttctgcggccagagaaatggagttgtttttttga
(amino acids)
(SEQ ID NO: 23)
MEKTLALIKPDAISKAGEIIEIINKAGFTITKLKMMMLSRKEALDFHVDH
QSRPFFNELIQFITTGPIIAMEILRDDAICEWKRLLGPANSGVARTDASE
SIRALFGTDGIRNAAHGPDSFASAAREMELFF-

Human NME7-A1:

(DNA)
(SEQ ID NO: 24)
atggaaaaaacgctagccctaattaaaccagatgcaatatcaaaggctgg
agaaataattgaaataataaacaaagctggatttactataaccaaactca
aaatgatgatgctttcaaggaaagaagcattggattttcatgtagatcac
cagtcaagaccctttttcaatgagctgatccagtttattacaactggtcc
tattattgccatggagattttaagagatgatgctatatgtgaatggaaaa
gactgctgggacctgcaaactctggagtggcacgcacagatgcttctgaa
agcattagagccctctttggaacagatggcataagaaatgcagcgcatgg
ccctgattcttttgcttctgcggccagagaaatggagttgttttttcctt
caagtggaggttgtgggccggcaaacactgctaaatttacttga
(amino acids)
(SEQ ID NO: 25)
MEKTLALIKPDAISKAGEIIEIINKAGFTITKLKMMMLSRKEALDFHVDH
QSRPFFNELIQFITTGPIIAMEILRDDAICEWKRLLGPANSGVARTDASE
SIRALFGTDGIRNAAHGPDSFASAAREMELFFPSSGGCGPANTAKFT-

Human NME7-A2:

(DNA)
(SEQ ID NO: 26)
atgaatcatagtgaaagattcgttttcattgcagagtggtatgatccaaa
tgcttcacttcttcgacgttatgagcttttattttacccaggggatggat
ctgttgaaatgcatgatgtaaagaatcatcgcacctttttaaagcggacc
aaatatgataacctgcacttggaagatttatttataggcaacaaagtgaa
tgtcttttctcgacaactggtattaattgactatggggatcaatatacag
ctcgccagctgggcagtaggaaagaaaaaacgctagccctaattaaacca
gatgcaatatcaaaggctggagaaataattgaaataataaacaaagctgg
atttactataaccaaactcaaaatgatgatgctttcaaggaaagaagcat
tggattttcatgtagatcaccagtcaagaccctttttcaatgagctgatc
cagtttattacaactggtcctattattgccatggagattttaagagatga
tgctatatgtgaatggaaaagactgctgggacctgcaaactctggagtgg
cacgcacagatgcttctgaaagcattagagccctctttggaacagatggc
ataagaaatgcagcgcatggccctgattcttttgcttctgcggccagaga
aatggagttgtttttttga
(amino acids)
(SEQ ID NO: 27)
MNHSERFVFIAEWYDPNASLLRRYELLFYPGDGSVEMHDVKNHRTFLKRT
KYDNLHLEDLFIGNKVNVFSRQLVLIDYGDQYTARQLGSRKEKTLALIKP
DAISKAGEIIEIINKAGFTITKLKMMMLSRKEALDFHVDHQSRPFFNELI
QFITTGPIIAMEILRDDAICEWKRLLGPANSGVARTDASESIRALFGTDG
IRNAAHGPDSFASAAREMELFF-

Human NME7-A3:

(DNA)
(SEQ ID NO: 28)
atgaatcatagtgaaagattcgttttcattgcagagtggtatgatccaaa
tgcttcacttcttcgacgttatgagcttttattttacccaggggatggat
ctgttgaaatgcatgatgtaaagaatcatcgcacctttttaaagcggacc
aaatatgataacctgcacttggaagatttatttataggcaacaaagtgaa
tgtcttttctcgacaactggtattaattgactatggggatcaatatacag
ctcgccagctgggcagtaggaaagaaaaaacgctagccctaattaaacca
gatgcaatatcaaaggctggagaaataattgaaataataaacaaagctgg
atttactataaccaaactcaaaatgatgatgctttcaaggaaagaagcat
tggattttcatgtagatcaccagtcaagaccctttttcaatgagctgatc
cagtttattacaactggtcctattattgccatggagattttaagagatga
tgctatatgtgaatggaaaagactgctgggacctgcaaactctggagtgg
cacgcacagatgcttctgaaagcattagagccctctttggaacagatggc
ataagaaatgcagcgcatggccctgattcttttgcttctgcggccagaga
aatggagttgttttttccttcaagtggaggttgtgggccggcaaacactg
ctaaatttacttga
(amino acids)
(SEQ ID NO: 29)
MNHSERFVFIAEWYDPNASLLRRYELLFYPGDGSVEMHDVKNHRTFLKRT
KYDNLHLEDLFIGNKVNVFSRQLVLIDYGDQYTARQLGSRKEKTLALIKP
DAISKAGEIIEIINKAGFTITKLKMMMLSRKEALDFHVDHQSRPFFNELI
QFITTGPIIAMEILRDDAICEWKRLLGPANSGVARTDASESIRALFGTDG
IRNAAHGPDSFASAAREMELFFPSSGGCGPANTAKFT-

Human NME7-B:

(DNA)
(SEQ ID NO: 30)
atgaattgtacctgttgcattgttaaaccccatgctgtcagtgaaggact
gttgggaaagatcctgatggctatccgagatgcaggttttgaaatctcag
ctatgcagatgttcaatatggatcgggttaatgttgaggaattctatgaa
gtttataaaggagtagtgaccgaatatcatgacatggtgacagaaatgta
ttctggcccttgtgtagcaatggagattcaacagaataatgctacaaaga
catttcgagaattttgtggacctgctgatcctgaaattgcccggcattta
cgccctggaactctcagagcaatctttggtaaaactaagatccagaatgc
tgttcactgtactgatctgccagaggatggcctattagaggttcaatact
tcttctga
(amino acids)
(SEQ ID NO: 31)
MNCTCCIVKPHAVSEGLLGKILMAIRDAGFEISAMQMFNMDRVNVEEFYE
VYKGVVTEYHDMVTEMYSGPCVAMEIQQNNATKTFREFCGPADPEIARHL
RPGTLRAIFGKTKIQNAVHCTDLPEDGLLEVQYFF-

Human NME7-B1:

(DNA)
(SEQ ID NO: 32)
atgaattgtacctgttgcattgttaaaccccatgctgtcagtgaaggact
gttgggaaagatcctgatggctatccgagatgcaggttttgaaatctcag
ctatgcagatgttcaatatggatcgggttaatgttgaggaattctatgaa
gtttataaaggagtagtgaccgaatatcatgacatggtgacagaaatgta
ttctggcccttgtgtagcaatggagattcaacagaataatgctacaaaga
catttcgagaattttgtggacctgctgatcctgaaattgcccggcattta
cgccctggaactctcagagcaatctttggtaaaactaagatccagaatgc
tgttcactgtactgatctgccagaggatggcctattagaggttcaatact
tcttcaagatcttggataattagtga
(amino acids)
(SEQ ID NO: 33)
MNCTCCIVKPHAVSEGLLGKILMAIRDAGFEISAMQMFNMDRVNVEEFYE
VYKGVVTEYHDMVTEMYSGPCVAMEIQQNNATKTFREFCGPADPEIARHL
RPGTLRAIFGKTKIQNAVHCTDLPEDGLLEVQYFFKILDN-

Human NME7-B2:

(DNA)
(SEQ ID NO: 34)
atgccttcaagtggaggttgtgggccggcaaacactgctaaatttactaa
ttgtacctgttgcattgttaaaccccatgctgtcagtgaaggactgttgg
gaaagatcctgatggctatccgagatgcaggttttgaaatctcagctatg
cagatgttcaatatggatcgggttaatgttgaggaattctatgaagttta
taaaggagtagtgaccgaatatcatgacatggtgacagaaatgtattctg
gcccttgtgtagcaatggagattcaacagaataatgctacaaagacattt
cgagaattttgtggacctgctgatcctgaaattgcccggcatttacgccc
tggaactctcagagcaatctttggtaaaactaagatccagaatgctgttc
actgtactgatctgccagaggatggcctattagaggttcaatacttcttc
tga
(amino acids)
(SEQ ID NO: 35)
MPSSGGCGPANTAKFTNCTCCIVKPHAVSEGLLGKILMAIRDAGFEISAM
QMFNMDRVNVEEFYEVYKGVVTEYHDMVTEMYSGPCVAMEIQQNNATKTF
REFCGPADPEIARHLRPGTLRAIFGKTKIQNAVHCTDLPEDGLLEVQ
YFF-

Human NME7-B3:

(DNA)
(SEQ ID NO: 36)
atgccttcaagtggaggttgtgggccggcaaacactgctaaatttactaa
ttgtacctgttgcattgttaaaccccatgctgtcagtgaaggactgttgg
gaaagatcctgatggctatccgagatgcaggttttgaaatctcagctatg
cagatgttcaatatggatcgggttaatgttgaggaattctatgaagttta
taaaggagtagtgaccgaatatcatgacatggtgacagaaatgtattctg
gcccttgtgtagcaatggagattcaacagaataatgctacaaagacattt
cgagaattttgtggacctgctgatcctgaaattgcccggcatttacgccc
tggaactctcagagcaatctttggtaaaactaagatccagaatgctgttc
actgtactgatctgccagaggatggcctattagaggttcaatacttcttc
aagatcttggataattagtga
(amino acids)
(SEQ ID NO: 37)
MPSSGGCGPANTAKFTNCTCCIVKPHAVSEGLLGKILMAIRDAGFEISAM
QMFNMDRVNVEEFYEVYKGVVTEYHDMVTEMYSGPCVAMEIQQNNATKTF
REFCGPADPEIARHLRPGTLRAIFGKTKIQNAVHCTDLPEDGLLEVQYFF
KILDN--

Human NME7-AB:

(DNA)
(SEQ ID NO: 38)
atggaaaaaacgctagccctaattaaaccagatgcaatatcaaaggctgg
agaaataattgaaataataaacaaagctggatttactataaccaaactca
aaatgatgatgctttcaaggaaagaagcattggattttcatgtagatcac
cagtcaagaccctttttcaatgagctgatccagtttattacaactggtcc
tattattgccatggagattttaagagatgatgctatatgtgaatggaaaa
gactgctgggacctgcaaactctggagtggcacgcacagatgcttctgaa
agcattagagccctctttggaacagatggcataagaaatgcagcgcatgg
ccctgattcttttgcttctgcggccagagaaatggagttgttttttcctt
caagtggaggttgtgggccggcaaacactgctaaatttactaattgtacc
tgttgcattgttaaaccccatgctgtcagtgaaggactgttgggaaagat
cctgatggctatccgagatgcaggttttgaaatctcagctatgcagatgt
tcaatatggatcgggttaatgttgaggaattctatgaagtttataaagga
gtagtgaccgaatatcatgacatggtgacagaaatgtattctggcccttg
tgtagcaatggagattcaacagaataatgctacaaagacatttcgagaat
tttgtggacctgctgatcctgaaattgcccggcatttacgccctggaact
ctcagagcaatctttggtaaaactaagatccagaatgctgttcactgtac
tgatctgccagaggatggcctattagaggttcaatacttcttcaagatct
tggataattagtga
(amino acids)
(SEQ ID NO: 39)
MEKTLALIKPDAISKAGEIIEIINKAGFTITKLKMMMLSRKEALDFHVDH
QSRPFFNELIQFITTGPIIAMEILRDDAICEWKRLLGPANSGVARTDASE
SIRALFGTDGIRNAAHGPDSFASAAREMELFFPSSGGCGPANTAKFTNCT
CCIVKPHAVSEGLLGKILMAIRDAGFEISAMQMFNMDRVNVEEFYEVYKG
VVTEYHDMVTEMYSGPCVAMEIQQNNATKTFREFCGPADPEIARHLRPGT
LRAIFGKTKIQNAVHCTDLPEDGLLEVQYFFKILDN--

Human NME7-ABI:

(DNA)
(SEQ ID NO: 40)
atggaaaaaacgctagccctaattaaaccagatgcaatatcaaaggctgg
agaaataattgaaataataaacaaagctggatttactataaccaaactca
aaatgatgatgctttcaaggaaagaagcattggattttcatgtagatcac
cagtcaagaccctttttcaatgagctgatccagtttattacaactggtcc
tattattgccatggagattttaagagatgatgctatatgtgaatggaaaa
gactgctgggacctgcaaactctggagtggcacgcacagatgcttctgaa
agcattagagccctctttggaacagatggcataagaaatgcagcgcatgg
ccctgattcttttgcttctgcggccagagaaatggagttgttttttcctt
caagtggaggttgtgggccggcaaacactgctaaatttactaattgtacc
tgttgcattgttaaaccccatgctgtcagtgaaggactgttgggaaagat
cctgatggctatccgagatgcaggttttgaaatctcagctatgcagatgt
tcaatatggatcgggttaatgttgaggaattctatgaagtttataaagga
gtagtgaccgaatatcatgacatggtgacagaaatgtattctggcccttg
tgtagcaatggagattcaacagaataatgctacaaagacatttcgagaat
tttgtggacctgctgatcctgaaattgcccggcatttacgccctggaact
ctcagagcaatctttggtaaaactaagatccagaatgctgttcactgtac
tgatctgccagaggatggcctattagaggttcaatacttcttctga
(amino acids)
(SEQ ID NO: 41)
MEKTLALIKPDAISKAGEIIEIINKAGFTITKLKMMMLSRKEALDFHVDH
QSRPFFNELIQFITTGPIIAMEILRDDAICEWKRLLGPANSGVARTDASE
SIRALFGTDGIRNAAHGPDSFASAAREMELFFPSSGGCGPANTAKFTNCT
CCIVKPHAVSEGLLGKILMAIRDAGFEISAMQMFNMDRVNVEEFYEVYKG
VVTEYHDMVTEMYSGPCVAMEIQQNNATKTFREFCGPADPEIARHLRPGT
LRAIFGKTKIQNAVHCTDLPEDGLLEVQYFF-

Human NME7-A sequence optimized for E. coli expression:

(DNA)
(SEQ ID NO: 42)
atggaaaaaacgctggccctgattaaaccggatgcaatctccaaagctgg
cgaaattatcgaaattatcaacaaagcgggtttcaccatcacgaaactga
aaatgatgatgctgagccgtaaagaagccctggattttcatgtcgaccac
cagtctcgcccgtttttcaatgaactgattcaattcatcaccacgggtcc
gattatcgcaatggaaattctgcgtgatgacgctatctgcgaatggaaac
gcctgctgggcccggcaaactcaggtgttgcgcgtaccgatgccagtgaa
tccattcgcgctctgtttggcaccgatggtatccgtaatgcagcacatgg
tccggactcattcgcatcggcagctcgtgaaatggaactgtttttctga
(amino acids)
(SEQ ID NO: 43)
MEKTLALIKPDAISKAGEIIEIINKAGFTITKLKMMMLSRKEALDFHVDH
QSRPFFNELIQFITTGPIIAMEILRDDAICEWKRLLGPANSGVARTDASE
SIRALFGTDGIRNAAHGPDSFASAAREMELFF-

Human NME7-A1 sequence optimized for E. coli expression:

(DNA)
(SEQ ID NO: 44)
atggaaaaaacgctggccctgattaaaccggatgcaatctccaaagctgg
cgaaattatcgaaattatcaacaaagcgggtttcaccatcacgaaactga
aaatgatgatgctgagccgtaaagaagccctggattttcatgtcgaccac
cagtctcgcccgtttttcaatgaactgattcaattcatcaccacgggtcc
gattatcgcaatggaaattctgcgtgatgacgctatctgcgaatggaaac
gcctgctgggcccggcaaactcaggtgttgcgcgtaccgatgccagtgaa
tccattcgcgctctgtttggcaccgatggtatccgtaatgcagcacatgg
tccggactcattcgcatcggcagctcgtgaaatggaactgtttttcccga
gctctggcggttgcggtccggcaaacaccgccaaatttacctga
(amino acids)
(SEQ ID NO: 45)
MEKTLALIKPDAISKAGEIIEIINKAGFTITKLKMMMLSRKEALDFHVDH
QSRPFFNELIQFITTGPIIAMEILRDDAICEWKRLLGPANSGVARTDASE
SIRALFGTDGIRNAAHGPDSFASAAREMELFFPSSGGCGPANTAKFT-

Human NME7-A2 sequence optimized for E. coli expression:

(DNA)
(SEQ ID NO: 46)
atgaatcactccgaacgctttgtttttatcgccgaatggtatgacccgaa
tgcttccctgctgcgccgctacgaactgctgttttatccgggcgatggta
gcgtggaaatgcatgacgttaaaaatcaccgtacctttctgaaacgcacg
aaatatgataatctgcatctggaagacctgtttattggcaacaaagtcaa
tgtgttctctcgtcagctggtgctgatcgattatggcgaccagtacaccg
cgcgtcaactgggtagtcgcaaagaaaaaacgctggccctgattaaaccg
gatgcaatctccaaagctggcgaaattatcgaaattatcaacaaagcggg
tttcaccatcacgaaactgaaaatgatgatgctgagccgtaaagaagccc
tggattttcatgtcgaccaccagtctcgcccgtttttcaatgaactgatt
caattcatcaccacgggtccgattatcgcaatggaaattctgcgtgatga
cgctatctgcgaatggaaacgcctgctgggcccggcaaactcaggtgttg
cgcgtaccgatgccagtgaatccattcgcgctctgtttggcaccgatggt
atccgtaatgcagcacatggtccggactcattcgcatcggcagctcgtga
aatggaactgtttttctga
(amino acids)
(SEQ ID NO: 47)
MNHSERFVFIAEWYDPNASLLRRYELLFYPGDGSVEMHDVKNHRTFLKRT
KYDNLHLEDLFIGNKVNVFSRQLVLIDYGDQYTARQLGSRKEKTLALIKP
DAISKAGEIIEIINKAGFTITKLKMMMLSRKEALDFHVDHQSRPFFNELI
QFITTGPIIAMEILRDDAICEWKRLLGPANSGVARTDASESIRALFGTDG
IRNAAHGPDSFASAAREMELFF-

Human NME7-A3 sequence optimized for E. coli expression:

(DNA)
(SEQ ID NO: 48)
atgaatcactccgaacgctttgtttttatcgccgaatggtatgacccgaa
tgcttccctgctgcgccgctacgaactgctgttttatccgggcgatggta
gcgtggaaatgcatgacgttaaaaatcaccgtacctttctgaaacgcacg
aaatatgataatctgcatctggaagacctgtttattggcaacaaagtcaa
tgtgttctctcgtcagctggtgctgatcgattatggcgaccagtacaccg
cgcgtcaactgggtagtcgcaaagaaaaaacgctggccctgattaaaccg
gatgcaatctccaaagctggcgaaattatcgaaattatcaacaaagcggg
tttcaccatcacgaaactgaaaatgatgatgctgagccgtaaagaagccc
tggattttcatgtcgaccaccagtctcgcccgtttttcaatgaactgatt
caattcatcaccacgggtccgattatcgcaatggaaattctgcgtgatga
cgctatctgcgaatggaaacgcctgctgggcccggcaaactcaggtgttg
cgcgtaccgatgccagtgaatccattcgcgctctgtttggcaccgatggt
atccgtaatgcagcacatggtccggactcattcgcatcggcagctcgtga
aatggaactgtttttcccgagctctggcggttgcggtccggcaaacaccg
ccaaatttacctga
(amino acids)
(SEQ ID NO: 49)
MNHSERFVFIAEWYDPNASLLRRYELLFYPGDGSVEMHDVKNHRTFLKRT
KYDNLHLEDLFIGNKVNVFSRQLVLIDYGDQYTARQLGSRKEKTLALIKP
DAISKAGEIIEIINKAGFTITKLKMMMLSRKEALDFHVDHQSRPFFNELI
QFITTGPIIAMEILRDDAICEWKRLLGPANSGVARTDASESIRALFGTDG
IRNAAHGPDSFASAAREMELFFPSSGGCGPANTAKFT-

Human NME7-B sequence optimized for E. coli expression:

(DNA)
(SEQ ID NO: 50)
atgaattgtacgtgctgtattgtcaaaccgcacgcagtgtcagaaggcct
gctgggtaaaattctgatggcaatccgtgatgctggctttgaaatctcgg
ccatgcagatgttcaacatggaccgcgttaacgtcgaagaattctacgaa
gtttacaaaggcgtggttaccgaatatcacgatatggttacggaaatgta
ctccggtccgtgcgtcgcgatggaaattcagcaaaacaatgccaccaaaa
cgtttcgtgaattctgtggtccggcagatccggaaatcgcacgtcatctg
cgtccgggtaccctgcgcgcaatttttggtaaaacgaaaatccagaacgc
tgtgcactgtaccgatctgccggaagacggtctgctggaagttcaatact
ttttctga
(amino acids)
(SEQ ID NO: 51)
MNCTCCIVKPHAVSEGLLGKILMAIRDAGFEISAMQMFNMDRVNVEEFYE
VYKGVVTEYHDMVTEMYSGPCVAMEIQQNNATKTFREFCGPADPEIARHL
RPGTLRAIFGKTKIQNAVHCTDLPEDGLLEVQYFF-

Human NME7-B1 sequence optimized for E. coli expression:

(DNA)
(SEQ ID NO: 52)
atgaattgtacgtgctgtattgtcaaaccgcacgcagtgtcagaaggcct
gctgggtaaaattctgatggcaatccgtgatgctggctttgaaatctcgg
ccatgcagatgttcaacatggaccgcgttaacgtcgaagaattctacgaa
gtttacaaaggcgtggttaccgaatatcacgatatggttacggaaatgta
ctccggtccgtgcgtcgcgatggaaattcagcaaaacaatgccaccaaaa
cgtttcgtgaattctgtggtccggcagatccggaaatcgcacgtcatctg
cgtccgggtaccctgcgcgcaatttttggtaaaacgaaaatccagaacgc
tgtgcactgtaccgatctgccggaagacggtctgctggaagttcaatact
ttttcaaaattctggataattga
(amino acids)
(SEQ ID NO: 53)
MNCTCCIVKPHAVSEGLLGKILMAIRDAGFEISAMQMFNMDRVNVEEFYE
VYKGVVTEYHDMVTEMYSGPCVAMEIQQNNATKTFREFCGPADPEIARHL
RPGTLRAIFGKTKIQNAVHCTDLPEDGLLEVQYFFKILDN-

Human NME7-B2 sequence optimized for E. coli expression:

(DNA)
(SEQ ID NO: 54)
atgccgagctctggcggttgcggtccggcaaacaccgccaaatttaccaa
ttgtacgtgctgtattgtcaaaccgcacgcagtgtcagaaggcctgctgg
gtaaaattctgatggcaatccgtgatgctggctttgaaatctcggccatg
cagatgttcaacatggaccgcgttaacgtcgaagaattctacgaagttta
caaaggcgtggttaccgaatatcacgatatggttacggaaatgtactccg
gtccgtgcgtcgcgatggaaattcagcaaaacaatgccaccaaaacgttt
cgtgaattctgtggtccggcagatccggaaatcgcacgtcatctgcgtcc
gggtaccctgcgcgcaatttttggtaaaacgaaaatccagaacgctgtgc
actgtaccgatctgccggaagacggtctgctggaagttcaatactttttc
tga
(amino acids)
(SEQ ID NO: 55)
MPSSGGCGPANTAKFTNCTCCIVKPHAVSEGLLGKILMAIRDAGFEISAM
QMFNMDRVNVEEFYEVYKGVVTEYHDMVTEMYSGPCVAMEIQQNNATKTF
REFCGPADPEIARHLRPGTLRAIFGKTKIQNAVHCTDLPEDGLLEVQYF
F-

Human NME7-B3 sequence optimized for E. coli expression:

(DNA)
(SEQ ID NO: 56)
atgccgagctctggcggttgcggtccggcaaacaccgccaaatttaccaa
ttgtacgtgctgtattgtcaaaccgcacgcagtgtcagaaggcctgctgg
gtaaaattctgatggcaatccgtgatgctggctttgaaatctcggccatg
cagatgttcaacatggaccgcgttaacgtcgaagaattctacgaagttta
caaaggcgtggttaccgaatatcacgatatggttacggaaatgtactccg
gtccgtgcgtcgcgatggaaattcagcaaaacaatgccaccaaaacgttt
cgtgaattctgtggtccggcagatccggaaatcgcacgtcatctgcgtcc
gggtaccctgcgcgcaatttttggtaaaacgaaaatccagaacgctgtgc
actgtaccgatctgccggaagacggtctgctggaagttcaatactttttc
aaaattctggataattga
(amino acids)
(SEQ ID NO: 57)
MPSSGGCGPANTAKFTNCTCCIVKPHAVSEGLLGKILMAIRDAGFEISAM
QMFNMDRVNVEEFYEVYKGVVTEYHDMVTEMYSGPCVAMEIQQNNATKTF
REFCGPADPEIARHLRPGTLRAIFGKTKIQNAVHCTDLPEDGLLEVQYFF
KILDN-

Human NME7-AB sequence optimized for E. coli expression:

(DNA)
(SEQ ID NO: 58)
atggaaaaaacgctggccctgattaaaccggatgcaatctccaaagctgg
cgaaattatcgaaattatcaacaaagcgggtttcaccatcacgaaactga
aaatgatgatgctgagccgtaaagaagccctggattttcatgtcgaccac
cagtctcgcccgtttttcaatgaactgattcaattcatcaccacgggtcc
gattatcgcaatggaaattctgcgtgatgacgctatctgcgaatggaaac
gcctgctgggcccggcaaactcaggtgttgcgcgtaccgatgccagtgaa
tccattcgcgctctgtttggcaccgatggtatccgtaatgcagcacatgg
tccggactcattcgcatcggcagctcgtgaaatggaactgtttttcccga
gctctggcggttgcggtccggcaaacaccgccaaatttaccaattgtacg
tgctgtattgtcaaaccgcacgcagtgtcagaaggcctgctgggtaaaat
tctgatggcaatccgtgatgctggctttgaaatctcggccatgcagatgt
tcaacatggaccgcgttaacgtcgaagaattctacgaagtttacaaaggc
gtggttaccgaatatcacgatatggttacggaaatgtactccggtccgtg
cgtcgcgatggaaattcagcaaaacaatgccaccaaaacgtttcgtgaat
tctgtggtccggcagatccggaaatcgcacgtcatctgcgtccgggtacc
ctgcgcgcaatttttggtaaaacgaaaatccagaacgctgtgcactgtac
cgatctgccggaagacggtctgctggaagttcaatactttttcaaaattc
tggataattga
(amino acids)
(SEQ ID NO: 59)
MEKTLALIKPDAISKAGEIIEIINKAGFTITKLKMMMLSRKEALDFHVDH
QSRPFFNELIQFITTGPIIAMEILRDDAICEWKRLLGPANSGVARTDASE
SIRALFGTDGIRNAAHGPDSFASAAREMELFFPSSGGCGPANTAKFTNCT
CCIVKPHAVSEGLLGKILMAIRDAGFEISAMQMFNMDRVNVEEFYEVYKG
VVTEYHDMVTEMYSGPCVAMEIQQNNATKTFREFCGPADPEIARHLRPGT
LRAIFGKTKIQNAVHCTDLPEDGLLEVQYFFKILDN-

Human NME7-ABI sequence optimized for E. coli expression:

(DNA)
(SEQ ID NO: 60)
Atggaaaaaacgctggccctgattaaaccggatgcaatctccaaagctgg
cgaaattatcgaaattatcaacaaagcgggtttcaccatcacgaaactga
aaatgatgatgctgagccgtaaagaagccctggattttcatgtcgaccac
cagtctcgcccgtttttcaatgaactgattcaattcatcaccacgggtcc
gattatcgcaatggaaattctgcgtgatgacgctatctgcgaatggaaac
gcctgctgggcccggcaaactcaggtgttgcgcgtaccgatgccagtgaa
tccattcgcgctctgtttggcaccgatggtatccgtaatgcagcacatgg
tccggactcattcgcatcggcagctcgtgaaatggaactgtttttcccga
gctctggcggttgcggtccggcaaacaccgccaaatttaccaattgtacg
tgctgtattgtcaaaccgcacgcagtgtcagaaggcctgctgggtaaaat
tctgatggcaatccgtgatgctggctttgaaatctcggccatgcagatgt
tcaacatggaccgcgttaacgtcgaagaattctacgaagtttacaaaggc
gtggttaccgaatatcacgatatggttacggaaatgtactccggtccgtg
cgtcgcgatggaaattcagcaaaacaatgccaccaaaacgtttcgtgaat
tctgtggtccggcagatccggaaatcgcacgtcatctgcgtccgggtacc
ctgcgcgcaatttttggtaaaacgaaaatccagaacgctgtgcactgtac
cgatctgccggaagacggtctgctggaagttcaatactttttctga
(amino acids)
(SEQ ID NO: 61)
MEKTLALIKPDAISKAGEIIEIINKAGFTITKLKMMMLSRKEALDFHVDH
QSRPFFNELIQFITTGPIIAMEILRDDAICEWKRLLGPANSGVARTDASE
SIRALFGTDGIRNAAHGPDSFASAAREMELFFPSSGGCGPANTAKFTNCT
CCIVKPHAVSEGLLGKILMAIRDAGFEISAMQMFNMDRVNVEEFYEVYKG
VVTEYHDMVTEMYSGPCVAMEIQQNNATKTFREFCGPADPEIARHLRPGT
LRAIFGKTKIQNAVHCTDLPEDGLLEVQYFF-

Mouse NME6

(DNA)
(SEQ ID NO: 62)
Atgacctccatcttgcgaagtccccaagctcttcagctcacactagccct
gatcaagcctgatgcagttgcccacccactgatcctggaggctgttcatc
agcagattctgagcaacaagttcctcattgtacgaacgagggaactgcag
tggaagctggaggactgccggaggttttaccgagagcatgaagggcgttt
tttctatcagcggctggtggagttcatgacaagtgggccaatccgagcct
atatccttgcccacaaagatgccatccaactttggaggacactgatggga
cccaccagagtatttcgagcacgctatatagccccagattcaattcgtgg
aagtttgggcctcactgacacccgaaatactacccatggctcagactccg
tggtttccgccagcagagagattgcagccttcttccctgacttcagtgaa
cagcgctggtatgaggaggaggaaccccagctgcggtgtggtcctgtgca
ctacagtccagaggaaggtatccactgtgcagctgaaacaggaggccaca
aacaacctaacaaaacctag
(amino acids)
(SEQ ID NO: 63)
MTSILRSPQALQLTLALIKPDAVAHPLILEAVHQQILSNKFLIVRTRELQ
WKLEDCRRFYREHEGRFFYQRLVEFMTSGPIRAYILAHKDAIQLWRTLMG
PTRVFRARYIAPDSIRGSLGLTDTRNTTHGSDSVVSASREIAAFFPDFSE
QRWYEEEEPQLRCGPVHYSPEEGIHCAAETGGHKQPNKT-

Human NME6:

(DNA)
(SEQ ID NO: 64)
Atgacccagaatctggggagtgagatggcctcaatcttgcgaagccctca
ggctctccagctcactctagccctgatcaagcctgacgcagtcgcccatc
cactgattctggaggctgttcatcagcagattctaagcaacaagttcctg
attgtacgaatgagagaactactgtggagaaaggaagattgccagaggtt
ttaccgagagcatgaagggcgttttttctatcagaggctggtggagttca
tggccagcgggccaatccgagcctacatccttgcccacaaggatgccatc
cagctctggaggacgctcatgggacccaccagagtgttccgagcacgcca
tgtggccccagattctatccgtgggagtttcggcctcactgacacccgca
acaccacccatggttcggactctgtggtttcagccagcagagagattgca
gccttcttccctgacttcagtgaacagcgctggtatgaggaggaagagcc
ccagttgcgctgtggccctgtgtgctatagcccagagggaggtgtccact
atgtagctggaacaggaggcctaggaccagcctga
(amino acids)
(SEQ ID NO: 65)
MTQNLGSEMASILRSPQALQLTLALIKPDAVAHPLILEAVHQQILSNKFL
IVRMRELLWRKEDCQRFYREHEGRFFYQRLVEFMASGPIRAYILAHKDAI
QLWRTLMGPTRVFRARHVAPDSIRGSFGLTDTRNTTHGSDSVVSASREIA
AFFPDFSEQRWYEEEEPQLRCGPVCYSPEGGVHYVAGTGGLGPA-

Human NME6 1:

(DNA)
(SEQ ID NO: 66)
Atgacccagaatctggggagtgagatggcctcaatcttgcgaagccctca
ggctctccagctcactctagccctgatcaagcctgacgcagtcgcccatc
cactgattctggaggctgttcatcagcagattctaagcaacaagttcctg
attgtacgaatgagagaactactgtggagaaaggaagattgccagaggtt
ttaccgagagcatgaagggcgttttttctatcagaggctggtggagttca
tggccagcgggccaatccgagcctacatccttgcccacaaggatgccatc
cagctctggaggacgctcatgggacccaccagagtgttccgagcacgcca
tgtggccccagattctatccgtgggagtttcggcctcactgacacccgca
acaccacccatggttcggactctgtggtttcagccagcagagagattgca
gccttcttccctgacttcagtgaacagcgctggtatgaggaggaagagcc
ccagttgcgctgtggccctgtgtga
(amino acids)
(SEQ ID NO: 67)
MTQNLGSEMASILRSPQALQLTLALIKPDAVAHPLILEAVHQQILSNKFL
IVRMRELLWRKEDCQRFYREHEGRFFYQRLVEFMASGPIRAYILAHKDAI
QLWRTLMGPTRVFRARHVAPDSIRGSFGLTDTRNTTHGSDSVVSASREIA
AFFPDFSEQRWYEEEEPQLRCGPV-

Human NME6 2:

(DNA)
(SEQ ID NO: 68)
Atgctcactctagccctgatcaagcctgacgcagtcgcccatccactgat
tctggaggctgttcatcagcagattctaagcaacaagttcctgattgtac
gaatgagagaactactgtggagaaaggaagattgccagaggttttaccga
gagcatgaagggcgttttttctatcagaggctggtggagttcatggccag
cgggccaatccgagcctacatccttgcccacaaggatgccatccagctct
ggaggacgctcatgggacccaccagagtgttccgagcacgccatgtggcc
ccagattctatccgtgggagtttcggcctcactgacacccgcaacaccac
ccatggttcggactctgtggtttcagccagcagagagattgcagccttct
tccctgacttcagtgaacagcgctggtatgaggaggaagagccccagttg
cgctgtggccctgtgtga
(amino acids)
(SEQ ID NO: 69)
MLTLALIKPDAVAHPLILEAVHQQILSNKFLIVRMRELLWRKEDCQRFYR
EHEGRFFYQRLVEFMASGPIRAYILAHKDAIQLWRTLMGPTRVFRARHVA
PDSIRGSFGLTDTRNTTHGSDSVVSASREIAAFFPDFSEQRWYEEEEPQL
RCGPV-

Human NME6 3:

(DNA)
(SEQ ID NO: 70)
Atgctcactctagccctgatcaagcctgacgcagtcgcccatccactgat
tctggaggctgttcatcagcagattctaagcaacaagttcctgattgtac
gaatgagagaactactgtggagaaaggaagattgccagaggttttaccga
gagcatgaagggcgttttttctatcagaggctggtggagttcatggccag
cgggccaatccgagcctacatccttgcccacaaggatgccatccagctct
ggaggacgctcatgggacccaccagagtgttccgagcacgccatgtggcc
ccagattctatccgtgggagtttcggcctcactgacacccgcaacaccac
ccatggttcggactctgtggtttcagccagcagagagattgcagccttct
tccctgacttcagtgaacagcgctggtatgaggaggaagagccccagttg
cgctgtggccctgtgtgctatagcccagagggaggtgtccactatgtagc
tggaacaggaggcctaggaccagcctga
(amino acids)
(SEQ ID NO: 71)
MLTLALIKPDAVAHPLILEAVHQQILSNKFLIVRMRELLWRKEDCQRFYR
EHEGRFFYQRLVEFMASGPIRAYILAHKDAIQLWRTLMGPTRVFRARHVA
PDSIRGSFGLTDTRNTTHGSDSVVSASREIAAFFPDFSEQRWYEEEEPQL
RCGPVCYSPEGGVHYVAGTGGLGPA-

Human NME6 sequence optimized for E. coli expression:

(DNA)
(SEQ ID NO: 72)
Atgacgcaaaatctgggctcggaaatggcaagtatcctgcgctccccgca
agcactgcaactgaccctggctctgatcaaaccggacgctgttgctcatc
cgctgattctggaagcggtccaccagcaaattctgagcaacaaatttctg
atcgtgcgtatgcgcgaactgctgtggcgtaaagaagattgccagcgttt
ttatcgcgaacatgaaggccgtttcttttatcaacgcctggttgaattca
tggcctctggtccgattcgcgcatatatcctggctcacaaagatgcgatt
cagctgtggcgtaccctgatgggtccgacgcgcgtctttcgtgcacgtca
tgtggcaccggactcaatccgtggctcgttcggtctgaccgatacgcgca
ataccacgcacggtagcgactctgttgttagtgcgtcccgtgaaatcgcg
gcctttttcccggacttctccgaacagcgttggtacgaagaagaagaacc
gcaactgcgctgtggcccggtctgttattctccggaaggtggtgtccatt
atgtggcgggcacgggtggtctgggtccggcatga
(amino acids)
(SEQ ID NO: 73)
MTQNLGSEMASILRSPQALQLTLALIKPDAVAHPLILEAVHQQILSNKFL
IVRMRELLWRKEDCQRFYREHEGRFFYQRLVEFMASGPIRAYILAHKDAI
QLWRTLMGPTRVFRARHVAPDSIRGSFGLTDTRNTTHGSDSVVSASREIA
AFFPDFSEQRWYEEEEPQLRCGPVCYSPEGGVHYVAGTGGLGPA-

Human NME6 1 sequence optimized for E. coli expression:

(DNA)
(SEQ ID NO: 74)
Atgacgcaaaatctgggctcggaaatggcaagtatcctgcgctccccgca
agcactgcaactgaccctggctctgatcaaaccggacgctgttgctcatc
cgctgattctggaagcggtccaccagcaaattctgagcaacaaatttctg
atcgtgcgtatgcgcgaactgctgtggcgtaaagaagattgccagcgttt
ttatcgcgaacatgaaggccgtttcttttatcaacgcctggttgaattca
tggcctctggtccgattcgcgcatatatcctggctcacaaagatgcgatt
cagctgtggcgtaccctgatgggtccgacgcgcgtctttcgtgcacgtca
tgtggcaccggactcaatccgtggctcgttcggtctgaccgatacgcgca
ataccacgcacggtagcgactctgttgttagtgcgtcccgtgaaatcgcg
gcctttttcccggacttctccgaacagcgttggtacgaagaagaagaacc
gcaactgcgctgtggcccggtctga
(amino acids)
(SEQ ID NO: 75)
MTQNLGSEMASILRSPQALQLTLALIKPDAVAHPLILEAVHQQILSNKFL
IVRMRELLWRKEDCQRFYREHEGRFFYQRLVEFMASGPIRAYILAHKDAI
QLWRTLMGPTRVFRARHVAPDSIRGSFGLTDTRNTTHGSDSVVSASREIA
AFFPDFSEQRWYEEEEPQLRCGPV-

Human NME6 2 sequence optimized for E. coli expression:

(DNA)
(SEQ ID NO: 76)
Atgctgaccctggctctgatcaaaccggacgctgttgctcatccgctgat
tctggaagcggtccaccagcaaattctgagcaacaaatttctgatcgtgc
gtatgcgcgaactgctgtggcgtaaagaagattgccagcgtttttatcgc
gaacatgaaggccgtttcttttatcaacgcctggttgaattcatggcctc
tggtccgattcgcgcatatatcctggctcacaaagatgcgattcagctgt
ggcgtaccctgatgggtccgacgcgcgtctttcgtgcacgtcatgtggca
ccggactcaatccgtggctcgttcggtctgaccgatacgcgcaataccac
gcacggtagcgactctgttgttagtgcgtcccgtgaaatcgcggcctttt
tcccggacttctccgaacagcgttggtacgaagaagaagaaccgcaactg
cgctgtggcccggtctga
(amino acids)
(SEQ ID NO: 77)
MLTLALIKPDAVAHPLILEAVHQQILSNKFLIVRMRELLWRKEDCQRFYR
EHEGRFFYQRLVEFMASGPIRAYILAHKDAIQLWRTLMGPTRVFRARHVA
PDSIRGSFGLTDTRNTTHGSDSVVSASREIAAFFPDFSEQRWYEEEEPQL
RCGPV-

Human NME6 3 sequence optimized for E. coli expression:

(DNA)
(SEQ ID NO: 78)
Atgctgaccctggctctgatcaaaccggacgctgttgctcatccgctgat
tctggaagcggtccaccagcaaattctgagcaacaaatttctgatcgtgc
gtatgcgcgaactgctgtggcgtaaagaagattgccagcgtttttatcgc
gaacatgaaggccgtttcttttatcaacgcctggttgaattcatggcctc
tggtccgattcgcgcatatatcctggctcacaaagatgcgattcagctgt
ggcgtaccctgatgggtccgacgcgcgtctttcgtgcacgtcatgtggca
ccggactcaatccgtggctcgttcggtctgaccgatacgcgcaataccac
gcacggtagcgactctgttgttagtgcgtcccgtgaaatcgcggcctttt
tcccggacttctccgaacagcgttggtacgaagaagaagaaccgcaactg
cgctgtggcccggtctgttattctccggaaggtggtgtccattatgtggc
gggcacgggtggtctgggtccggcatga
(amino acids)
(SEQ ID NO: 79)
MLTLALIKPDAVAHPLILEAVHQQILSNKFLIVRMRELLWRKEDCQRFYR
EHEGRFFYQRLVEFMASGPIRAYILAHKDAIQLWRTLMGPTRVFRARHVA
PDSIRGSFGLTDTRNTTHGSDSVVSASREIAAFFPDFSEQRWYEEEEPQL
RCGPVCYSPEGGVHYVAGTGGLGPA-

OriGene-NME7-1 full length

(DNA)
(SEQ ID NO: 80)
gacgttgtatacgactcctatagggcggccgggaattcgtcgactggatc
cggtaccgaggagatctgccgccgcgatcgccatgaatcatagtgaaaga
ttcgttttcattgcagagtggtatgatccaaatgcttcacttcttcgacg
ttatgagcttttattttacccaggggatggatctgttgaaatgcatgatg
taaagaatcatcgcacctttttaaagcggaccaaatatgataacctgcac
ttggaagatttatttataggcaacaaagtgaatgtcttctctcgacaact
ggtattaattgactatggggatcaatatacagctcgccagctgggcagta
ggaaagaaaaaacgctagccctaattaaaccagatgcaatatcaaaggct
ggagaaataattgaaataataaacaaagctggatttactataaccaaact
caaaatgatgatgctttcaaggaaagaagcattggattttcatgtagatc
accagtcaagaccctttttcaatgagctgatccagtttattacaactggt
cctattattgccatggagattttaagagatgatgctatatgtgaatggaa
aagactgctgggacctgcaaactctggagtggcacgcacagatgcttctg
aaagcattagagccctctttggaacagatggcataagaaatgcagcgcat
ggccctgattcttttgcttctgcggccagagaaatggagttgttttttcc
ttcaagtggaggttgtgggccggcaaacactgctaaatttactaattgta
cctgttgcattgttaaaccccatgctgtcagtgaaggactgttgggaaag
atcctgatggctatccgagatgcaggttttgaaatctcagctatgcagat
gttcaatatggatcgggttaatgttgaggaattctatgaagtttataaag
gagtagtgaccgaatatcatgacatggtgacagaaatgtattctggccct
tgtgtagcaatggagattcaacagaataatgctacaaagacatttcgaga
attttgtggacctgctgatcctgaaattgcccggcatttacgccctggaa
ctctcagagcaatctttggtaaaactaagatccagaatgctgttcactgt
actgatctgccagaggatggcctattagaggttcaatacttcttcaagat
cttggataatacgcgtacgcggccgctcgagcagaaactcatctcagaag
aggatctggcagcaaatgatatcctggattacaaggatgacgacgataag
gtttaa
(amino acids)
(SEQ ID NO: 81)
MNHSERFVFIAEWYDPNASLLRRYELLFYPGDGSVEMHDVKNHRTFLKRT
KYDNLHLEDLFIGNKVNVFSRQLVLIDYGDQYTARQLGSRKEKTLALIKP
DAISKAGEIIEIINKAGFTITKLKMMMLSRKEALDFHVDHQSRPFFNELI
QFITTGPIIAMEILRDDAICEWKRLLGPANSGVARTDASESIRALFGTDG
IRNAAHGPDSFASAAREMELFFPSSGGCGPANTAKFTNCTCCIVKPHAVS
EGLLGKILMAIRDAGFEISAMQMFNMDRVNVEEFYEVYKGVVTEYHDMVT
EMYSGPCVAMEIQQNNATKTFREFCGPADPEIARHLRPGTLRAIFGKTKI
QNAVHCTDLPEDGLLEVQYFFKILDNTRTRRLEQKLISEEDLAANDILDY
KDDDDKV

Abnova NME7-1 Full length

(amino acids)
(SEQ ID NO: 82)
MNHSERFVFIAEWYDPNASLLRRYELLFYPGDGSVEMHDVKNHRTFLKRT
KYDNLHLEDLFIGNKVNVFSRQLVLIDYGDQYTARQLGSRKEKTLALIKP
DAISKAGEIIEIINKAGFTITKLKMMMLSRKEALDFHVDHQSRPFFNELI
QFITTGPIIAMEILRDDAICEWKRLLGPANSGVARTDASESIRALFGTDG
IRNAAHGPDSFASAAREMELFFPSSGGCGPANTAKFTNCTCCIVKPHAVS
EGLLGKILMAIRDAGFEISAMQMFNMDRVNVEEFYEVYKGVVTEYHDMVT
EMYSGPCVAMEIQQNNATKTFREFCGPADPEIARHLRPGTLRAIFGKTKI
QNAVHCTDLPEDGLLEVQYFFKILDN

Abnova Partial NME7-B

(amino acids)
(SEQ ID NO: 83)
DRVNVEEFYEVYKGVVTEYHDMVTEMYSGPCVAMEIQQNNATKTFREFCG
PADPEIARHLRPGTLRAIFGKTKIQNAVHCTDLPEDGLLEVQYFFKIL

Histidine Tag

(SEQ ID NO: 84)
(ctcgag)caccaccaccaccaccactga

Strept II Tag

(SEQ ID NO: 85)
(accggt)tggagccatcctcagttcgaaaagtaatga

N-10 peptide:

(SEQ ID NO: 86)
QFNQYKTEAASRYNLTISDVSVSDVPFPFSAQSGA

C-10 peptide

(SEQ ID NO: 87)
GTINVHDVETQFNQYKTEAASRYNLTISDVSVSDV

Immunizing peptides derived from human NME7:

(SEQ ID NO: 88)

LALIKPDA

(SEQ ID NO: 89)

MMMLSRKEALDFHVDHQS

(SEQ ID NO: 90)

ALDFHVDHQS

(SEQ ID NO: 91)

EILRDDAICEWKRL

(SEQ ID NO: 92)

FNELIQFITTGP

(SEQ ID NO: 93)

RDDAICEW

(SEQ ID NO: 94)

SGVARTDASESIRALFGTDGIRNAA

(SEQ ID NO: 95)

ELFFPSSGG

(SEQ ID NO: 96)

KFTNCTCCIVKPHAVSEGLLGKILMA

(SEQ ID NO: 97)

LMAIRDAGFEISAMQMFNMDRVNVEEFYEVYKGVVT

(SEQ ID NO: 98)

EFYEVYKGVVTEYHD

(SEQ ID NO: 99)

EIQQNNATKTFREFCGPADPEIARHLRPGTLRAIFGKTKIQNA

(SEQ ID NO: 100)

YSGPCVAM

(SEQ ID NO: 101)

FREFCGP

(SEQ ID NO: 102)

VHCTDLPEDGLLEVQYFFKILDN

(SEQ ID NO: 103)

IQNAVHCTD

(SEQ ID NO: 104)

TDLPEDGLLEVQYFFKILDN

(SEQ ID NO: 105)

PEDGLLEVQYFFK

(SEQ ID NO: 106)

EIINKAGFTITK

(SEQ ID NO: 107)

MLSRKEALDFHVDHQS

(SEQ ID NO: 108)

NELIQFITT

(SEQ ID NO: 109)

EILRDDAICEWKRL

(SEQ ID NO: 110)

SGVARTDASESIRALFGTDGI

(SEQ ID NO: 111)

SGVARTDASES

(SEQ ID NO: 112)

ALFGTDGI

(SEQ ID NO: 113)

NCTCCIVKPHAVSE

(SEQ ID NO: 114)

LGKILMAIRDA

(SEQ ID NO: 115)

EISAMQMFNMDRVNVE

(SEQ ID NO: 116)

EVYKGVVT

(SEQ ID NO: 117)

EYHDMVTE

(SEQ ID NO: 118)

EFCGPADPEIARHLR

(SEQ ID NO: 119)

AIFGKTKIQNAV

(SEQ ID NO: 120)

LPEDGLLEVQYFFKILDN

(SEQ ID NO: 121)

GPDSFASAAREMELFFP

Immunizing peptides derived from human NME7

| (SEQ ID NO: 122) | |
|---|---|
| | ICEWKRL |
| (SEQ ID NO: 123) | |
| | LGKILMAIRDA |
| (SEQ ID NO: 124) | |
| | HAVSEGLLGK |
| (SEQ ID NO: 125) | |
| | VTEMYSGP |
| (SEQ ID NO: 126) | |
| | NATKTFREF |
| (SEQ ID NO: 127) | |
| | AIRDAGFEI |
| (SEQ ID NO: 128) | |
| | AICEWKRLLGPAN |
| (SEQ ID NO: 129) | |
| | DHQSRPFF |
| (SEQ ID NO: 130) | |
| | AICEWKRLLGPAN |
| (SEQ ID NO: 131) | |
| | VDHQSRPF |
| (SEQ ID NO: 132) | |
| | PDSFAS |
| (SEQ ID NO: 133) | |
| | KAGEIIEIINKAGFTITK |

Immunizing peptides derived from human NME1

| (SEQ ID NO: 134) | |
|---|---|
| | MANCERTFIAIKPDGVQRGLVGEIIKRFE |
| (SEQ ID NO: 135) | |
| | VDLKDRPF |
| (SEQ ID NO: 136) | |
| | HGSDSVESAEKEIGLWF |
| (SEQ ID NO: 137) | |
| | ERTFIAIKPDGVQRGLVGEIIKRFE |
| (SEQ ID NO: 138) | |
| | VDLKDRPFFAGLVKYMHSGPVVAMVWEGLN |
| (SEQ ID NO: 139) | |
| | NIIHGSDSVESAEKEIGLWFHPEELV |
| (SEQ ID NO: 140) | |
| | KPDGVQRGLVGEII |

### NME Inhibition

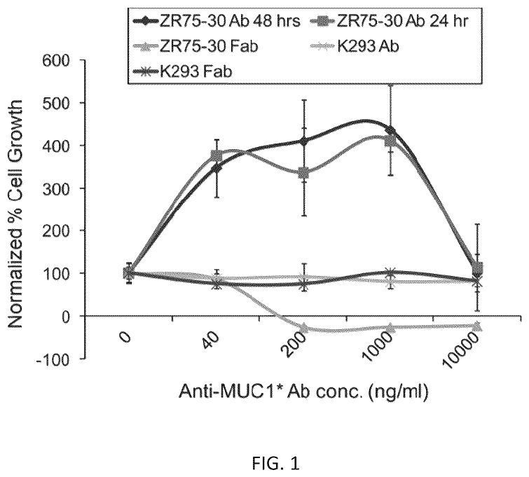

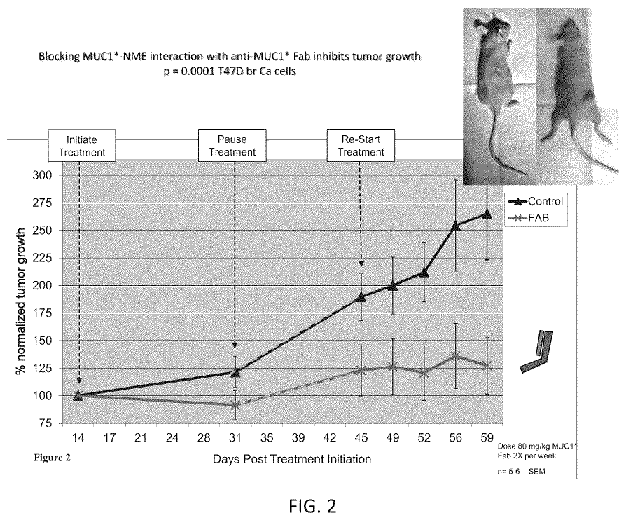

Applicants previously discovered that a key growth factor receptor, MUC1*, and its activating ligand, NM23-H1 (also called NME1) in dimer form, mediates the growth of most solid tumor cancers. We subsequently discovered that this same growth factor/growth factor receptor pair also mediates the growth of pluripotent stem cells. MUC1*, an alternative splice variant or an enzymatically cleaved form of the transmembrane protein, MUC1, is expressed on all pluripotent human stem cells (Hikita et al, 2008) and on the majority of solid tumor cancers (Mahanta et al, 2008). When stem cells differentiate, cleavage of MUC1 subsides and MUC1 reverts to its full-length quiescent form. On stem cells and cancer cells, MUC1* functions as a growth factor receptor. Ligand-induced dimerization of MUC1* promotes cancer cell growth, survival and can make cancer cells resistant to chemotherapy drugs (Fessler et al, 2009). Inhibition of ligand-induced dimerization of MUC1*'s extracellular domain greatly inhibits cancer cell growth in vitro (FIG. 1) and in vivo (FIG. 2). In stem cells, ligand-induced dimerization of MUC1* stimulates growth and survival, while inhibiting differentiation. Both stem cells and cancer cells secrete NM23-H1. In dimeric form, NM23-H1 dimerizes the extra cellular domain of MUC1* to make stem cells proliferate and to inhibit their differentiation.

NME1 in dimer form not only promotes growth and pluripotency of stem cells, but also induces human stem cells to revert to the earliest, most pluripotent state called the “naïve” state (Nichols J, Smith A (2009); Hanna et al, 2010; Amit M, et al, 2000; Ludwig T E, et al 2006; Xu C, et al, 2005; Xu R H, et al, 2005; Smagghe et al 2013). To date, this is the only natural factor that has been shown to maintain human stem cells in the naïve state, as they exist in the inner mass of the very early embryo.

Here, we report the discovery that other molecules, previously thought to be specific to stem cells, are also expressed in cancer cells. Growth factors and growth factor receptors that are expressed during embryogenesis, but are errantly expressed again in cancer cells make excellent therapeutic targets, since disabling them should not have a significantly negative effect on the patient. Thus, it would be a great improvement over the state of the art to identify stem cell growth factors and receptors that are active in embryogenesis, in embryonic stem (ES) cells or in induced pluripotent stem (iPS) cells but are errantly reactivated in cancer cells, and to develop therapeutics that disable them or cause their expression to be suppressed.

Applicants also recently discovered that many genes and gene products that are expressed in human stem cells, are also expressed in human cancers. For example MUC1*, an alternative splice variant or an enzymatically cleaved form of the transmembrane protein, MUC1*, is expressed on all pluripotent human stem cells and on the majority of solid tumor cancers. When stem cells differentiate, cleavage of MUC1 stops and MUC1 reverts to its full-length quiescent form. On stem cells and cancer cells, MUC1* functions as a growth factor receptor. Dimerization of MUC1* promotes stem and cancer cell growth; it inhibits differentiation of stem cells and causes cancer cells to revert to a less differentiated state. Both stem cells and cancer cells secrete NM23-H1 in dimeric form, and dimerizes the extra cellular domain of MUC1* to make stem cells proliferate and to inhibit their differentiation.

We have discovered that NME family members that are actively expressed in stem cells are errantly up-regulated in cancer cells. In addition to NM23-H1, other NME family proteins act as growth factors and transcription factors that promote stem and cancer cell growth and inhibit their differentiation. NME1 promotes stem cell growth and pluripotency when it is a dimer. NME1 dimers also mediate the growth and de-differentiation of MUC1-positive cancer cells. As the density of stem cells increases and more and more NME1 is secreted from the stem cells, the NME1 forms hexamers, which actually induce differentiation, thus stopping pluripotent stem cell growth. It is known that cancer cells can appear to be less differentiated than normal adult cells. In fact, the degree to which cancer cells morphologically appear to have de-differentiated correlates to the degree of cancer aggressiveness. Therefore, it is a valid therapeutic approach to treat patients with cancer, or patients who are at risk of developing cancer, with NME1 in hexamer form, which will induce differentiation of cancer cells and limit their ability to self-replicate.

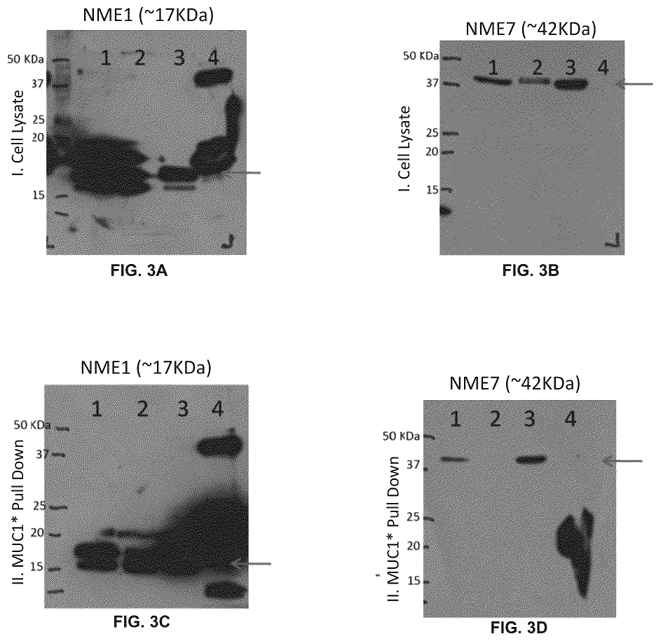

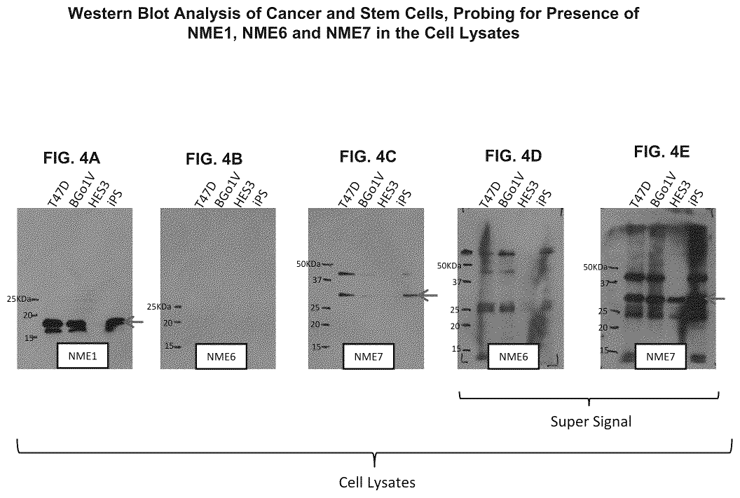

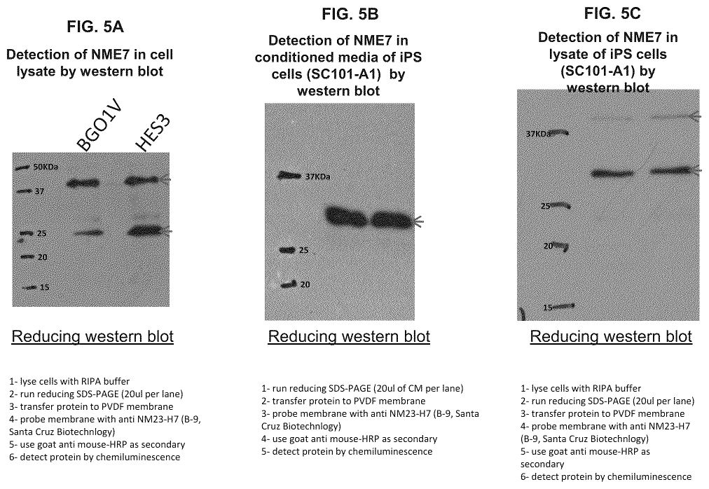

In addition to NME1, NME6 and NME7 are expressed in early stage stem cells and in cancer cells. Western blot analysis was performed on a wide variety of human stem cells and cancer cell lines, which showed that both stem cells and cancer cells express and secrete NME1, NME6 and NME7. In one such experiment, human embryonic stem cells (BGO1v) and human MUC1*-positive breast cancer cells (T47D), wherein the cell lysates were probed for the presence of NME1 (NM23-H1), NME6 (data not shown), or NME7. FIGS. 3A and 3B show that NME1 and NME7 were readily detected in the lysate of stem cells and cancer cells. NME6, ˜22 kDa, was detected in a more sensitive assay shown in FIG. 4D. A pull-down assay was performed on the stem cells and the cancer cells using an antibody that binds to the cytoplasmic domain of MUC1. The Western of FIGS. 3C and 3D show that both NME1 and NME7 bind to MUC1 as it exists in stem and cancer cells. Although NME7 is produced in both stem cells and cancer cells, we discovered that intra-cellularly, it exists as the full-length protein ˜42 kDa. However, NME7 must be cleaved before it is secreted. The secreted form appears to be devoid of its leader sequence DM10 and runs with an apparent molecular weight of ˜33 kDa. FIG. 5 is a panel of photos of Western blots of human embryonic stem (ES) cells (A) and induced pluripotent stem (iPS) cells (B, C) probed for the presence of NME7. Western blots show the presence of three forms of NME7 in the cell lysates. One with an apparent molecular weight of ˜42 kDa (full length), ˜33 kDa (NME7-AB domains devoid of the N-terminal DH domain) and a small ˜25 kDa species. However, only the lower molecular weight species are secreted in the conditioned media (C).

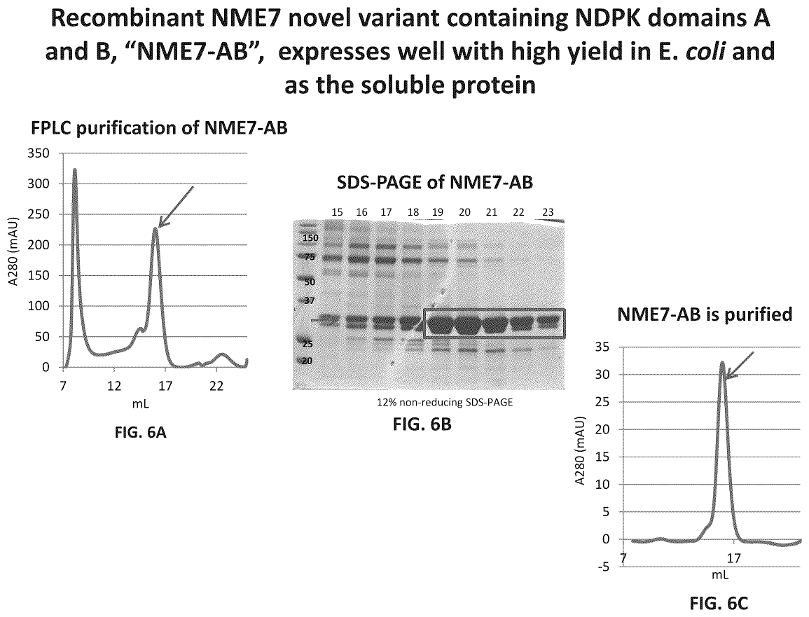

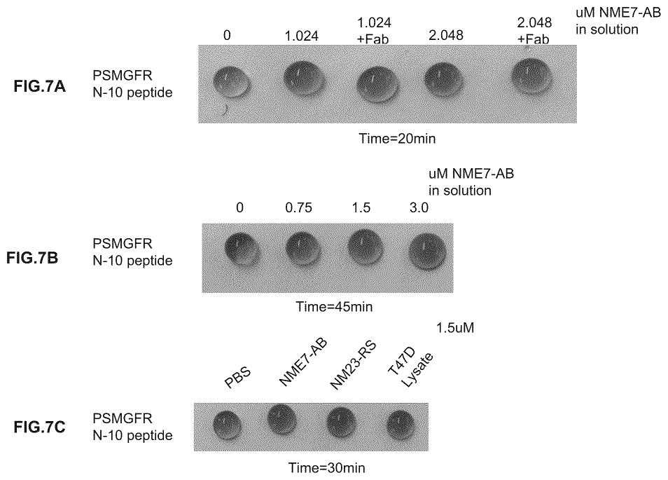

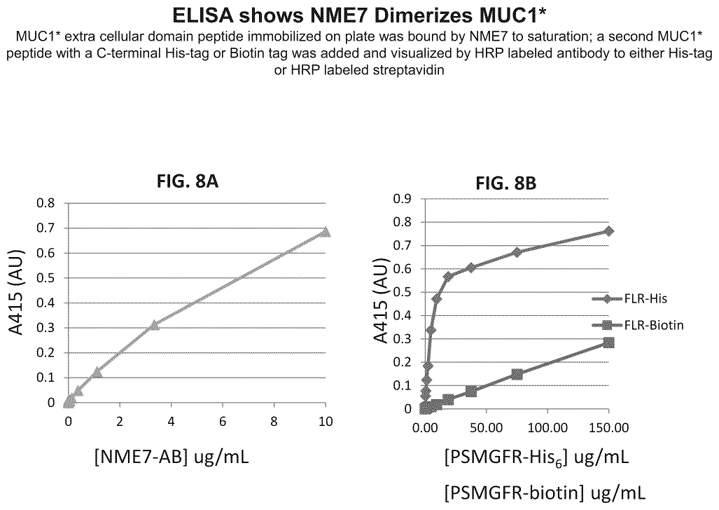

We made several constructs for expression of a human NME7. One of these constructs expressed well in E. coli, was secreted in soluble form as a monomer and functioned approximately as NME1 dimers did for promoting stem cell growth, pluripotency and inhibition of differentiation. In this construct, the leader sequence “DM10” was omitted from the sequence. This generated a species that was approximately the same molecular weight, 33 kDa, as the secreted form of the protein. The protein was made as a Histidine tagged protein and first purified over an NTA-Ni column, then by FPLC with greater than 98% purity. We call this form of NME7, NME7-AB. It is not intended that the invention be limited by the exact nature of the NME7 protein. The NME7-AB protein that we generated may simply be the minimal portion of the natural protein that is required for its stem/cancer promotion function. We have demonstrated that NME7-AB functions in a way that is essentially the same as the naturally processed NME7, as is demonstrated in the experiments and examples contained herein. However the naturally occurring cleavage site of NME7 may be different from where we started the NME7-AB N-terminus. Inhibitors of NME7 may act on the native protein that contains the DM10 at the N-terminus or may act to inhibit cleavage of NME7 to the secreted form. FIG. 6A-C shows the FPLC trace of the NME7-AB following purification by the nickel column (A), an SDS-PAGE gel of the unpurified protein (B) and an FPLC trace of the final product (C). A nanoparticle assay was performed that showed that NME7 as a monomer can simultaneously bind to two PSMGFR peptides (SEQ ID NO:6) of the MUC1* extra cellular domain. Histidine-tagged PSMGFR peptides were immobilized onto NTA-SAM-coated nanoparticles. Recombinant NME7-AB (expressed devoid of the DM10 N-terminal leader sequence), which had been verified to be monomeric by FPLC and native gel, was added to the nanoparticles. The addition of the NME7 caused the gold nanoparticle solution to turn from pink to blue indicating that the NME7 simultaneously bound to two peptides on two separate nanoparticles which caused the particles to be drawn close together, thus inducing the characteristic color change (FIG. 7). Another ELISA experiment was performed that demonstrated that NME7 monomers dimerize two MUC1* extra cellular domain peptides. A first PSMGFR peptide was coupled to BSA and immobilized on a multi-well plate. Recombinant NME7-AB was added. After the appropriate wash steps, a second PSMGFR peptide, modified with biotin was added. A labeled streptavidin was then added which clearly showed that NME7 monomers can simultaneously bind two MUC1* extra cellular domain peptides (FIG. 8). These results indicate that NME7 via its two NDPK domains binds to and dimerizes MUC1* on stem cells and cancer cells.

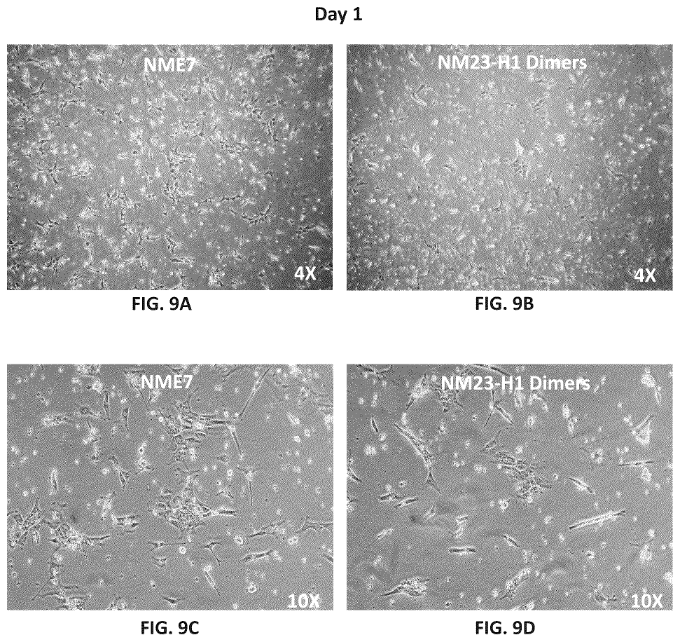

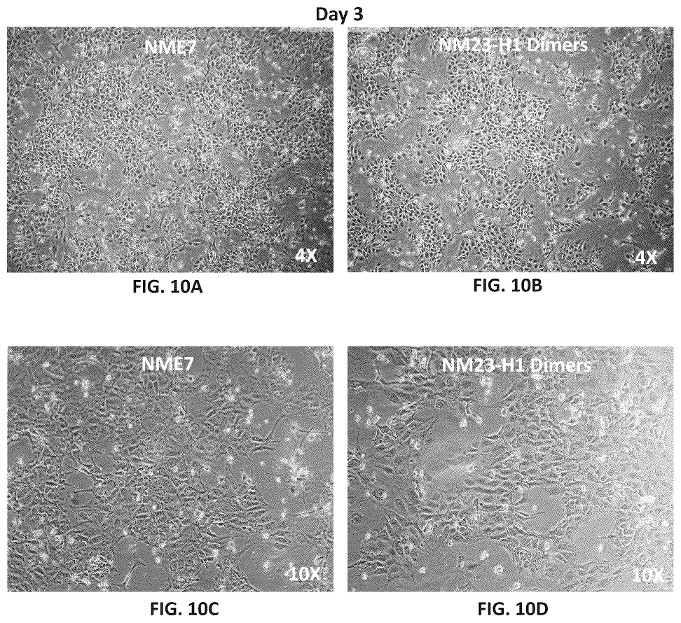

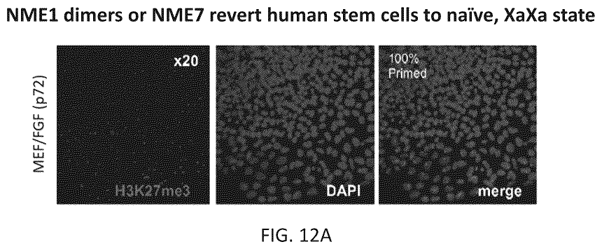

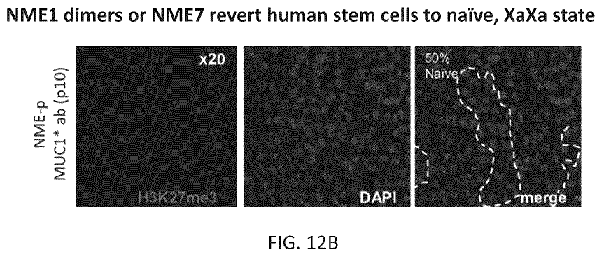

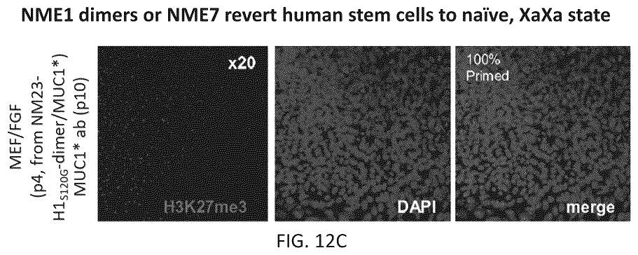

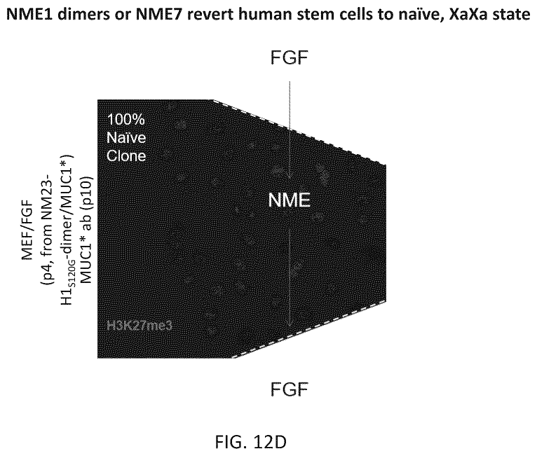

NME7 functions approximately the same as NME1 dimers. Like NME1 dimers, NME7 fully supports human stem cell growth. A panel of human stem cells (embryonic ‘ES’ and induced pluripotent ‘iPS’) were cultured in a minimal serum-free base media with either NME1 dimers or NME7-AB added as the only growth factor or cytokine. The stem cells grew faster than growth in the traditional FGF-containing media, did not spontaneously differentiate, and were reverted to the naïve state, as evidenced by having two active X chromosomes. FIG. 9 and FIG. 10 show photographs of human HES-3 embryonic stem cells that were cultured in either NME1 dimers or NME7-AB on Day 1 and Day 3 respectively. As can be clearly seen, the stem cells appear to be growing equivalently with no signs of differentiation. Note that naïve stem cells do not grow in colonies but rather grow in monolayers that become sheets as confluency is reached. FIG. 11 shows photographs of immunocytochemistry (ICC) experiments that confirm that stem cells cultured in NME7-AB for more than 10 passages stain positive for the standard pluripotency markers. FIG. 12 shows photographs of ICC experiments that confirm that stem cells cultured in NME7-AB are in the naïve state. The cells of panel (A) were cultured in FGF on mouse feeder cells as is standard practice. The staining antibody produced a red dot where it bound to condensed tri-methylated Lysine 27 on Histone 3 (H3K27me), indicating one X chromosome is inactive (XaXi) and that the stem cells have progressed to the “primed” state. The cells of panel (B) are the same cells as photographed in (A) except that they were cultured for 10 passages in NME7-AB. As can be seen in the insert, the H3K27me antibody produced the “cloud” staining pattern, indicating that both X chromosomes were active (XaXa), evidencing that the cells had reverted to the naïve state. Thus, we have demonstrated that NME7 fully supports stem cell growth and pluripotency and also reverts them to the naïve or ground state, being a less mature state than the later, primed state.

We next sought to determine whether NME7 was also an active growth factor driving the growth of cancer cells. If so, then cancer growth could be inhibited or prevented in a patient by a therapeutic agent that blocks the interaction of NME7 to MUC1* extra cellular domain. A rabbit polyclonal antibody raised against the NME7 A and B domains was added to T47D, MUC1*-positive breast cancer cells and cell growth was measured. Even at very low, nanomolar concentrations, anti-NME7 inhibited the growth of cancer cells (FIG. 13-15). In a preferred embodiment, a therapeutic agent for the treatment of cancers is an antibody that binds to the NDPK A domain of NME7. In a more preferred embodiment, the therapeutic agent is an antibody that binds to the NDPK B domain of NME7. In a still more preferred embodiment, the therapeutic agent is an antibody that binds a sequence in the A or B domain of NME7 that is not present in NME1. In a still more preferred embodiment, the therapeutic agent is an antibody that inhibits the interaction between NME7 and MUC1*. In a most preferred embodiment, the therapeutic agent is an antibody that inhibits the function of NME7 wherein said function is the promotion of cancerous growth or reversion to a cancer-like state.

Recall that one way that NME ligands function as growth factors is by binding to and dimerizing the extra cellular domain of MUC1*. NME family proteins have one or more NDPK domains. These NDPK domains have a catalytic function that is independent of, and not required for, their function as growth factors and transcription factors. NME family proteins bind to the extra cellular domain of MUC1* via their NDPK domain.

Different NME family proteins are expressed at different times during normal embryo development. NME7 is the most primitive of the NME family proteins that regulate stem cell growth and in vivo is only expressed in very early embryogenesis. NME7 is a single ˜42 kDa protein that has two NDPK domains, A and B plus an N-terminal leader sequence called the DM10 domain. ELISA assays show that NME7 binds to and dimerizes MUC1* transmembrane receptor. Since NME7 has 2 NDPK domains, it is a pseudo dimer that is always able to dimerize the MUC1* receptor.

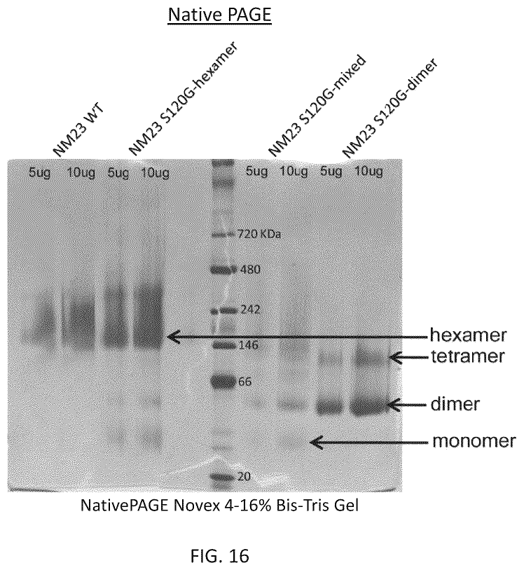

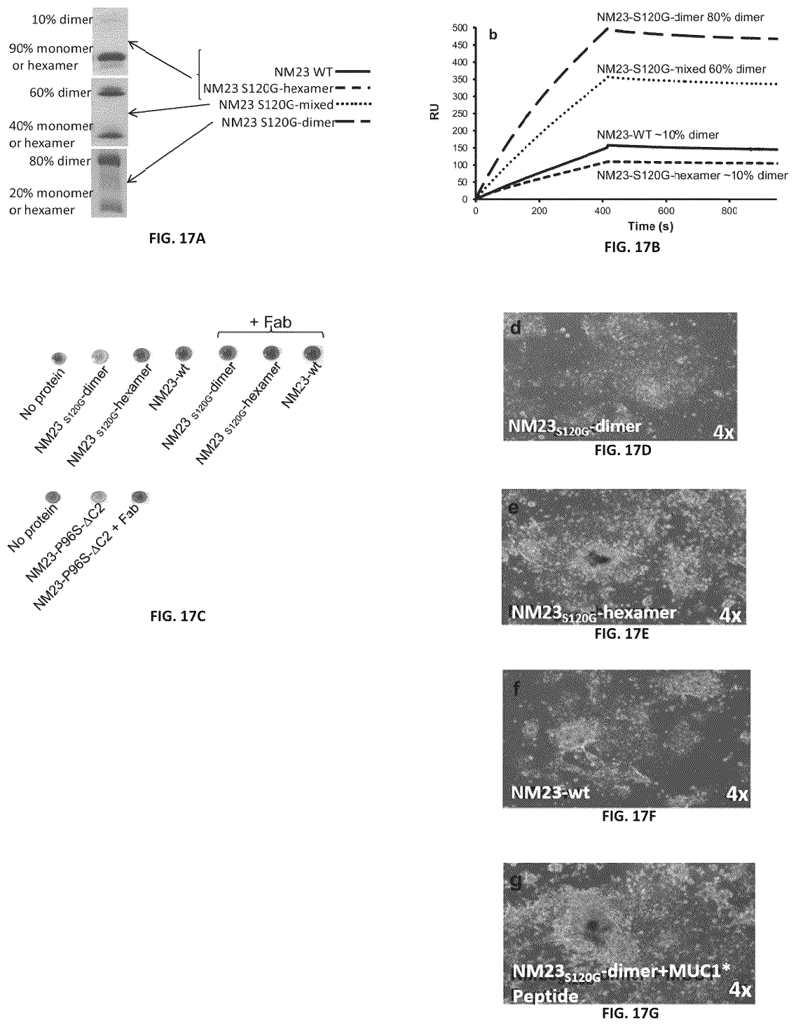

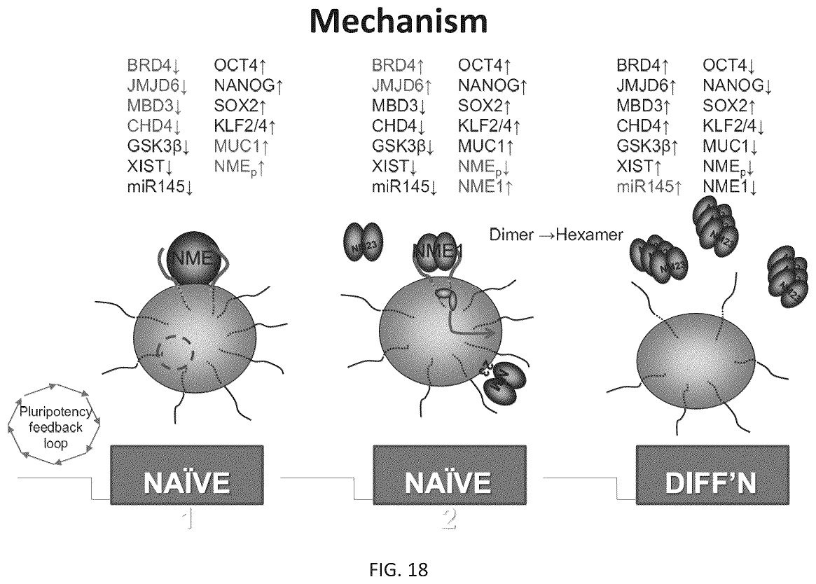

By contrast, NME1 is roughly half the molecular weight of NME7 (˜17 kDa) and has only one NDPK domain. NME1 acts as a growth factor that promotes growth and inhibits differentiation only when it is a dimer. At higher concentrations, NME1 can form hexamers. In contrast to the dimers, NME1 hexamers induce differentiation. Thus, NME1, which is expressed later in embryogenesis, has the ability to turn itself off, thus limiting self-replication, while NME7 cannot. Wild type (wt) NME1 exists primarily as a hexamer at measurable concentrations. Mutant NME1 proteins, such as S120G that form stable dimer populations have been isolated from cancers and thus are continuously activating the MUC1* receptor. We made recombinant NME1-wt, and the S120G mutant. By varying refolding protocols we were able to stabilize populations that were essentially 100% hexamer or 100% dimer. In addition we isolated populations of NME1-S120G that were a mixture of dimer, tetramer and hexamer. FIG. 16 shows these various multimers on a native gel. FIG. 17 (A) shows gels of NME1 proteins used in an SPR experiment (B) wherein the PSMGFR peptide of the MUC1* extracellular domain is immobilized on the chip and different NME1 proteins are flowed over the surface. The results show that the dimer form of NME1 is the form that binds to the MUC1* extracellular domain. Panel (C) shows a nanoparticle experiment wherein the PSMGFR peptide was attached to NTA-Ni-SAM coated nanoparticles and recombinant NME1 dimers or hexamers are added to the nanoparticles. Gold nanoparticles turn blue if the interaction takes place and remain pink if it does not. As can be seen, only the dimer binds to the MUC1* peptide on the nanoparticles and dimerizes two peptides in two different nanoparticles, essentially cross-linking the particles. The Fab of the MN-C2 anti-MUC1* antibody when added to the solution disrupts the interaction between NME1 dimers and the MUC1* PSMGFR peptide. Panel (D) shows photos of human stem cells cultured in either NME1 dimers (NM23), hexamers, or the dimers plus a free PSMGFR peptide to competitively inhibit the interaction. As can be seen in the photos, only NME1 dimers promote pluripotent stem cell growth. In nature, when the concentration of stem cells reaches critical mass and their secretions of NME1 reaches the concentration at which they form hexamers, differentiation is induced. FIG. 18 is a cartoon depicting the mechanism, supported by experiments described herein, by which NME7 and NME1 function to promote pluripotency wherein NME1 regulates itself.

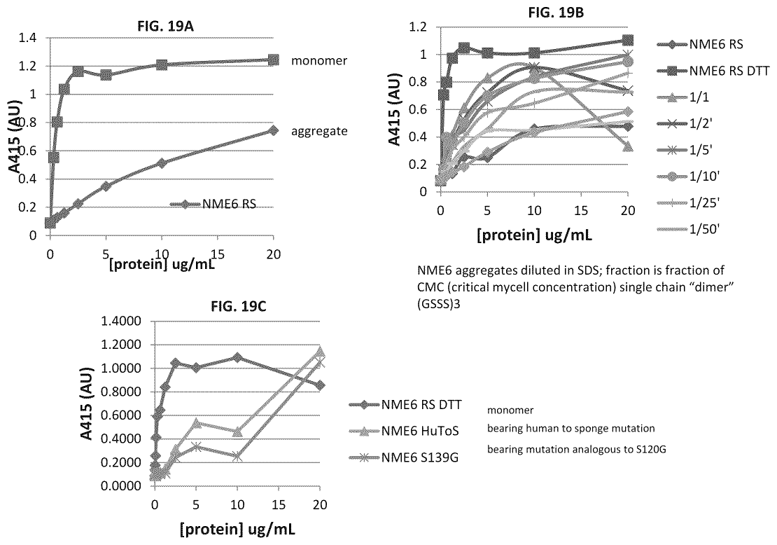

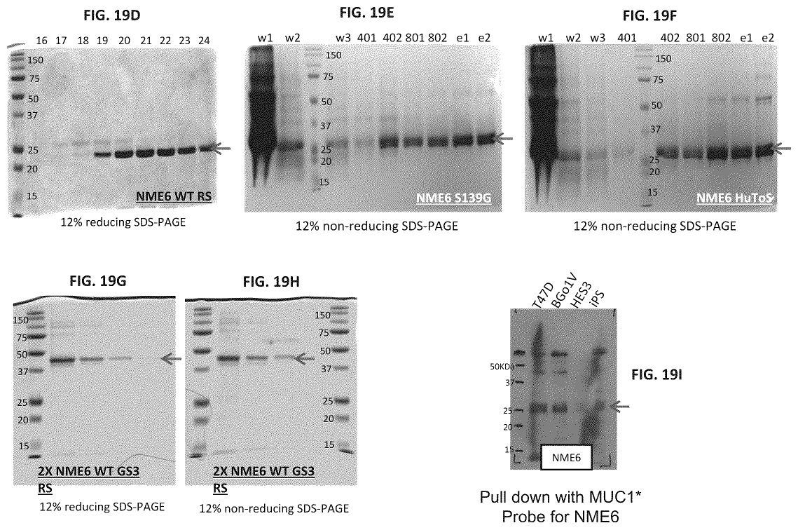

NME6 is also expressed in very early embryogenesis. NME6 is reportedly a dimer in some species such as sea sponge. NME6 must be expressed at high enough levels that it can form dimers before it can activate growth and inhibit differentiation. Thus, it is expressed at a later stage than NME7. NME6 also binds to the PSMGFR peptide of the MUC1* extra cellular domain. In a pull-down assay, NME6 was shown to bind to MUC1* in cancer cells and in stem cells. We made recombinant NME6 as the wild type protein, or with a single point mutation S139G, which mimics the S120G mutation that causes NME1 to prefer dimer formation. In addition, another NME6 variant was made so that in this sensitive area, the human NME6 would look like sea sponge NME6, which reportedly exists as a dimer. These mutations are S139A plus V142D and V143A. The ELISA assays shown in FIGS. 19A, B, and C show that NME6 binds to the PSMGFR peptide of the MUC1* extra cellular domain. In part A, NME6-wt is purified as the monomer or as a high molecular weight multimer. The ELISA assay, in which the surface is coated with the PSMGFR MUC1* peptide, shows preferential binding of the NME6 monomer to the MUC1* peptides. In part B, the NME6 multimers are dissociated by dilution in SDS. The ELISA shows that as the multimers are dissociated, binding to the MUC1* peptide increases. The figure shows that NME6-wt and the two mutants that prefer dimer formation, bind to the MUC1* peptides. The gels of FIG. 19 D-H show expression of NME6-wt (D), NME6 with the S139G mutation that corresponds to the mutation S120G which in human NME1 increases dimer formation (E), NME6 bearing three mutations that make the human form mimic the sea sponge form that is reported to be a dimer (F), and a single chain protein linking two NME6 proteins (G,H). Panel I shows that in a pull-down assay using an antibody against the cytoplasmic tail of MUC1, NME6 was shown to bind to MUC1 in cancer cells and in stem cells. Thus an effective anti-cancer agent would be an antibody, small molecule or other agent that disrupts binding of NME6 dimers to MUC1* extra cellular domain peptide. In a preferred embodiment, the therapeutic agent for the treatment of cancers is an antibody that binds to the NDPK A domain of NME6. In a more preferred embodiment, the therapeutic antibody binds to sequences of NME6 that are not present in NME1.

Human stem cells that mimic embryonic stem cells of the inner mass of the blastocyst, which are the very earliest stage stem cells, are called “naïve” state stem cells. Until recently, researchers were unable to maintain or generate genetically unmodified naïve state human stem cells in vitro. We recently succeeded in generating genetically unmodified human stem cells in the naïve state by culturing cells in NME1 dimers or in NME7 and in the absence of other growth factors or cytokines, particularly in the absence of bFGF. In addition, we showed that these naïve state stem cells progress to the more mature “primed” state as soon as they are exposed to bFGF. To demonstrate that NME7 is expressed at very high levels in very early stage stem cells, we performed Western blot analysis on human stem cells cultured in either NME1 or NME7 (naive) or cultured in bFGF (primed), then probed for the presence of NME7. Embryonic stem cells in the primed state, which is more differentiated than stem cells in the naïve state, express only trace amounts of NME7. By stark contrast, stem cells in the earlier “naïve” state (also called the “ground” state) express high levels of NME7 (FIG. 3B, compare lane 1 (naïve) to lane 2 (primed). NME7 is expressed in cancer cells to a level comparable to its expression in early stage stem cells (FIG. 3B, compare lane 3 (cancer cell) to lane 1 (naïve stem cell)). For this reason, NME7 and NME6 can be therapeutically disabled without significant side effects because their primary role is in early embryogenesis rather than in adult life.

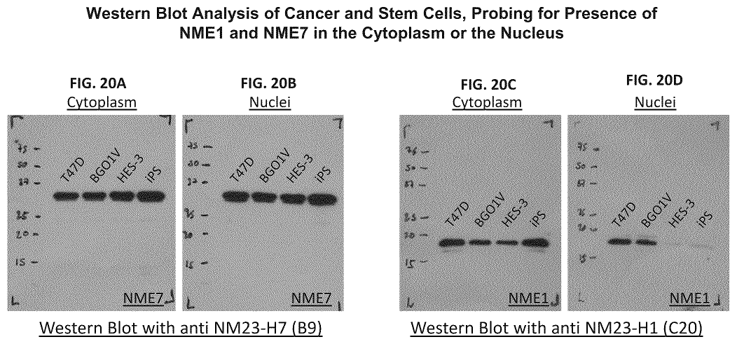

NME7 is a single molecule that has two NDPK domains and so, in one aspect, functions as NME1 dimers do. One of the binding partners of NME7 is MUC1*. Our studies show that NME7 binds and dimerizes the extra cellular domain of MUC1*-positive cells to promote growth and to inhibit differentiation of both human stem cells and cancer cells. NME7 is also detected in cancer cells, in the conditioned media, cytoplasm and nucleus, indicating that it functions as a secreted growth factor and also as a transcription factor that directly or indirectly binds DNA. The Western blots of FIG. 20 show that both NME1 and NME7 are present in both the cytoplasm and in the nucleus of human cancer cells (T47D), embryonic stem cells (BGO1v and HES-3) and induced pluripotent stem (iPS) cells. These data show that NME1 and NME7 can function directly or indirectly to affect transcription of genes. Therefore in one aspect of the invention, the function of NME1 or NME7 is inhibited by adding agents which can be small molecules, that inhibit the binding of NME1 or NME7 to DNA, and agents that inhibit the transcription function of NME1 or NME7 are anti-cancer agents that can be administered to a patient with cancer or at risk of developing cancer.

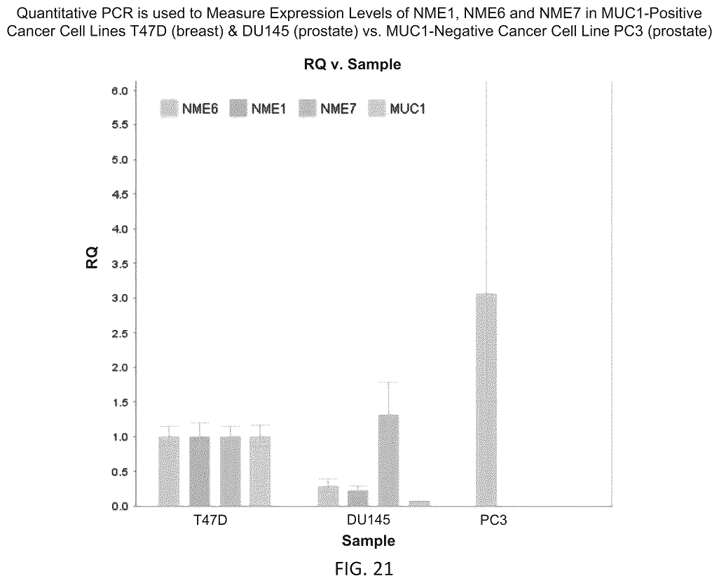

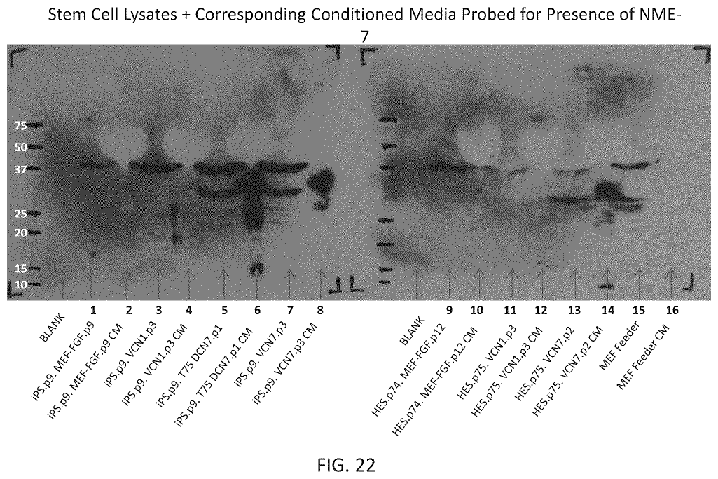

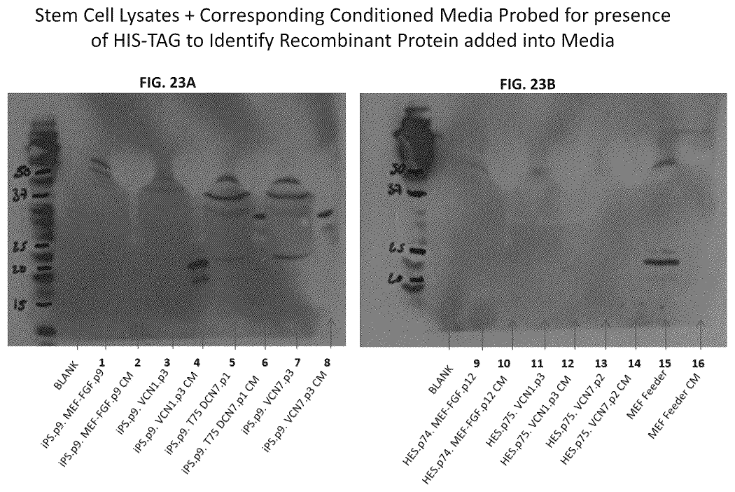

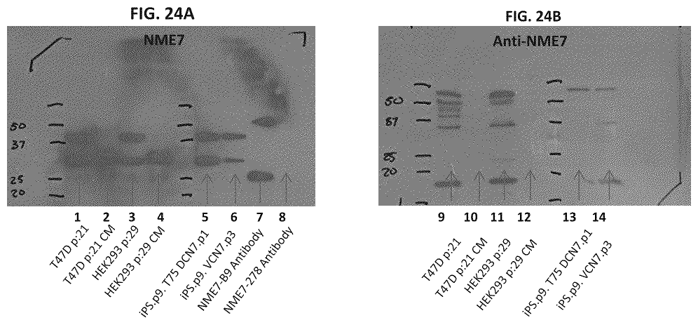

NME proteins likely are expressed to different levels in different cancer cells. Most cancers that are MUC1*-positive and show high expression of NME1, NME6 and NME7. DU145 prostate cancer cells had higher expression of NME7 than NME1 or NME6. PC3 prostate cancer cells, which are MUC1*-negative, had no detectable NME1 or NME7 but had high expression of NME6 (FIG. 21).

### NME7 Exists in Different Forms

NME7 is expressed as different species. Some of these species are specific to cancer cells. Full length NME7 is 42 kDa and is comprised of two non-identical NDPK domains and a DM10 leader sequence at its N-terminus. Full length NME7 can be found in the cytoplasm. A ˜33 kDa NME7 species, consistent with a species comprised of the NDPK A and B domains but devoid of the DM10 leader sequence is found exclusively in the conditioned media of both stem cells and cancer cells (FIG. 5 and FIG. 22). Note that these findings are independent of recombinant NME1 in dimer form added to culture the stem cells. FIG. 23 shows that when the gel of FIG. 22 was stripped and re-probed for the presence of the Histidine tag on the recombinant protein, none was detected. These results argue that a smaller molecular weight NME7 is the secreted growth factor form. We made an NME7 variant comprised of the NDPK A and B domains but without the DM10 domain, having molecular weight of ˜33 kDa, that we called NME7-AB. This recombinant NME7-AB is able to fully support pluripotent human stem cell growth in serum-free media, devoid of other growth factors or cytokines. NME7-AB also fully supported the growth of MUC1*-positive cancer cells. These experiments demonstrate that the secreted form of NME7 is the growth factor form and that it is comprised of NDPK A and B domains and devoid of most or all of the DM10 domain and has a molecular weight of ˜33 kDa. FIG. 24 shows photos of Western blots of various cell lysates and corresponding conditioned media probed for the presence of NME7 using a mouse monoclonal antibody (A) or another monoclonal antibody that only recognizes the N-terminal DM10 sequence (B). The lack of binding of the DM10 specific antibody to the ˜33 kDa NME7 species in the samples from the conditioned media of the cells indicates that the secreted form of NME7 is devoid of most if not all of the N-terminal DM10 leader sequence.

Another smaller ˜25 kDa NME7 species is also sometimes present. Western blot shows presence of lower molecular weight species ˜25 kDa from the outset. This ˜25 kDa NME7 is comprised of the NDPK A domain and has a single binding site for MUC1*. The ˜25 kDa band was excised and analyzed by mass spectrometry. Mass spec showed that the ˜25 kDa species was comprised essentially of the NDPK A domain.

It has been reported that NME7 is expressed in other human tissues, albeit at low levels. However, we have discovered that it is the secreted form of NME7 that functions as a growth factor and although some adult tissues may express NME7, the critical aspect is whether or not it is secreted. Stem cells that express and secrete NME7 are those stem cells that are in an earlier and thus more pluripotent state than stem cells that do not secrete NME7, which are in an earlier and more pluripotent state than stem cells that do not express or secrete NME7. Cancer cells that express and secrete NME7 are those cancer cells that are less differentiated and more aggressive than cancer cells that do not secrete NME7. Thus, measuring levels of NME7 and secreted NME7 can be used to predict tumor aggressiveness, design therapies, monitor efficacy of therapies and to stratify patient populations for clinical trials. Therefore, antibodies that detect NME1, NME6 or NME7 can be used as diagnostic tools to detect the occurrence of cancer or to assess the aggressiveness of the cancer, wherein high levels of NME1, NME6 or NME7 correlate with tumor aggressiveness and poor outcome. High levels of NME7 and NME6 are especially correlated to tumor aggressiveness and therefore poor prognosis. Patient samples that can be probed with antibodies against NME1, NME6 or NME7 can be samples of bodily fluids, including blood, tissue biopsies, needle biopsies and the like.

### NME Family Member Proteins can Function to Promote Cancer

The inventors previously reported that NME proteins promote growth and pluripotency of embryonic and iPS cells as well as inducing cells to revert to a stem-like state. Because much of the genetic signature of a stem-like state and a cancerous state is now shared, we conclude that NME family member proteins are also able to induce a cancerous state. In a preferred embodiment the NME family member protein is NME1 or an NME protein having greater than 30%, 35%, 40%, 45%, 50%, 55%, 60%, 65%, 70%, 75%, 80%, 85%, 90%, 95%, or 97% sequence identity to NME1, wherein said protein is a dimer. In a more preferred embodiment, the NME family member protein is NME7 or an NME protein having greater than 30%, 35%, 40%, 45%, 50%, 55%, 60%, 65%, 70%, 75%, 80%, 85%, 90%, 95%, or 97% sequence identity to at least one of the NME7 domains A or B and able to dimerize the MUC1* growth factor receptor.

Here, we report that NME1 in dimer form, a bacterial NME1 in dimer form, NME7 or NME7-AB were able to: a) fully support human ES or iPS growth and pluripotency, while inhibiting differentiation; b) revert somatic cells to a more stem-like or cancer-like state; and c) transform cancer cells to the highly metastatic cancer stem cell state, also referred to as tumor initiating cells.

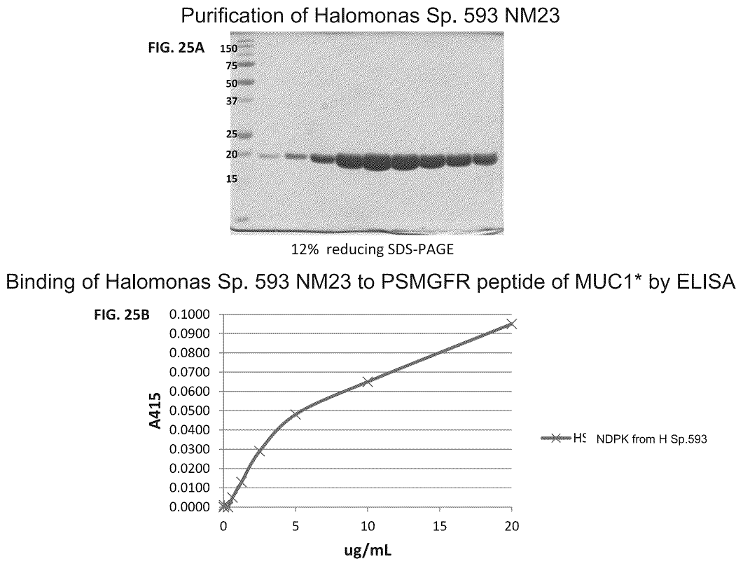

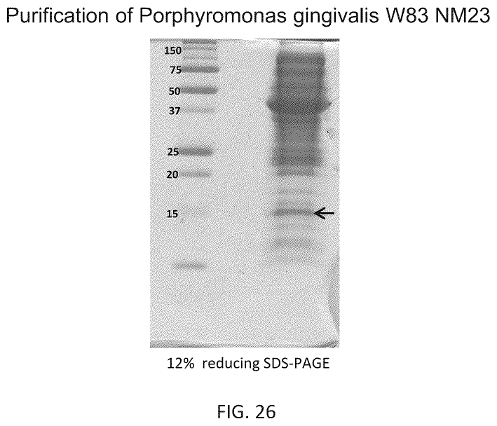

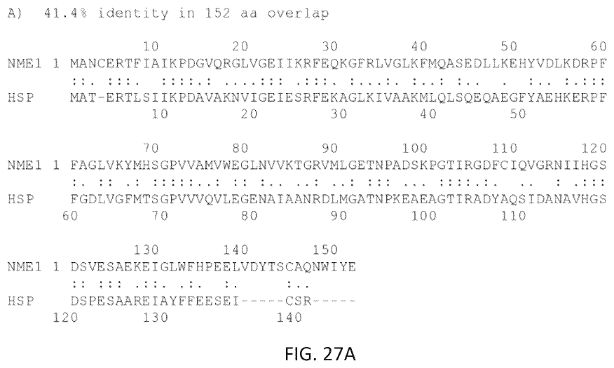

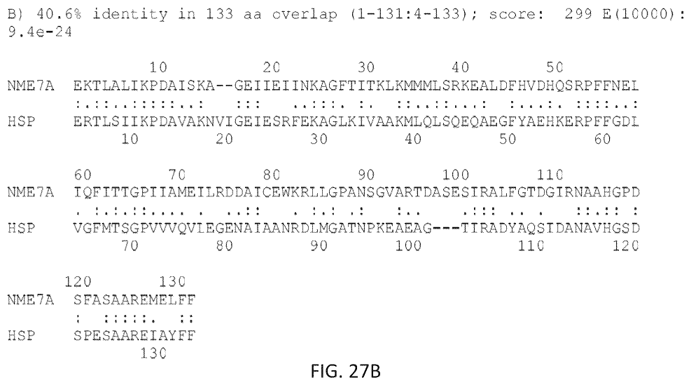

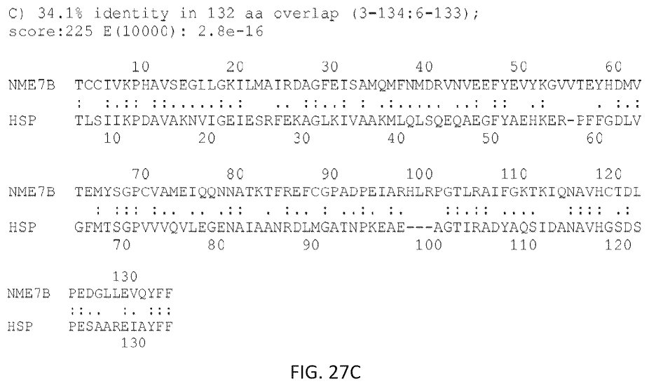

We made recombinant bacterial NME proteins found in Halomonas Sp. 593 (′HSP 593′) and in Porphyromonas gingivalis W83 that had high sequence homology to human NME1 and had been reported to exist in dimer state (FIG. 25 and FIG. 26). HSP 593 expressed well in E. coli and a significant portion was present as a dimer, which population was then purified by FPLC and confirmed the dimer population (FIG. 25A). A direct binding experiment was performed that showed that bacterial NME from Halomonas Sp. 593 bound to the PSMGFR peptide of the MUC1* extracellular domain (FIG. 25B). Sequence alignment between HSP 593 and human NME1 or human NME7 domain A or B showed that the bacterial NME that bound to MUC1* extracellular domain was 40-41% identical to human NME1 and human NME7-A, and 34% identical to NME7-B (FIG. 27A-C).

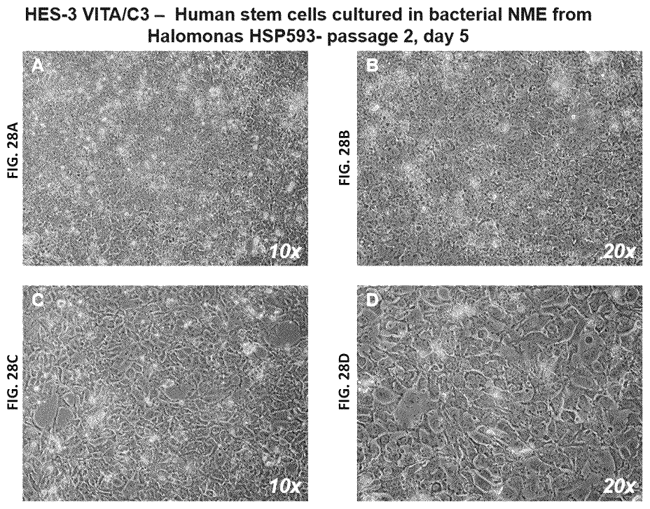

Additional experiments were performed that showed that bacterial NMEs with greater than 30%, or more preferably 40%, identity to human NME1 or NME7 function like the human NMEs that promote cancer and stem cell growth and survival. Many of the bacterial NMEs that had this high sequence identity to the human NMEs were reported to be implicated in human cancers. We therefore sought to test the idea that many bacteria were either inducing cancer in humans or making existing cancers worse. The bacterial NME was tested in functional assays against human NME1 and NME7. Human HES-3 embryonic stem cells were cultured in a serum-free minimal base media with either HSP 593, human NME1 dimers or human NME7-AB as the only growth factor or cytokine. Just as human NME1 and NME7 fully supported human stem cell growth, so did bacterial NME from HSP 593 (FIG. 28A-F, compared to FIG. 9 and FIG. 10).

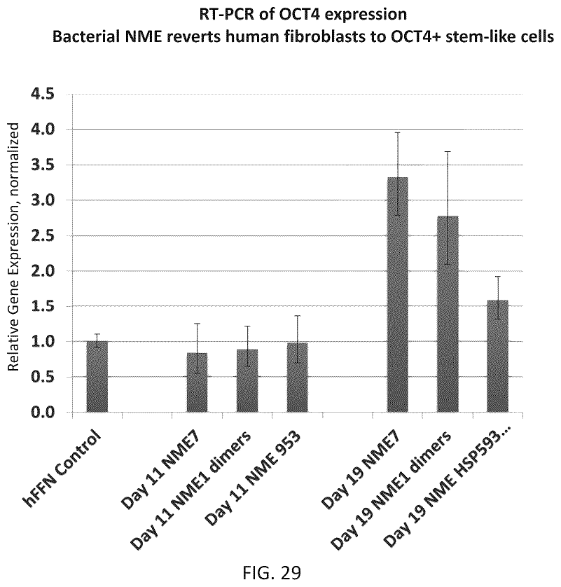

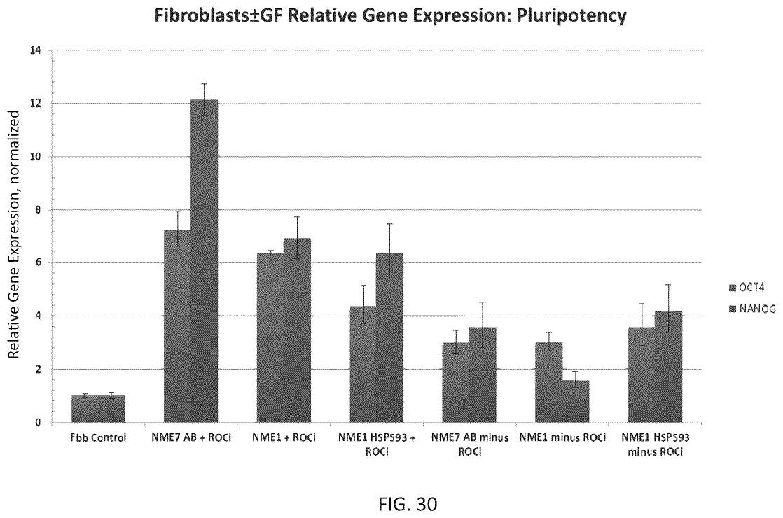

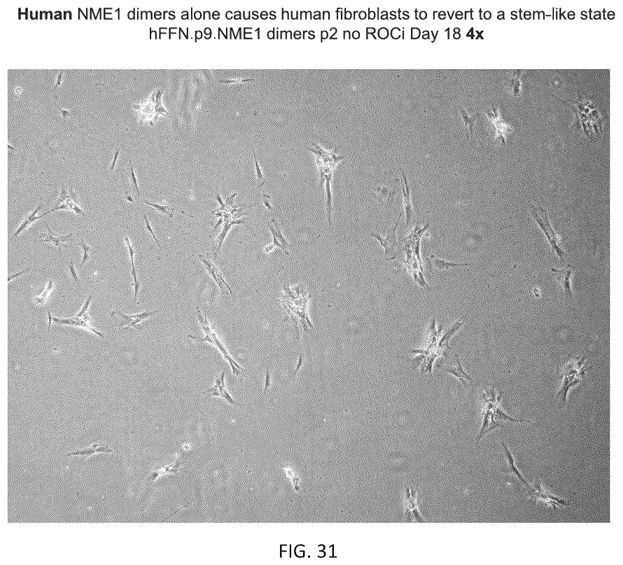

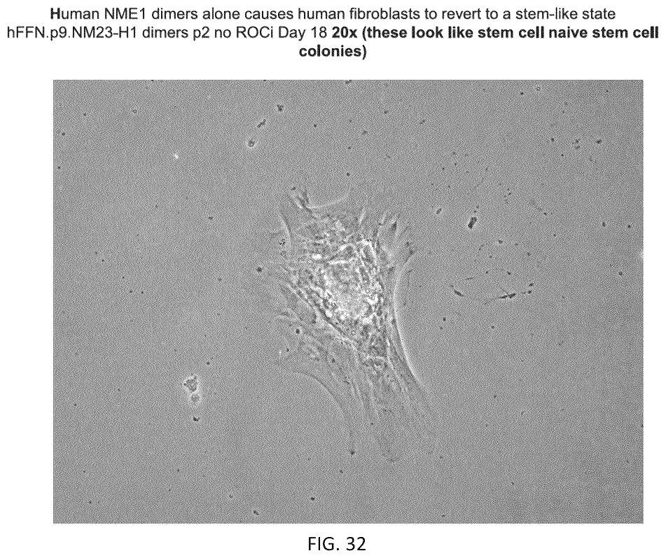

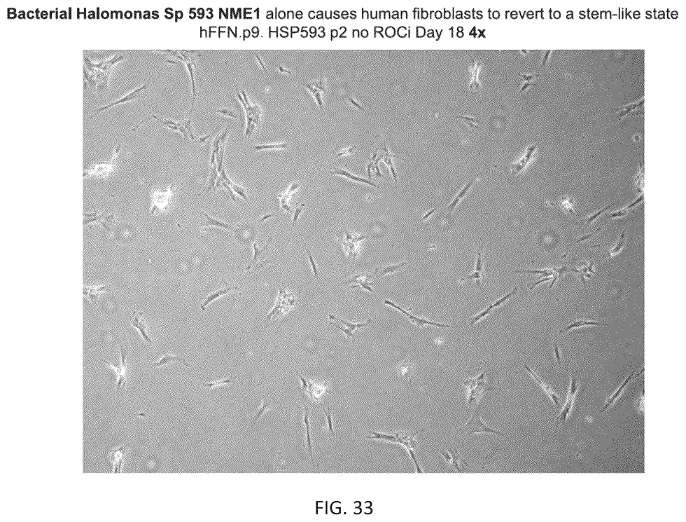

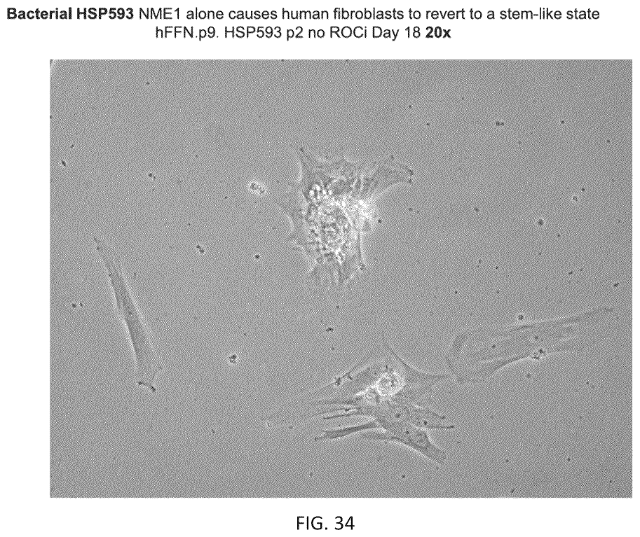

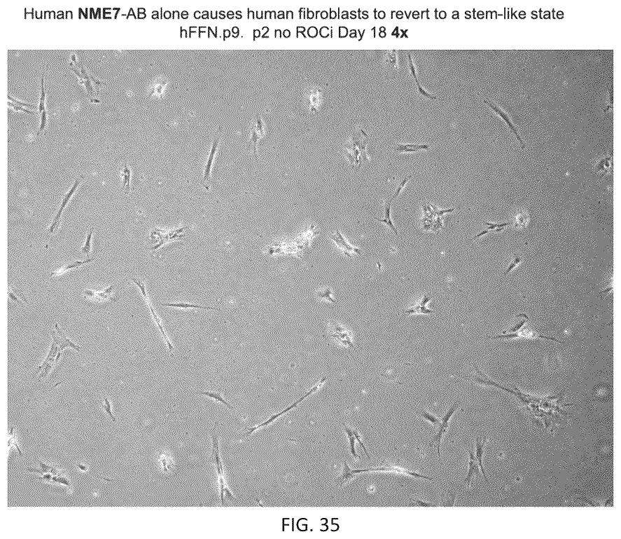

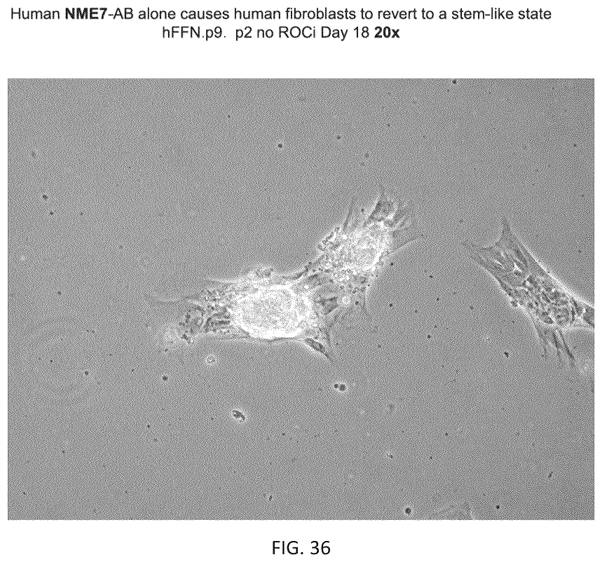

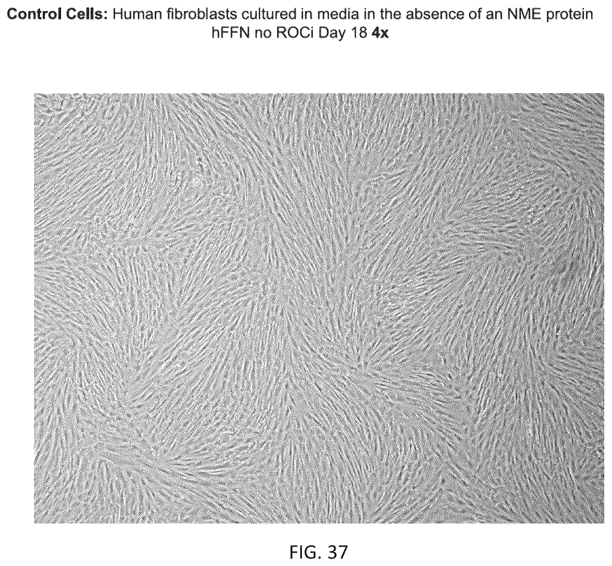

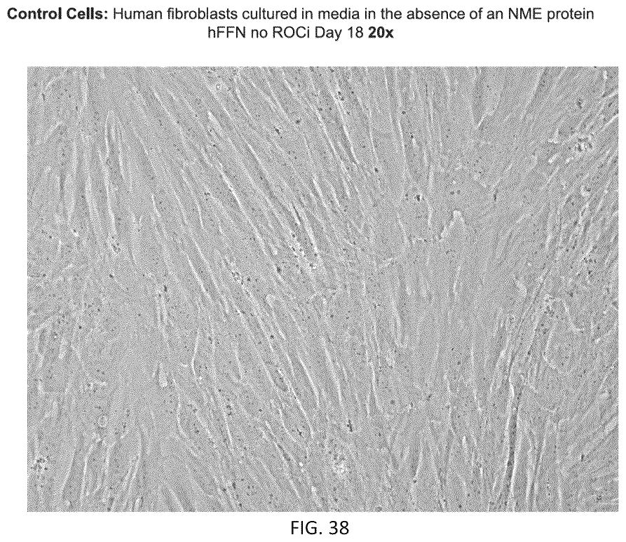

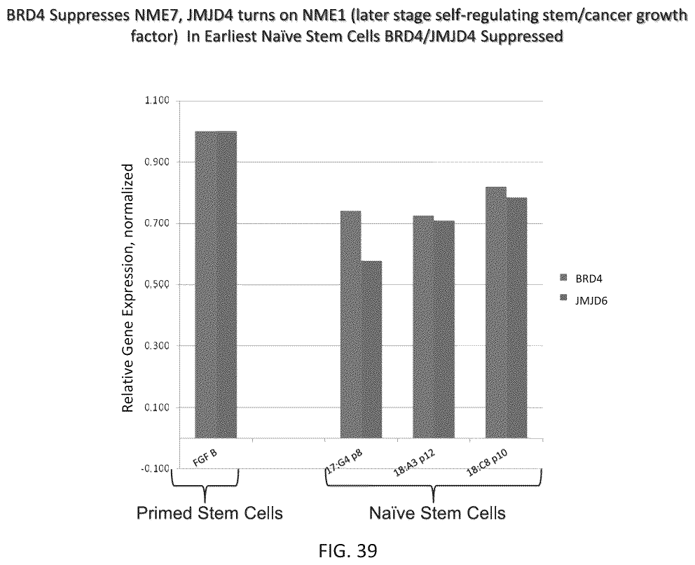

Human NME1 dimer or human NME7 are able to make somatic cells revert to a less mature state, expressing stem and cancer cell markers. Bacterial NME from HSP 593 was tested alongside the human homologs to determine if it could mimic their function by being able to revert somatic cells to a cancer-like state. Human fibroblasts were cultured in a serum-free minimal base media with either HSP 593, human NME1 dimers or human NME7-AB as the only growth factor or cytokine. RT-PCR measurement showed that like the human NMEs, bacterial NME1 HSP 593 reverted somatic cells to an OCT4-positive stage by Day 19 (FIG. 29). Recalling that stem cells and metastatic cancer cells can grow anchorage-independently, we repeated the experiments but this time a rho kinase inhibitor was added to one set of cells to make the cells adhere to the surface. When the floating cells were forced to adhere to the surface, RT-PCR showed that there had actually been a 7-fold increase in stem/cancer marker OCT4 and as high as a 12-fold increase in the stem/cancer markers Nanog (FIG. 30). Photos of the experiment show the dramatic change in morphology as the fibroblasts revert when cultured in human or bacterial NME (FIGS. 31-38). The relative order of efficiency of reverting somatic cells to a less mature state was NME7>NME1 dimers>NME1 bacterial. Transcription factors BRD4 and co-factor JMJD6 reportedly suppress NME7 and up-regulate NME1 (Lui With et al, 2013). We found that these factors were expressed at lower levels in naïve stem cells than they were in the later stage primed stem cells (FIG. 39). This result supports our hypothesis that NME7 is an earlier expressed stem cell growth factor than NME1 because the former cannot turn itself off or regulate self-replication the way NME1 does; as a dimer it activates stem cell growth but when the cells secrete more and it forms hexamers, the hexamers do not bind MUC1* and differentiation is induced.

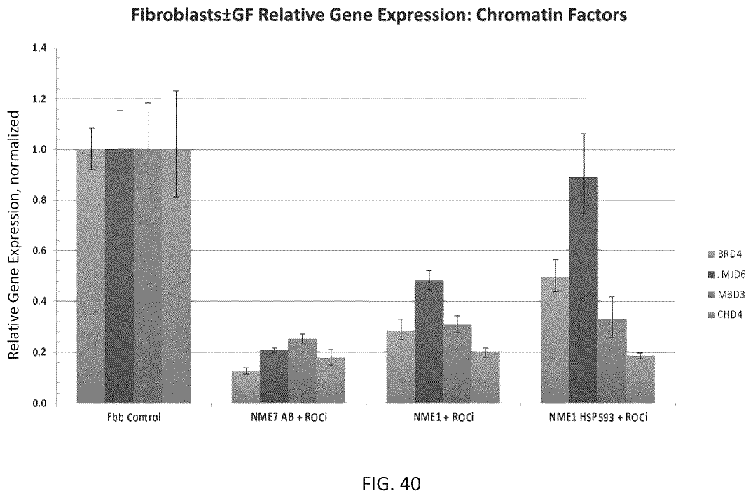

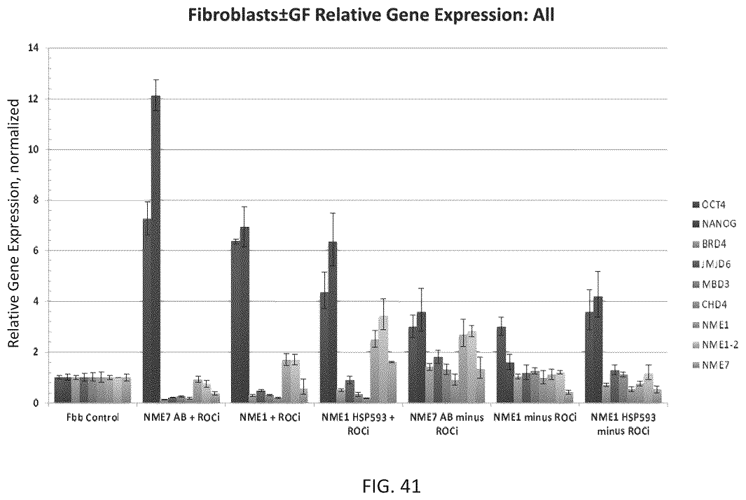

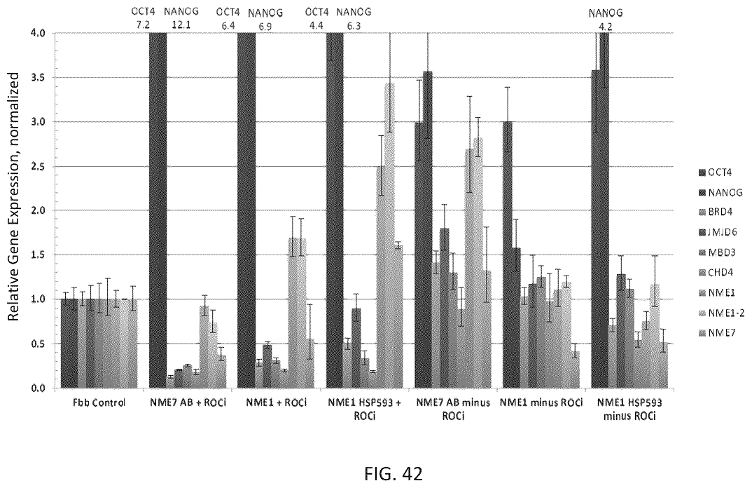

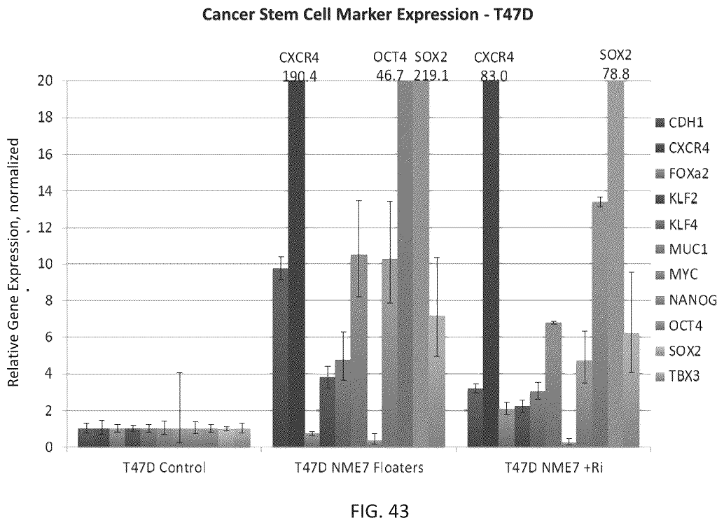

Chromatin re-arrangement factors MBD3 and CHD4 were recently reported to block the induction of pluripotency (Rais Y et al, 2013). RT-PCR measurements of human fibroblasts grown in the human NME1 or NME7 or bacterial NME1 show that the NME protein suppress all four (BRD4, JMJD6, MBD3 and CHD4) blockers of pluripotency (FIG. 40). Composite graphs of RT-PCR experiments show that the relative potency of increasing pluripotency genes and decreasing pluripotency blockers is NME7>NME1>HSP 593 NME. However, Bacterial NME from HSP 593 apparently up-regulates expression of human NME7 and NME1 (FIG. 41 and FIG. 42). Thus, NME1 dimers, NME7 and bacterial NME1 dimers cause somatic cells to revert to a less mature cancer/stem-like state.

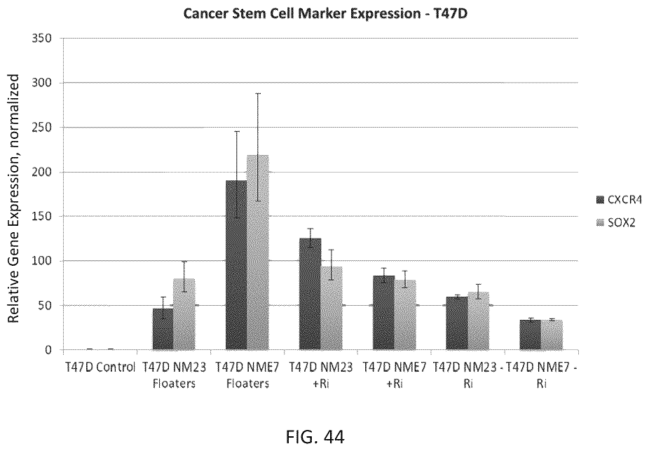

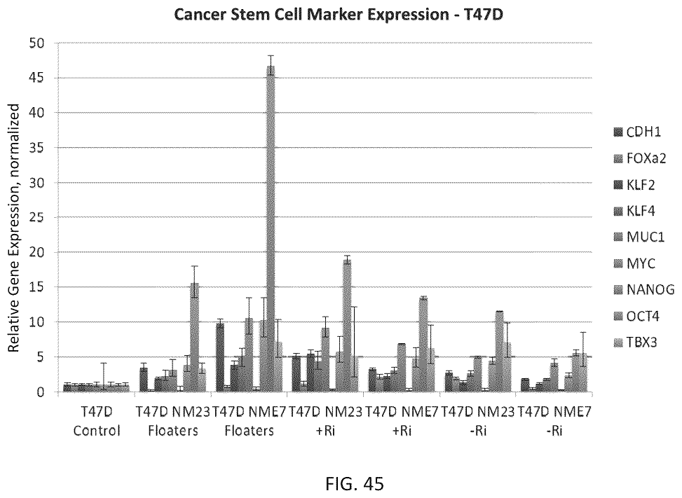

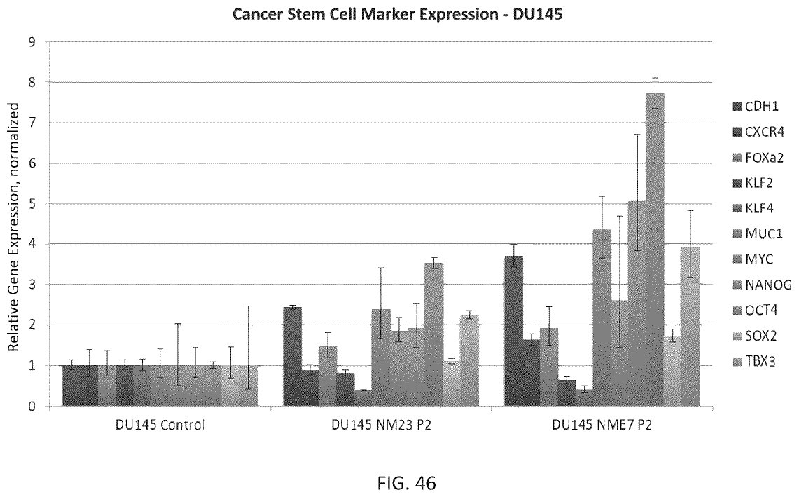

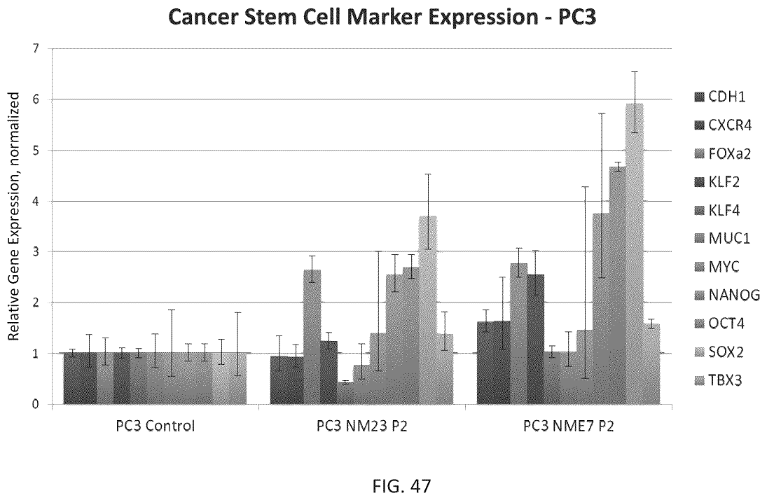

Another function that NME1 and NME7 have is the ability to transform cancer cells to the more metastatic cancer stem cell state, also called tumor initiating cells. A panel of cancer cells were cultured in a serum-free minimal base media with human NME7-AB or human NME1 dimers (‘NM23’ in figures) as the only growth factor or cytokine. After several days in this media, cells began to float off the surface and continued to grow in solution. The ‘floaters’ were collected and separately analyzed by PCR. Cells in other wells were treated with a rho kinase inhibitor (‘Ri in figures’). Quantitative PCR measurements show an increase of the cancer stem cell markers, some of which used to be thought of as stem cell markers only (Miki J et al 2007, Jeter C R et al 2011, Hong X et al 2012Faber A et al 2013, Mukherjee D et al 2013, Herreros-Villanueva M et al, 2013, Sefah K et al, 2013; Su H-T et al 2013). FIGS. 44-47 show that culture with the NME proteins reverts the cancer cells to the highly metastatic tumor initiating cells, with the ‘metastasis receptor’ CXCR4 up-regulated by more than 200-fold, SOX2 up-regulated by more than 200-fold, E-cadherin (CDH1), NANOG and MUC1 up by 10-fold. In conclusion, cancer cells secrete NME7 and NME1 which activate MUC1 and up-regulate a host of cancer and cancer stem cell genes. We observed a more modest, but trending, increase in markers of cancer stem cells and metastasis even in cells that were MUC1-negative (FIG. 47). We therefore conclude that NME7 likely is able to enter these cells by a route other than MUC1* wherein it can still act as a transcription factor and affect the expression levels of these genes. NME1 must be a dimer to function this way, because as a hexamer it does not activate stem or cancer growth. However, many cancers mutate NME1 so that it resists the formation of the self-replication limiting hexamer. Unlike NME1, NME7 is always active.

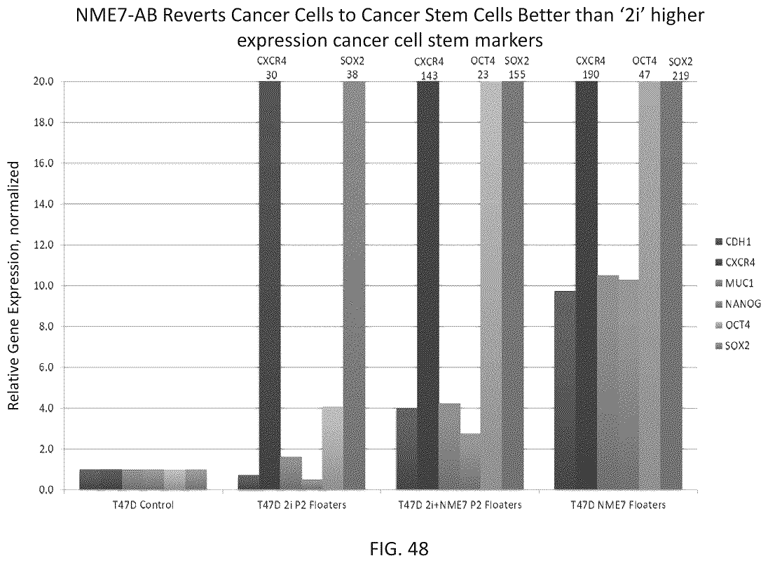

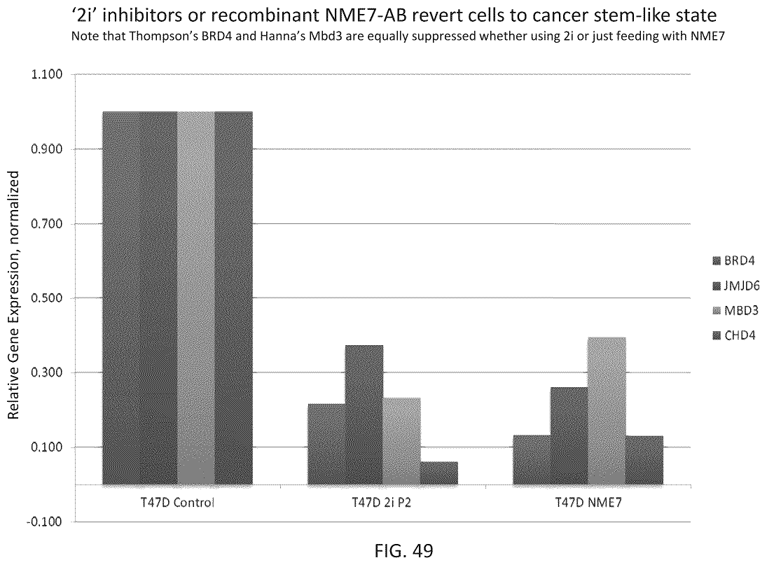

2i inhibitors, which are small molecule inhibitors of GSK3-beta and MEK of the MAP kinase signaling pathway, have been reported (Silva J et al, 2008) to revert mouse primed stem cells to the naïve state. We wondered whether these inhibitors could also revert human cancer cells to the cancer stem cell state. T47D breast cancer cells were cultured for 10 days in a serum-free minimal base media with the 2i inhibitors added or in the same base media with human recombinant NME7-AB added, or both NME7-AB and 2i. The results shown in FIG. 48 show that the cancer stem cell markers E-cadherin (CDH1), the metastasis receptor CXCR4 as well as stem/cancer markers OCT4, SOX2 and NANOG were greatly up-regulated by 2i, 2i+NME7-AB, NME7-AB alone. The relative potency of inducing the cancer stem cell markers was NME7>NME7+2i>2i (FIG. 48). Expression of the pluripotency-blocking chromatin regulators and transcription factors BRD4, JMJD6, MBD3 and CHD4 were similarly down-regulated when the cancer cells were treated with either 2i or NME7-AB (FIG. 49).

Thus, agents that disable any of these functions of NME1 dimers, human or bacterial or of NME7—ability to promote stem cell growth, ability to bind to MUC1* peptide PSMGFR, ability to revert somatic cells to a less mature state, ability to transform cancer cells to cancer stem cell state—are potent anti-cancer agents and can be administered to patients for the treatment or prevention of cancers.

In support of the idea that NME inhibitors are potent anti-cancer agents, we performed an experiment and contend that it can be extended to many other antibodies that bind to NME7, NME7-AB as well as other NME proteins. We grew MUC1*-positive cancer cells in the presence or absence of a rabbit polyclonal antibody raised against human NME7. Tumor cell growth was greatly decreased in a concentration dependent manner and is shown in FIGS. 13-15. Polyclonal anti-NME7 may not be the ideal anti-cancer agent in that it is a collection of antibodies produced by rabbits. For a therapeutic agent, monoclonal antibodies would be generated and selected for their ability to specifically inhibit cancer cell growth and ideally select a monoclonal antibody that disrupts binding of NME7 to MUC1* extracellular domain. Finally a human or humanized antibody would be selected.

NME Function 1: One way that NME proteins function to promote cancer is by binding to a clipped form of the MUC1 transmembrane protein, herein referred to as MUC1*, which consists primarily of the PSMGFR sequence. Dimerization of the MUC1* extracellular domain stimulates growth and de-differentiation of stem and cancer cells.

NME Function 2: Another way that NME proteins function to promote cancer, de-differentiation, pluripotency, growth or survival is that they can be transported to the nucleus where they function directly or indirectly to stimulate or suppress other genes. It has been previously reported (Boyer et al, 2005) that OCT4 and SOX2 bind to the promoter sites of MUC1 and its cleavage enzyme MMP16. The same study reported that SOX2 and NANOG bind to the promoter site of NME7. We conclude, on the basis of our experiments that these ‘Yamanaka’ pluripotency factors (Takahashi and Yamanaka, 2006) up-regulate MUC1, its cleavage enzyme MMP16 and its activating ligand NME7. It has also been previously reported that BRD4 suppresses NME7, while its co-factor JMJD6 up-regulates NME1 (Thompson et al) that we determined is a self-regulating stem cell growth factor that is expressed later than NME7 in embryogenesis. Still others recently reported that siRNA suppression of Mbd3 or Chd4 greatly reduced resistance to iPS generation (Rais Y et al 2013 et al.) Our evidence is that there is a reciprocal feedback loop wherein NME7 suppresses BRD4 and JMJD6, while also suppressing inhibitors of pluripotency Mbd3 and CHD4. We note that in naïve human stem cells, these four factors BRD4, JMJD6, Mbd3 and CHD4 are suppressed compared to their expression in later stage ‘primed’ stem cells. We also note that the 2i inhibitors (inhibitors of Gsk3β and MEK) that revert mouse primed stem cells to the naïve state, also down regulated the same four factors BRD4, JMJD6, Mbd3 and CHD4.

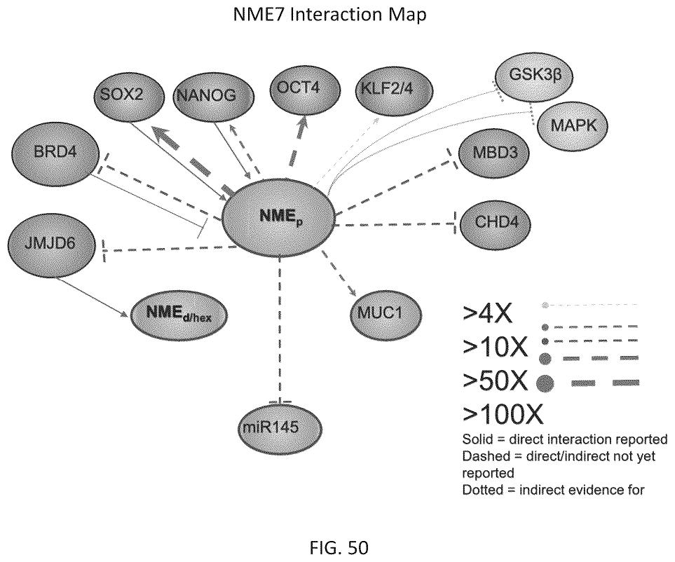

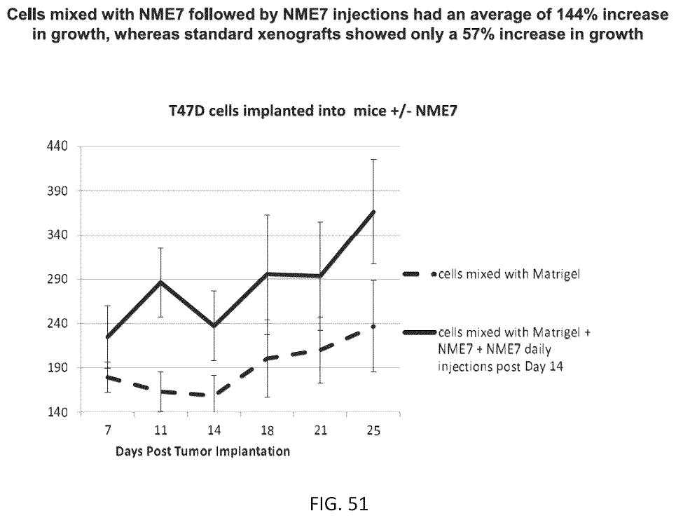

We have also discovered that NME7 up-regulates SOX2 (>150X), NANOG (˜10X), OCT4 (˜50X), KLF4 (4X) and MUC1 (10X). Importantly, we have shown that NME7 up-regulates cancer stem cell markers including CXCR4 (˜200X) and E-cadherin (CDH1). Taken together these multiple lines of evidence point to the conclusion that NME7 is the most primitive stem cell growth and pluripotency mediator and that it is a powerful factor in the transformation of somatic cells to a cancerous state as well as transforming cancer cells to the more metastatic cancer stem cells. FIG. 50 is a cartoon of the interaction map of NME7 and the associated regulators of the stem/cancer state as evidenced by the experiments described herein. NME1 in dimer form functioned approximately the same as NME7 in being able to convert somatic cells to a stem/cancer-like state and being able to transform cancer cells to metastatic cancer stem cells, albeit to a slightly lesser degree. Similarly, bacterial NME dimers with high homology to human NME1 or NME7 such as Halomonas Sp 593 was, like NME1 dimers and NME7 monomers, able to fully support human stem cell growth, pluripotency and survival, cancer cell growth and survival, reverted somatic cells to a cancer/stem cell state and transformed cancer cells to the more metastatic cancer stem cells.

We therefore conclude that agents that disable the function of NME proteins that support human stem cell growth, pluripotency and survival, cancer cell growth and survival, that are able to revert somatic cells to a cancer/stem cell state and that are able to transform cancer cells to the more metastatic cancer stem cells are ideal targets for anti-cancer therapies, wherein the therapeutic agent disables the NME protein, blocks its binding to MUC1*, blocks its function as a direct or indirect transcription factor or blocks its function as described above. In a preferred embodiment, the agent that blocks the function of the NME protein is an antibody. In another preferred embodiment the agent blocks the function of NME1 dimers or dimerization. In a yet more preferred embodiment the agent blocks the function of NME7. An anti-cancer agent that blocks the function of one of these NME proteins can alternatively be a nucleic acid. For example a nucleic acid that inhibits expression of the NME such as sh- or siRNA, antisense nucleic acid and the like. Alternatively, the agent may indirectly suppress expression of the NME. For example, increased expression of BRD4 would suppress NME7 and thus act as an anti-cancer agent. In another embodiment, the agent that inhibits function of the targeted NME protein is a synthetic chemical such as a small molecule that either acts on the NME protein directly or inhibits its expression. Separately or in combinations, these agents are potent anti-cancer agents for the treatment or prevention of cancers.

In one case, an agent that inhibits the targeted NME protein is an antibody and is an anti-cancer agent that is administered directly to a patient for the treatment or prevention of cancers. In a preferred embodiment the primary cancer or its progeny is a MUC1* positive cancer. The antibody may be an antibody per se or may be an engineered antibody-like molecule. The antibody or antibody-like molecule can be linked to a cytotoxic entity or an entity that activates an immune response. For example, portions of the anti-NME antibody can be engineered to be a part of a therapeutic molecule as described in the CAR (chimeric antigen receptor) T cell technology (Porter D et al, 2011). The antibody can be bivalent, monovalent, bi-specific humanized or partially humanized. The antibody or antibody-like molecule may be generated using in vitro binding assays, phage display techniques and the like, including those used by Tiller T et al, 2013, and for example using randomized human antibody epitope libraries such as the Ylanthia® system as well as others.

In another aspect of the invention, the agent that inhibits the targeted NME protein is an antibody that is generated by the patient, wherein the patient is immunized with portions of the targeted NME protein(s) such that the patient mounts an immune response which includes anti-NME antibodies. Such immunization is performed for the treatment or prevention of cancers, for example as a vaccine.

In another aspect, the present invention involves the identification of peptide sequences derived from MUC1*, NME1 human, NME1 bacterial and NME7 that will give rise to antibodies that are anti-cancer agents. These peptide sequences can be used for generating therapeutic antibodies as well as for vaccines, nucleic acid sequences for anti-sense type therapies, methods for the identification of cancer-causing bacteria, diagnostic methods and drug screening methods. In one aspect of the invention, peptides of sequence described herein may be augmented with adjuvant or fused to other peptides which stimulate the immune system and then used to generate anti-cancer antibodies either in a host animal or in a human as a vaccine to immunize against cancer by inducing the patient to raise antibodies against the targeted NME protein. In a preferred embodiment, the targeted NME protein is bacterial NME having 30% or greater sequence identity to human NME1 or NME7 domain A or B. In a more preferred embodiment, the targeted NME protein is human NME1, wherein the antibody may specifically target NME1 with mutations that make it prefer dimer formation such as the S120G mutation, the P69S mutation or C-terminal truncations. In a still more preferred embodiment, the targeted NME protein is NME7 (SEQ ID NO:13), including the cleaved form substantially as set forth as NME7-AB (SEQ ID NO:39).

A transgenic mouse expressing human NME7, human NME1 or mutants that prefer dimerization or bacterial NME would be of great use in drug discovery, for growing cancer cells in vivo and for testing the effects of immunizing NME-derived peptides as elements of an anti-cancer vaccine. For example, murine NME proteins differ from human NME proteins. Mouse stem cells grow using the single growth factor LIF, while LIF cannot support the growth of human stem cells. We now know that cancer cells and stem cells grow by similar mechanisms. Therefore, implanting human cancer cells into a mouse poses problems besides just an immune response in the mouse to human cancer cells; the mouse does not produce human NME7 or dimeric NME1 which are the growth factors that singly promote cancer growth and their transformation to cancer stem cells.

We have found that animals injected with human NME7 develop cancers more easily than mice that are not injected. For example, some cancer cells are very difficult to engraft in animals. We increased the engraftment rate of cancer cells by several fold by injecting the animal with human NME7 or NME7-AB. Immune-compromised mice were implanted with T47D breast cancer cells that were mixed 50/50 vol/vol with either Matrigel or NME7-AB. After 10 days, the mice that had received the NME7 mixed cells were additionally injected with NME7-AB every day (FIG. 51). The group that was additionally injected with NME7-AB (dashed line) had larger tumors that grew at an accelerated rate. Engraftment rates, decreased numbers of required cells and a faster tumor growth rate resulted when NME7-AB was mixed with the cancer cells when implanted and when the mice were injected every 24 or 48 hours after implantation. A range of ratios of cancer cells to NME7 or the injection schedule of NME7 is expected to vary from one mouse strain to another and from one tumor type to another. In an improvement over this method, animals that are transgenic for human NME7 or NME7-AB greatly increase engraftment rates of cancer cells and thus, decrease the number of cells required to develop into a tumor in an animal. This allows growth of primary patient cancer cells in an animal expressing human NME7 or NME7-AB.

In one example, cancer cells are implanted into an animal and the animal is administered NME7 or NME7-AB. In a preferred embodiment, the animal is a transgenic animal that expresses human NME7-AB. In a preferred embodiment, the cancer cells are primary cells from a patient. In this way, the animal, which can be a mouse, provides the NME growth factor that causes the patient cancer cells to revert to a less mature, more metastatic state. In one embodiment, the host animal is injected with candidate drugs or compounds and efficacy is assessed in order to predict the patient's response to treatment with the candidate drug or compound. In another instance, the first line treatments or drugs that are being administered to the patient or are being considered for treatment of the patient, are administered to the animal bearing the patient's cancer cells which are being reverted to a less mature state. The first line treatments likely influence which mutations the cancer cells adopt in order to escape the first line treatments. The resultant cancer cells can then be removed from the host animal and analyzed or characterized to identify mutations that are likely to occur in response to certain treatments. Alternatively, the cancer cells can remain in the host animal and the host animal is then treated with other therapeutic agents to determine which agents inhibit or kill the resistant cells or cancer stem cells.

Our experiments have shown that the differences between murine NME proteins and human NME proteins is a major reason why engraftment of human cancer cells into mice is so inefficient. Injecting the mouse with recombinant human NME7 at the time of cancer cell implantation greatly increased the rate of tumor engraftment and the rate of tumor growth. Thus a transgenic mouse that expresses human NME7, or more preferably human NME7-AB, would greatly increase the rate of tumor engraftment, making it possible to engraft patient cells in a mouse model for drug discovery, dosage testing or to determine how the patient's cancer cells might evolve or mutate in response to drug treatment. It would be advantageous to have the human NME7 on an inducible promoter, for example to avoid potential problems of NME7 expression during development of the animal. Alternatively, cancer cells, including patient cells can be cultured in NME7, NME1 dimers or bacterial NME that mimics human NMEs such that the cells are transformed to the cancer stem cells that require as few as 50-200 cells to initiate a tumor in an animal. These cells would then be tested in vitro or in vivo, including in a transgenic animal bearing NME7, NME1 dimers, bacterial NMEs or single chain NME1 pseudo dimers.

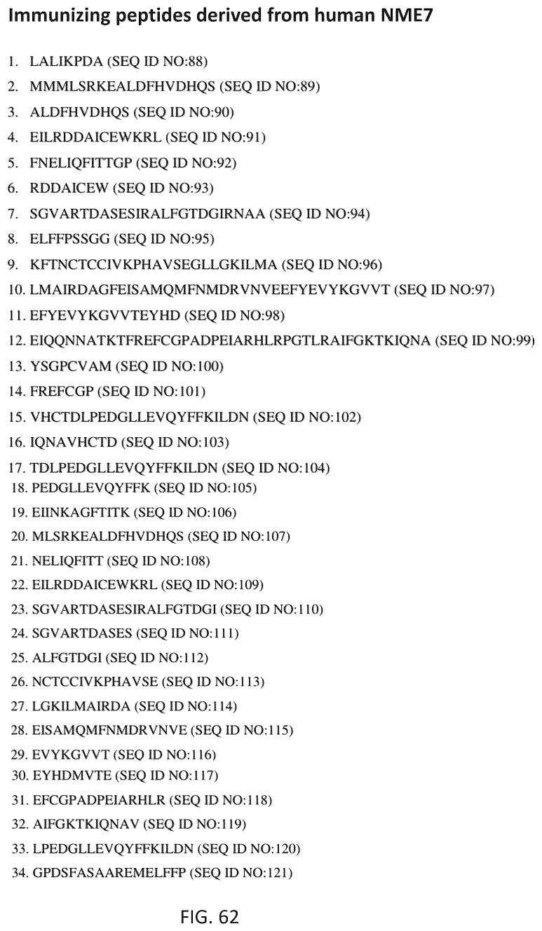

A transgenic animal expressing human NME, especially NME7-AB, would also be useful for assessing which immunizing peptides could safely be used for the generation of antibodies against NME proteins, including NME1, bacterial NME and NME7. For example, mice transgenic for human NME1, NME7, or NME7-AB could be immunized with one or more of the immunizing peptides set forth as in FIGS. 62-64, peptide numbers 1-53. Control group mice are analyzed to ensure that anti-NME antibodies were produced. Human tumor cells would then be implanted into the transgenic mouse, wherein expression of the human NME protein in the host animal is induced, if using an inducible promoter. The efficacy and potential toxicities of the immunizing peptides is then assessed by comparing the tumor engraftment, tumor growth rate and tumor initiating potential of cells transplanted into the transgenic mouse compared to the control mouse or a mouse wherein the inducible NME promoter was not turned on. Toxicities are assessed by examining organs such as heart, liver and the like, in addition to determining overall bone marrow numbers, number and type of circulating blood cells and response time to regeneration of bone marrow cells in response to treatment with agents cytotoxic to bone marrow cells. Immunizing peptides derived from those listed in FIGS. 62-64, peptide numbers 1-53 that significantly reduced tumor engraftment, tumor growth rate, or tumor initiating potential with tolerable side effects are selected as immunizing peptides for the generation of antibodies outside of the patient or in a human as an anti-cancer treatment, preventative or vaccine.

Therefore, a mouse or other mammal that would spontaneously form tumors, or respond more like a human to drugs being tested or that would better allow human tumor engraftment, is generated by using any one of the many methods for introducing human genes into an animal. Such methods are often referred to as knock-in, knock-out, CRISPR, TALENs and the like. The invention envisions using any method for making the mammal express human NME7 or NME7-AB. NME7 or NME7-AB can be inducible as one of many methods for controlling expression of transgenes are known in the art. Alternatively, the expression or timing of expression, of NME7 may be controlled by the expression of another gene which may be naturally expressed by the mammal. For example, it may be desirable for the NME7 or NME7 variant to be expressed in a certain tissue, such as the heart. The gene for the NME7 is then operably linked to the expression of a protein expressed in the heart such as MHC. In this instance, the expression of NME7 is turned on when and where the MHC gene product is expressed. Similarly, one may want to have the expression of human NME6 or NME7 turn on in the prostate such that the location and timing of its expression is controlled by the expression of for example, a prostate specific protein. Similarly, the expression of human NME6 or NME7 in a non-human mammal can be controlled by genes expressed in mammary tissues. For example, in a transgenic mouse, human NME6 or human NME7 is expressed from the prolactin promoter, or a similar gene.

### Inhibitors of NME Proteins as Anti-Cancer Agents

Which NME proteins to target with inhibitors that will act as anti-cancer agents may depend on the type of cancer. For example, tumors that are shown to harbor bacterial NME of high sequence homology to human NME1 or NME7-A or -B domains or bacterial NMEs that are shown to mimic the function of human NME1 dimers or NME7-AB would be treated with antibodies or other agents that target the bacterial NME protein and inhibit its ability to dimerize, its ability to bind to MUC1* or its ability to promote cancer growth or transform cancer cells to cancer stem cells. Alternatively, in some cancer cells NME proteins that prefer dimerization may be errantly re-activated or mutated such that they resist formation of the hexameric form. Still other cancer may errantly re-activate expression of NME7 or the cleaved form NME7-AB. Thus therapeutic antibodies that recognize NME1, bacterial NMEs that mimic NME1 dimers and/or NME7-AB may be useful for the prevention or treatment of cancers. Alternatively, diagnostic assays are performed to determine which NME inhibitor is effective for a cancer or a subset of cancers.

Antibodies that bind to NME7 and inhibit its tumorigenic potential are potent anti-cancer agents and can be administered to patients for the treatment or prevention of cancers. Antibodies that inhibit tumorigenic potential of NME7 or NME7-AB are those antibodies that inhibit the ability of NME7 to bind to its cognate binding partners, which in one case is the PSMGFR portion of the MUC1* receptor. In another case, NME7 can function by entering the cell, translocating to the nucleus and acting as a direct or indirect transcription factor, turning on genes that promote tumorigenesis such as CXCR4, SOX2, MUC1, E-cadherin, OCT4. NME7 or NME7-AB down-regulates BRD4, JMJD6, MBD3 and CHD4, all of which results in increased tumorigenic potential of a cell. Therefore antibodies for the treatment or prevention of cancer are those that when tested in vitro or in vivo inhibit NME7 binding to MUC1* or inhibit NME7 or its co-factors from binding to the nucleic acid promoter sites of CXCR4, SOX2, MUC1, E-cadherin, OCT4, BRD4, JMJD6, MBD3 or CHD4. These antibodies can be administered to a patient with cancer or at risk of developing cancer. As is well known in the art, antibodies and antibody-like molecules can be generated using the entire NME1, NME6 or NME7 protein. Alternatively, peptides or portions of the proteins can be used. Still in other methods, peptides are injected into a host animal along with carrier molecules or adjuvant to elicit an immune response. Antibodies may be harvested from an animal in the standard ways, including monoclonal antibodies produced from antibody-producing cells harvested from an animal which can then be humanized. The invention also envisions using NME1, NME6 or NME7 proteins, or peptides whose sequences are derived from them, in screening assays. In one such example, antibody libraries can be screened for their ability to bind to NME1, NME6 or NME7, wherein antibodies that bind to the targeted NME protein are then used to treat or prevent cancers. Moreover, the library need not be comprised of antibodies per se. Libraries of antibody epitopes or fragments can be screened for binding to portions of the NME proteins in order to identify therapeutic antibodies for the treatment of persons with cancer or at risk of developing cancers. One or more of the immunogenic peptides listed in FIGS. 62-64 are ideal for generating antibodies in a host animal or for identifying and selecting antibody epitope which can later be engineered into antibody-like molecules for administration to a patient. In another embodiment, peptides whose sequences are derived from NME1, NME6 or preferably NME7 are directly administered to a human, such that the recipient generates an immune response including antibody production as a cancer vaccine. One or more of the peptides listed in FIGS. 62-64 (SEQ ID NO. 88-140) are preferred for antibody generation or selection, whether for antibody generation in a host animal, for use as a vaccine, for bait to screen libraries of synthetic peptides or antibody epitopes such as the Ylanthia® system, wherein said antibodies will inhibit cancers by inhibiting the function of NME7 or marking it for degradation. In another aspect, the invention is directed to peptide fragments of NME family proteins, and using these peptides to generate or select anti-cancer antibodies or antibody epitopes that bind to and inhibit NME7, selected from SEQ ID NOS:88-140, more preferably 88-133, more preferably 88-121.

NME7 may be an ideal therapeutic target for the treatment or prevention of cancers because its primary role appears to be in very early embryogenesis and is not expressed at significant levels in adult tissues. Therefore, agents that disable NME7 are expected to prevent or greatly inhibit cancers while having minor if any adverse effects on healthy adult tissues. Our studies show that cancer cells and naïve stem cells secrete NME7, which can function as their only required growth factor. In addition, we showed that the population of cancer cells that are metastatic cancer cells, also called cancer stem cells or tumor initiating cells, are preferentially expanded by contacting them with NME1 dimers, bacterial NME dimers, or NME7, wherein NME7 produced the greatest number of cancer stem cells. Therefore agents that disable NME proteins are excellent anti-cancer therapeutics, particularly useful for the inhibition or prevention of cancer stem cells or tumor initiating cells. In a preferred embodiment, the NME protein that is targeted by the therapeutic agent is NME1, human or bacterial, wherein the therapeutic agent inhibits dimerization, inhibits binding to MUC1*, or inhibits its ability to up-regulate pluripotency genes or cancer stem cell genes such as CXCR4. In a more preferred embodiment the NME protein that is targeted by the therapeutic agents is NME7, wherein the therapeutic agent inhibits expression of NME7, inhibits NME7 binding to MUC1*, inhibits cleavage of the DM10 domain or inhibits its ability to up-regulate pluripotency genes or cancer stem cell genes such as CXCR4.

Thus, a targeted therapeutic to inhibit growth and de-differentiation of cancer cells is an agent that disables NME7 function. NME7 function that therapeutic agents would disable for the treatment of cancer include but are not limited to: 1) ability to bind to MUC1* extra cellular domain; 2) ability to bind to DNA; 3) ability to promote stem cell proliferation; 4) ability to inhibit differentiation; 5) ability to act as a transcription factor; and 6) ability to be secreted by the cell.

Agents that disable NME7 functions as listed above include but are not limited to: antibodies, chemical entities, small molecules, microRNAs, anti-sense nucleic acids, inhibitory RNA, RNAi, siRNA. In one instance the therapeutic agent is an antibody, which can be monovalent, bivalent, bispecific, polyclonal, monoclonal or may be antibody-like in that they contain regions that mimic variable domains of antibodies. In another instance, the therapeutic agent is a chemical entity such as a small molecule. Agents that cause suppression of NME7 such as RNAi or siRNA are also envisioned as anti-cancer treatments. In a preferred embodiment, these agents block the interaction of NME7 with the extra cellular domain of MUC1*.

In an alternate approach, agents that up-regulate BRD4 are administered to a patient for the treatment or prevention of cancer, as BRD4 suppresses NME7.

### Immunizing NME Peptides to Generate Therapeutic Antibodies

Until now, very little has been known about NME proteins and their function, especially the newly identified NME proteins such as NME7. Until recently, NME1 was believed to be a hexamer. Crystal structures of NME1 and NME2 as hexamers have been published (Webb P A et al, 1995; Min K et al, 2002) but provides little information about how NME dimers or NME7 may fold. However, based on the published hexameric structure of NME1, sequence alignments among human NME1, human NME7 and bacterial NME that can mimic human NME1 and NME7 function, specifically Halomonas Sp. 593, we identify certain peptide sequences from Human NME1, human NME7 and Halomonas Sp. 593 that are predicted to give rise to antibodies for therapeutic use for the treatment or prevention of cancers as previously described herein.

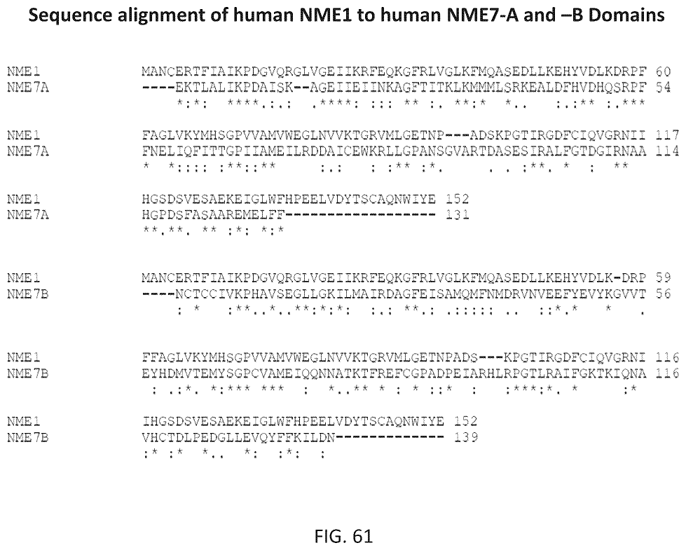

FIG. 61 is a sequence alignment between human NME1 and human NME7-A or -B domain. FIG. 27 is a sequence alignment between human NME1 and bacterial NME from Halomonas Sp 593 and between human NME7-A or -B domain and bacterial NME from Halomonas Sp 593 (‘HSP 593’).

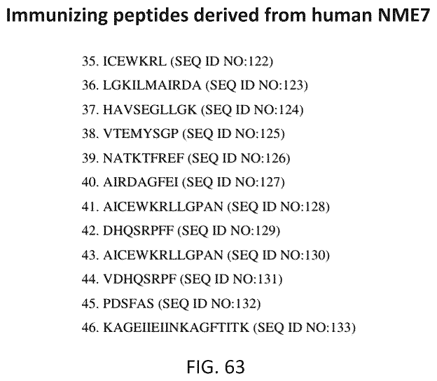

The peptides 1 to 34 listed in FIG. 62 having SEQ ID NOS:88-121) are peptides from human NME7 that were chosen because of their low homology to human NME1. NME7 peptides 35 to 46 (SEQ ID NOS:122-133) (FIG. 63) were selected because they are somewhat unique sequences regarding regions of NME7 that appear to be structurally important to the integrity of the protein or for their ability to bind to MUC1* peptide. Both sets of NME7 sequences are expected to give rise to antibodies that bind to NME7, whereas the second set of NME7 peptides may function to disable NME7 or its ability to bind to MUC1* peptide. These peptides are expected to give rise to antibodies that would recognize NME7 or could also recognize human NME1 or bacterial NMEs and thus can be used for the treatment or prevention of cancers.

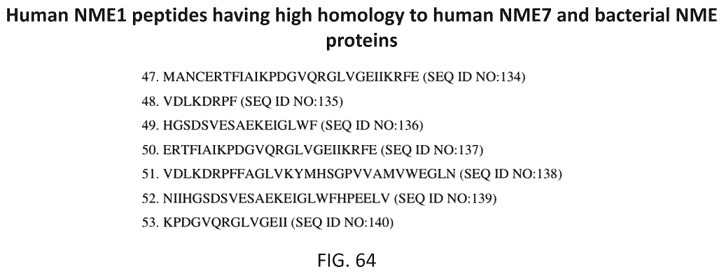

The peptides 47 to 53 (SEQ ID NOS: 134-140) listed in FIG. 64 are human NME1 sequences chosen for their high sequence homology to both human NME7 and bacterial HSP 593 NME, so are inferred to be important for structure or binding to MUC1*. These peptides are expected to give rise to antibodies that could recognize NME1, NME7 or bacterial NMEs and thus can be used for the treatment or prevention of cancers.

The peptide sequences that have low homology to human NME1 but high homology to human NME7-A or NME7-B are listed in FIG. 62, peptides 1 to 34 (SEQ ID NOS:88-121) should give rise to antibodies that prefer to bind to human NME7, which should have limited if any role in adult tissue, except in cancerous tissue in which case it is desired to inhibit its activity.

The peptides 35 to 46 (SEQ ID NOS: 122-133) listed in FIG. 63 are peptide sequences from NME7 wherein they appear to be important for structural integrity or binding to MUC1* based on sequence homology, the published crystal structure of the NME1 hexamer and the knowledge that C-terminal truncations prefer dimerization and do not inhibit binding to MUC1* or the function of the protein in stem and cancer growth. The peptides 47 to 53 (SEQ ID NOS: 134-140) listed in FIG. 64 are peptide sequences from NME1 wherein they appear to be important for structural integrity or binding to MUC1* based on sequence homology, the published crystal structure of the NME1 hexamer and the knowledge that C-terminal truncations prefer dimerization and do not inhibit binding to MUC1* or the function of the protein in stem and cancer growth. Antibodies generated from peptides or peptide mimics containing these sequences will give rise to antibodies that can be administered to a patient for the treatment or prevention of cancers. Peptides or peptide mimics containing these sequences will give rise to antibodies in a host and thus constitute an anti-cancer vaccine that can be administered to a patient for the treatment or prevention of cancers.

### Diagnostic Assays

In yet another aspect of the invention, diagnostic assays are described that can determine whether the predominant NME in a patient's cancer, or in a patient at risk of developing a cancer, is NME1, bacterial NME or NME7 full-length or cleaved to the NME7-AB form. The diagnostic assay involves standard assays such as IHC, ICC, FISH, RNA-Seq and other detection or sequencing techniques, but unlike standard cancer diagnostic tests, the assays would be performed to determine whether NME1, NME7 or bacterial NME is present in amounts greater than those measured in a control group. Based on such determination of the type of NME protein that is expressed by the patient's cancer or by a subset of cancers afflicting many patients, anti-NME antibodies or other NME disabling agents that will specifically inhibit or disable the NME protein(s) present in the patient, or group of patients are selected and administered to the patient(s). Similarly, diagnostic assay are employed to determine if the patient's NME protein bears a mutation that makes the protein favor dimerization and if so, agents that disable that particular mutant NME are administered to the patient for the treatment or prevention of cancer.

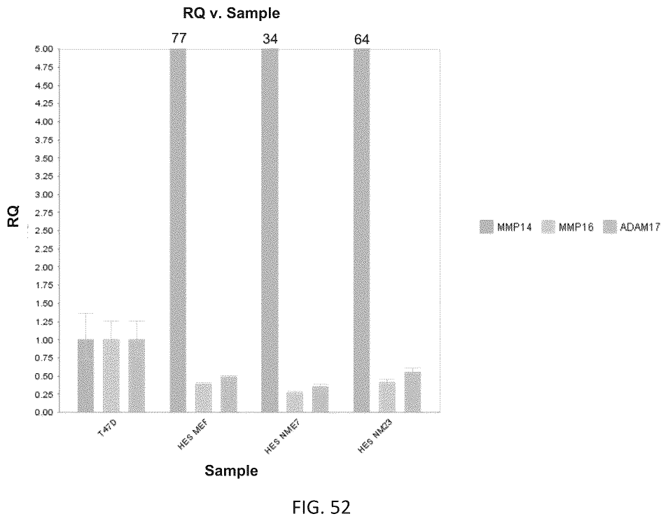

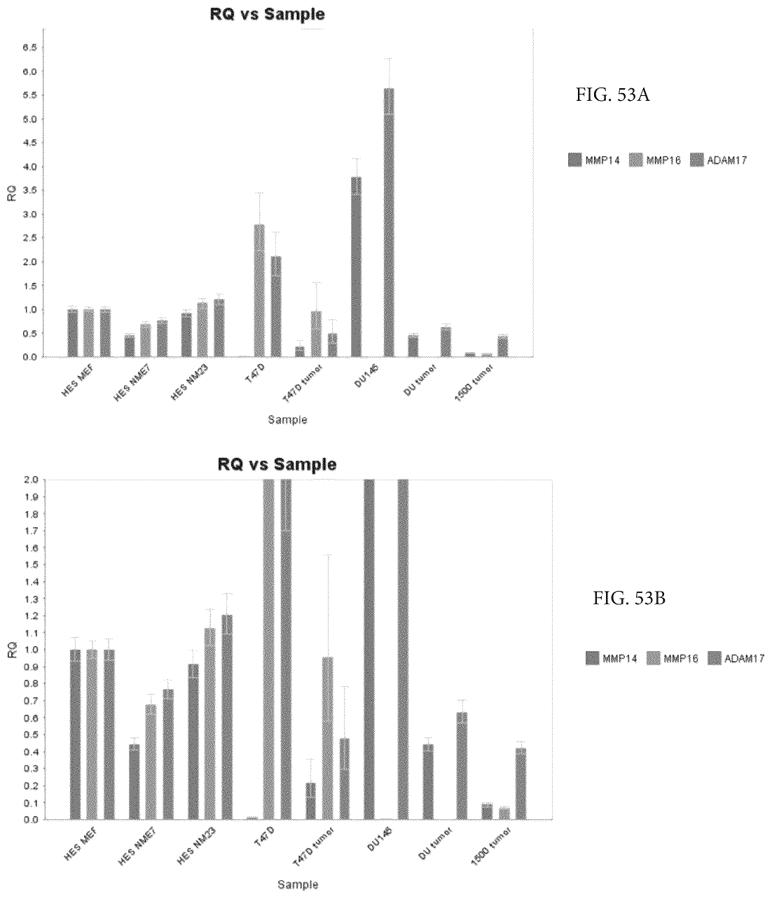

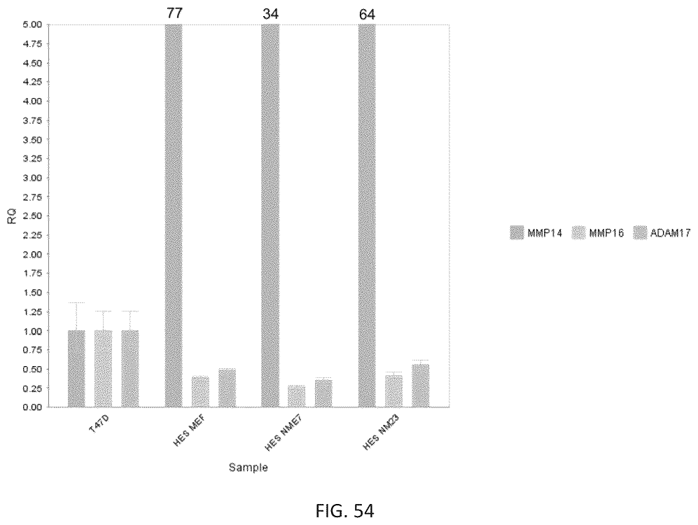

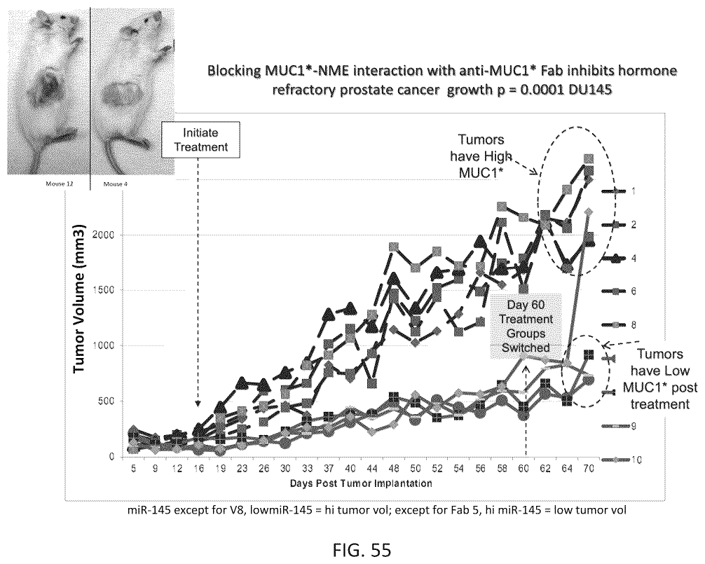

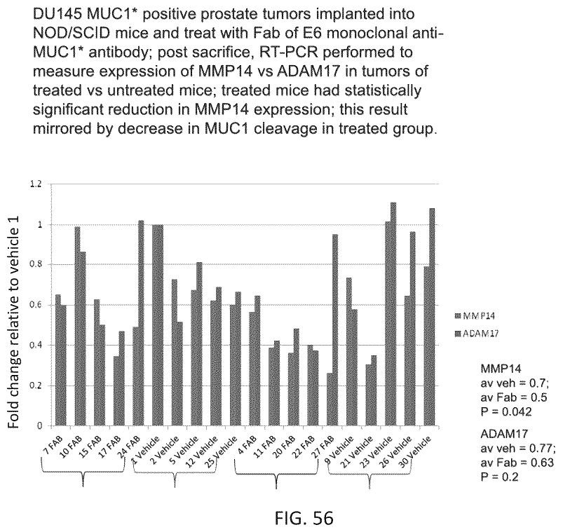

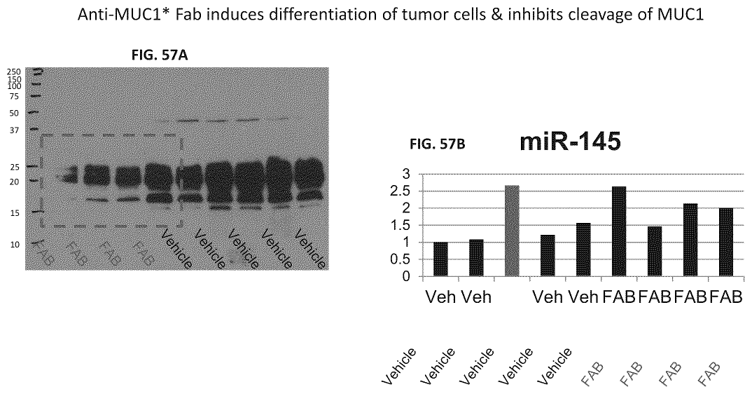

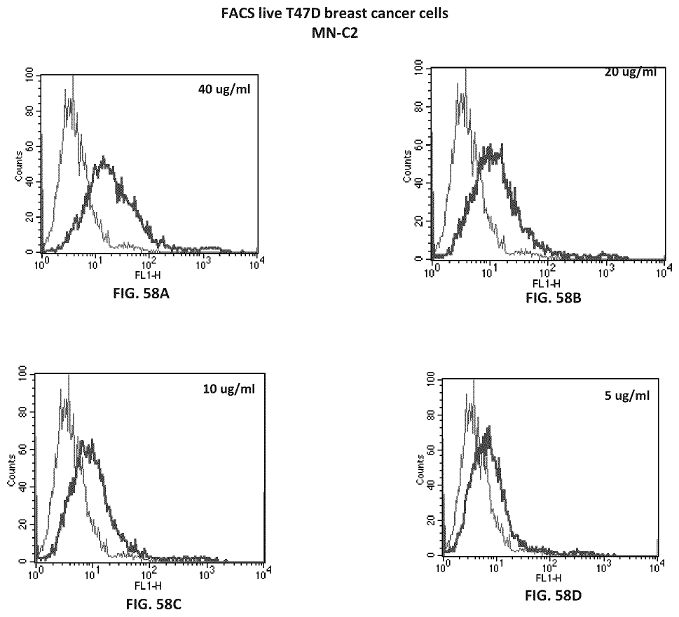

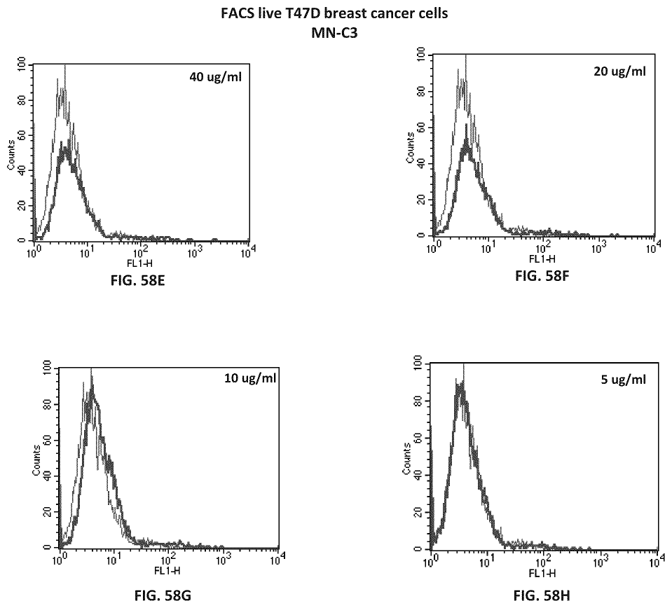

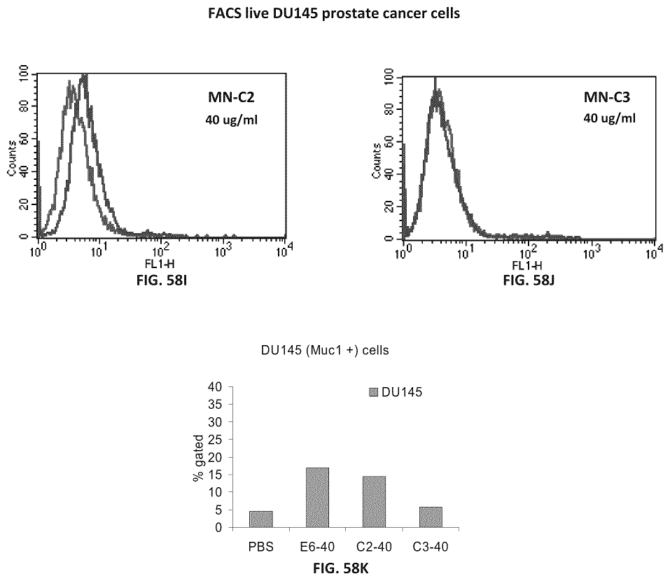

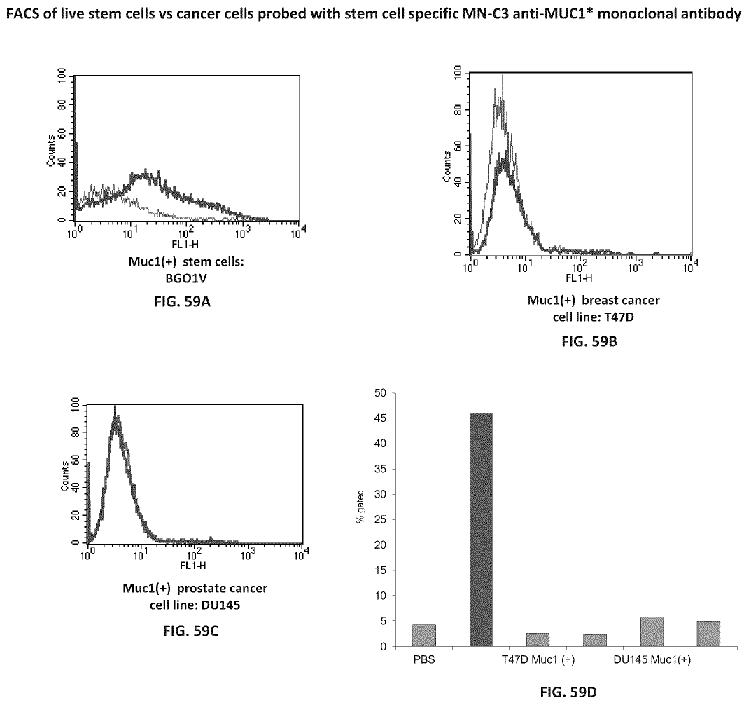

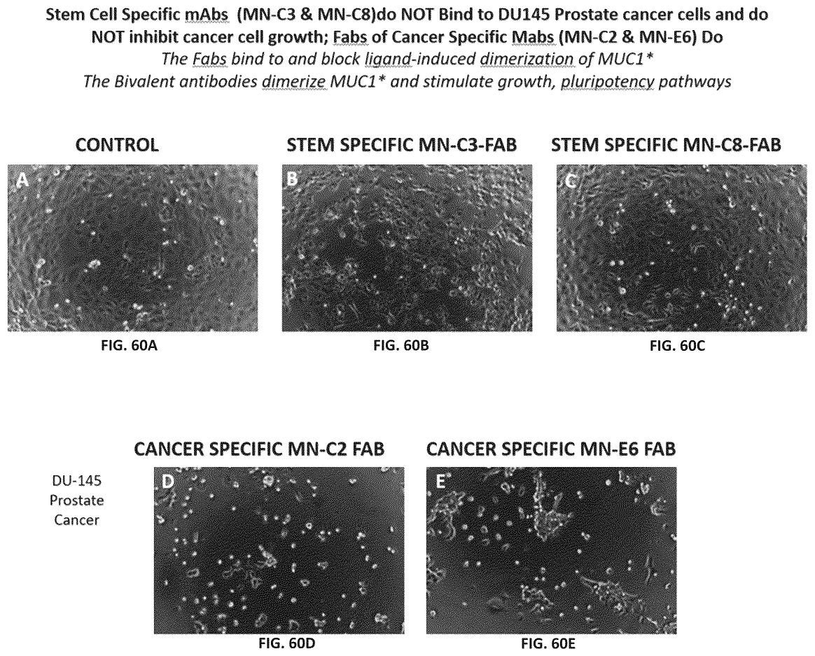

Antibodies that disable the function of the targeted NME protein, or its cognate receptor MUC1*, may be further screened to identify those antibodies that preferentially target cancer cells and do not target stem or progenitor cells or do so to a much lesser degree. MUC1 is cleaved to the MUC1* form by a variety of cleavage enzymes, wherein which enzyme cleaves MUC1 may be due to the tissue type or the timing of development of the cell or the organism. For example, MMP14 is expressed at higher levels in stem cells than it is on breast cancer cells (FIG. 52). Conversely, MMP14 and ADAM17, also MUC1 cleavage enzymes are expressed on DU145 prostate cancer cells 3- and 5-times higher than they are in human stem cells; in T47D breast cancer cells MMP16 and ADAM17 are 2-times higher than they are in stem cells (FIGS. 53 and 54). Indeed, when mice implanted with DU145 prostate cancer cells are treated with the Fab of the anti-MUC1* antibody MN-E6, tumor growth was greatly inhibited (FIG. 55), expression of MMP14 and ADAM17 was reduced (FIG. 56), MUC1 cleavage was reduced and expression of microRNA-145 that signals differentiation was increased (FIG. 57A,B) Thus, MUC1* may vary at its distal, N-terminus by 10 or more amino acids. The C-terminus of MUC1 is intracellular and its N-terminus is extracellular. Our experiments show that NME1 dimers bind to the N-10 version of the PSMGFR peptide. That is to say that omitting the first 10 amino acids of the PSMGFR peptide, which corresponds to the majority of the MUC1* extracellular domain, did not affect the ability of NME1 dimers to bind to the MUC1* peptide. Antibodies that preferentially bind to the N-10 peptide (SEQ ID NO:86) preferentially bind to MUC1* as it exists on cancer cells. Conversely, antibodies that preferentially bind to the C-10 peptide (SEQ ID NO:87), preferentially bind to stem cells and cell of the bone marrow rather than cancer cells (FIGS. 58-60). Therefore, antibodies that target MUC1* for the treatment or prevention of cancer may be generated by immunization with the PSMGFR peptide, the N-10 peptide or the C-10 peptide. Alternatively, therapeutic antibodies or antibody-like molecules for the treatment or prevention of cancers can be identified by selecting those that bind to MUC1* as it appears on cancer cells as opposed to how it appears on stem and progenitor cells. In a preferred embodiment, the antibody prefers binding to the N-10 peptide. In a yet more preferred embodiment, the therapeutic antibody is selected for its ability to bind to cancer cells but not stem or progenitor cells. In one example, antibodies were first selected for their ability to bind to the PSMGFR peptide, the N-10 peptide or the C-10 peptide by ELISA or similar direct binding assay, then confirmed to be able to bind to MUC1* positive cancer cells of many different types, wherein one antibody may bind prostate cancer cells better than breast cancer cells or vice versa, in support of the hypothesis of different cleavage sites on different tissue types. Then, hybridoma supernatants were coated onto multi-well plates and stem cells were plated over them. Since human stem cells are non-adherent, wells that were coated with an antibody that bound to stem cells (or progenitor cells) caused the stem cells to adhere, while antibodies that did not cause the stem cells to adhere were selected as preferred anti-MUC1* antibodies for the treatment or prevention of cancers.

Therefore, in another aspect, the invention is directed to a method for classifying cancers or stratifying patients, having or suspected of having cancer, including the steps of: (i) analyzing a patient sample for the presence of stem or progenitor cell genes or gene products; and (ii) grouping patients who share similar expression or expression levels of stem or progenitor cell genes or gene products. In this way, the patients can then be treated with agents that inhibit those stem or progenitor cell genes or gene products.

In another case, the expression levels of the stem or progenitor genes or gene products are measured to assess severity of the cancer, wherein expression of, or higher expression of, genes or gene products that are characteristic of earlier stem or progenitor states indicate more aggressive cancers and expression of, or higher expression of, genes or gene products that are characteristic of later progenitor states indicate less aggressive cancers. Such determination would then allow the physician to design a therapy commensurate with treating a patient with cancer more or less aggressive cancer.

These methods for classifying cancers or stratifying cancers can be accomplished with a blood sample, bodily fluid, or biopsy. The gene or gene products whose high expression level would indicate a very aggressive cancer would include NME1, more preferably NME6 and still more preferably NME7.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. The following examples are offered by way of illustration of the present invention, and not by way of limitation.

### EXAMPLES

### Example 1—Components of Minimal Serum-Free Base (“MM”) (500 mls)

400 ml DME/F12/GlutaMAX I (Invitrogen #10565-018)
100 ml Knockout Serum Replacement (KO-SR, Invitrogen #10828-028)
5 ml 100×MEM Non-essential Amino Acid Solution (Invitrogen #11140-050)
0.9 ml (0.1 mM) β-mercaptoethanol (55 mM stock, Invitrogen #21985-023)

### Example 2—Probing Cancer and Stem Cells for the Presence of NME1, NME6 and NME7

In this series of experiments, we probed the expression of NME6 and NME7 in stem cells and cancer cells. In addition, we identified MUC1* as the target of NME7. We first performed Western blot assays on cell lysates to determine the presence or absence of NME1, NME6 and NME7. In FIG. 3A, lysates from BGO1v human embryonic stem cells that had been cultured in NME1 dimers over a surface coated with anti-MUC1* antibodies (Lane 1), or cultured in bFGF over MEFs (Lane 2) or T47D human breast cancer cell lysates (Lane 3) or NME1-wt as a positive control, were separated by SDS-PAGE then probed with an anti-NME1 specific antibody. The results show that NME1 is strongly expressed in human ES cells whether cultured in NME1 dimers or bFGF, and in T47D cancer cells. The same cell lysates are separated by SDS-PAGE and then probed with an anti-NME6 specific antibody (anti-NME6 from Abnova). No NME6 was detected (data not shown), however it was detected later in a more concentrated sample (see FIG. 4).

In FIG. 3B, the same cell lysates are separated by SDS-PAGE and then probed with an anti-NME7 specific antibody (nm23-H7 B9 from Santa Cruz Biotechnology, Inc). The results show that NME7 is strongly expressed in human ES cells cultured in NME1 dimers over an anti-MUC1* antibody surface (Lane 1), weakly expressed in the same ES cells that were cultured in bFGF over MEFs (Lane 2), and strongly expressed in breast cancer cells (Lane 3). Lane 4 in which NME1 was added is blank indicating that the NME7 antibody does not cross react with NME1. The fact that NME7 is expressed to a greater degree in stem cells cultured in NME1 dimers, which we have shown express markers indicating that they are in a more naïve state than cells cultured in bFGF, means that NME7 is expressed at a higher level in naïve cells, compared to its expression in primed cells.

To determine whether NME7 also functions as a growth factor with MUC1* as its target receptor, we performed pull-down assays. In these experiments, a synthetic MUC1* extra cellular domain peptide (His-tagged PSMGFR sequence) was immobilized on NTA-Ni magnetic beads. These beads were incubated with the cell lysates of BGO1v human embryonic stem cells that had been cultured in NME1 dimers over a surface coated with anti-MUC1* antibodies (Lane 1), or cultured in bFGF over MEFs (Lane 2) or T47D human breast cancer cell lysates (Lane 3). Beads were rinsed and captured proteins were released by addition of imidazole. Proteins were separated by SDS-PAGE and then probed with either an anti-NME1 antibody (FIG. 3C), an anti-NME6 antibody (data not shown) or an NME7 antibody (FIG. 3D). The results show that NME7 binds to the MUC1* extra cellular domain peptide. This means that in stem cells and cancer cells, NME7 via its portions of its two NDPK domains, activates pluripotency pathways by dimerizing the MUC1* extra cellular domain.

### Example 3—Generation of Protein Constructs

Generating recombinant NME7—First constructs were made to make a recombinant NME7 that could be expressed efficiently and in soluble form. The first approach was to make a construct that would encode the native NME7 (−1) or an alternative splice variant NME7 (−2), which has an N-terminal deletion. In some cases, the constructs carried a histidine tag or a strep tag to aid in purification. NME7-1 expressed poorly in E. coli and NME7-2 did not express at all in E. coli. However, a novel construct was made in which the targeting sequence was deleted and the NME7 comprised essentially the NDPK A and B domains having a calculated molecular weight of 31 kDa. This novel NME7-AB expressed very well in E. coli and existed as the soluble protein. A construct in which a single NDPK domain was expressed, NME-A, did not express in E. coli. NME7-AB was first purified over an NTA-Ni column and then further purified by size exclusion chromatography (FPLC) over a Sephadex 200 column. The purified NME7-AB protein was then tested for its ability to promote pluripotency and inhibit differentiation of stem cells.

### Example 4—Functional Testing of Human Recombinant NME7-AB

Testing recombinant NME7 for ability to maintain pluripotency and inhibit differentiation. A soluble variant of NME7, NME7-AB, was generated and purified. Human stem cells (iPS cat #SC101a-1, System Biosciences) were grown per the manufacturer's directions in 4 ng/ml bFGF over a layer of mouse fibroblast feeder cells for four passages. These source stem cells were then plated into 6-well cell culture plates (Vita™, Thermo Fisher) that had been coated with 12.5 ug/well of a monoclonal anti-MUC1* antibody, MN-C3. Cells were plated at a density of 300,000 cells per well. The base media was Minimal Stem Cell Media consisting of: 400 ml DME/F12/GlutaMAX I (Invitrogen #10565-018), 100 ml Knockout Serum Replacement (KO-SR, Invitrogen #10828-028), 5 ml 100×MEM Non-essential Amino Acid Solution (Invitrogen#11140-050) and 0.9 ml (0.1 mM) β-mercaptoethanol (55 mM stock, Invitrogen #21985-023). The base media can be any media. In a preferred embodiment, the base media is free of other growth factors and cytokines. To the base media was added either 8 nM of NME7-AB or 8 nM NM23-H1 refolded and purified as stable dimers. Media was changed every 48 hours and due to accelerated growth had to be harvested and passaged at Day 3 post-plating. FIGS. 9 and 10 document the day by day comparison of growth in NM23-H1 dimers to growth in NME7 monomers. NME7 and NM23-H1 (NME1) dimers both grew pluripotently and had no differentiation even when 100% confluent. As can be seen in the photos, NME7 cells grew faster than the cells grown in NM23-H1 dimers. Cell counts at the first harvest verified that culture in NME7 produced 1.4-times more cells than culture in NM23-H1 dimers. ICC staining for the typical pluripotent markers confirmed that NME7-AB fully supported human stem cell growth, pluripotency and resisted differentiation (FIG. 11).

### Example 5—Generating Variants of NME6 and NME7

The following novel NME6 and NME7 variants were designed and generated:

Human NM23-H7-2 sequence optimized for E. coli expression:

(DNA)
(SEQ ID NO: 20)
atgcatgacgttaaaaatcaccgtacctttctgaaacgcacgaaatatga
taatctgcatctggaagacctgtttattggcaacaaagtcaatgtgttct
ctcgtcagctggtgctgatcgattatggcgaccagtacaccgcgcgtcaa
ctgggtagtcgcaaagaaaaaacgctggccctgattaaaccggatgcaat
ctccaaagctggcgaaattatcgaaattatcaacaaagcgggtttcacca
tcacgaaactgaaaatgatgatgctgagccgtaaagaagccctggatttt
catgtcgaccaccagtctcgcccgtttttcaatgaactgattcaattcat
caccacgggtccgattatcgcaatggaaattctgcgtgatgacgctatct
gcgaatggaaacgcctgctgggcccggcaaactcaggtgttgcgcgtacc
gatgccagtgaatccattcgcgctctgtttggcaccgatggtatccgtaa
tgcagcacatggtccggactcattcgcatcggcagctcgtgaaatggaac
tgtttttcccgagctctggcggttgcggtccggcaaacaccgccaaattt
accaattgtacgtgctgtattgtcaaaccgcacgcagtgtcagaaggcct
gctgggtaaaattctgatggcaatccgtgatgctggctttgaaatctcgg
ccatgcagatgttcaacatggaccgcgttaacgtcgaagaattctacgaa
gtttacaaaggcgtggttaccgaatatcacgatatggttacggaaatgta
ctccggtccgtgcgtcgcgatggaaattcagcaaaacaatgccaccaaaa
cgtttcgtgaattctgtggtccggcagatccggaaatcgcacgtcatctg
cgtccgggtaccctgcgcgcaatttttggtaaaacgaaaatccagaacgc
tgtgcactgtaccgatctgccggaagacggtctgctggaagttcaatact
ttttcaaaattctggataattga
(amino acids)
(SEQ ID NO: 21)
MHDVKNHRTFLKRTKYDNLHLEDLFIGNKVNVFSRQLVLIDYGDQYTARQ
LGSRKEKTLALIKPDAISKAGEIIEIINKAGFTITKLKMMMLSRKEALDF
HVDHQSRPFFNELIQFITTGPIIAMEILRDDAICEWKRLLGPANSGVART
DASESIRALFGTDGIRNAAHGPDSFASAAREMELFFPSSGGCGPANTAKF
TNCTCCIVKPHAVSEGLLGKILMAIRDAGFEISAMQMFNMDRVNVEEFYE
VYKGVVTEYHDMVTEMYSGPCVAMEIQQNNATKTFREFCGPADPEIARHL
RPGTLRAIFGKTKIQNAVHCTDLPEDGLLEVQYFFKILDN-

Human NME7-A:

(DNA)
(SEQ ID NO: 22)
atggaaaaaacgctagccctaattaaaccagatgcaatatcaaaggctgg
agaaataattgaaataataaacaaagctggatttactataaccaaactca
aaatgatgatgctttcaaggaaagaagcattggattttcatgtagatcac
cagtcaagaccctttttcaatgagctgatccagtttattacaactggtcc
tattattgccatggagattttaagagatgatgctatatgtgaatggaaaa
gactgctgggacctgcaaactctggagtggcacgcacagatgcttctgaa
agcattagagccctctttggaacagatggcataagaaatgcagcgcatgg
ccctgattcttttgcttctgcggccagagaaatggagttgtttttttga
(amino acids)
(SEQ ID NO: 23)
MEKTLALIKPDAISKAGEIIEIINKAGFTITKLKMMMLSRKEALDFHVDH
QSRPFFNELIQFITTGPIIAMEILRDDAICEWKRLLGPANSGVARTDASE
SIRALFGTDGIRNAAHGPDSFASAAREMELFF-

Human NME7-A1:

(DNA)
(SEQ ID NO: 24)
atggaaaaaacgctagccctaattaaaccagatgcaatatcaaaggctgg
agaaataattgaaataataaacaaagctggatttactataaccaaactca
aaatgatgatgctttcaaggaaagaagcattggattttcatgtagatcac
cagtcaagaccctttttcaatgagctgatccagtttattacaactggtcc
tattattgccatggagattttaagagatgatgctatatgtgaatggaaaa
gactgctgggacctgcaaactctggagtggcacgcacagatgcttctgaa
agcattagagccctctttggaacagatggcataagaaatgcagcgcatgg
ccctgattcttttgcttctgcggccagagaaatggagttgttttttcctt
caagtggaggttgtgggccggcaaacactgctaaatttacttga
(amino acids)
(SEQ ID NO: 25)
MEKTLALIKPDAISKAGEIIEIINKAGFTITKLKMMMLSRKEALDFHVDH
QSRPFFNELIQFITTGPIIAMEILRDDAICEWKRLLGPANSGVARTDASE
SIRALFGTDGIRNAAHGPDSFASAAREMELFFPSSGGCGPANTAKFT-

Human NME7-A2:

(DNA)
(SEQ ID NO: 26)
atgaatcatagtgaaagattcgttttcattgcagagtggtatgatccaaa
tgcttcacttcttcgacgttatgagcttttattttacccaggggatggat
ctgttgaaatgcatgatgtaaagaatcatcgcacctttttaaagcggacc
aaatatgataacctgcacttggaagatttatttataggcaacaaagtgaa
tgtcttttctcgacaactggtattaattgactatggggatcaatatacag
ctcgccagctgggcagtaggaaagaaaaaacgctagccctaattaaacca
gatgcaatatcaaaggctggagaaataattgaaataataaacaaagctgg
atttactataaccaaactcaaaatgatgatgctttcaaggaaagaagcat
tggattttcatgtagatcaccagtcaagaccctttttcaatgagctgatc
cagtttattacaactggtcctattattgccatggagattttaagagatga
tgctatatgtgaatggaaaagactgctgggacctgcaaactctggagtgg
cacgcacagatgcttctgaaagcattagagccctctttggaacagatggc
ataagaaatgcagcgcatggccctgattcttttgcttctgcggccagaga
aatggagttgtttttttga
(amino acids)
(SEQ ID NO: 27)
MNHSERFVFIAEWYDPNASLLRRYELLFYPGDGSVEMHDVKNHRTFLKRT
KYDNLHLEDLFIGNKVNVFSRQLVLIDYGDQYTARQLGSRKEKTLALIKP
DAISKAGEIIEIINKAGFTITKLKMMMLSRKEALDFHVDHQSRPFFNELI
QFITTGPIIAMEILRDDAICEWKRLLGPANSGVARTDASESIRALFGTDG
IRNAAHGPDSFASAAREMELFF-

Human NME7-A3:

(DNA)
(SEQ ID NO: 28)
atgaatcatagtgaaagattcgttttcattgcagagtggtatgatccaaa
tgcttcacttcttcgacgttatgagcttttattttacccaggggatggat
ctgttgaaatgcatgatgtaaagaatcatcgcacctttttaaagcggacc
aaatatgataacctgcacttggaagatttatttataggcaacaaagtgaa
tgtcttttctcgacaactggtattaattgactatggggatcaatatacag
ctcgccagctgggcagtaggaaagaaaaaacgctagccctaattaaacca
gatgcaatatcaaaggctggagaaataattgaaataataaacaaagctgg
atttactataaccaaactcaaaatgatgatgctttcaaggaaagaagcat
tggattttcatgtagatcaccagtcaagaccctttttcaatgagctgatc
cagtttattacaactggtcctattattgccatggagattttaagagatga
tgctatatgtgaatggaaaagactgctgggacctgcaaactctggagtgg
cacgcacagatgcttctgaaagcattagagccctctttggaacagatggc
ataagaaatgcagcgcatggccctgattcttttgcttctgcggccagaga
aatggagttgttttttccttcaagtggaggttgtgggccggcaaacactg
ctaaatttacttga
(amino acids)
(SEQ ID NO: 29)
MNHSERFVFIAEWYDPNASLLRRYELLFYPGDGSVEMHDVKNHRTFLKRT
KYDNLHLEDLFIGNKVNVFSRQLVLIDYGDQYTARQLGSRKEKTLALIKP
DAISKAGEIIEIINKAGFTITKLKMMMLSRKEALDFHVDHQSRPFFNELI
QFITTGPIIAMEILRDDAICEWKRLLGPANSGVARTDASESIRALFGTDG
IRNAAHGPDSFASAAREMELFFPSSGGCGPANTAKFT-

Human NME7-B:

(DNA)
(SEQ ID NO: 30)
atgaattgtacctgttgcattgttaaaccccatgctgtcagtgaaggact
gttgggaaagatcctgatggctatccgagatgcaggttttgaaatctcag
ctatgcagatgttcaatatggatcgggttaatgttgaggaattctatgaa
gtttataaaggagtagtgaccgaatatcatgacatggtgacagaaatgta
ttctggcccttgtgtagcaatggagattcaacagaataatgctacaaaga
catttcgagaattttgtggacctgctgatcctgaaattgcccggcattta
cgccctggaactctcagagcaatctttggtaaaactaagatccagaatgc
tgttcactgtactgatctgccagaggatggcctattagaggttcaatact
tcttctga
(amino acids)
(SEQ ID NO: 31)
MNCTCCIVKPHAVSEGLLGKILMAIRDAGFEISAMQMFNMDRVNVEEFYE
VYKGVVTEYHDMVTEMYSGPCVAMEIQQNNATKTFREFCGPADPEIARHL
RPGTLRAIFGKTKIQNAVHCTDLPEDGLLEVQYFF-

Human NME7-B1:

(DNA)
(SEQ ID NO: 32)
atgaattgtacctgttgcattgttaaaccccatgctgtcagtgaaggact
gttgggaaagatcctgatggctatccgagatgcaggttttgaaatctcag
ctatgcagatgttcaatatggatcgggttaatgttgaggaattctatgaa
gtttataaaggagtagtgaccgaatatcatgacatggtgacagaaatgta
ttctggcccttgtgtagcaatggagattcaacagaataatgctacaaaga
catttcgagaattttgtggacctgctgatcctgaaattgcccggcattta
cgccctggaactctcagagcaatctttggtaaaactaagatccagaatgc
tgttcactgtactgatctgccagaggatggcctattagaggttcaatact
tcttcaagatcttggataattagtga
(amino acids)
(SEQ ID NO: 33)
MNCTCCIVKPHAVSEGLLGKILMAIRDAGFEISAMQMFNMDRVNVEEFYE
VYKGVVTEYHDMVTEMYSGPCVAMEIQQNNATKTFREFCGPADPEIARHL
RPGTLRAIFGKTKIQNAVHCTDLPEDGLLEVQYFFKILDN-

Human NME7-B2:

(DNA)
(SEQ ID NO: 34)
atgccttcaagtggaggttgtgggccggcaaacactgctaaatttactaa
ttgtacctgttgcattgttaaaccccatgctgtcagtgaaggactgttgg
gaaagatcctgatggctatccgagatgcaggttttgaaatctcagctatg
cagatgttcaatatggatcgggttaatgttgaggaattctatgaagttta
taaaggagtagtgaccgaatatcatgacatggtgacagaaatgtattctg
gcccttgtgtagcaatggagattcaacagaataatgctacaaagacattt
cgagaattttgtggacctgctgatcctgaaattgcccggcatttacgccc
tggaactctcagagcaatctttggtaaaactaagatccagaatgctgttc
actgtactgatctgccagaggatggcctattagaggttcaatacttcttc
tga
(amino acids)
(SEQ ID NO: 35)
MPSSGGCGPANTAKFTNCTCCIVKPHAVSEGLLGKILMAIRDAGFEISA
MQMFNMDRVNVEEFYEVYKGVVTEYHDMVTEMYSGPCVAMEIQQNNATK
TFREFCGPADPEIARHLRPGTLRAIFGKTKIQNAVHCTDLPEDGLLEVQ
YFF-

Human NME7-B3:

(DNA)
(SEQ ID NO: 36)
atgccttcaagtggaggttgtgggccggcaaacactgctaaatttactaa
ttgtacctgttgcattgttaaaccccatgctgtcagtgaaggactgttgg
gaaagatcctgatggctatccgagatgcaggttttgaaatctcagctatg
cagatgttcaatatggatcgggttaatgttgaggaattctatgaagttta
taaaggagtagtgaccgaatatcatgacatggtgacagaaatgtattctg
gcccttgtgtagcaatggagattcaacagaataatgctacaaagacattt
cgagaattttgtggacctgctgatcctgaaattgcccggcatttacgccc
tggaactctcagagcaatctttggtaaaactaagatccagaatgctgttc
actgtactgatctgccagaggatggcctattagaggttcaatacttcttc
aagatcttggataattagtga
(amino acids)
(SEQ ID NO: 37)
MPSSGGCGPANTAKFTNCTCCIVKPHAVSEGLLGKILMAIRDAGFEISAM
QMFNMDRVNVEEFYEVYKGVVTEYHDMVTEMYSGPCVAMEIQQNNATKTF
REFCGPADPEIARHLRPGTLRAIFGKTKIQNAVHCTDLPEDGLLEVQYFF
KILDN--

Human NME7-AB:

(DNA)
(SEQ ID NO: 38)
atggaaaaaacgctagccctaattaaaccagatgcaatatcaaaggctgg
agaaataattgaaataataaacaaagctggatttactataaccaaactca
aaatgatgatgctttcaaggaaagaagcattggattttcatgtagatcac
cagtcaagaccctttttcaatgagctgatccagtttattacaactggtcc
tattattgccatggagattttaagagatgatgctatatgtgaatggaaaa
gactgctgggacctgcaaactctggagtggcacgcacagatgcttctgaa
agcattagagccctctttggaacagatggcataagaaatgcagcgcatgg
ccctgattcttttgcttctgcggccagagaaatggagttgttttttcctt
caagtggaggttgtgggccggcaaacactgctaaatttactaattgtacc
tgttgcattgttaaaccccatgctgtcagtgaaggactgttgggaaagat
cctgatggctatccgagatgcaggttttgaaatctcagctatgcagatgt
tcaatatggatcgggttaatgttgaggaattctatgaagtttataaagga
gtagtgaccgaatatcatgacatggtgacagaaatgtattctggcccttg
tgtagcaatggagattcaacagaataatgctacaaagacatttcgagaat
tttgtggacctgctgatcctgaaattgcccggcatttacgccctggaact
ctcagagcaatctttggtaaaactaagatccagaatgctgttcactgtac
tgatctgccagaggatggcctattagaggttcaatacttcttcaagatct
tggataattagtga
(amino acids)
(SEQ ID NO: 39)
MEKTLALIKPDAISKAGEIIEIINKAGFTITKLKMMMLSRKEALDFHVDH
QSRPFFNELIQFITTGPIIAMEILRDDAICEWKRLLGPANSGVARTDASE
SIRALFGTDGIRNAAHGPDSFASAAREMELFFPSSGGCGPANTAKFTNCT
CCIVKPHAVSEGLLGKILMAIRDAGFEISAMQMFNMDRVNVEEFYEVYKG
VVTEYHDMVTEMYSGPCVAMEIQQNNATKTFREFCGPADPEIARHLRPGT
LRAIFGKTKIQNAVHCTDLPEDGLLEVQYFFKILDN--

Human NME7-ABI:

(DNA)

(SEQ ID NO: 40)

atggaaaaaacgctagccctaattaaaccagatgcaatatcaaaggctgg
agaaataattgaaataataaacaaagctggatttactataaccaaactca
aaatgatgatgctttcaaggaaagaagcattggattttcatgtagatcac
cagtcaagaccctttttcaatgagctgatccagtttattacaactggtcc
tattattgccatggagattttaagagatgatgctatatgtgaatggaaaa
gactgctgggacctgcaaactctggagtggcacgcacagatgcttctgaa
agcattagagccctctttggaacagatggcataagaaatgcagcgcatgg
ccctgattcttttgcttctgcggccagagaaatggagttgttttttcctt
caagtggaggttgtgggccggcaaacactgctaaatttactaattgtacc
tgttgcattgttaaaccccatgctgtcagtgaaggactgttgggaaagat
cctgatggctatccgagatgcaggttttgaaatctcagctatgcagatgt
tcaatatggatcgggttaatgttgaggaattctatgaagtttataaagga
gtagtgaccgaatatcatgacatggtgacagaaatgtattctggcccttg
tgtagcaatggagattcaacagaataatgctacaaagacatttcgagaat
tttgtggacctgctgatcctgaaattgcccggcatttacgccctggaact
ctcagagcaatctttggtaaaactaagatccagaatgctgttcactgtac
tgatctgccagaggatggcctattagaggttcaatacttcttctga
(amino acids)

(SEQ ID NO: 41)

MEKTLALIKPDAISKAGEIIEIINKAGFTITKLKMMMLSRKEALDFHVDH
QSRPFFNELIQFITTGPIIAMEILRDDAICEWKRLLGPANSGVARTDASE
SIRALFGTDGIRNAAHGPDSFASAAREMELFFPSSGGCGPANTAKFTNCT
CCIVKPHAVSEGLLGKILMAIRDAGFEISAMQMFNMDRVNVEEFYEVYKG
VVTEYHDMVTEMYSGPCVAMEIQQNNATKTFREFCGPADPEIARHLRPGT
LRAIFGKTKIQNAVHCTDLPEDGLLEVQYFF-

Human NME7-A sequence optimized for E. coli expression:

(DNA)

(SEQ ID NO: 42)

atggaaaaaacgctggccctgattaaaccggatgcaatctccaaagctgg
cgaaattatcgaaattatcaacaaagcgggtttcaccatcacgaaactga
aaatgatgatgctgagccgtaaagaagccctggattttcatgtcgaccac
cagtctcgcccgtttttcaatgaactgattcaattcatcaccacgggtcc
gattatcgcaatggaaattctgcgtgatgacgctatctgcgaatggaaac
gcctgctgggcccggcaaactcaggtgttgcgcgtaccgatgccagtgaa
tccattcgcgctctgtttggcaccgatggtatccgtaatgcagcacatgg
tccggactcattcgcatcggcagctcgtgaaatggaactgtttttctga
(amino acids)

(SEQ ID NO: 43)

MEKTLALIKPDAISKAGEIIEIINKAGFTITKLKMMMLSRKEALDFHVDH
QSRPFFNELIQFITTGPIIAMEILRDDAICEWKRLLGPANSGVARTDASE
SIRALFGTDGIRNAAHGPDSFASAAREMELFF-

Human NME7-A1 sequence optimized for E. coli expression:

(DNA)

(SEQ ID NO: 44)

atggaaaaaacgctggccctgattaaaccggatgcaatctccaaagctgg
cgaaattatcgaaattatcaacaaagcgggtttcaccatcacgaaactga
aaatgatgatgctgagccgtaaagaagccctggattttcatgtcgaccac
cagtctcgcccgtttttcaatgaactgattcaattcatcaccacgggtcc
gattatcgcaatggaaattctgcgtgatgacgctatctgcgaatggaaac
gcctgctgggcccggcaaactcaggtgttgcgcgtaccgatgccagtgaa
tccattcgcgctctgtttggcaccgatggtatccgtaatgcagcacatgg
tccggactcattcgcatcggcagctcgtgaaatggaactgtttttcccga
gctctggcggttgcggtccggcaaacaccgccaaatttacctga
(amino acids)

(SEQ ID NO: 45)

MEKTLALIKPDAISKAGEIIEIINKAGFTITKLKMMMLSRKEALDFHVDH
QSRPFFNELIQFITTGPIIAMEILRDDAICEWKRLLGPANSGVARTDASE
SIRALFGTDGIRNAAHGPDSFASAAREMELFFPSSGGCGPANTAKFT-

Human NME7-A2 sequence optimized for E. coli expression:

(DNA)

(SEQ ID NO: 46)

atgaatcactccgaacgctttgtttttatcgccgaatggtatgacccgaa
tgcttccctgctgcgccgctacgaactgctgttttatccgggcgatggta
gcgtggaaatgcatgacgttaaaaatcaccgtacctttctgaaacgcacg
aaatatgataatctgcatctggaagacctgtttattggcaacaaagtcaa
tgtgttctctcgtcagctggtgctgatcgattatggcgaccagtacaccg
cgcgtcaactgggtagtcgcaaagaaaaaacgctggccctgattaaaccg
gatgcaatctccaaagctggcgaaattatcgaaattatcaacaaagcggg
tttcaccatcacgaaactgaaaatgatgatgctgagccgtaaagaagccc
tggattttcatgtcgaccaccagtctcgcccgtttttcaatgaactgatt
caattcatcaccacgggtccgattatcgcaatggaaattctgcgtgatga
cgctatctgcgaatggaaacgcctgctgggcccggcaaactcaggtgttg
cgcgtaccgatgccagtgaatccattcgcgctctgtttggcaccgatggt
atccgtaatgcagcacatggtccggactcattcgcatcggcagctcgtga
aatggaactgtttttctga
(amino acids)

(SEQ ID NO: 47)

MNHSERFVFIAEWYDPNASLLRRYELLFYPGDGSVEMHDVKNHRTFLKRT
KYDNLHLEDLFIGNKVNVFSRQLVLIDYGDQYTARQLGSRKEKTLALIKP
DAISKAGEIIEIINKAGFTITKLKMMMLSRKEALDFHVDHQSRPFFNELI
QFITTGPIIAMEILRDDAICEWKRLLGPANSGVARTDASESIRALFGTDG
IRNAAHGPDSFASAAREMELFF-

Human NME7-A3 sequence optimized for E. coli expression:

(DNA)

(SEQ ID NO: 48)

atgaatcactccgaacgctttgtttttatcgccgaatggtatgacccgaa
tgcttccctgctgcgccgctacgaactgctgttttatccgggcgatggta
gcgtggaaatgcatgacgttaaaaatcaccgtacctttctgaaacgcacg
aaatatgataatctgcatctggaagacctgtttattggcaacaaagtcaa
tgtgttctctcgtcagctggtgctgatcgattatggcgaccagtacaccg
cgcgtcaactgggtagtcgcaaagaaaaaacgctggccctgattaaaccg
gatgcaatctccaaagctggcgaaattatcgaaattatcaacaaagcggg
tttcaccatcacgaaactgaaaatgatgatgctgagccgtaaagaagccc
tggattttcatgtcgaccaccagtctcgcccgtttttcaatgaactgatt
caattcatcaccacgggtccgattatcgcaatggaaattctgcgtgatga
cgctatctgcgaatggaaacgcctgctgggcccggcaaactcaggtgttg
cgcgtaccgatgccagtgaatccattcgcgctctgtttggcaccgatggt
atccgtaatgcagcacatggtccggactcattcgcatcggcagctcgtga
aatggaactgtttttcccgagctctggcggttgcggtccggcaaacaccg
ccaaatttacctga
(amino acids)

(SEQ ID NO: 49)

MNHSERFVFIAEWYDPNASLLRRYELLFYPGDGSVEMHDVKNHRTFLKRT
KYDNLHLEDLFIGNKVNVFSRQLVLIDYGDQYTARQLGSRKEKTLALIKP
DAISKAGEIIEIINKAGFTITKLKMMMLSRKEALDFHVDHQSRPFFNELI
QFITTGPIIAMEILRDDAICEWKRLLGPANSGVARTDASESIRALFGTDG
IRNAAHGPDSFASAAREMELFFPSSGGCGPANTAKFT-

Human NME7-B sequence optimized for E. coli expression:

(DNA)

(SEQ ID NO: 50)

atgaattgtacgtgctgtattgtcaaaccgcacgcagtgtcagaaggcct
gctgggtaaaattctgatggcaatccgtgatgctggctttgaaatctcgg
ccatgcagatgttcaacatggaccgcgttaacgtcgaagaattctacgaa
gtttacaaaggcgtggttaccgaatatcacgatatggttacggaaatgta
ctccggtccgtgcgtcgcgatggaaattcagcaaaacaatgccaccaaaa
cgtttcgtgaattctgtggtccggcagatccggaaatcgcacgtcatctg
cgtccgggtaccctgcgcgcaatttttggtaaaacgaaaatccagaacgc
tgtgcactgtaccgatctgccggaagacggtctgctggaagttcaatact
ttttctga
(amino acids)

(SEQ ID NO: 51)

MNCTCCIVKPHAVSEGLLGKILMAIRDAGFEISAMQMFNMDRVNVEEFYE
VYKGVVTEYHDMVTEMYSGPCVAMEIQQNNATKTFREFCGPADPEIARHL
RPGTLRAIFGKTKIQNAVHCTDLPEDGLLEVQYFF-

Human NME7-B1 sequence optimized for E. coli expression:

(DNA)

(SEQ ID NO: 52)

atgaattgtacgtgctgtattgtcaaaccgcacgcagtgtcagaaggcct
gctgggtaaaattctgatggcaatccgtgatgctggctttgaaatctcgg
ccatgcagatgttcaacatggaccgcgttaacgtcgaagaattctacgaa
gtttacaaaggcgtggttaccgaatatcacgatatggttacggaaatgta
ctccggtccgtgcgtcgcgatggaaattcagcaaaacaatgccaccaaaa
cgtttcgtgaattctgtggtccggcagatccggaaatcgcacgtcatctg
cgtccgggtaccctgcgcgcaatttttggtaaaacgaaaatccagaacgc
tgtgcactgtaccgatctgccggaagacggtctgctggaagttcaatact
ttttcaaaattctggataattga
(amino acids)

(SEQ ID NO: 53)

MNCTCCIVKPHAVSEGLLGKILMAIRDAGFEISAMQMFNMDRVNVEEFYE
VYKGVVTEYHDMVTEMYSGPCVAMEIQQNNATKTFREFCGPADPEIARHL
RPGTLRAIFGKTKIQNAVHCTDLPEDGLLEVQYFFKILDN-

Human NME7-B2 sequence optimized for E. coli expression:

(DNA)

(SEQ ID NO: 54)

atgccgagctctggcggttgcggtccggcaaacaccgccaaatttaccaa
ttgtacgtgctgtattgtcaaaccgcacgcagtgtcagaaggcctgctgg
gtaaaattctgatggcaatccgtgatgctggctttgaaatctcggccatg
cagatgttcaacatggaccgcgttaacgtcgaagaattctacgaagttta
caaaggcgtggttaccgaatatcacgatatggttacggaaatgtactccg
gtccgtgcgtcgcgatggaaattcagcaaaacaatgccaccaaaacgttt
cgtgaattctgtggtccggcagatccggaaatcgcacgtcatctgcgtcc
gggtaccctgcgcgcaatttttggtaaaacgaaaatccagaacgctgtgc
actgtaccgatctgccggaagacggtctgctggaagttcaatactttttc
tga
(amino acids)

(SEQ ID NO: 55)

MPSSGGCGPANTAKFTNCTCCIVKPHAVSEGLLGKILMAIRDAGFEISA
MQMFNMDRVNVEEFYEVYKGVVTEYHDMVTEMYSGPCVAMEIQQNNATK
TFREFCGPADPEIARHLRPGTLRAIFGKTKIQNAVHCTDLPEDGLLEVQ
YFF-

Human NME7-B3 sequence optimized for E. coli expression:

(DNA)

(SEQ ID NO: 56)

atgccgagctctggcggttgcggtccggcaaacaccgccaaatttaccaa
ttgtacgtgctgtattgtcaaaccgcacgcagtgtcagaaggcctgctgg
gtaaaattctgatggcaatccgtgatgctggctttgaaatctcggccatg
cagatgttcaacatggaccgcgttaacgtcgaagaattctacgaagttta
caaaggcgtggttaccgaatatcacgatatggttacggaaatgtactccg
gtccgtgcgtcgcgatggaaattcagcaaaacaatgccaccaaaacgttt
cgtgaattctgtggtccggcagatccggaaatcgcacgtcatctgcgtcc
gggtaccctgcgcgcaatttttggtaaaacgaaaatccagaacgctgtgc
actgtaccgatctgccggaagacggtctgctggaagttcaatactttttc
aaaattctggataattga
(amino acids)

(SEQ ID NO: 57)

MPSSGGCGPANTAKFTNCTCCIVKPHAVSEGLLGKILMAIRDAGFEISAM
QMFNMDRVNVEEFYEVYKGVVTEYHDMVTEMYSGPCVAMEIQQNNATKTF
REFCGPADPEIARHLRPGTLRAIFGKTKIQNAVHCTDLPEDGLLEVQYFF
KILDN-

Human NME7-AB sequence optimized for E. coli expression:

(DNA)

(SEQ ID NO: 58)

atggaaaaaacgctggccctgattaaaccggatgcaatctccaaagctgg
cgaaattatcgaaattatcaacaaagcgggtttcaccatcacgaaactga
aaatgatgatgctgagccgtaaagaagccctggattttcatgtcgaccac
cagtctcgcccgtttttcaatgaactgattcaattcatcaccacgggtcc
gattatcgcaatggaaattctgcgtgatgacgctatctgcgaatggaaac
gcctgctgggcccggcaaactcaggtgttgcgcgtaccgatgccagtgaa
tccattcgcgctctgtttggcaccgatggtatccgtaatgcagcacatgg
tccggactcattcgcatcggcagctcgtgaaatggaactgtttttcccga
gctctggcggttgcggtccggcaaacaccgccaaatttaccaattgtacg
tgctgtattgtcaaaccgcacgcagtgtcagaaggcctgctgggtaaaat
tctgatggcaatccgtgatgctggctttgaaatctcggccatgcagatgt
tcaacatggaccgcgttaacgtcgaagaattctacgaagtttacaaaggc
gtggttaccgaatatcacgatatggttacggaaatgtactccggtccgtg
cgtcgcgatggaaattcagcaaaacaatgccaccaaaacgtttcgtgaat
tctgtggtccggcagatccggaaatcgcacgtcatctgcgtccgggtacc
ctgcgcgcaatttttggtaaaacgaaaatccagaacgctgtgcactgtac
cgatctgccggaagacggtctgctggaagttcaatactttttcaaaattc
tggataattga
(amino acids)

(SEQ ID NO: 59)

MEKTLALIKPDAISKAGEIIEIINKAGFTITKLKMMMLSRKEALDFHVDH
QSRPFFNELIQFITTGPIIAMEILRDDAICEWKRLLGPANSGVARTDASE
SIRALFGTDGIRNAAHGPDSFASAAREMELFFPSSGGCGPANTAKFTNCT
CCIVKPHAVSEGLLGKILMAIRDAGFEISAMQMFNMDRVNVEEFYEVYKG
VVTEYHDMVTEMYSGPCVAMEIQQNNATKTFREFCGPADPEIARHLRPGT
LRAIFGKTKIQNAVHCTDLPEDGLLEVQYFFKILDN-

Human NME7-ABI sequence optimized for E. coli expression:

(DNA)

(SEQ ID NO: 60)

Atggaaaaaacgctggccctgattaaaccggatgcaatctccaaagctgg
cgaaattatcgaaattatcaacaaagcgggtttcaccatcacgaaactga
aaatgatgatgctgagccgtaaagaagccctggattttcatgtcgaccac
cagtctcgcccgtttttcaatgaactgattcaattcatcaccacgggtcc
gattatcgcaatggaaattctgcgtgatgacgctatctgcgaatggaaac
gcctgctgggcccggcaaactcaggtgttgcgcgtaccgatgccagtgaa
tccattcgcgctctgtttggcaccgatggtatccgtaatgcagcacatgg
tccggactcattcgcatcggcagctcgtgaaatggaactgtttttcccga
gctctggcggttgcggtccggcaaacaccgccaaatttaccaattgtacg
tgctgtattgtcaaaccgcacgcagtgtcagaaggcctgctgggtaaaat
tctgatggcaatccgtgatgctggctttgaaatctcggccatgcagatgt
tcaacatggaccgcgttaacgtcgaagaattctacgaagtttacaaaggc
gtggttaccgaatatcacgatatggttacggaaatgtactccggtccgtg
cgtcgcgatggaaattcagcaaaacaatgccaccaaaacgtttcgtgaat
tctgtggtccggcagatccggaaatcgcacgtcatctgcgtccgggtacc
ctgcgcgcaatttttggtaaaacgaaaatccagaacgctgtgcactgtac
cgatctgccggaagacggtctgctggaagttcaatactttttctga
(amino acids)

(SEQ ID NO: 61)

MEKTLALIKPDAISKAGEIIEIINKAGFTITKLKMMMLSRKEALDFHVDH
QSRPFFNELIQFITTGPIIAMEILRDDAICEWKRLLGPANSGVARTDASE
SIRALFGTDGIRNAAHGPDSFASAAREMELFFPSSGGCGPANTAKFTNCT
CCIVKPHAVSEGLLGKILMAIRDAGFEISAMQMFNMDRVNVEEFYEVYKG
VVTEYHDMVTEMYSGPCVAMEIQQNNATKTFREFCGPADPEIARHLRPGT
LRAIFGKTKIQNAVHCTDLPEDGLLEVQYFF-

Mouse NME6

(DNA)

(SEQ ID NO: 62)

Atgacctccatcttgcgaagtccccaagctcttcagctcacactagccct
gatcaagcctgatgcagttgcccacccactgatcctggaggctgttcatc
agcagattctgagcaacaagttcctcattgtacgaacgagggaactgcag
tggaagctggaggactgccggaggttttaccgagagcatgaagggcgttt
tttctatcagcggctggtggagttcatgacaagtgggccaatccgagcct
atatccttgcccacaaagatgccatccaactttggaggacactgatggga
cccaccagagtatttcgagcacgctatatagccccagattcaattcgtgg
aagtttgggcctcactgacacccgaaatactacccatggctcagactccg
tggtttccgccagcagagagattgcagccttcttccctgacttcagtgaa
cagcgctggtatgaggaggaggaaccccagctgcggtgtggtcctgtgca
ctacagtccagaggaaggtatccactgtgcagctgaaacaggaggccaca
aacaacctaacaaaacctag
(amino acids)

(SEQ ID NO: 63)

MTSILRSPQALQLTLALIKPDAVAHPLILEAVHQQILSNKFLIVRTRELQ
WKLEDCRRFYREHEGRFFYQRLVEFMTSGPIRAYILAHKDAIQLWRTLMG
PTRVFRARYIAPDSIRGSLGLTDTRNTTHGSDSVVSASREIAAFFPDFSE
QRWYEEEEPQLRCGPVHYSPEEGIHCAAETGGHKQPNKT-

Human NME6:

(DNA)

(SEQ ID NO: 64)

Atgacccagaatctggggagtgagatggcctcaatcttgcgaagccctca
ggctctccagctcactctagccctgatcaagcctgacgcagtcgcccatc
cactgattctggaggctgttcatcagcagattctaagcaacaagttcctg
attgtacgaatgagagaactactgtggagaaaggaagattgccagaggtt
ttaccgagagcatgaagggcgttttttctatcagaggctggtggagttca
tggccagcgggccaatccgagcctacatccttgcccacaaggatgccatc
cagctctggaggacgctcatgggacccaccagagtgttccgagcacgcca
tgtggccccagattctatccgtgggagtttcggcctcactgacacccgca
acaccacccatggttcggactctgtggtttcagccagcagagagattgca
gccttcttccctgacttcagtgaacagcgctggtatgaggaggaagagcc
ccagttgcgctgtggccctgtgtgctatagcccagagggaggtgtccact
atgtagctggaacaggaggcctaggaccagcctga
(amino acids)

(SEQ ID NO: 65)

MTQNLGSEMASILRSPQALQLTLALIKPDAVAHPLILEAVHQQILSNKFL
IVRMRELLWRKEDCQRFYREHEGRFFYQRLVEFMASGPIRAYILAHKDAI
QLWRTLMGPTRVFRARHVAPDSIRGSFGLTDTRNTTHGSDSVVSASREIA
AFFPDFSEQRWYEEEEPQLRCGPVCYSPEGGVHYVAGTGGLGPA-

Human NME6 1:

(DNA)

(SEQ ID NO: 66)

Atgacccagaatctggggagtgagatggcctcaatcttgcgaagccctca
ggctctccagctcactctagccctgatcaagcctgacgcagtcgcccatc
cactgattctggaggctgttcatcagcagattctaagcaacaagttcctg
attgtacgaatgagagaactactgtggagaaaggaagattgccagaggtt
ttaccgagagcatgaagggcgttttttctatcagaggctggtggagttca
tggccagcgggccaatccgagcctacatccttgcccacaaggatgccatc
cagctctggaggacgctcatgggacccaccagagtgttccgagcacgcca
tgtggccccagattctatccgtgggagtttcggcctcactgacacccgca
acaccacccatggttcggactctgtggtttcagccagcagagagattgca
gccttcttccctgacttcagtgaacagcgctggtatgaggaggaagagcc
ccagttgcgctgtggccctgtgtga
(amino acids)

(SEQ ID NO: 67)

MTQNLGSEMASILRSPQALQLTLALIKPDAVAHPLILEAVHQQILSNKFL
IVRMRELLWRKEDCQRFYREHEGRFFYQRLVEFMASGPIRAYILAHKDAI
QLWRTLMGPTRVFRARHVAPDSIRGSFGLTDTRNTTHGSDSVVSASREIA
AFFPDFSEQRWYEEEEPQLRCGPV-

Human NME6 2:

(DNA)

(SEQ ID NO: 68)

Atgctcactctagccctgatcaagcctgacgcagtcgcccatccactgat
tctggaggctgttcatcagcagattctaagcaacaagttcctgattgtac
gaatgagagaactactgtggagaaaggaagattgccagaggttttaccga
gagcatgaagggcgttttttctatcagaggctggtggagttcatggccag
cgggccaatccgagcctacatccttgcccacaaggatgccatccagctct
ggaggacgctcatgggacccaccagagtgttccgagcacgccatgtggcc
ccagattctatccgtgggagtttcggcctcactgacacccgcaacaccac
ccatggttcggactctgtggtttcagccagcagagagattgcagccttct
tccctgacttcagtgaacagcgctggtatgaggaggaagagccccagttg
cgctgtggccctgtgtga
(amino acids)

(SEQ ID NO: 69)

MLTLALIKPDAVAHPLILEAVHQQILSNKFLIVRMRELLWRKEDCQRFYR
EHEGRFFYQRLVEFMASGPIRAYILAHKDAIQLWRTLMGPTRVFRARHVA
PDSIRGSFGLTDTRNTTHGSDSVVSASREIAAFFPDFSEQRWYEEEEPQL
RCGPV-

Human NME6 3:

(DNA)
(SEQ ID NO: 70)
Atgctcactctagccctgatcaagcctgacgcagtcgcccatccactgat
tctggaggctgttcatcagcagattctaagcaacaagttcctgattgtac
gaatgagagaactactgtggagaaaggaagattgccagaggttttaccga
gagcatgaagggcgttttttctatcagaggctggtggagttcatggccag
cgggccaatccgagcctacatccttgcccacaaggatgccatccagctct
ggaggacgctcatgggacccaccagagtgttccgagcacgccatgtggcc
ccagattctatccgtgggagtttcggcctcactgacacccgcaacaccac
ccatggttcggactctgtggtttcagccagcagagagattgcagccttct
tccctgacttcagtgaacagcgctggtatgaggaggaagagccccagttg
cgctgtggccctgtgtgctatagcccagagggaggtgtccactatgtagc
tggaacaggaggcctaggaccagcctga
(amino acids)
(SEQ ID NO: 71)
MLTLALIKPDAVAHPLILEAVHQQILSNKFLIVRMRELLWRKEDCQRFYR
EHEGRFFYQRLVEFMASGPIRAYILAHKDAIQLWRTLMGPTRVFRARHVA
PDSIRGSFGLTDTRNTTHGSDSVVSASREIAAFFPDFSEQRWYEEEEPQL
RCGPVCYSPEGGVHYVAGTGGLGPA-

Human NME6 sequence optimized for E. coli expression:

(DNA)
(SEQ ID NO: 72)
Atgacgcaaaatctgggctcggaaatggcaagtatcctgcgctccccgca
agcactgcaactgaccctggctctgatcaaaccggacgctgttgctcatc
cgctgattctggaagcggtccaccagcaaattctgagcaacaaatttctg
atcgtgcgtatgcgcgaactgctgtggcgtaaagaagattgccagcgttt
ttatcgcgaacatgaaggccgtttcttttatcaacgcctggttgaattca
tggcctctggtccgattcgcgcatatatcctggctcacaaagatgcgatt
cagctgtggcgtaccctgatgggtccgacgcgcgtctttcgtgcacgtca
tgtggcaccggactcaatccgtggctcgttcggtctgaccgatacgcgca
ataccacgcacggtagcgactctgttgttagtgcgtcccgtgaaatcgcg
gcctttttcccggacttctccgaacagcgttggtacgaagaagaagaacc
gcaactgcgctgtggcccggtctgttattctccggaaggtggtgtccatt
atgtggcgggcacgggtggtctgggtccggcatga
(amino acids)
(SEQ ID NO: 73)
MTQNLGSEMASILRSPQALQLTLALIKPDAVAHPLILEAVHQQILSNKFL
IVRMRELLWRKEDCQRFYREHEGRFFYQRLVEFMASGPIRAYILAHKDAI
QLWRTLMGPTRVFRARHVAPDSIRGSFGLTDTRNTTHGSDSVVSASREIA
AFFPDFSEQRWYEEEEPQLRCGPVCYSPEGGVHYVAGTGGLGPA-

Human NME6 I sequence optimized for E. coli expression:

(DNA)
(SEQ ID NO: 74)
Atgacgcaaaatctgggctcggaaatggcaagtatcctgcgctccccgca
agcactgcaactgaccctggctctgatcaaaccggacgctgttgctcatc
cgctgattctggaagcggtccaccagcaaattctgagcaacaaatttctg
atcgtgcgtatgcgcgaactgctgtggcgtaaagaagattgccagcgttt
ttatcgcgaacatgaaggccgtttcttttatcaacgcctggttgaattca
tggcctctggtccgattcgcgcatatatcctggctcacaaagatgcgatt
cagctgtggcgtaccctgatgggtccgacgcgcgtctttcgtgcacgtca
tgtggcaccggactcaatccgtggctcgttcggtctgaccgatacgcgca
ataccacgcacggtagcgactctgttgttagtgcgtcccgtgaaatcgcg
gcctttttcccggacttctccgaacagcgttggtacgaagaagaagaacc
gcaactgcgctgtggcccggtctga
(amino acids)
(SEQ ID NO: 75)
MTQNLGSEMASILRSPQALQLTLALIKPDAVAHPLILEAVHQQILSNKFL
IVRMRELLWRKEDCQRFYREHEGRFFYQRLVEFMASGPIRAYILAHKDAI
QLWRTLMGPTRVFRARHVAPDSIRGSFGLTDTRNTTHGSDSVVSASREIA
AFFPDFSEQRWYEEEEPQLRCGPV-

Human NME6 2 sequence optimized for E. coli expression:

(DNA)
(SEQ ID NO: 76)
Atgctgaccctggctctgatcaaaccggacgctgttgctcatccgctgat
tctggaagcggtccaccagcaaattctgagcaacaaatttctgatcgtgc
gtatgcgcgaactgctgtggcgtaaagaagattgccagcgtttttatcgc
gaacatgaaggccgtttcttttatcaacgcctggttgaattcatggcctc
tggtccgattcgcgcatatatcctggctcacaaagatgcgattcagctgt
ggcgtaccctgatgggtccgacgcgcgtctttcgtgcacgtcatgtggca
ccggactcaatccgtggctcgttcggtctgaccgatacgcgcaataccac
gcacggtagcgactctgttgttagtgcgtcccgtgaaatcgcggcctttt
tcccggacttctccgaacagcgttggtacgaagaagaagaaccgcaactg
cgctgtggcccggtctga
(amino acids)
(SEQ ID NO: 77)
MLTLALIKPDAVAHPLILEAVHQQILSNKFLIVRMRELLWRKEDCQRFYR
EHEGRFFYQRLVEFMASGPIRAYILAHKDAIQLWRTLMGPTRVFRARHVA
PDSIRGSFGLTDTRNTTHGSDSVVSASREIAAFFPDFSEQRWYEEEEPQL
RCGPV-

Human NME6 3 sequence optimized for E. coli expression:

(DNA)
(SEQ ID NO: 78)
Atgctgaccctggctctgatcaaaccggacgctgttgctcatccgctgat
tctggaagcggtccaccagcaaattctgagcaacaaatttctgatcgtgc
gtatgcgcgaactgctgtggcgtaaagaagattgccagcgtttttatcgc
gaacatgaaggccgtttcttttatcaacgcctggttgaattcatggcctc
tggtccgattcgcgcatatatcctggctcacaaagatgcgattcagctgt
ggcgtaccctgatgggtccgacgcgcgtctttcgtgcacgtcatgtggca
ccggactcaatccgtggctcgttcggtctgaccgatacgcgcaataccac
gcacggtagcgactctgttgttagtgcgtcccgtgaaatcgcggcctttt
tcccggacttctccgaacagcgttggtacgaagaagaagaaccgcaactg
cgctgtggcccggtctgttattctccggaaggtggtgtccattatgtggc
gggcacgggtggtctgggtccggcatga
(amino acids)
(SEQ ID NO: 79)
MLTLALIKPDAVAHPLILEAVHQQILSNKFLIVRMRELLWRKEDCQRFYR
EHEGRFFYQRLVEFMASGPIRAYILAHKDAIQLWRTLMGPTRVFRARHVA
PDSIRGSFGLTDTRNTTHGSDSVVSASREIAAFFPDFSEQRWYEEEEPQL
RCGPVCYSPEGGVHYVAGTGGLGPA-

NME6 and NME7 as well as novel variants may be expressed with any affinity tag but were expressed with the following tags:

Histidine Tag

(SEQ ID NO: 84)

(ctcgag)caccaccaccaccaccactga

Strept II Tag

(SEQ ID NO: 85)

(accggt)tggagccatcctcagttcgaaaagtaatga

### Example 6—Human NME7-1 Sequence Optimized for E. coli Expression

NME7 wt-cDNA, codon optimized for expression in E. coli was generated per our request by Genscript (NJ). NME7-1 was amplified by polymerase chain reaction (PCR) using the following primers:

Forward

(SEQ ID NO: 141)

5′-atcgatcatatgaatcactccgaacgc-3′
Reverse

(SEQ ID NO: 142)

5′-agagcctcgagattatccagaattttgaaaaagtattg-3′

The fragment was then purified, digested (NdeI, XhoI) and cloned between NdeI and XhoI restriction sites of the expression vector pET21b.

### Example 7—Human NME7-2 Sequence Optimized for E. coli Expression

NME7-2 was amplified by polymerase chain reaction (PCR) using the following primers:

Forward

(SEQ ID NO: 143)

5′-atcgatcatatgcatgacgttaaaaatcac-3′
Reverse

(SEQ ID NO: 144)

5′-agagcctcgagattatccagaattttgaaaaagtattg-3′

The fragment was then purified, digested (NdeI, XhoI) and cloned between NdeI and XhoI restriction sites of the expression vector pET21b.

### Example 8—Human NME7-A Sequence Optimized for E. coli Expression

NME7-A was amplified by polymerase chain reaction (PCR) using the following primers:

Forward
(SEQ ID NO: 145)
5′-atcgacatatggaaaaaacgctggccctgattaaaccggatg-3′
Reverse
(SEQ ID NO: 146)
5′-actgcctcgaggaaaaacagttccatttcacgagctgccgatg-3′

The fragment was then purified, digested (NdeI, XhoI) and cloned between NdeI and XhoI restriction sites of the expression vector pET21b.

### Example 9—Human NME7-AB Sequence Optimized for E. coli Expression

NME7-AB was amplified by polymerase chain reaction (PCR) using the following primers:

Forward
(SEQ ID NO: 147)
5′-atcgacatatggaaaaaacgctggccctgattaaaccggatg-3′
Reverse
(SEQ ID NO: 148)
5′-agagcctcgagattatccagaattttgaaaaagtattg-3′

The fragment was then purified, digested (NdeI, XhoI) and cloned between NdeI and XhoI restriction sites of the expression vector pET21b. The protein is expressed with a C-Term His Tag.

NME7-AB was amplified by polymerase chain reaction (PCR) using the following primers:

Forward
(SEQ ID NO: 149)
5′-atcgacatatggaaaaaacgctggccctgattaaaccggatg-3′
Reverse
(SEQ ID NO: 150)
5′-agagcaccggtattatccagaattttgaaaaagtattg-3′

The fragment was then purified, digested (NdeI, AgeI) and cloned between NdeI and AgeI restriction sites of the expression vector pET21b where XhoI was replaced by AgeI followed by the Strep Tag II and two stop codon before the His Tag. The protein is expressed with a C-Term Strep Tag II.

### Example 10—Human NME6 Sequence Optimized for E. coli Expression

NME6 was amplified by polymerase chain reaction (PCR) using the following primers:

Forward

(SEQ ID NO: 151)

5′-atcgacatatgacgcaaaatctgggctcggaaatg-3′
Reverse

(SEQ ID NO: 152)

5′-actgcctcgagtgccggacccagaccacccgtgc-3′

The fragment was then purified, digested (NdeI, XhoI) and cloned between NdeI and XhoI restriction sites of the expression vector pET21b. The protein is expressed with a C-Term His Tag.

NME6 was amplified by polymerase chain reaction (PCR) using the following primers:

Forward

(SEQ ID NO: 153)

5′-atcgacatatgacgcaaaatctgggctcggaaatg-3′
Reverse

(SEQ ID NO: 154)

5′-actgcaccggttgccggacccagaccacccgtgcg-3′

The fragment was then purified, digested (NdeI, AgeI) and cloned between NdeI and AgeI restriction sites of the expression vector pET21b where XhoI was replaced by AgeI followed by the Strep Tag II and two stop codon before the His Tag. The protein is expressed with a C-Term Strep Tag II.

### Example 11—Generating Recombinant NME7-AB

LB broth (Luria-Bertani broth) is inoculated with 1/10 of an overnight culture and cultured at 37° C. until OD600 reached ˜0.5. At this point, recombinant protein expression is induced with 0.4 mM Isopropyl-β-D-thio-galactoside (IPTG, Gold Biotechnology) and culture is stopped after 5h. After harvesting the cells by centrifugation (6000 rpm for 10 min at 4° C.), cell pellet is resuspended with running buffer: PBS pH7.4, 360 mM NaCl and 80 mM imidazole. Then lysozyme (1 mg/mL, Sigma), MgCl₂ (0.5 mM) and DNAse (0.5 ug/mL, Sigma) is added. Cell suspension is incubated on a rotating platform (275 rpm) for 30 min at 37° ° C. and sonicated on ice for 5 min. Insoluble cell debris are removed by centrifugation (20000 rpm for 30 min at 4ºc). The cleared lysate is then applied to a Ni-NTA column (Qiagen) equilibrated with the running buffer. The column was washed with 4CV of running buffer, then 4CV of running buffer supplemented with 30 mM imidazole before eluting the protein off the column with the running buffer (6CV) supplemented with 70 mM imidazole followed by a second elution with the running buffer (4CV) supplemented with 490 mM imidazole. NME7-AB is further purified by size exclusion chromatography (Superdex 200) “FPLC”.

### Example 12—Generating Recombinant NME6

LB broth (Luria-Bertani broth) is inoculated with 1/10 of an overnight culture and cultured at 37° C. until OD600 reached ˜0.5. At this point, recombinant protein expression is induced with 0.4 mM Isopropyl-ß-D-thio-galactoside (IPTG, Gold Biotechnology) and culture is stopped after 5h. After harvesting the cells by centrifugation (6000 rpm for 10 min at 4° C.), cell pellet is resuspended with running buffer: PBS pH7.4, 360 mM NaCl and 80 mM imidazole. Then lysozyme (1 mg/mL, Sigma), MgCl₂ (0.5 mM) and DNAse (0.5 ug/mL, Sigma) is added. Cell suspension is incubated on a rotating platform (275 rpm) for 30 min at 37° C. and sonicated on ice for 5 min. Insoluble cell debris are removed by centrifugation (20000 rpm for 30 min at 4ºc). The cleared lysate is then applied to a Ni-NTA column (Qiagen) equilibrated with the running buffer. The column is washed (8CV) before eluting the protein off the column with the running buffer (6CV) supplemented with 420 mM imidazole. NME6 is further purified by size exclusion chromatography (Superdex 200) “FPLC”.

### Example 13—Quantitative PCR Analysis of Naïve and Primed Genes

Standard methods were used to perform RT-PCR. The primers used are listed below: RNA was isolated using the Trizol® Reagent (Invitrogen) and cDNA was reverse transcribed with Random Hexamers (Invitrogren) using Super Script II (Invitrogen) and subsequently assayed for the genes FOXA2, XIST, KLF2, KLF4, NANOG and OCT4, using Applied Biosystems gene expression assays (OCT4 P/N Hs00999634_gH, Nanog P/N Hs02387400_g1, KLF2 P/N Hs00360439_g1, KLF4 P/N Hs00358836_m1, FOXa2 P/N Hs00232764_m1, OTX2 P/N Hs00222238_m1, LHX2 P/N Hs00180351_m1, XIST P/N Hs01079824 ml and GAPDH P/N 4310884E), on an Applied Biosystems 7500 real-time instrument. Each sample was run in triplicate. Gene expression was normalized to GAPDH. Data are expressed as a fold change relative to control.

### Example 14—a MUC1 Pull Down Assay Shows that NME1, NME6 and NME7 Bind to a MUC1 Species Protein

A pull down assay using an antibody to the MUC1* cytoplasmic tail (Ab-5) was performed on a panel of cells. The proteins pulled down by the MUC1 antibody were separated by SDS-PAGE then probed with antibodies specific for NME1, NME6 and NME7, using Western blot technique. MUC1*-positive breast cancer cell line T47D cells (ATCC), human embryonic stem cell line BGO1v (LifeTechnologies), human ES cells (HES-3, BioTime Inc.) and human iPS cells (SC101A-1, System Biosciences Inc.) T47D cancer cells were grown according to ATCC protocol in RPMI-1640 (ATCC) plus 10% FBS (VWR). All stem cells were cultured in minimal stem cell media “MM” with 8 nM NM23-RS (recombinant NME1 S120G dimers). Stem cells were grown on plasticware coated with 12.5 ug/mL anti-MUC1* C3 mab. Cells were lysed with 200 uL RIPA buffer for 10 min on ice. After removal of cell debris by centrifugation, the supernatant was used in a co-immunoprecipitation assay. MUC1* was pulled down using the Ab-5 antibody (anti-MUC-1 Ab-5, Thermo Scientific), which recognizes the MUC1 cytoplasmic tail, coupled to Dynabeads protein G (Life Technologies). The beads were washed twice with RIPA buffer and resuspended in reducing buffer. A sample of the supernatant was subjected to a reducing SDS-PAGE followed by transfer of the protein to a PVDF membrane. The membrane was then probed with: A) an anti-NM23-H1 (NME1) Antibody (C-20, Santa Cruz Biotechnology); B) anti-NME6 (Abnova); or C) anti NM23-H7 Antibody (B-9, Santa Cruz Biotechnology); D) the staining of NME6 was enhanced using Supersignal (Pierce); and E) the staining of NME7 was enhanced using Supersignal. After incubation with their respective secondary antibody coupled to HRP, the proteins were detected by chemiluminescence. The photos show that native NME1, NME6 and NME7 are present in MUC1*-positive breast cancer cells, in human ES cells and in human iPS cells and that they bind to MUC1*. Note that the number of cells present in the HES-3 pellet was less than the number present in the other samples.

### Example 15—Recombinant NM23 (S120G Mutant H1 Dimers), NME7-AB, as Well as Native NME7 Bind to the MUC1* Extra Cellular Domain Peptide and can Induce Receptor Dimerization

Gold nanoparticles of a diameter of 30.0 nm were coated with an NTA-SAM surface according to Thompson et al. (ACS Appl. Mater. Interfaces, 2011, 3 (8), pp 2979-2987). The NTA-SAM coated gold nanoparticles were then activated with an equal volume of 180 uM NiSO₄, incubated for 10 min at room temperature, washed, and resuspended in a 10 mM phosphate buffer (pH 74). The gold nanoparticles were then loaded with PSMGER N-10 peptide (QFNQYKTEAASRYNLTISDVSVSDVPFPFSAQSGAHHHHHH (SEQ ID NO:155)) at 0.5 uM final concentration, and incubated at room temperature for 10 min. Recombinant NME7-AB protein expressed and purified from E. coli was added free in solution at the concentrations indicated. When particle-immobilized proteins bind to each other, or simultaneously bind to two different peptides on two different particles, the particle solution color changes from pink/red to purple/blue. If the protein added free in solution causes particle aggregation, it is strong evidence that the free protein dimerizes the cognate peptide, since binding to a single peptide would not induce two or more particles to be brought into close proximity to each other.

FIG. 7 (A) shows NTA-Ni-SAM coated nanoparticles loaded with the PSMGFR N-10 peptide. The NME7-AB is added free in solution at the concentrations indicated. Solution color change from pink to purple/blue from particle aggregation indicates binding between the MUC1* peptide on the particles and NME7 free in solution. This result shows that NME7 in solution has two binding sites for the MUC1* peptide. The Fab of the anti-MUC1* antibody fully inhibits the binding, showing that particle aggregation is due to the specific interaction of MUC1* peptide and NME7. (B) shows NME7-AB added free in solution over a wider range of concentrations. Particle aggregation, indicating NME7 can simultaneously bind to two peptides is observed. (C) shows all proteins added in solution. NME7-AB turned purple almost immediately. NM23-RS (H1 dimer) also began to change almost immediately to purple. The T47D breast cancer cell line Lysate, which contains native NME7 turns noticeably purple also.

### Example 16—Human ES and iPS Cells Cultured in NME1 Dimers or NME7 are in the Naïve State as Evidenced by Lack of Condensed Histone-3 in the Nucleus which would have Indicated X-Inactivation, a Hallmark of the Primed State

Human ES (HES-3 stem cells, BioTime Inc) and iPS (SC101A-Ipsc, System Biosciences) cells were cultured in Minimal Media (“MM”) plus either NME1 dimers (NM23-RS) or NME7 (NME7-AB construct) for 8-10 passages. The cells were plated onto a Vita™ plate (ThermoFisher) that had been coated with 12.5 ug/mL of an anti-MUC1* monoclonal antibody (MN-C3) that binds to the distal portion of the PSMGFR sequence of the MUC1* receptor. Periodically throughout the 10 passages, samples of the stem cells were assayed by immunocytochemistry (ICC) and analyzed on a confocal microscope (Zeiss LSM 510 confocal microscope) to determine the cellular localization of Histone-3. If Histone-3 is condensed in the nucleus (appears as single dot), then a copy of the X chromosome has been inactivated and the cells are no longer in the pure ground state or naïve state. If the stem cells have reverted from the primed state (all commercially available stem cells have been driven to the primed state by culturing in FGF) to the naïve state, then Histone-3 will be seen as a “cloud,” speckled throughout or not detectable. FIG. 12 shows the control cells, from the same source except that they have been grown in FGF on MEFs according to standard protocols, all show Histone-3 (H3K27me3) condensed in the nucleus, confirming that they are all 100% in the primed state and not in the naïve state. Conversely, the same source cells that were cultured in NME7 for 10 passages had mostly stem cells that do not have condensed Histone-3, indicating that they are pre-X-inactivation and in the true naïve state. The insert shown in FIG. 12 is one of many clones isolated that were 100% naïve.

### Example 17—Detection of NME7 in Embryonic Stem Cells and iPS Cells

Human ES cells (BGO1v and HES-3) as well as iPS cells (SC101-A1) were cultured in NME-based media wherein cells were plated over a layer of anti-MUC1* antibody. To identify NME7 species, cells were harvested and lysed with RIPA buffer (Pierce), supplemented with protease inhibitor (Pierce). Cell lysates (20 uL) were separated by electrophoresis on a 12% SDS-PAGE reducing gel and transferred to a PVDF membrane (GE Healthcare). The blot was blocked with PBS-T containing 3% milk and then incubated with primary antibody (anti NM23-H7 clone B-9, Santa Cruz Biotechnology) at 4° C. overnight. After washing with PBS-T, the membrane was incubated with horseradish peroxidase (HRP)-conjugated secondary antibody (goat anti mouse, Pierce) for 1 hr at room temperature. Signals were detected with Immun-Star Chemiluminescence kit (Bio-Rad). The Western blots show NME7 exist as ˜40 kDa species as well as a lower molecular weight NME7 species of ˜25-33 kDa, which may be an alternative splice isoform or a post translational modification such as cleavage.

### Example 18—Detection of NME7 in iPS Conditioned Media

iPS Conditioned media (20 uL) was separated by electrophoresis on either a 12% SDS-PAGE reducing gel and transferred to a PVDF membrane (GE Healthcare). The blot was blocked with PBS-T containing 3% milk and then incubated with primary antibody (anti NM23-H7 clone B-9, Santa Cruz Biotechnology) at 4ºC overnight. After washing with PBS-T, the membrane was incubated with horseradish peroxidase (HRP)-conjugated secondary antibody (goat anti mouse, Pierce) for 1 hr at room temperature. Signals were detected with Immun-Star Chemiluminescence kit (Bio-Rad). Western blots show secreted NME7 species having an approximate molecular weight of 30 kDa. Note that the recombinant NME7-AB has a molecular weight of 33 kDa and as such can simultaneously bind to two MUC1* peptides and also fully supports pluripotent stem cell growth, induction of pluripotency and inhibits differentiation. The NME7 species of ˜25-30 kDa may be an alternative splice isoform or a post translational modification such as cleavage, which may enable secretion from the cell.

### Example 19—NME7 Immuno-Precipitation and Analysis by Mass Spectrophotometry

A pull down assay was performed using an NME7 specific antibody (NM23 H7 B9, Santa Cruz) on a panel of MUC1*-positive cells. Breast cancer cells (T47D) as well as human ES (BGO1v and HES-3) and iPS (SC101-A1) cells were cultured according to standard protocol (T47D) or cultured in NME-based media over a surface of anti-MUC1* antibody. Cells were lysed with RIPA buffer (Pierce), supplemented with protease inhibitor (Pierce). Cell lysates were supplemented with 10 ug of recombinant NME7-AB incubated at 4ºC for 2h. Then NME7 was immuno-precipitated at 4° C. overnight with anti NM23-H7 (B-9, Santa Cruz Biotechnology) coupled to Dynabeads protein G (Life technologies). Beads were washed twice with PBS and immuno-precipitated proteins were separated by electrophoresis on a 12% SDS-PAGE reducing gel. Proteins were detected by silver staining (Pierce). The ˜23 kDa bands of proteins that co-immunoprecipitated along with NME7, from the T47D sample and the BGO1v cells, were excised and analyzed by mass spec (Taplin Mass Spectrometry Facility, Harvard Medical School). Mass spec analysis showed that the protein bands that were excised all contained sequences from the NME7 NDPK A domain as shown below. The underlined sequences in the A domain of NME7 were identified by mass spec.

(SEQ ID NO: 156)
MNHSERFVFIAEWYDPNASLLRRYELLFYPGDGSVEMHDVKNHRTFLKRT
KYDNLHLEDLFIGNKVNVFSRQLVLIDYGDQYTARQLGSRKEKTLALIKP
DAISKAGEIIEIINKAGFTITKLKMMMLSRKEALDFHVDHQSRPFFNELI
QFITTGPIIAMEILRDDAICEWKRLLGPANSGVARTDASESIRALFGTDG
IRNAAHGPDSFASAAREMELFFPSSGGCGPANTAKFTNCTCCIVKPHAVS
EGLLGKILMAIRDAGFEISAMQMFNMDRVNVEEFYEVYKGVVTEYHDMVT
EMYSGPCVAMEIQQNNATKTFREFCGPADPEIARHLRPGTLRAIFGKTKI
QNAVHCTDLPEDGLLEVQYFFKILDN

The higher molecular weight protein bands, ˜30 kDa, that immunoprecipitated with NME7 were not analyzed by mass spec and may correspond to either an endogenous NME7 protein that may be a cleavage product or an alternative splice isoform or alternatively could be NME7-AB ˜33 kDa that was added to the cell lysates.

### Example 20—ELISA Assay Showing NME7-AB Simultaneously Binds to Two MUC1* Extra Cellular Domain Peptides

The PSMGFR peptide bearing a C-terminal Cysteine (PSMGFR-Cys) was covalently coupled to BSA using Imject Maleimide activated BSA kit (Thermo Fisher). PSMGFR-Cys coupled BSA was diluted to 10 ug/mL in 0.1M carbonate/bicarbonate buffer pH 9.6 and 50 uL was added to each well of a 96 well plate. After overnight incubation at 4° C., the plate was wash twice with PBS-T and a 3% BSA solution was added to block remaining binding site on the well. After 1h at RT the plate was washed twice with PBS-T and NME7, diluted in PBS-T+1% BSA, was added at different concentrations. After 1h at RT the plate was washed 3× with PBS-T and anti-NM23-H7 (B-9, Santa Cruz Biotechnology), diluted in PBS-T+1% BSA, was added at 1/500 dilution. After 1h at RT the plate was washed 3× with PBS-T and goat anti mouse-HRP, diluted in PBS-T+1% BSA, was added at 1/3333 dilution. After 1h at RT the plate was washed 3× with PBS-T and binding of NME7 was measured at 415 nm using a ABTS solution (Pierce).

ELISA MUC1* dimerization: The protocol for NME7 binding was used and NME7 was used at 11.6 ug/mL.

After 1h at RT the plate was washed 3× with PBS-T and HisTagged PSMGFR peptide (PSMGFR-His) or biotinylated PSMGFR peptide (PSMGFR-biotin), diluted in PBS-T+1% BSA, was added at different concentration. After 1h at RT the plate was washed 3× with PBS-T and anti Histag-HRP (Abcam) or streptavidin-HRP (Pierce), diluted in PBS-T+1% BSA, was added at a concentration of 1/5000. After 1h at RT the plate was washed 3× with PBS-T and binding of PSMGFR peptide to NME7 already bound to another PSMGFR peptide (which could not signal by anti-His antibody or by streptavidin) coupled BSA was measured at 415 nm using a ABTS solution (Pierce).

### Example 21—NME6 Cloning, Expression and Purification

WT NME6 cDNA, codon optimized for expression in E. coli was synthesized by our request by Genscript, NJ. The WT NME6 cDNA was then amplified by polymerase chain reaction (PCR) using the following primer: 5′-atcgacatatgacgcaaaatctgggctcggaaatg-3′ (SEQ ID NO:157) and 5′-actgcctcgagtgccggacccagaccacccgtgc-3′ (SEQ ID NO: 158). After digestion with NdeI and XhoI restriction enzymes (New England Biolabs), the purified fragment was cloned into the pET21b vector (Novagen) digested with the same restriction enzymes.

### Example 22—NME6 Protein Expression/Purification

LB broth (Luria-Bertani broth) was inoculated with 1/10 of an overnight culture and cultured at 37° C. until OD600 reached ˜0.5. At this point, recombinant protein expression was induced with 0.4 mM Isopropyl-ß-D-thio-galactoside (IPTG, Gold Biotechnology) and culture was stopped after 5h. After harvesting the cells by centrifugation (6000 rpm for 10 min at 4° C.), cell pellet was resupended with running buffer: PBS pH7.4, 360 mM NaCl, 10 mM imidazole and 8M urea. Cell suspension was incubated on a rotating platform (275 rpm) for 30 min at 37° C. and sonicated on ice for 5 min. Insoluble cell debris was removed by centrifugation (20000 rpm for 30 min at 4° c.). The cleared lysate was then applied to a Ni-NTA column (Qiagen) equilibrated with the running buffer. The column was washed with 4CV of running buffer, then 4CV of running buffer supplemented with 30 mM imidazole before eluting the protein off the column with the running buffer (8CV) supplemented with 420 mM imidazole. The protein was then refolded by dialysis.

### Example 23—Refolding Protocol

1. Dialyse overnight against 100 mM Tris pH 8.0, 4M urea, 0.2 mM imidazole, 0.4M L-arginine, 1 mM EDTA and 5% glycerol

2. Dialyse 24h against 100 mM Tris pH 8.0, 2M urea, 0.2 mM imidazole, 0.4M L-arginine, 1 mM EDTA and 5% glycerol

3. Dialyse 24h against 100 mM Tris pH 8.0, 1M urea, 0.2 mM imidazole, 0.4M L-arginine, 1 mM EDTA and 5% glycerol

4. Dialyse 8h against 100 mM Tris pH 8.0, 0.2 mM imidazole, 0.4M L-arginine, 1 mM EDTA and 5% glycerol

5. Dialyse overnight against 25 mM Tris pH 8.0, 0.2 mM imidazole, 0.1M L-arginine, 1 mM EDTA and 5% glycerol

6. Dialyse 3×3 h against PBS pH 7.4, 0.2 mM imidazole, 1 mM EDTA and 5% glycerol

7. Dialyse overnight against PBS pH 7.4, 0.2 mM imidazole, 1 mM EDTA and 5% glycerol

8. Centrifuge refolded protein (18,500 rpm) 30 min at 4° C. and collect supernatant for further purification. The protein was further purified by size exclusion chromatography (Superdex 200).

All of the references cited herein are incorporated by reference in their entirety.

### CITED REFERENCES LIST

1. Mahanta, S., et al. (2008). “A minimal fragment of MUC1 mediates growth of cancer cells.” PLOS One. 3(4): e2054.
2. Hikita, S. T., et al. (2008). “MUC1* mediates the growth of human pluripotent stem cells.” PLOS One. 3(10): e3312.
3. Smagghe, B. J., et al. (2013). “MUC1* ligand, NM23-H1, is a novel growth factor that maintains human stem cells in a more naive state.” PLOS One. 8(3): e58601.
4. Lombardi, D., et al. (1995). “The association of the Nm23-M1 protein and beta-tubulin correlates with cell differentiation.” Exp Cell Res 217(2): 267-71.
5. Okabe-Kado, J., et al. (1985). “Characterization of a differentiation-inhibitory activity from nondifferentiating mouse myeloid leukemia cells.” Cancer Res 45(10): 4848-52.
6. Okabe-Kado, J., et al. (1992). “Identity of a differentiation inhibiting factor for mouse myeloid leukemia cells with NM23/nucleoside diphosphate kinase.” Biochem Biophys Res Commun 182(3): 987-94.
7. Okabe-Kado, J., et al. (1995). “Inhibitory action of nm23 proteins on induction of erythroid differentiation of human leukemia cells.” Biochim Biophys Acta 1267(2-3): 101-6.
8. Ligtenberg M J L, Vos H L, Genissen, A M C and Hilkens J. (1990) J. Biol. Chem. 265, 15573-15578
9. Baruch A et al. (1999) The breast cancer-associated MUC1 gene generates both a receptor and its cognate binding protein. Cancer Res 59:1552.
10. Kroemer G, et al. (2013) Victories and deceptions in tumor immunology, OncoImmunology 2:1 e23687.
11. Rath N, Olson M F. Rho-associated kinases in tumorigenesis: re-considering ROCK inhibition for cancer therapy. EMBO Rep. 2012; 13(10):900-8
12. Fessler S, Wotkowicz M, Mahanta S, Bamdad C (2009) MUC1* is a determinant of trastuzumab (Herceptin) resistance in breast cancer cells, Breast Cancer Res Treat 118:113-124 DOI 10.1007/s10549-009-0412-3
13. Nichols J, Smith A (2009) Naive and primed pluripotent states. Cell Stem Cell 4: 487-492.
14. Hanna J, Cheng A W, Saha K, Kim J, Lengner C J, et al. (2010) Human embryonic stem cells with biological and epigenetic characteristics similar to those of mouse ESCs. Proc Natl Acad Sci USA 107: 9222-9227.
15. Amit M, Carpenter M K, Inokuma M S, Chiu C-P, Harris C P, et al. (2000) Clonally Derived Human Embryonic Stem Cell Lines Maintain Pluripotency and Proliferative Potential for Prolonged Periods of Culture. Developmental Biology 227: 271-278.
16. Ludwig T E, Levenstein M E, Jones J M, Berggren W T, Mitchen E R, et al. (2006) Derivation of human embryonic stem cells in defined conditions. Nat Biotechnol 24: 185-187.
17. Xu C, Rosler E, Jiang J, Lebkowski J S, Gold J D, et al. (2005) Basic Fibroblast Growth Factor Supports Undifferentiated Human Embryonic Stem Cell Growth Without Conditioned Medium. STEM CELLS 23: 315-323.
18. Xu R H, Peck R M, Li D S, Feng X, Ludwig T, et al. (2005) Basic FGF and suppression of BMP signaling sustain undifferentiated proliferation of human ES cells. Nat Methods 2: 185-190.
19. Liu W, Ma Q, Wong K, Li W, Ohgi K, Zhang J, Aggarwal A K, Rosenfeld M G. Brd4 and JMJD6-Associated Anti-Pause Enhancers in Regulation of Transcriptional Pause Release. Cell. 2013 Dec. 19; 155(7):1581-95. PMCID: PMC3886918.
20. Rais Y, Zviran A, Geula S, Gafni O, Chomsky E, Viukov S, Mansour A A, Caspi I, Krupalnik V, Zerbib M, Maza I, Mor N, Baran D, Weinberger L, Jaitin D A, Lara-Astiaso D, Blecher-Gonen R, Shipony Z, Mukamel Z, Hagai T, Gilad S, Amann-Zalcenstein D, Tanay A, Amit I, Novershtern N, Hanna J H. Deterministic direct reprogramming of somatic cells to pluripotency. 2013 Sep. 18, Nature 502, 65-70 DOI: 10.1038/nature12587
21. Faber A, Goessler U R, Hoermann K, Schultz J D, Umbreit C, Stern-Straeter J. SDF-1-CXCR4 axis: cell trafficking in the cancer stem cell niche of head and neck squamous cell carcinoma. Oncol. Rep. 2013 June; 29(6):2325-31.
22. Miki J, Furusato B, Li H, Gu Y, Takahashi H, Egawa S, Sesterhenn I A, McLeod D G, Srivastava S, Rhim J S. Identification of putative stem cell markers, CD133 and CXCR4, in hTERT-immortalized primary nonmalignant and malignant tumor-derived human prostate epithelial cell lines and in prostate cancer specimens. Cancer Res. 2007 Apr. 1; 67(7):3153-61.
23. Mukherjee D, Zhao J. The Role of chemokine receptor CXCR4 in breast cancer metastasis. Am J Cancer Res. 2013; 3(1):46-57. PMCID: PMC3555200
24. Herreros-Villanueva M, Zhang J-S, Koenig A, Abel E V, Smyrk T C, Bamlet W R, de Narvajas A A-M, Gomez T S, Simeone D M, Bujanda L, Billadeau D D. SOX2 promotes dedifferentiation and imparts stem cell-like features to pancreatic cancer cells. Oncogenesis. 2013; 2:e61. PMCID: PMC3759123
25. Sefah K, Bae K-M, Phillips J A, Siemann D W, Su Z, Mcclellan S, Vieweg J, Tan W. Cell-based selection provides novel molecular probes for cancer stem cells. Int. J. Cancer. 2013 Jun. 1; 132(11):2578-88.
26. Jeter C R, Liu B, Liu X, Chen X, Liu C, Calhoun-Davis T, Repass J, Zaehres H, Shen J J, Tang D G. NANOG promotes cancer stem cell characteristics and prostate cancer resistance to androgen deprivation. Oncogene. 2011 Sep. 8; 30(36):3833-45. PMCID: PMC3140601
27. Herreros-Villanueva M, Zhang J-S, Koenig A, Abel E V, Smyrk T C, Bamlet W R, de Narvajas A A-M, Gomez T S, Simeone D M, Bujanda L, Billadeau D D. SOX2 promotes dedifferentiation and imparts stem cell-like features to pancreatic cancer cells. Oncogenesis. 2013; 2:e61. PMCID: PMC3759123
28. Hong X, Chedid K, Kalkanis S N. Glioblastoma cell line-derived spheres in serum-containing medium versus serum-free medium: a comparison of cancer stem cell properties. Int. J. Oncol. 2012 November; 41(5):1693-700.
29. Hong X, Chedid K, Kalkanis S N. Glioblastoma cell line-derived spheres in serum-containing medium versus serum-free medium: a comparison of cancer stem cell properties. Int. J. Oncol. 2012 November; 41(5):1693-700.
30. Su H-T, Weng C-C, Hsiao P-J, Chen L-H, Kuo T-L, Chen Y-W, Kuo K-K, Cheng K-H. Stem cell marker nestin is critical for TGF-B1-mediated tumor progression in pancreatic cancer. Mol. Cancer Res. 2013 July; 11(7): 768-79.
31. Silva J, Barrandon O, Nichols J, Kawaguchi J, Theunissen T W, Smith A. Promotion of reprogramming to ground state pluripotency by signal inhibition. PLOS Biol. 2008 Oct. 21; 6(10): e253. PMCID: PMC2570424
32. Boyer et al, 2005, “Core Transcriptional Regulatory Circuitry in Human Embryonic Stem Cells”, Cell, Vol. 122, 947-956
33. Takahashi K and Yamanaka S (2006) Induction of pluripotent stem cells from mouse embryonic and adult fibroblast cultures by defined factors. Cell 126(4):663-676.
34. Porter D et al. (2011) Chimeric antigen receptor-modified T cells in chronic lymphoid leukemia. N Engl J Med 365:725-733 DOI: 10.1056/NEJMoa1103849
35. Tiller T et al. (2013) A fully synthetic human Fab antibody library based on fixed VH/VL framework pairings with favorable biophysical properties. MABs 9:5(3) PMID: 23571156
36. Webb P A, Perisic O, Mendola C E, Backer J M and Williams R L. The crystal structure of a human nucleoside diphosphate kinase, NM23-H2. J Mol Biol. 1995, 251:574-587.
37. Min K, Song H K, Chang C, Kim S Y, Lee K J and Suh S W. Crystal structure of human nucleoside diphosphate kinase A, a metastasis suppressor. Proteins. 2002, 46:340-342.

Those skilled in the art will recognize, or be able to ascertain using no more than routine experimentation, many equivalents to the specific embodiments of the invention specifically described herein. Such equivalents are intended to be encompassed in the scope of the claims.

What is claimed:

1. A method of transforming a cancer cell to a more metastatic cancer stem cell comprising contacting the cancer cell with an NM23 protein that activates MUC1*.

2. The method of claim 1, wherein the NM23 protein is an NME1 dimer.

3. The method of claim 1, wherein the NM23 protein is NME7.

4. The method of claim 3, wherein the NME7 comprises an NDP kinase A domain and an NDP kinase B domain and is devoid of a DM10 leader sequence.

5. The method of claim 3, wherein the NME7 is NME7-AB.

6. The method of claim 5, wherein the NME7-AB comprises SEQ ID NO: 39 or SEQ ID NO: 41.

7. The method of claim 1, wherein the transforming a cancer cell comprises upregulating CXCR4 and SOX2 expression.

8. The method of claim 7, wherein expression of CXCR4 and SOX2 is upregulated by about 200-fold or more.

9. A method of preventing cancer or cancer metastasis by vaccinating a person with an NME family protein or peptide fragment or fragments thereof.

10. The method of claim 9, wherein the peptide fragment or fragments comprise one or more peptides whose sequence is present in an NME family protein, which is optionally mixed with a carrier, adjuvant or attached to an immunogenic agent.

11. The method of claim 9, wherein the sequence of the peptide is not present in human NME-H1.

12. The method of claim 9, wherein the NME family protein is a bacterial NME.

13. The method of claim 9, wherein a dimer of the bacterial NME can bind to PSMGFR peptide of MUC1* by ELISA and induce OCT4 expression by a human fibroblast.

14. The method of claim 9, wherein the NME family protein is the NME protein of Hsp. 593.

15. The method of claim 9, wherein the NME family protein is the NME protein of Porphyromonas gingivalis W83.

16. A method of generating or selecting an antibody or antibody-like molecule that specifically binds to a bacterial NME family protein or peptide fragment thereof, comprising:
(i) screening an antibody library or library of antibody fragments or epitopes with the bacterial NME family protein or peptide fragment;
(ii) assaying for binding to the bacterial NME family protein or a peptide fragment thereof; and
(iii) identifying the specifically bound antibody or antibody-like molecule.

17. The method of claim 16, further comprising formulating the identified antibody or antibody-like molecule for administration to a patient for the treatment or prevention of cancer.

18. The method of claim 16, wherein a dimer of the bacterial NME can bind to a PSMGFR peptide of MUC1* by ELISA and can induce OCT4 expression by a human fibroblast.

19. The method of claim 16, wherein the NME family protein is the NME protein of Hsp. 593.

20. The method of claim 16, wherein the NME family protein is the NME protein of Porphyromonas gingivalis W83.


SEQUENCE LISTING (166 sequences)

SEQ ID NO: 1 - protein, 1255 aa, Homo sapiens
  REGION 1..1255: note = full-length MUC1 Receptor
MTPGTQSPFFLLLLLTVLTVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSSVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAAASANL

SEQ ID NO: 2 - protein, 19 aa, synthetic construct
  REGION 1..19: note = Description of Artificial Sequence: Synthetic peptide
  REGION 1..19: note = N-terminal MUC-1 signaling sequence
MTPGTQSPFFLLLLLTVLT

SEQ ID NO: 3 - protein, 23 aa, synthetic construct
  REGION 1..23: note = Description of Artificial Sequence: Synthetic peptide
  REGION 1..23: note = N-terminal MUC-1 signaling sequence
MTPGTQSPFFLLLLLTVLTVVTA

SEQ ID NO: 4 - protein, 23 aa, synthetic construct
  REGION 1..23: note = Description of Artificial Sequence: Synthetic peptide
  REGION 1..23: note = N-terminal MUC-1 signaling sequence
MTPGTQSPFFLLLLLTVLTVVTG

SEQ ID NO: 5 - protein, 146 aa, synthetic construct
  REGION 1..146: note = Description of Artificial Sequence: Synthetic polypeptide
  REGION 1..146: note = truncated MUC1 receptor isoform having nat-PSMGFR at its N-terminus
GTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAAASANL

SEQ ID NO: 6 - protein, 45 aa, Homo sapiens
  REGION 1..45: note = Native Primary Sequence of the MUC1 Growth Factor Receptor
GTINVHDVETQFNQYKTEAASRYNLTISDVSVSDVPFPFSAQSGA

SEQ ID NO: 7 - protein, 44 aa, Homo sapiens
  REGION 1..44: note = Native Primary Sequence of the MUC1 Growth Factor Receptor
TINVHDVETQFNQYKTEAASRYNLTISDVSVSDVPFPFSAQSGA

SEQ ID NO: 8 - protein, 45 aa, synthetic construct
  REGION 1..45: note = Description of Artificial Sequence: Synthetic polypeptide
  REGION 1..45: note = "SPY" functional variant of the native Primary Sequence of the MUC1 Growth Factor Receptor
GTINVHDVETQFNQYKTEAASPYNLTISDVSVSDVPFPFSAQSGA

SEQ ID NO: 9 - protein, 44 aa, synthetic construct
  REGION 1..44: note = Description of Artificial Sequence: Synthetic polypeptide
  REGION 1..44: note = SPY" functional variant of the native Primary Sequence of the MUC1 Growth Factor Receptor
TINVHDVETQFNQYKTEAASPYNLTISDVSVSDVPFPFSAQSGA

SEQ ID NO: 10 - DNA, 216 bp, Homo sapiens
  misc_feature 1..216: note = MUC1 cytoplasmic domain nucleotide sequence
  source 1..216: mol_type = other DNA
tgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaacggtggcagcagcctctcttacacaaacccagcagtggcagccgcttctgccaacttg

SEQ ID NO: 11 - protein, 72 aa, Homo sapiens
  REGION 1..72: note = MUC1 cytoplasmic domain amino acid sequence
CQCRRKNYGQLDIFPARDTYHPMSEYPTYHTHGRYVPPSSTDRSPYEKVSAGNGGSSLSYTNPAVAAASANL

SEQ ID NO: 12 - DNA, 854 bp, Homo sapiens
  misc_feature 1..854: note = NME7 nucleotide sequence
  source 1..854: mol_type = other DNA
gagatcctgagacaatgaatcatagtgaaagattcgttttcattgcagagtggtatgatccaaatgcttcacttcttcgacgttatgagcttttattttacccaggggatggatctgttgaaatgcatgatgtaaagaatcatcgcacctttttaaagcggaccaaatatgataacctgcacttggaagatttatttataggcaacaaagtgaatgtcttttctcgacaactggtattaattgactatggggatcaatatacagctcgccagctgggcagtaggaaagaaaaaacgctagccctaattaaaccagatgcaatatcaaaggctggagaaataattgaaataataaacaaagctggatttactataaccaaactcaaaatgatgatgctttcaaggaaagaagcattggattttcatgtagatcaccagtcaagaccctttttcaatgagctgatccagtttattacaactggtcctattattgccatggagattttaagagatgatgctatatgtgaatggaaaagactgctgggacctgcaaactctggagtggcacgcacagatgcttctgaaagcattagagccctctttggaacagatggcataagaaatgcagcgcatggccctgattcttttgcttctgcggccagagaaatggagttgttttttccttcaagtggaggttgtgggccggcaaacactgctaaatttactaattgtacctgttgcattgttaaaccccatgctgtcagtgaaggtatgttgaatacactatattcagtacattttgttaataggagagcaatgtttattttcttgatgtactttatgtatagaaaataa

SEQ ID NO: 13 - protein, 283 aa, Homo sapiens
  REGION 1..283: note = NME7 amino acid sequence
DPETMNHSERFVFIAEWYDPNASLLRRYELLFYPGDGSVEMHDVKNHRTFLKRTKYDNLHLEDLFIGNKVNVFSRQLVLIDYGDQYTARQLGSRKEKTLALIKPDAISKAGEIIEIINKAGFTITKLKMMMLSRKEALDFHVDHQSRPFFNELIQFITTGPIIAMEILRDDAICEWKRLLGPANSGVARTDASESIRALFGTDGIRNAAHGPDSFASAAREMELFFPSSGGCGPANTAKFTNCTCCIVKPHAVSEGMLNTLYSVHFVNRRAMFIFLMYFMYRK

SEQ ID NO: 14 - DNA, 534 bp, Homo sapiens
  misc_feature 1..534: note = NM23-H1 nucleotide sequence
  source 1..534: mol_type = other DNA
atggtgctactgtctactttagggatcgtctttcaaggcgaggggcctcctatctcaagctgtgatacaggaaccatggccaactgtgagcgtaccttcattgcgatcaaaccagatggggtccagcggggtcttgtgggagagattatcaagcgttttgagcagaaaggattccgccttgttggtctgaaattcatgcaagcttccgaagatcttctcaaggaacactacgttgacctgaaggaccgtccattctttgccggcctggtgaaatacatgcactcagggccggtagttgccatggtctgggaggggctgaatgtggtgaagacgggccgagtcatgctcggggagaccaaccctgcagactccaagcctgggaccatccgtggagacttctgcatacaagttggcaggaacattatacatggcagtgattctgtggagagtgcagagaaggagatcggcttgtggtttcaccctgaggaactggtagattacacgagctgtgctcagaactggatctatgaatga

SEQ ID NO: 15 - protein, 177 aa, Homo sapiens
  REGION 1..177: note = NM23-H1 describes amino acid sequence
MVLLSTLGIVFQGEGPPISSCDTGTMANCERTFIAIKPDGVQRGLVGEIIKRFEQKGFRLVGLKFMQASEDLLKEHYVDLKDRPFFAGLVKYMHSGPVVAMVWEGLNVVKTGRVMLGETNPADSKPGTIRGDFCIQVGRNIIHGSDSVESAEKEIGLWFHPEELVDYTSCAQNWIYE

SEQ ID NO: 16 - DNA, 534 bp, synthetic construct
  misc_feature 1..534: note = Description of Artificial Sequence: Synthetic polynucleotide
  misc_feature 1..534: note = NM23-H1 S120G mutant nucleotide sequence
  source 1..534: mol_type = other DNA
atggtgctactgtctactttagggatcgtctttcaaggcgaggggcctcctatctcaagctgtgatacaggaaccatggccaactgtgagcgtaccttcattgcgatcaaaccagatggggtccagcggggtcttgtgggagagattatcaagcgttttgagcagaaaggattccgccttgttggtctgaaattcatgcaagcttccgaagatcttctcaaggaacactacgttgacctgaaggaccgtccattctttgccggcctggtgaaatacatgcactcagggccggtagttgccatggtctgggaggggctgaatgtggtgaagacgggccgagtcatgctcggggagaccaaccctgcagactccaagcctgggaccatccgtggagacttctgcatacaagttggcaggaacattatacatggcggtgattctgtggagagtgcagagaaggagatcggcttgtggtttcaccctgaggaactggtagattacacgagctgtgctcagaactggatctatgaatga

SEQ ID NO: 17 - protein, 177 aa, synthetic construct
  REGION 1..177: note = Description of Artificial Sequence: Synthetic polypeptide
  REGION 1..177: note = NM23-H1 S120G mutant amino acid sequence
MVLLSTLGIVFQGEGPPISSCDTGTMANCERTFIAIKPDGVQRGLVGEIIKRFEQKGFRLVGLKFMQASEDLLKEHYVDLKDRPFFAGLVKYMHSGPVVAMVWEGLNVVKTGRVMLGETNPADSKPGTIRGDFCIQVGRNIIHGGDSVESAEKEIGLWFHPEELVDYTSCAQNWIYE

SEQ ID NO: 18 - DNA, 459 bp, Homo sapiens
  misc_feature 1..459: note = NM23-H2 nucleotide sequence
  source 1..459: mol_type = other DNA
atggccaacctggagcgcaccttcatcgccatcaagccggacggcgtgcagcgcggcctggtgggcgagatcatcaagcgcttcgagcagaagggattccgcctcgtggccatgaagttcctccgggcctctgaagaacacctgaagcagcactacattgacctgaaagaccgaccattcttccctgggctggtgaagtacatgaactcagggccggttgtggccatggtctgggaggggctgaacgtggtgaagacaggccgagtgatgcttggggagaccaatccagcagattcaaagccaggcaccattcgtggggacttctgcattcaggttggcaggaacatcattcatggcagtgattcagtaaaaagtgctgaaaaagaaatcagcctatggtttaagcctgaagaactggttgactacaagtcttgtgctcatgactgggtctatgaataa

SEQ ID NO: 19 - protein, 152 aa, Homo sapiens
  REGION 1..152: note = NM23-H2 amino acid sequence
MANLERTFIAIKPDGVQRGLVGEIIKRFEQKGFRLVAMKFLRASEEHLKQHYIDLKDRPFFPGLVKYMNSGPVVAMVWEGLNVVKTGRVMLGETNPADSKPGTIRGDFCIQVGRNIIHGSDSVKSAEKEISLWFKPEELVDYKSCAHDWVYE

SEQ ID NO: 20 - DNA, 1023 bp, synthetic construct
  misc_feature 1..1023: note = Description of Artificial Sequence: Synthetic polynucleotide
  misc_feature 1..1023: note = Human NM23-H7-2 sequence optimized for E. coli expression
  source 1..1023: mol_type = other DNA
atgcatgacgttaaaaatcaccgtacctttctgaaacgcacgaaatatgataatctgcatctggaagacctgtttattggcaacaaagtcaatgtgttctctcgtcagctggtgctgatcgattatggcgaccagtacaccgcgcgtcaactgggtagtcgcaaagaaaaaacgctggccctgattaaaccggatgcaatctccaaagctggcgaaattatcgaaattatcaacaaagcgggtttcaccatcacgaaactgaaaatgatgatgctgagccgtaaagaagccctggattttcatgtcgaccaccagtctcgcccgtttttcaatgaactgattcaattcatcaccacgggtccgattatcgcaatggaaattctgcgtgatgacgctatctgcgaatggaaacgcctgctgggcccggcaaactcaggtgttgcgcgtaccgatgccagtgaatccattcgcgctctgtttggcaccgatggtatccgtaatgcagcacatggtccggactcattcgcatcggcagctcgtgaaatggaactgtttttcccgagctctggcggttgcggtccggcaaacaccgccaaatttaccaattgtacgtgctgtattgtcaaaccgcacgcagtgtcagaaggcctgctgggtaaaattctgatggcaatccgtgatgctggctttgaaatctcggccatgcagatgttcaacatggaccgcgttaacgtcgaagaattctacgaagtttacaaaggcgtggttaccgaatatcacgatatggttacggaaatgtactccggtccgtgcgtcgcgatggaaattcagcaaaacaatgccaccaaaacgtttcgtgaattctgtggtccggcagatccggaaatcgcacgtcatctgcgtccgggtaccctgcgcgcaatttttggtaaaacgaaaatccagaacgctgtgcactgtaccgatctgccggaagacggtctgctggaagttcaatactttttcaaaattctggataattga

SEQ ID NO: 21 - protein, 340 aa, synthetic construct
  REGION 1..340: note = Description of Artificial Sequence: Synthetic polypeptide
  REGION 1..340: note = Human NM23-H7-2 sequence optimized for E. coli expression
MHDVKNHRTFLKRTKYDNLHLEDLFIGNKVNVFSRQLVLIDYGDQYTARQLGSRKEKTLALIKPDAISKAGEIIEIINKAGFTITKLKMMMLSRKEALDFHVDHQSRPFFNELIQFITTGPIIAMEILRDDAICEWKRLLGPANSGVARTDASESIRALFGTDGIRNAAHGPDSFASAAREMELFFPSSGGCGPANTAKFTNCTCCIVKPHAVSEGLLGKILMAIRDAGFEISAMQMFNMDRVNVEEFYEVYKGVVTEYHDMVTEMYSGPCVAMEIQQNNATKTFREFCGPADPEIARHLRPGTLRAIFGKTKIQNAVHCTDLPEDGLLEVQYFFKILDN

SEQ ID NO: 22 - DNA, 399 bp, Homo sapiens
  misc_feature 1..399: note = Human NME7-A
  source 1..399: mol_type = other DNA
atggaaaaaacgctagccctaattaaaccagatgcaatatcaaaggctggagaaataattgaaataataaacaaagctggatttactataaccaaactcaaaatgatgatgctttcaaggaaagaagcattggattttcatgtagatcaccagtcaagaccctttttcaatgagctgatccagtttattacaactggtcctattattgccatggagattttaagagatgatgctatatgtgaatggaaaagactgctgggacctgcaaactctggagtggcacgcacagatgcttctgaaagcattagagccctctttggaacagatggcataagaaatgcagcgcatggccctgattcttttgcttctgcggccagagaaatggagttgtttttttga

SEQ ID NO: 23 - protein, 132 aa, Homo sapiens
  REGION 1..132: note = Human NME7-A
MEKTLALIKPDAISKAGEIIEIINKAGFTITKLKMMMLSRKEALDFHVDHQSRPFFNELIQFITTGPIIAMEILRDDAICEWKRLLGPANSGVARTDASESIRALFGTDGIRNAAHGPDSFASAAREMELFF

SEQ ID NO: 24 - DNA, 444 bp, Homo sapiens
  misc_feature 1..444: note = Human NME7-A1
  source 1..444: mol_type = other DNA
atggaaaaaacgctagccctaattaaaccagatgcaatatcaaaggctggagaaataattgaaataataaacaaagctggatttactataaccaaactcaaaatgatgatgctttcaaggaaagaagcattggattttcatgtagatcaccagtcaagaccctttttcaatgagctgatccagtttattacaactggtcctattattgccatggagattttaagagatgatgctatatgtgaatggaaaagactgctgggacctgcaaactctggagtggcacgcacagatgcttctgaaagcattagagccctctttggaacagatggcataagaaatgcagcgcatggccctgattcttttgcttctgcggccagagaaatggagttgttttttccttcaagtggaggttgtgggccggcaaacactgctaaatttacttga

SEQ ID NO: 25 - protein, 147 aa, Homo sapiens
  REGION 1..147: note = Human NME7-A1
MEKTLALIKPDAISKAGEIIEIINKAGFTITKLKMMMLSRKEALDFHVDHQSRPFFNELIQFITTGPIIAMEILRDDAICEWKRLLGPANSGVARTDASESIRALFGTDGIRNAAHGPDSFASAAREMELFFPSSGGCGPANTAKFT

SEQ ID NO: 26 - DNA, 669 bp, Homo sapiens
  misc_feature 1..669: note = Human NME7-A2
  source 1..669: mol_type = other DNA
atgaatcatagtgaaagattcgttttcattgcagagtggtatgatccaaatgcttcacttcttcgacgttatgagcttttattttacccaggggatggatctgttgaaatgcatgatgtaaagaatcatcgcacctttttaaagcggaccaaatatgataacctgcacttggaagatttatttataggcaacaaagtgaatgtcttttctcgacaactggtattaattgactatggggatcaatatacagctcgccagctgggcagtaggaaagaaaaaacgctagccctaattaaaccagatgcaatatcaaaggctggagaaataattgaaataataaacaaagctggatttactataaccaaactcaaaatgatgatgctttcaaggaaagaagcattggattttcatgtagatcaccagtcaagaccctttttcaatgagctgatccagtttattacaactggtcctattattgccatggagattttaagagatgatgctatatgtgaatggaaaagactgctgggacctgcaaactctggagtggcacgcacagatgcttctgaaagcattagagccctctttggaacagatggcataagaaatgcagcgcatggccctgattcttttgcttctgcggccagagaaatggagttgtttttttga

SEQ ID NO: 27 - protein, 222 aa, Homo sapiens
  REGION 1..222: note = Human NME7-A2
MNHSERFVFIAEWYDPNASLLRRYELLFYPGDGSVEMHDVKNHRTFLKRTKYDNLHLEDLFIGNKVNVFSRQLVLIDYGDQYTARQLGSRKEKTLALIKPDAISKAGEIIEIINKAGFTITKLKMMMLSRKEALDFHVDHQSRPFFNELIQFITTGPIIAMEILRDDAICEWKRLLGPANSGVARTDASESIRALFGTDGIRNAAHGPDSFASAAREMELFF

SEQ ID NO: 28 - DNA, 714 bp, Homo sapiens
  misc_feature 1..714: note = Human NME7-A3
  source 1..714: mol_type = other DNA
atgaatcatagtgaaagattcgttttcattgcagagtggtatgatccaaatgcttcacttcttcgacgttatgagcttttattttacccaggggatggatctgttgaaatgcatgatgtaaagaatcatcgcacctttttaaagcggaccaaatatgataacctgcacttggaagatttatttataggcaacaaagtgaatgtcttttctcgacaactggtattaattgactatggggatcaatatacagctcgccagctgggcagtaggaaagaaaaaacgctagccctaattaaaccagatgcaatatcaaaggctggagaaataattgaaataataaacaaagctggatttactataaccaaactcaaaatgatgatgctttcaaggaaagaagcattggattttcatgtagatcaccagtcaagaccctttttcaatgagctgatccagtttattacaactggtcctattattgccatggagattttaagagatgatgctatatgtgaatggaaaagactgctgggacctgcaaactctggagtggcacgcacagatgcttctgaaagcattagagccctctttggaacagatggcataagaaatgcagcgcatggccctgattcttttgcttctgcggccagagaaatggagttgttttttccttcaagtggaggttgtgggccggcaaacactgctaaatttacttga

SEQ ID NO: 29 - protein, 237 aa, Homo sapiens
  REGION 1..237: note = Human NME7-A3
MNHSERFVFIAEWYDPNASLLRRYELLFYPGDGSVEMHDVKNHRTFLKRTKYDNLHLEDLFIGNKVNVFSRQLVLIDYGDQYTARQLGSRKEKTLALIKPDAISKAGEIIEIINKAGFTITKLKMMMLSRKEALDFHVDHQSRPFFNELIQFITTGPIIAMEILRDDAICEWKRLLGPANSGVARTDASESIRALFGTDGIRNAAHGPDSFASAAREMELFFPSSGGCGPANTAKFT

SEQ ID NO: 30 - DNA, 408 bp, Homo sapiens
  misc_feature 1..408: note = Human NME7-B
  source 1..408: mol_type = other DNA
atgaattgtacctgttgcattgttaaaccccatgctgtcagtgaaggactgttgggaaagatcctgatggctatccgagatgcaggttttgaaatctcagctatgcagatgttcaatatggatcgggttaatgttgaggaattctatgaagtttataaaggagtagtgaccgaatatcatgacatggtgacagaaatgtattctggcccttgtgtagcaatggagattcaacagaataatgctacaaagacatttcgagaattttgtggacctgctgatcctgaaattgcccggcatttacgccctggaactctcagagcaatctttggtaaaactaagatccagaatgctgttcactgtactgatctgccagaggatggcctattagaggttcaatacttcttctga

SEQ ID NO: 31 - protein, 135 aa, Homo sapiens
  REGION 1..135: note = Human NME7-B
MNCTCCIVKPHAVSEGLLGKILMAIRDAGFEISAMQMFNMDRVNVEEFYEVYKGVVTEYHDMVTEMYSGPCVAMEIQQNNATKTFREFCGPADPEIARHLRPGTLRAIFGKTKIQNAVHCTDLPEDGLLEVQYFF

SEQ ID NO: 32 - DNA, 426 bp, Homo sapiens
  misc_feature 1..426: note = Human NME7-B1
  source 1..426: mol_type = other DNA
atgaattgtacctgttgcattgttaaaccccatgctgtcagtgaaggactgttgggaaagatcctgatggctatccgagatgcaggttttgaaatctcagctatgcagatgttcaatatggatcgggttaatgttgaggaattctatgaagtttataaaggagtagtgaccgaatatcatgacatggtgacagaaatgtattctggcccttgtgtagcaatggagattcaacagaataatgctacaaagacatttcgagaattttgtggacctgctgatcctgaaattgcccggcatttacgccctggaactctcagagcaatctttggtaaaactaagatccagaatgctgttcactgtactgatctgccagaggatggcctattagaggttcaatacttcttcaagatcttggataattagtga

SEQ ID NO: 33 - protein, 140 aa, Homo sapiens
  REGION 1..140: note = Human NME7-B1
MNCTCCIVKPHAVSEGLLGKILMAIRDAGFEISAMQMFNMDRVNVEEFYEVYKGVVTEYHDMVTEMYSGPCVAMEIQQNNATKTFREFCGPADPEIARHLRPGTLRAIFGKTKIQNAVHCTDLPEDGLLEVQYFFKILDN

SEQ ID NO: 34 - DNA, 453 bp, Homo sapiens
  misc_feature 1..453: note = Human NME7-B2
  source 1..453: mol_type = other DNA
atgccttcaagtggaggttgtgggccggcaaacactgctaaatttactaattgtacctgttgcattgttaaaccccatgctgtcagtgaaggactgttgggaaagatcctgatggctatccgagatgcaggttttgaaatctcagctatgcagatgttcaatatggatcgggttaatgttgaggaattctatgaagtttataaaggagtagtgaccgaatatcatgacatggtgacagaaatgtattctggcccttgtgtagcaatggagattcaacagaataatgctacaaagacatttcgagaattttgtggacctgctgatcctgaaattgcccggcatttacgccctggaactctcagagcaatctttggtaaaactaagatccagaatgctgttcactgtactgatctgccagaggatggcctattagaggttcaatacttcttctga

SEQ ID NO: 35 - protein, 150 aa, Homo sapiens
  REGION 1..150: note = Human NME7-B2
MPSSGGCGPANTAKFTNCTCCIVKPHAVSEGLLGKILMAIRDAGFEISAMQMFNMDRVNVEEFYEVYKGVVTEYHDMVTEMYSGPCVAMEIQQNNATKTFREFCGPADPEIARHLRPGTLRAIFGKTKIQNAVHCTDLPEDGLLEVQYFF

SEQ ID NO: 36 - DNA, 471 bp, Homo sapiens
  misc_feature 1..471: note = Human NME7-B3
  source 1..471: mol_type = other DNA
atgccttcaagtggaggttgtgggccggcaaacactgctaaatttactaattgtacctgttgcattgttaaaccccatgctgtcagtgaaggactgttgggaaagatcctgatggctatccgagatgcaggttttgaaatctcagctatgcagatgttcaatatggatcgggttaatgttgaggaattctatgaagtttataaaggagtagtgaccgaatatcatgacatggtgacagaaatgtattctggcccttgtgtagcaatggagattcaacagaataatgctacaaagacatttcgagaattttgtggacctgctgatcctgaaattgcccggcatttacgccctggaactctcagagcaatctttggtaaaactaagatccagaatgctgttcactgtactgatctgccagaggatggcctattagaggttcaatacttcttcaagatcttggataattagtga

SEQ ID NO: 37 - protein, 155 aa, Homo sapiens
  REGION 1..155: note = Human NME7-B3
MPSSGGCGPANTAKFTNCTCCIVKPHAVSEGLLGKILMAIRDAGFEISAMQMFNMDRVNVEEFYEVYKGVVTEYHDMVTEMYSGPCVAMEIQQNNATKTFREFCGPADPEIARHLRPGTLRAIFGKTKIQNAVHCTDLPEDGLLEVQYFFKILDN

SEQ ID NO: 38 - DNA, 864 bp, Homo sapiens
  misc_feature 1..864: note = Human NME7-AB
  source 1..864: mol_type = other DNA
atggaaaaaacgctagccctaattaaaccagatgcaatatcaaaggctggagaaataattgaaataataaacaaagctggatttactataaccaaactcaaaatgatgatgctttcaaggaaagaagcattggattttcatgtagatcaccagtcaagaccctttttcaatgagctgatccagtttattacaactggtcctattattgccatggagattttaagagatgatgctatatgtgaatggaaaagactgctgggacctgcaaactctggagtggcacgcacagatgcttctgaaagcattagagccctctttggaacagatggcataagaaatgcagcgcatggccctgattcttttgcttctgcggccagagaaatggagttgttttttccttcaagtggaggttgtgggccggcaaacactgctaaatttactaattgtacctgttgcattgttaaaccccatgctgtcagtgaaggactgttgggaaagatcctgatggctatccgagatgcaggttttgaaatctcagctatgcagatgttcaatatggatcgggttaatgttgaggaattctatgaagtttataaaggagtagtgaccgaatatcatgacatggtgacagaaatgtattctggcccttgtgtagcaatggagattcaacagaataatgctacaaagacatttcgagaattttgtggacctgctgatcctgaaattgcccggcatttacgccctggaactctcagagcaatctttggtaaaactaagatccagaatgctgttcactgtactgatctgccagaggatggcctattagaggttcaatacttcttcaagatcttggataattagtga

SEQ ID NO: 39 - protein, 286 aa, Homo sapiens
  REGION 1..286: note = Human NME7-AB
MEKTLALIKPDAISKAGEIIEIINKAGFTITKLKMMMLSRKEALDFHVDHQSRPFFNELIQFITTGPIIAMEILRDDAICEWKRLLGPANSGVARTDASESIRALFGTDGIRNAAHGPDSFASAAREMELFFPSSGGCGPANTAKFTNCTCCIVKPHAVSEGLLGKILMAIRDAGFEISAMQMFNMDRVNVEEFYEVYKGVVTEYHDMVTEMYSGPCVAMEIQQNNATKTFREFCGPADPEIARHLRPGTLRAIFGKTKIQNAVHCTDLPEDGLLEVQYFFKILDN

SEQ ID NO: 40 - DNA, 846 bp, Homo sapiens
  misc_feature 1..846: note = Human NME7-AB1
  source 1..846: mol_type = other DNA
atggaaaaaacgctagccctaattaaaccagatgcaatatcaaaggctggagaaataattgaaataataaacaaagctggatttactataaccaaactcaaaatgatgatgctttcaaggaaagaagcattggattttcatgtagatcaccagtcaagaccctttttcaatgagctgatccagtttattacaactggtcctattattgccatggagattttaagagatgatgctatatgtgaatggaaaagactgctgggacctgcaaactctggagtggcacgcacagatgcttctgaaagcattagagccctctttggaacagatggcataagaaatgcagcgcatggccctgattcttttgcttctgcggccagagaaatggagttgttttttccttcaagtggaggttgtgggccggcaaacactgctaaatttactaattgtacctgttgcattgttaaaccccatgctgtcagtgaaggactgttgggaaagatcctgatggctatccgagatgcaggttttgaaatctcagctatgcagatgttcaatatggatcgggttaatgttgaggaattctatgaagtttataaaggagtagtgaccgaatatcatgacatggtgacagaaatgtattctggcccttgtgtagcaatggagattcaacagaataatgctacaaagacatttcgagaattttgtggacctgctgatcctgaaattgcccggcatttacgccctggaactctcagagcaatctttggtaaaactaagatccagaatgctgttcactgtactgatctgccagaggatggcctattagaggttcaatacttcttctga

SEQ ID NO: 41 - protein, 281 aa, Homo sapiens
  REGION 1..281: note = Human NME7-AB1
MEKTLALIKPDAISKAGEIIEIINKAGFTITKLKMMMLSRKEALDFHVDHQSRPFFNELIQFITTGPIIAMEILRDDAICEWKRLLGPANSGVARTDASESIRALFGTDGIRNAAHGPDSFASAAREMELFFPSSGGCGPANTAKFTNCTCCIVKPHAVSEGLLGKILMAIRDAGFEISAMQMFNMDRVNVEEFYEVYKGVVTEYHDMVTEMYSGPCVAMEIQQNNATKTFREFCGPADPEIARHLRPGTLRAIFGKTKIQNAVHCTDLPEDGLLEVQYFF

SEQ ID NO: 42 - DNA, 399 bp, synthetic construct
  misc_feature 1..399: note = Description of Artificial Sequence: Synthetic polynucleotide
  misc_feature 1..399: note = Human NME7-A sequence optimized for E. coli expression
  source 1..399: mol_type = other DNA
atggaaaaaacgctggccctgattaaaccggatgcaatctccaaagctggcgaaattatcgaaattatcaacaaagcgggtttcaccatcacgaaactgaaaatgatgatgctgagccgtaaagaagccctggattttcatgtcgaccaccagtctcgcccgtttttcaatgaactgattcaattcatcaccacgggtccgattatcgcaatggaaattctgcgtgatgacgctatctgcgaatggaaacgcctgctgggcccggcaaactcaggtgttgcgcgtaccgatgccagtgaatccattcgcgctctgtttggcaccgatggtatccgtaatgcagcacatggtccggactcattcgcatcggcagctcgtgaaatggaactgtttttctga

SEQ ID NO: 43 - protein, 132 aa, synthetic construct; identical to SEQ ID NO: 23
  REGION 1..132: note = Description of Artificial Sequence: Synthetic polypeptide
  REGION 1..132: note = Human NME7-A sequence optimized for E. coli expression

SEQ ID NO: 44 - DNA, 444 bp, synthetic construct
  misc_feature 1..444: note = Description of Artificial Sequence: Synthetic polynucleotide
  misc_feature 1..444: note = Human NME7-A1 sequence optimized for E. coli expression
  source 1..444: mol_type = other DNA
atggaaaaaacgctggccctgattaaaccggatgcaatctccaaagctggcgaaattatcgaaattatcaacaaagcgggtttcaccatcacgaaactgaaaatgatgatgctgagccgtaaagaagccctggattttcatgtcgaccaccagtctcgcccgtttttcaatgaactgattcaattcatcaccacgggtccgattatcgcaatggaaattctgcgtgatgacgctatctgcgaatggaaacgcctgctgggcccggcaaactcaggtgttgcgcgtaccgatgccagtgaatccattcgcgctctgtttggcaccgatggtatccgtaatgcagcacatggtccggactcattcgcatcggcagctcgtgaaatggaactgtttttcccgagctctggcggttgcggtccggcaaacaccgccaaatttacctga

SEQ ID NO: 45 - protein, 147 aa, synthetic construct; identical to SEQ ID NO: 25
  REGION 1..147: note = Description of Artificial Sequence: Synthetic polypeptide
  REGION 1..147: note = Human NME7-A1 sequence optimized for E. coli expression

SEQ ID NO: 46 - DNA, 669 bp, synthetic construct
  misc_feature 1..669: note = Description of Artificial Sequence: Synthetic polynucleotide
  misc_feature 1..669: note = Human NME7-A2 sequence optimized for E. coli expression
  source 1..669: mol_type = other DNA
atgaatcactccgaacgctttgtttttatcgccgaatggtatgacccgaatgcttccctgctgcgccgctacgaactgctgttttatccgggcgatggtagcgtggaaatgcatgacgttaaaaatcaccgtacctttctgaaacgcacgaaatatgataatctgcatctggaagacctgtttattggcaacaaagtcaatgtgttctctcgtcagctggtgctgatcgattatggcgaccagtacaccgcgcgtcaactgggtagtcgcaaagaaaaaacgctggccctgattaaaccggatgcaatctccaaagctggcgaaattatcgaaattatcaacaaagcgggtttcaccatcacgaaactgaaaatgatgatgctgagccgtaaagaagccctggattttcatgtcgaccaccagtctcgcccgtttttcaatgaactgattcaattcatcaccacgggtccgattatcgcaatggaaattctgcgtgatgacgctatctgcgaatggaaacgcctgctgggcccggcaaactcaggtgttgcgcgtaccgatgccagtgaatccattcgcgctctgtttggcaccgatggtatccgtaatgcagcacatggtccggactcattcgcatcggcagctcgtgaaatggaactgtttttctga

SEQ ID NO: 47 - protein, 222 aa, synthetic construct; identical to SEQ ID NO: 27
  REGION 1..222: note = Description of Artificial Sequence: Synthetic polypeptide
  REGION 1..222: note = Human NME7-A2 sequence optimized for E. coli expression

SEQ ID NO: 48 - DNA, 714 bp, synthetic construct
  misc_feature 1..714: note = Description of Artificial Sequence: Synthetic polynucleotide
  misc_feature 1..714: note = Human NME7-A3 sequence optimized for E. coli expression
  source 1..714: mol_type = other DNA
atgaatcactccgaacgctttgtttttatcgccgaatggtatgacccgaatgcttccctgctgcgccgctacgaactgctgttttatccgggcgatggtagcgtggaaatgcatgacgttaaaaatcaccgtacctttctgaaacgcacgaaatatgataatctgcatctggaagacctgtttattggcaacaaagtcaatgtgttctctcgtcagctggtgctgatcgattatggcgaccagtacaccgcgcgtcaactgggtagtcgcaaagaaaaaacgctggccctgattaaaccggatgcaatctccaaagctggcgaaattatcgaaattatcaacaaagcgggtttcaccatcacgaaactgaaaatgatgatgctgagccgtaaagaagccctggattttcatgtcgaccaccagtctcgcccgtttttcaatgaactgattcaattcatcaccacgggtccgattatcgcaatggaaattctgcgtgatgacgctatctgcgaatggaaacgcctgctgggcccggcaaactcaggtgttgcgcgtaccgatgccagtgaatccattcgcgctctgtttggcaccgatggtatccgtaatgcagcacatggtccggactcattcgcatcggcagctcgtgaaatggaactgtttttcccgagctctggcggttgcggtccggcaaacaccgccaaatttacctga

SEQ ID NO: 49 - protein, 237 aa, synthetic construct; identical to SEQ ID NO: 29
  REGION 1..237: note = Description of Artificial Sequence: Synthetic polypeptide
  REGION 1..237: note = Human NME7-A3 sequence optimized for E. coli expression

SEQ ID NO: 50 - DNA, 408 bp, synthetic construct
  misc_feature 1..408: note = Description of Artificial Sequence: Synthetic polynucleotide
  misc_feature 1..408: note = Human NME7-B sequence optimized for E. coli expression
  source 1..408: mol_type = other DNA
atgaattgtacgtgctgtattgtcaaaccgcacgcagtgtcagaaggcctgctgggtaaaattctgatggcaatccgtgatgctggctttgaaatctcggccatgcagatgttcaacatggaccgcgttaacgtcgaagaattctacgaagtttacaaaggcgtggttaccgaatatcacgatatggttacggaaatgtactccggtccgtgcgtcgcgatggaaattcagcaaaacaatgccaccaaaacgtttcgtgaattctgtggtccggcagatccggaaatcgcacgtcatctgcgtccgggtaccctgcgcgcaatttttggtaaaacgaaaatccagaacgctgtgcactgtaccgatctgccggaagacggtctgctggaagttcaatactttttctga

SEQ ID NO: 51 - protein, 135 aa, synthetic construct; identical to SEQ ID NO: 31
  REGION 1..135: note = Description of Artificial Sequence: Synthetic polypeptide
  REGION 1..135: note = Human NME7-B sequence optimized for E. coli expression

SEQ ID NO: 52 - DNA, 423 bp, synthetic construct
  misc_feature 1..423: note = Description of Artificial Sequence: Synthetic polynucleotide
  misc_feature 1..423: note = Human NME7-B1 sequence optimized for E. coli expression
  source 1..423: mol_type = other DNA
atgaattgtacgtgctgtattgtcaaaccgcacgcagtgtcagaaggcctgctgggtaaaattctgatggcaatccgtgatgctggctttgaaatctcggccatgcagatgttcaacatggaccgcgttaacgtcgaagaattctacgaagtttacaaaggcgtggttaccgaatatcacgatatggttacggaaatgtactccggtccgtgcgtcgcgatggaaattcagcaaaacaatgccaccaaaacgtttcgtgaattctgtggtccggcagatccggaaatcgcacgtcatctgcgtccgggtaccctgcgcgcaatttttggtaaaacgaaaatccagaacgctgtgcactgtaccgatctgccggaagacggtctgctggaagttcaatactttttcaaaattctggataattga

SEQ ID NO: 53 - protein, 140 aa, synthetic construct; identical to SEQ ID NO: 33
  REGION 1..140: note = Description of Artificial Sequence: Synthetic polypeptide
  REGION 1..140: note = Human NME7-B1 sequence optimized for E. coli expression

SEQ ID NO: 54 - DNA, 453 bp, synthetic construct
  misc_feature 1..453: note = Description of Artificial Sequence: Synthetic polynucleotide
  misc_feature 1..453: note = Human NME7-B2 sequence optimized for E. coli expression
  source 1..453: mol_type = other DNA
atgccgagctctggcggttgcggtccggcaaacaccgccaaatttaccaattgtacgtgctgtattgtcaaaccgcacgcagtgtcagaaggcctgctgggtaaaattctgatggcaatccgtgatgctggctttgaaatctcggccatgcagatgttcaacatggaccgcgttaacgtcgaagaattctacgaagtttacaaaggcgtggttaccgaatatcacgatatggttacggaaatgtactccggtccgtgcgtcgcgatggaaattcagcaaaacaatgccaccaaaacgtttcgtgaattctgtggtccggcagatccggaaatcgcacgtcatctgcgtccgggtaccctgcgcgcaatttttggtaaaacgaaaatccagaacgctgtgcactgtaccgatctgccggaagacggtctgctggaagttcaatactttttctga

SEQ ID NO: 55 - protein, 150 aa, synthetic construct; identical to SEQ ID NO: 35
  REGION 1..150: note = Description of Artificial Sequence: Synthetic polypeptide
  REGION 1..150: note = Human NME7-B2 sequence optimized for E. coli expression

SEQ ID NO: 56 - DNA, 468 bp, synthetic construct
  misc_feature 1..468: note = Description of Artificial Sequence: Synthetic polynucleotide
  misc_feature 1..468: note = Human NME7-B3 sequence optimized for E. coli expression
  source 1..468: mol_type = other DNA
atgccgagctctggcggttgcggtccggcaaacaccgccaaatttaccaattgtacgtgctgtattgtcaaaccgcacgcagtgtcagaaggcctgctgggtaaaattctgatggcaatccgtgatgctggctttgaaatctcggccatgcagatgttcaacatggaccgcgttaacgtcgaagaattctacgaagtttacaaaggcgtggttaccgaatatcacgatatggttacggaaatgtactccggtccgtgcgtcgcgatggaaattcagcaaaacaatgccaccaaaacgtttcgtgaattctgtggtccggcagatccggaaatcgcacgtcatctgcgtccgggtaccctgcgcgcaatttttggtaaaacgaaaatccagaacgctgtgcactgtaccgatctgccggaagacggtctgctggaagttcaatactttttcaaaattctggataattga

SEQ ID NO: 57 - protein, 155 aa, synthetic construct; identical to SEQ ID NO: 37
  REGION 1..155: note = Description of Artificial Sequence: Synthetic polypeptide
  REGION 1..155: note = Human NME7-B3 sequence optimized for E. coli expression

SEQ ID NO: 58 - DNA, 861 bp, synthetic construct
  misc_feature 1..861: note = Description of Artificial Sequence: Synthetic polynucleotide
  misc_feature 1..861: note = Human NME7-AB sequence optimized for E. coli expression
  source 1..861: mol_type = other DNA
atggaaaaaacgctggccctgattaaaccggatgcaatctccaaagctggcgaaattatcgaaattatcaacaaagcgggtttcaccatcacgaaactgaaaatgatgatgctgagccgtaaagaagccctggattttcatgtcgaccaccagtctcgcccgtttttcaatgaactgattcaattcatcaccacgggtccgattatcgcaatggaaattctgcgtgatgacgctatctgcgaatggaaacgcctgctgggcccggcaaactcaggtgttgcgcgtaccgatgccagtgaatccattcgcgctctgtttggcaccgatggtatccgtaatgcagcacatggtccggactcattcgcatcggcagctcgtgaaatggaactgtttttcccgagctctggcggttgcggtccggcaaacaccgccaaatttaccaattgtacgtgctgtattgtcaaaccgcacgcagtgtcagaaggcctgctgggtaaaattctgatggcaatccgtgatgctggctttgaaatctcggccatgcagatgttcaacatggaccgcgttaacgtcgaagaattctacgaagtttacaaaggcgtggttaccgaatatcacgatatggttacggaaatgtactccggtccgtgcgtcgcgatggaaattcagcaaaacaatgccaccaaaacgtttcgtgaattctgtggtccggcagatccggaaatcgcacgtcatctgcgtccgggtaccctgcgcgcaatttttggtaaaacgaaaatccagaacgctgtgcactgtaccgatctgccggaagacggtctgctggaagttcaatactttttcaaaattctggataattga

SEQ ID NO: 59 - protein, 286 aa, synthetic construct; identical to SEQ ID NO: 39
  REGION 1..286: note = Description of Artificial Sequence: Synthetic polypeptide
  REGION 1..286: note = Human NME7-AB sequence optimized for E. coli expression

SEQ ID NO: 60 - DNA, 846 bp, synthetic construct
  misc_feature 1..846: note = Description of Artificial Sequence: Synthetic polynucleotide
  misc_feature 1..846: note = Human NME7-AB1 sequence optimized for E. coli expression
  source 1..846: mol_type = other DNA
atggaaaaaacgctggccctgattaaaccggatgcaatctccaaagctggcgaaattatcgaaattatcaacaaagcgggtttcaccatcacgaaactgaaaatgatgatgctgagccgtaaagaagccctggattttcatgtcgaccaccagtctcgcccgtttttcaatgaactgattcaattcatcaccacgggtccgattatcgcaatggaaattctgcgtgatgacgctatctgcgaatggaaacgcctgctgggcccggcaaactcaggtgttgcgcgtaccgatgccagtgaatccattcgcgctctgtttggcaccgatggtatccgtaatgcagcacatggtccggactcattcgcatcggcagctcgtgaaatggaactgtttttcccgagctctggcggttgcggtccggcaaacaccgccaaatttaccaattgtacgtgctgtattgtcaaaccgcacgcagtgtcagaaggcctgctgggtaaaattctgatggcaatccgtgatgctggctttgaaatctcggccatgcagatgttcaacatggaccgcgttaacgtcgaagaattctacgaagtttacaaaggcgtggttaccgaatatcacgatatggttacggaaatgtactccggtccgtgcgtcgcgatggaaattcagcaaaacaatgccaccaaaacgtttcgtgaattctgtggtccggcagatccggaaatcgcacgtcatctgcgtccgggtaccctgcgcgcaatttttggtaaaacgaaaatccagaacgctgtgcactgtaccgatctgccggaagacggtctgctggaagttcaatactttttctga

SEQ ID NO: 61 - protein, 281 aa, synthetic construct; identical to SEQ ID NO: 41
  REGION 1..281: note = Description of Artificial Sequence: Synthetic polypeptide
  REGION 1..281: note = Human NME7-AB1 sequence optimized for E. coli expression

SEQ ID NO: 62 - DNA, 570 bp, Mus sp.
  misc_feature 1..570: note = DNA encoding Mouse NME6
  source 1..570: mol_type = other DNA
atgacctccatcttgcgaagtccccaagctcttcagctcacactagccctgatcaagcctgatgcagttgcccacccactgatcctggaggctgttcatcagcagattctgagcaacaagttcctcattgtacgaacgagggaactgcagtggaagctggaggactgccggaggttttaccgagagcatgaagggcgttttttctatcagcggctggtggagttcatgacaagtgggccaatccgagcctatatccttgcccacaaagatgccatccaactttggaggacactgatgggacccaccagagtatttcgagcacgctatatagccccagattcaattcgtggaagtttgggcctcactgacacccgaaatactacccatggctcagactccgtggtttccgccagcagagagattgcagccttcttccctgacttcagtgaacagcgctggtatgaggaggaggaaccccagctgcggtgtggtcctgtgcactacagtccagaggaaggtatccactgtgcagctgaaacaggaggccacaaacaacctaacaaaacctag

SEQ ID NO: 63 - protein, 189 aa, Mus sp.
  REGION 1..189: note = Mouse NME6
MTSILRSPQALQLTLALIKPDAVAHPLILEAVHQQILSNKFLIVRTRELQWKLEDCRRFYREHEGRFFYQRLVEFMTSGPIRAYILAHKDAIQLWRTLMGPTRVFRARYIAPDSIRGSLGLTDTRNTTHGSDSVVSASREIAAFFPDFSEQRWYEEEEPQLRCGPVHYSPEEGIHCAAETGGHKQPNKT

SEQ ID NO: 64 - DNA, 585 bp, Homo sapiens
  misc_feature 1..585: note = DNA encoding Human NME6
  source 1..585: mol_type = other DNA
atgacccagaatctggggagtgagatggcctcaatcttgcgaagccctcaggctctccagctcactctagccctgatcaagcctgacgcagtcgcccatccactgattctggaggctgttcatcagcagattctaagcaacaagttcctgattgtacgaatgagagaactactgtggagaaaggaagattgccagaggttttaccgagagcatgaagggcgttttttctatcagaggctggtggagttcatggccagcgggccaatccgagcctacatccttgcccacaaggatgccatccagctctggaggacgctcatgggacccaccagagtgttccgagcacgccatgtggccccagattctatccgtgggagtttcggcctcactgacacccgcaacaccacccatggttcggactctgtggtttcagccagcagagagattgcagccttcttccctgacttcagtgaacagcgctggtatgaggaggaagagccccagttgcgctgtggccctgtgtgctatagcccagagggaggtgtccactatgtagctggaacaggaggcctaggaccagcctga

SEQ ID NO: 65 - protein, 194 aa, Homo sapiens
  REGION 1..194: note = Human NME6
MTQNLGSEMASILRSPQALQLTLALIKPDAVAHPLILEAVHQQILSNKFLIVRMRELLWRKEDCQRFYREHEGRFFYQRLVEFMASGPIRAYILAHKDAIQLWRTLMGPTRVFRARHVAPDSIRGSFGLTDTRNTTHGSDSVVSASREIAAFFPDFSEQRWYEEEEPQLRCGPVCYSPEGGVHYVAGTGGLGPA

SEQ ID NO: 66 - DNA, 525 bp, Homo sapiens
  misc_feature 1..525: note = DNA encoding Human NME6 1
  source 1..525: mol_type = other DNA
atgacccagaatctggggagtgagatggcctcaatcttgcgaagccctcaggctctccagctcactctagccctgatcaagcctgacgcagtcgcccatccactgattctggaggctgttcatcagcagattctaagcaacaagttcctgattgtacgaatgagagaactactgtggagaaaggaagattgccagaggttttaccgagagcatgaagggcgttttttctatcagaggctggtggagttcatggccagcgggccaatccgagcctacatccttgcccacaaggatgccatccagctctggaggacgctcatgggacccaccagagtgttccgagcacgccatgtggccccagattctatccgtgggagtttcggcctcactgacacccgcaacaccacccatggttcggactctgtggtttcagccagcagagagattgcagccttcttccctgacttcagtgaacagcgctggtatgaggaggaagagccccagttgcgctgtggccctgtgtga

SEQ ID NO: 67 - protein, 174 aa, Homo sapiens
  REGION 1..174: note = Human NME6 1
MTQNLGSEMASILRSPQALQLTLALIKPDAVAHPLILEAVHQQILSNKFLIVRMRELLWRKEDCQRFYREHEGRFFYQRLVEFMASGPIRAYILAHKDAIQLWRTLMGPTRVFRARHVAPDSIRGSFGLTDTRNTTHGSDSVVSASREIAAFFPDFSEQRWYEEEEPQLRCGPV

SEQ ID NO: 68 - DNA, 468 bp, Homo sapiens
  misc_feature 1..468: note = DNA encoding Human NME6 2
  source 1..468: mol_type = other DNA
atgctcactctagccctgatcaagcctgacgcagtcgcccatccactgattctggaggctgttcatcagcagattctaagcaacaagttcctgattgtacgaatgagagaactactgtggagaaaggaagattgccagaggttttaccgagagcatgaagggcgttttttctatcagaggctggtggagttcatggccagcgggccaatccgagcctacatccttgcccacaaggatgccatccagctctggaggacgctcatgggacccaccagagtgttccgagcacgccatgtggccccagattctatccgtgggagtttcggcctcactgacacccgcaacaccacccatggttcggactctgtggtttcagccagcagagagattgcagccttcttccctgacttcagtgaacagcgctggtatgaggaggaagagccccagttgcgctgtggccctgtgtga

SEQ ID NO: 69 - protein, 155 aa, Homo sapiens
  REGION 1..155: note = Human NME6 2
MLTLALIKPDAVAHPLILEAVHQQILSNKFLIVRMRELLWRKEDCQRFYREHEGRFFYQRLVEFMASGPIRAYILAHKDAIQLWRTLMGPTRVFRARHVAPDSIRGSFGLTDTRNTTHGSDSVVSASREIAAFFPDFSEQRWYEEEEPQLRCGPV

SEQ ID NO: 70 - DNA, 528 bp, Homo sapiens
  misc_feature 1..528: note = DNA encoding Human NME6 3
  source 1..528: mol_type = other DNA
atgctcactctagccctgatcaagcctgacgcagtcgcccatccactgattctggaggctgttcatcagcagattctaagcaacaagttcctgattgtacgaatgagagaactactgtggagaaaggaagattgccagaggttttaccgagagcatgaagggcgttttttctatcagaggctggtggagttcatggccagcgggccaatccgagcctacatccttgcccacaaggatgccatccagctctggaggacgctcatgggacccaccagagtgttccgagcacgccatgtggccccagattctatccgtgggagtttcggcctcactgacacccgcaacaccacccatggttcggactctgtggtttcagccagcagagagattgcagccttcttccctgacttcagtgaacagcgctggtatgaggaggaagagccccagttgcgctgtggccctgtgtgctatagcccagagggaggtgtccactatgtagctggaacaggaggcctaggaccagcctga

SEQ ID NO: 71 - protein, 175 aa, Homo sapiens
  REGION 1..175: note = Human NME6 3
MLTLALIKPDAVAHPLILEAVHQQILSNKFLIVRMRELLWRKEDCQRFYREHEGRFFYQRLVEFMASGPIRAYILAHKDAIQLWRTLMGPTRVFRARHVAPDSIRGSFGLTDTRNTTHGSDSVVSASREIAAFFPDFSEQRWYEEEEPQLRCGPVCYSPEGGVHYVAGTGGLGPA

SEQ ID NO: 72 - DNA, 585 bp, synthetic construct
  misc_feature 1..585: note = Description of Artificial Sequence: Synthetic polynucleotide
  misc_feature 1..585: note = Human NME6 sequence optimized for E. coli expression
  source 1..585: mol_type = other DNA
atgacgcaaaatctgggctcggaaatggcaagtatcctgcgctccccgcaagcactgcaactgaccctggctctgatcaaaccggacgctgttgctcatccgctgattctggaagcggtccaccagcaaattctgagcaacaaatttctgatcgtgcgtatgcgcgaactgctgtggcgtaaagaagattgccagcgtttttatcgcgaacatgaaggccgtttcttttatcaacgcctggttgaattcatggcctctggtccgattcgcgcatatatcctggctcacaaagatgcgattcagctgtggcgtaccctgatgggtccgacgcgcgtctttcgtgcacgtcatgtggcaccggactcaatccgtggctcgttcggtctgaccgatacgcgcaataccacgcacggtagcgactctgttgttagtgcgtcccgtgaaatcgcggcctttttcccggacttctccgaacagcgttggtacgaagaagaagaaccgcaactgcgctgtggcccggtctgttattctccggaaggtggtgtccattatgtggcgggcacgggtggtctgggtccggcatga

SEQ ID NO: 73 - protein, 194 aa, synthetic construct; identical to SEQ ID NO: 65
  REGION 1..194: note = Description of Artificial Sequence: Synthetic polypeptide
  REGION 1..194: note = Human NME6 sequence optimized for E. coli expression

SEQ ID NO: 74 - DNA, 525 bp, synthetic construct
  misc_feature 1..525: note = Description of Artificial Sequence: Synthetic polynucleotide
  misc_feature 1..525: note = Human NME6 1 sequence optimized for E. coli expression
  source 1..525: mol_type = other DNA
atgacgcaaaatctgggctcggaaatggcaagtatcctgcgctccccgcaagcactgcaactgaccctggctctgatcaaaccggacgctgttgctcatccgctgattctggaagcggtccaccagcaaattctgagcaacaaatttctgatcgtgcgtatgcgcgaactgctgtggcgtaaagaagattgccagcgtttttatcgcgaacatgaaggccgtttcttttatcaacgcctggttgaattcatggcctctggtccgattcgcgcatatatcctggctcacaaagatgcgattcagctgtggcgtaccctgatgggtccgacgcgcgtctttcgtgcacgtcatgtggcaccggactcaatccgtggctcgttcggtctgaccgatacgcgcaataccacgcacggtagcgactctgttgttagtgcgtcccgtgaaatcgcggcctttttcccggacttctccgaacagcgttggtacgaagaagaagaaccgcaactgcgctgtggcccggtctga

SEQ ID NO: 75 - protein, 174 aa, synthetic construct; identical to SEQ ID NO: 67
  REGION 1..174: note = Description of Artificial Sequence: Synthetic polypeptide
  REGION 1..174: note = Human NME6 1 sequence optimized for E. coli expression

SEQ ID NO: 76 - DNA, 468 bp, synthetic construct
  misc_feature 1..468: note = Description of Artificial Sequence: Synthetic polynucleotide
  misc_feature 1..468: note = Human NME6 2 sequence optimized for E. coli expression
  source 1..468: mol_type = other DNA
atgctgaccctggctctgatcaaaccggacgctgttgctcatccgctgattctggaagcggtccaccagcaaattctgagcaacaaatttctgatcgtgcgtatgcgcgaactgctgtggcgtaaagaagattgccagcgtttttatcgcgaacatgaaggccgtttcttttatcaacgcctggttgaattcatggcctctggtccgattcgcgcatatatcctggctcacaaagatgcgattcagctgtggcgtaccctgatgggtccgacgcgcgtctttcgtgcacgtcatgtggcaccggactcaatccgtggctcgttcggtctgaccgatacgcgcaataccacgcacggtagcgactctgttgttagtgcgtcccgtgaaatcgcggcctttttcccggacttctccgaacagcgttggtacgaagaagaagaaccgcaactgcgctgtggcccggtctga

SEQ ID NO: 77 - protein, 155 aa, synthetic construct; identical to SEQ ID NO: 69
  REGION 1..155: note = Description of Artificial Sequence: Synthetic polypeptide
  REGION 1..155: note = Human NME6 2 sequence optimized for E. coli expression

SEQ ID NO: 78 - DNA, 528 bp, synthetic construct
  misc_feature 1..528: note = Description of Artificial Sequence: Synthetic polynucleotide
  misc_feature 1..528: note = Human NME6 3 sequence optimized for E. coli expression
  source 1..528: mol_type = other DNA
atgctgaccctggctctgatcaaaccggacgctgttgctcatccgctgattctggaagcggtccaccagcaaattctgagcaacaaatttctgatcgtgcgtatgcgcgaactgctgtggcgtaaagaagattgccagcgtttttatcgcgaacatgaaggccgtttcttttatcaacgcctggttgaattcatggcctctggtccgattcgcgcatatatcctggctcacaaagatgcgattcagctgtggcgtaccctgatgggtccgacgcgcgtctttcgtgcacgtcatgtggcaccggactcaatccgtggctcgttcggtctgaccgatacgcgcaataccacgcacggtagcgactctgttgttagtgcgtcccgtgaaatcgcggcctttttcccggacttctccgaacagcgttggtacgaagaagaagaaccgcaactgcgctgtggcccggtctgttattctccggaaggtggtgtccattatgtggcgggcacgggtggtctgggtccggcatga

SEQ ID NO: 79 - protein, 175 aa, synthetic construct; identical to SEQ ID NO: 71
  REGION 1..175: note = Description of Artificial Sequence: Synthetic polypeptide
  REGION 1..175: note = Human NME6 3 sequence optimized for E. coli expression

SEQ ID NO: 80 - DNA, 1306 bp, synthetic construct
  misc_feature 1..1306: note = Description of Artificial Sequence: Synthetic polynucleotide
  misc_feature 1..1306: note = OriGene-NME7-1 full length
  source 1..1306: mol_type = other DNA
gacgttgtatacgactcctatagggcggccgggaattcgtcgactggatccggtaccgaggagatctgccgccgcgatcgccatgaatcatagtgaaagattcgttttcattgcagagtggtatgatccaaatgcttcacttcttcgacgttatgagcttttattttacccaggggatggatctgttgaaatgcatgatgtaaagaatcatcgcacctttttaaagcggaccaaatatgataacctgcacttggaagatttatttataggcaacaaagtgaatgtcttctctcgacaactggtattaattgactatggggatcaatatacagctcgccagctgggcagtaggaaagaaaaaacgctagccctaattaaaccagatgcaatatcaaaggctggagaaataattgaaataataaacaaagctggatttactataaccaaactcaaaatgatgatgctttcaaggaaagaagcattggattttcatgtagatcaccagtcaagaccctttttcaatgagctgatccagtttattacaactggtcctattattgccatggagattttaagagatgatgctatatgtgaatggaaaagactgctgggacctgcaaactctggagtggcacgcacagatgcttctgaaagcattagagccctctttggaacagatggcataagaaatgcagcgcatggccctgattcttttgcttctgcggccagagaaatggagttgttttttccttcaagtggaggttgtgggccggcaaacactgctaaatttactaattgtacctgttgcattgttaaaccccatgctgtcagtgaaggactgttgggaaagatcctgatggctatccgagatgcaggttttgaaatctcagctatgcagatgttcaatatggatcgggttaatgttgaggaattctatgaagtttataaaggagtagtgaccgaatatcatgacatggtgacagaaatgtattctggcccttgtgtagcaatggagattcaacagaataatgctacaaagacatttcgagaattttgtggacctgctgatcctgaaattgcccggcatttacgccctggaactctcagagcaatctttggtaaaactaagatccagaatgctgttcactgtactgatctgccagaggatggcctattagaggttcaatacttcttcaagatcttggataatacgcgtacgcggccgctcgagcagaaactcatctcagaagaggatctggcagcaaatgatatcctggattacaaggatgacgacgataaggtttaa

SEQ ID NO: 81 - protein, 407 aa, synthetic construct
  REGION 1..407: note = Description of Artificial Sequence: Synthetic polypeptide
  REGION 1..407: note = OriGene-NME7-1 full length
MNHSERFVFIAEWYDPNASLLRRYELLFYPGDGSVEMHDVKNHRTFLKRTKYDNLHLEDLFIGNKVNVFSRQLVLIDYGDQYTARQLGSRKEKTLALIKPDAISKAGEIIEIINKAGFTITKLKMMMLSRKEALDFHVDHQSRPFFNELIQFITTGPIIAMEILRDDAICEWKRLLGPANSGVARTDASESIRALFGTDGIRNAAHGPDSFASAAREMELFFPSSGGCGPANTAKFTNCTCCIVKPHAVSEGLLGKILMAIRDAGFEISAMQMFNMDRVNVEEFYEVYKGVVTEYHDMVTEMYSGPCVAMEIQQNNATKTFREFCGPADPEIARHLRPGTLRAIFGKTKIQNAVHCTDLPEDGLLEVQYFFKILDNTRTRRLEQKLISEEDLAANDILDYKDDDDKV

SEQ ID NO: 82 - protein, 376 aa, synthetic construct
  REGION 1..376: note = Description of Artificial Sequence: Synthetic polypeptide
  REGION 1..376: note = Abnova NME7-1 Full length
MNHSERFVFIAEWYDPNASLLRRYELLFYPGDGSVEMHDVKNHRTFLKRTKYDNLHLEDLFIGNKVNVFSRQLVLIDYGDQYTARQLGSRKEKTLALIKPDAISKAGEIIEIINKAGFTITKLKMMMLSRKEALDFHVDHQSRPFFNELIQFITTGPIIAMEILRDDAICEWKRLLGPANSGVARTDASESIRALFGTDGIRNAAHGPDSFASAAREMELFFPSSGGCGPANTAKFTNCTCCIVKPHAVSEGLLGKILMAIRDAGFEISAMQMFNMDRVNVEEFYEVYKGVVTEYHDMVTEMYSGPCVAMEIQQNNATKTFREFCGPADPEIARHLRPGTLRAIFGKTKIQNAVHCTDLPEDGLLEVQYFFKILDN

SEQ ID NO: 83 - protein, 98 aa, synthetic construct
  REGION 1..98: note = Description of Artificial Sequence: Synthetic polypeptide
  REGION 1..98: note = Abnova Partial NME7-B
DRVNVEEFYEVYKGVVTEYHDMVTEMYSGPCVAMEIQQNNATKTFREFCGPADPEIARHLRPGTLRAIFGKTKIQNAVHCTDLPEDGLLEVQYFFKIL

SEQ ID NO: 84 - DNA, 27 bp, synthetic construct
  misc_feature 1..27: note = Description of Artificial Sequence: Synthetic oligonucleotide
  misc_feature 1..27: note = DNA encoding Histidine Tag
  source 1..27: mol_type = other DNA
ctcgagcaccaccaccaccaccactga

SEQ ID NO: 85 - DNA, 36 bp, synthetic construct
  misc_feature 1..36: note = Description of Artificial Sequence: Synthetic oligonucleotide
  misc_feature 1..36: note = DNA encoding Strept II Tag
  source 1..36: mol_type = other DNA
accggttggagccatcctcagttcgaaaagtaatga

SEQ ID NO: 86 - protein, 35 aa, synthetic construct
  REGION 1..35: note = Description of Artificial Sequence: Synthetic polypeptide
  REGION 1..35: note = N-10 peptide
QFNQYKTEAASRYNLTISDVSVSDVPFPFSAQSGA

SEQ ID NO: 87 - protein, 35 aa, synthetic construct
  REGION 1..35: note = Description of Artificial Sequence: Synthetic polypeptide
  REGION 1..35: note = C-10 peptide
GTINVHDVETQFNQYKTEAASRYNLTISDVSVSDV

SEQ ID NO: 88 - protein, 8 aa, synthetic construct
  REGION 1..8: note = Description of Artificial Sequence: Synthetic peptide
  REGION 1..8: note = Immunizing peptides derived from human NME7
LALIKPDA

SEQ ID NO: 89 - protein, 18 aa, synthetic construct
  REGION 1..18: note = Description of Artificial Sequence: Synthetic peptide
  REGION 1..18: note = Immunizing peptides derived from human NME7
MMMLSRKEALDFHVDHQS

SEQ ID NO: 90 - protein, 10 aa, synthetic construct
  REGION 1..10: note = Description of Artificial Sequence: Synthetic peptide
  REGION 1..10: note = Immunizing peptides derived from human NME7
ALDFHVDHQS

SEQ ID NO: 91 - protein, 14 aa, synthetic construct
  REGION 1..14: note = Description of Artificial Sequence: Synthetic peptide
  REGION 1..14: note = Immunizing peptides derived from human NME7
EILRDDAICEWKRL

SEQ ID NO: 92 - protein, 12 aa, synthetic construct
  REGION 1..12: note = Description of Artificial Sequence: Synthetic peptide
  REGION 1..12: note = Immunizing peptides derived from human NME7
FNELIQFITTGP

SEQ ID NO: 93 - protein, 8 aa, synthetic construct
  REGION 1..8: note = Description of Artificial Sequence: Synthetic peptide
  REGION 1..8: note = Immunizing peptides derived from human NME7
RDDAICEW

SEQ ID NO: 94 - protein, 25 aa, synthetic construct
  REGION 1..25: note = Description of Artificial Sequence: Synthetic peptide
  REGION 1..25: note = Immunizing peptides derived from human NME7
SGVARTDASESIRALFGTDGIRNAA

SEQ ID NO: 95 - protein, 9 aa, synthetic construct
  REGION 1..9: note = Description of Artificial Sequence: Synthetic peptide
  REGION 1..9: note = Immunizing peptides derived from human NME7
ELFFPSSGG

SEQ ID NO: 96 - protein, 26 aa, synthetic construct
  REGION 1..26: note = Description of Artificial Sequence: Synthetic peptide
  REGION 1..26: note = Immunizing peptides derived from human NME7
KFTNCTCCIVKPHAVSEGLLGKILMA

SEQ ID NO: 97 - protein, 36 aa, synthetic construct
  REGION 1..36: note = Description of Artificial Sequence: Synthetic polypeptide
  REGION 1..36: note = Immunizing peptides derived from human NME7
LMAIRDAGFEISAMQMFNMDRVNVEEFYEVYKGVVT

SEQ ID NO: 98 - protein, 15 aa, synthetic construct
  REGION 1..15: note = Description of Artificial Sequence: Synthetic peptide
  REGION 1..15: note = Immunizing peptides derived from human NME7
EFYEVYKGVVTEYHD

SEQ ID NO: 99 - protein, 43 aa, synthetic construct
  REGION 1..43: note = Description of Artificial Sequence: Synthetic polypeptide
  REGION 1..43: note = Immunizing peptides derived from human NME7
EIQQNNATKTFREFCGPADPEIARHLRPGTLRAIFGKTKIQNA

SEQ ID NO: 100 - protein, 8 aa, synthetic construct
  REGION 1..8: note = Description of Artificial Sequence: Synthetic peptide
  REGION 1..8: note = Immunizing peptides derived from human NME7
YSGPCVAM

SEQ ID NO: 101 - protein, 7 aa, synthetic construct
  REGION 1..7: note = Description of Artificial Sequence: Synthetic peptide
  REGION 1..7: note = Immunizing peptides derived from human NME7
FREFCGP

SEQ ID NO: 102 - protein, 23 aa, synthetic construct
  REGION 1..23: note = Description of Artificial Sequence: Synthetic peptide
  REGION 1..23: note = Immunizing peptides derived from human NME7
VHCTDLPEDGLLEVQYFFKILDN

SEQ ID NO: 103 - protein, 9 aa, synthetic construct
  REGION 1..9: note = Description of Artificial Sequence: Synthetic peptide
  REGION 1..9: note = Immunizing peptides derived from human NME7
IQNAVHCTD

SEQ ID NO: 104 - protein, 20 aa, synthetic construct
  REGION 1..20: note = Description of Artificial Sequence: Synthetic peptide
  REGION 1..20: note = Immunizing peptides derived from human NME7
TDLPEDGLLEVQYFFKILDN

SEQ ID NO: 105 - protein, 13 aa, synthetic construct
  REGION 1..13: note = Description of Artificial Sequence: Synthetic peptide
  REGION 1..13: note = Immunizing peptides derived from human NME7
PEDGLLEVQYFFK

SEQ ID NO: 106 - protein, 12 aa, synthetic construct
  REGION 1..12: note = Description of Artificial Sequence: Synthetic peptide
  REGION 1..12: note = Immunizing peptides derived from human NME7
EIINKAGFTITK

SEQ ID NO: 107 - protein, 16 aa, synthetic construct
  REGION 1..16: note = Description of Artificial Sequence: Synthetic peptide
  REGION 1..16: note = Immunizing peptides derived from human NME7
MLSRKEALDFHVDHQS

SEQ ID NO: 108 - protein, 9 aa, synthetic construct
  REGION 1..9: note = Description of Artificial Sequence: Synthetic peptide
  REGION 1..9: note = Immunizing peptides derived from human NME7
NELIQFITT

SEQ ID NO: 109 - protein, 14 aa, synthetic construct
  REGION 1..14: note = Description of Artificial Sequence: Synthetic peptide
  REGION 1..14: note = Immunizing peptides derived from human NME7
EILRDDAICEWKRL

SEQ ID NO: 110 - protein, 21 aa, synthetic construct
  REGION 1..21: note = Description of Artificial Sequence: Synthetic peptide
  REGION 1..21: note = Immunizing peptides derived from human NME7
SGVARTDASESIRALFGTDGI

SEQ ID NO: 111 - protein, 11 aa, synthetic construct
  REGION 1..11: note = Description of Artificial Sequence: Synthetic peptide
  REGION 1..11: note = Immunizing peptides derived from human NME7
SGVARTDASES

SEQ ID NO: 112 - protein, 8 aa, synthetic construct
  REGION 1..8: note = Description of Artificial Sequence: Synthetic peptide
  REGION 1..8: note = Immunizing peptides derived from human NME7
ALFGTDGI

SEQ ID NO: 113 - protein, 14 aa, synthetic construct
  REGION 1..14: note = Description of Artificial Sequence: Synthetic peptide
  REGION 1..14: note = Immunizing peptides derived from human NME7
NCTCCIVKPHAVSE

SEQ ID NO: 114 - protein, 11 aa, synthetic construct
  REGION 1..11: note = Description of Artificial Sequence: Synthetic peptide
  REGION 1..11: note = Immunizing peptides derived from human NME7
LGKILMAIRDA

SEQ ID NO: 115 - protein, 16 aa, synthetic construct
  REGION 1..16: note = Description of Artificial Sequence: Synthetic peptide
  REGION 1..16: note = Immunizing peptides derived from human NME7
EISAMQMFNMDRVNVE

SEQ ID NO: 116 - protein, 8 aa, synthetic construct
  REGION 1..8: note = Description of Artificial Sequence: Synthetic peptide
  REGION 1..8: note = Immunizing peptides derived from human NME7
EVYKGVVT

SEQ ID NO: 117 - protein, 8 aa, synthetic construct
  REGION 1..8: note = Description of Artificial Sequence: Synthetic peptide
  REGION 1..8: note = Immunizing peptides derived from human NME7
EYHDMVTE

SEQ ID NO: 118 - protein, 15 aa, synthetic construct
  REGION 1..15: note = Description of Artificial Sequence: Synthetic peptide
  REGION 1..15: note = Immunizing peptides derived from human NME7
EFCGPADPEIARHLR

SEQ ID NO: 119 - protein, 12 aa, synthetic construct
  REGION 1..12: note = Description of Artificial Sequence: Synthetic peptide
  REGION 1..12: note = Immunizing peptides derived from human NME7
AIFGKTKIQNAV

SEQ ID NO: 120 - protein, 18 aa, synthetic construct
  REGION 1..18: note = Description of Artificial Sequence: Synthetic peptide
  REGION 1..18: note = Immunizing peptides derived from human NME7
LPEDGLLEVQYFFKILDN

SEQ ID NO: 121 - protein, 17 aa, synthetic construct
  REGION 1..17: note = Description of Artificial Sequence: Synthetic peptide
  REGION 1..17: note = Immunizing peptides derived from human NME7
GPDSFASAAREMELFFP

SEQ ID NO: 122 - protein, 7 aa, synthetic construct
  REGION 1..7: note = Description of Artificial Sequence: Synthetic peptide
  REGION 1..7: note = Immunizing peptides derived from human NME7
ICEWKRL

SEQ ID NO: 123 - protein, 11 aa, synthetic construct
  REGION 1..11: note = Description of Artificial Sequence: Synthetic peptide
  REGION 1..11: note = Immunizing peptides derived from human NME7
LGKILMAIRDA

SEQ ID NO: 124 - protein, 10 aa, synthetic construct
  REGION 1..10: note = Description of Artificial Sequence: Synthetic peptide
  REGION 1..10: note = Immunizing peptides derived from human NME7
HAVSEGLLGK

SEQ ID NO: 125 - protein, 8 aa, synthetic construct
  REGION 1..8: note = Description of Artificial Sequence: Synthetic peptide
  REGION 1..8: note = Immunizing peptides derived from human NME7
VTEMYSGP

SEQ ID NO: 126 - protein, 9 aa, synthetic construct
  REGION 1..9: note = Description of Artificial Sequence: Synthetic peptide
  REGION 1..9: note = Immunizing peptides derived from human NME7
NATKTFREF

SEQ ID NO: 127 - protein, 9 aa, synthetic construct
  REGION 1..9: note = Description of Artificial Sequence: Synthetic peptide
  REGION 1..9: note = Immunizing peptides derived from human NME7
AIRDAGFEI

SEQ ID NO: 128 - protein, 13 aa, synthetic construct
  REGION 1..13: note = Description of Artificial Sequence: Synthetic peptide
  REGION 1..13: note = Immunizing peptides derived from human NME7
AICEWKRLLGPAN

SEQ ID NO: 129 - protein, 8 aa, synthetic construct
  REGION 1..8: note = Description of Artificial Sequence: Synthetic peptide
  REGION 1..8: note = Immunizing peptides derived from human NME7
DHQSRPFF

SEQ ID NO: 130 - protein, 13 aa, synthetic construct
  REGION 1..13: note = Description of Artificial Sequence: Synthetic peptide
  REGION 1..13: note = Immunizing peptides derived from human NME7
AICEWKRLLGPAN

SEQ ID NO: 131 - protein, 8 aa, synthetic construct
  REGION 1..8: note = Description of Artificial Sequence: Synthetic peptide
  REGION 1..8: note = Immunizing peptides derived from human NME7
VDHQSRPF

SEQ ID NO: 132 - protein, 6 aa, synthetic construct
  REGION 1..6: note = Description of Artificial Sequence: Synthetic peptide
  REGION 1..6: note = Immunizing peptides derived from human NME7
PDSFAS

SEQ ID NO: 133 - protein, 18 aa, synthetic construct
  REGION 1..18: note = Description of Artificial Sequence: Synthetic peptide
  REGION 1..18: note = Immunizing peptides derived from human NME7
KAGEIIEIINKAGFTITK

SEQ ID NO: 134 - protein, 29 aa, synthetic construct
  REGION 1..29: note = Description of Artificial Sequence: Synthetic peptide
  REGION 1..29: note = Immunizing peptides derived from human NME1
MANCERTFIAIKPDGVQRGLVGEIIKRFE

SEQ ID NO: 135 - protein, 8 aa, synthetic construct
  REGION 1..8: note = Description of Artificial Sequence: Synthetic peptide
  REGION 1..8: note = Immunizing peptides derived from human NME1
VDLKDRPF

SEQ ID NO: 136 - protein, 17 aa, synthetic construct
  REGION 1..17: note = Description of Artificial Sequence: Synthetic peptide
  REGION 1..17: note = Immunizing peptides derived from human NME1
HGSDSVESAEKEIGLWF

SEQ ID NO: 137 - protein, 25 aa, synthetic construct
  REGION 1..25: note = Description of Artificial Sequence: Synthetic peptide
  REGION 1..25: note = Immunizing peptides derived from human NME1
ERTFIAIKPDGVQRGLVGEIIKRFE

SEQ ID NO: 138 - protein, 30 aa, synthetic construct
  REGION 1..30: note = Description of Artificial Sequence: Synthetic polypeptide
  REGION 1..30: note = Immunizing peptides derived from human NME1
VDLKDRPFFAGLVKYMHSGPVVAMVWEGLN

SEQ ID NO: 139 - protein, 26 aa, synthetic construct
  REGION 1..26: note = Description of Artificial Sequence: Synthetic peptide
  REGION 1..26: note = Immunizing peptides derived from human NME1
NIIHGSDSVESAEKEIGLWFHPEELV

SEQ ID NO: 140 - protein, 14 aa, synthetic construct
  REGION 1..14: note = Description of Artificial Sequence: Synthetic peptide
  REGION 1..14: note = Immunizing peptides derived from human NME1
KPDGVQRGLVGEII

SEQ ID NO: 141 - DNA, 27 bp, synthetic construct
  misc_feature 1..27: note = Description of Artificial Sequence: Synthetic Primer
  source 1..27: mol_type = other DNA
atcgatcatatgaatcactccgaacgc

SEQ ID NO: 142 - DNA, 38 bp, synthetic construct
  misc_feature 1..38: note = Description of Artificial Sequence: Synthetic Primer
  source 1..38: mol_type = other DNA
agagcctcgagattatccagaattttgaaaaagtattg

SEQ ID NO: 143 - DNA, 30 bp, synthetic construct
  misc_feature 1..30: note = Description of Artificial Sequence: Synthetic Primer
  source 1..30: mol_type = other DNA
atcgatcatatgcatgacgttaaaaatcac

SEQ ID NO: 144 - DNA, 38 bp, synthetic construct; identical to SEQ ID NO: 142
  misc_feature 1..38: note = Description of Artificial Sequence: Synthetic Primer
  source 1..38: mol_type = other DNA

SEQ ID NO: 145 - DNA, 42 bp, synthetic construct
  misc_feature 1..42: note = Description of Artificial Sequence: Synthetic Primer
  source 1..42: mol_type = other DNA
atcgacatatggaaaaaacgctggccctgattaaaccggatg

SEQ ID NO: 146 - DNA, 43 bp, synthetic construct
  misc_feature 1..43: note = Description of Artificial Sequence: Synthetic Primer
  source 1..43: mol_type = other DNA
actgcctcgaggaaaaacagttccatttcacgagctgccgatg

SEQ ID NO: 147 - DNA, 42 bp, synthetic construct; identical to SEQ ID NO: 145
  misc_feature 1..42: note = Description of Artificial Sequence: Synthetic Primer
  source 1..42: mol_type = other DNA

SEQ ID NO: 148 - DNA, 38 bp, synthetic construct; identical to SEQ ID NO: 142
  misc_feature 1..38: note = Description of Artificial Sequence: Synthetic Primer
  source 1..38: mol_type = other DNA

SEQ ID NO: 149 - DNA, 42 bp, synthetic construct; identical to SEQ ID NO: 145
  misc_feature 1..42: note = Description of Artificial Sequence: Synthetic Primer
  source 1..42: mol_type = other DNA

SEQ ID NO: 150 - DNA, 38 bp, synthetic construct
  misc_feature 1..38: note = Description of Artificial Sequence: Synthetic Primer
  source 1..38: mol_type = other DNA
agagcaccggtattatccagaattttgaaaaagtattg

SEQ ID NO: 151 - DNA, 35 bp, synthetic construct
  misc_feature 1..35: note = Description of Artificial Sequence: Synthetic Primer
  source 1..35: mol_type = other DNA
atcgacatatgacgcaaaatctgggctcggaaatg

SEQ ID NO: 152 - DNA, 34 bp, synthetic construct
  misc_feature 1..34: note = Description of Artificial Sequence: Synthetic Primer
  source 1..34: mol_type = other DNA
actgcctcgagtgccggacccagaccacccgtgc

SEQ ID NO: 153 - DNA, 35 bp, synthetic construct; identical to SEQ ID NO: 151
  misc_feature 1..35: note = Description of Artificial Sequence: Synthetic Primer
  source 1..35: mol_type = other DNA

SEQ ID NO: 154 - DNA, 35 bp, synthetic construct
  misc_feature 1..35: note = Description of Artificial Sequence: Synthetic Primer
  source 1..35: mol_type = other DNA
actgcaccggttgccggacccagaccacccgtgcg

SEQ ID NO: 155 - protein, 41 aa, synthetic construct
  REGION 1..41: note = Description of Artificial Sequence: Synthetic polypeptide
  REGION 1..41: note = PSMGFR N-10 peptide
QFNQYKTEAASRYNLTISDVSVSDVPFPFSAQSGAHHHHHH

SEQ ID NO: 156 - protein, 376 aa, Homo sapiens; identical to SEQ ID NO: 82
  REGION 1..376: note = A domain of NME7

SEQ ID NO: 157 - DNA, 35 bp, synthetic construct; identical to SEQ ID NO: 151
  misc_feature 1..35: note = Description of Artificial Sequence: Synthetic Primer
  source 1..35: mol_type = other DNA

SEQ ID NO: 158 - DNA, 34 bp, synthetic construct; identical to SEQ ID NO: 152
  misc_feature 1..34: note = Description of Artificial Sequence: Synthetic Primer
  source 1..34: mol_type = other DNA

SEQ ID NO: 159 - protein, 152 aa, Homo sapiens
MANCERTFIAIKPDGVQRGLVGEIIKRFEQKGFRLVGLKFMQASEDLLKEHYVDLKDRPFFAGLVKYMHSGPVVAMVWEGLNVVKTGRVMLGETNPADSKPGTIRGDFCIQVGRNIIHGSDSVESAEKEIGLWFHPEELVDYTSCAQNWIYE

SEQ ID NO: 160 - protein, 141 aa, Halomonas sp.
MATERTLSIIKPDAVAKNVIGEIESRFEKAGLKIVAAKMLQLSQEQAEGFYAEHKERPFFGDLVGFMTSGPVVVQVLEGENAIAANRDLMGATNPKEAEAGTIRADYAQSIDANAVHGSDSPESAAREIAYFFEESEICSR

SEQ ID NO: 161 - protein, 131 aa, Homo sapiens
EKTLALIKPDAISKAGEIIEIINKAGFTITKLKMMMLSRKEALDFHVDHQSRPFFNELIQFITTGPIIAMEILRDDAICEWKRLLGPANSGVARTDASESIRALFGTDGIRNAAHGPDSFASAAREMELFF

SEQ ID NO: 162 - protein, 130 aa, Halomonas sp.
ERTLSIIKPDAVAKNVIGEIESRFEKAGLKIVAAKMLQLSQEQAEGFYAEHKERPFFGDLVGFMTSGPVVVQVLEGENAIAANRDLMGATNPKEAEAGTIRADYAQSIDANAVHGSDSPESAAREIAYFF

SEQ ID NO: 163 - protein, 132 aa, Homo sapiens
TCCIVKPHAVSEGILGKILMAIRDAGFEISAMQMFNMDRVNVEEFYEVYKGVVTEYHDMVTEMYSGPCVAMEIQQNNATKTFREFCGPADPEIARHLRPGTLRAIFGKTKIQNAVHCTDLPEDGLLEVQYFF

SEQ ID NO: 164 - protein, 128 aa, Halomonas sp.
TLSIIKPDAVAKNVIGEIESRFEKAGLKIVAAKMLQLSQEQAEGFYAEHKERPFFGDLVGFMTSGPVVVQVLEGENAIAANRDLMGATNPKEAEAGTIRADYAQSIDANAVHGSDSPESAAREIAYFF

SEQ ID NO: 165 - protein, 139 aa, Homo sapiens
NCTCCIVKPHAVSEGLLGKILMAIRDAGFEISAMQMFNMDRVNVEEFYEVYKGVVTEYHDMVTEMYSGPCVAMEIQQNNATKTFREFCGPADPEIARHLRPGTLRAIFGKTKIQNAVHCTDLPEDGLLEVQYFFKILDN

SEQ ID NO: 166 - protein, 12 aa, synthetic construct
  REGION 1..12: note = Description of Artificial Sequence: Synthetic peptide
GSSSGSSSGSSS